(19) United States
(12) Patent Application Publication
(10) Pub. No.: US 2026/0006984 A1
(43) Pub. Date: Jan. 1, 2026

(54) Organic Compound, Film, Electronic Device, and Light-Emitting Device

(71) Applicant: Semiconductor Energy Laboratory Co., Ltd., Kanagawa-ken (JP)

(72) Inventors: Takao TOSU, Atsugi (JP); Tomohiro KUBOTA, Atsugi (JP); Takeyoshi WATABE, Atsugi, (JP); Nobuharu OHSAWA, Zama (JP); Satoshi SEO, Sagamihara (JP)

(73) Assignee: Semiconductor Energy Laboratory Co., Ltd., Kanagawa-ken (JP)

(21) Appl. No.: 19/248,079

(22) Filed: Jun. 24, 2025

(30) Foreign Application Priority Data
Jun. 28, 2024 (JP) 2024-105618

(51) Int. Cl. H10K 50/858; C07C 211/54; H10K 50/15; H10K 85/30; H10K 85/60

(52) U.S. Cl. CPC H10K 50/858; C07C 211/54; H10K 50/156; H10K 85/342; H10K 85/346; H10K 85/626; H10K 85/633; H10K 85/636; H10K 85/654; H10K 85/656; H10K 85/6572; H10K 85/6574

(57) ABSTRACT

A carrier-transport material or a hole-transport material that enables formation of a film having a low refractive index and a large GSP_slope is provided. An organic compound represented by General Formula (G1) is provided. In the organic compound represented by General Formula (G1), Ar¹ represents a phenyl group or a biphenyl group having at least one alkyl group having 1 to 6 carbon atoms. Furthermore, R¹ represents an alkyl group having 3 to 7 carbon atoms, and n represents 2 or 3. R² and R³ each independently represent any one of an alkyl group having 1 to 6 carbon atoms, an unsubstituted phenyl group, and a phenyl group having an alkyl group having 1 to 6 carbon atoms, and R² and R³ may be bonded to each other to form a ring. R⁴ represents hydrogen or an alkyl group having 1 to 6 carbon atoms.

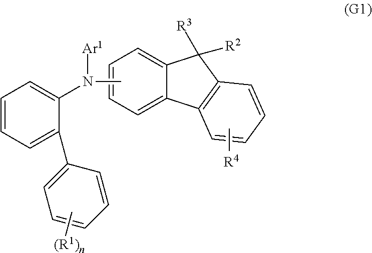

### BACKGROUND OF THE INVENTION

### 1. Field of the Invention

One embodiment of the present invention relates to an organic compound, an organic electronic device, a light-emitting device, an organic EL device, and an electronic device.

Note that one embodiment of the present invention is not limited to the above technical field. Examples of the technical field of one embodiment of the present invention include a compound, a light-emitting device, an organic EL device, a semiconductor device, a display device, a light-emitting apparatus, a power storage device, a memory device, an electronic device, a lighting device, an input device (e.g., a touch sensor), an input/output device (e.g., a touch panel), a method for driving any of them, and a method for manufacturing any of them.

### 2. Description of the Related Art

Recently, display devices have been expected to be applied to a variety of uses. Usage examples of large-sized display devices include a television device for home use (also referred to as TV or television receiver), digital signage, and a public information display (PID). In addition, a smartphone and a tablet terminal each including a touch panel, for example, are being developed as portable information terminals.

An increase in the resolution of display devices is also required. For example, devices for virtual reality (VR), augmented reality (AR), substitutional reality (SR), or mixed reality (MR) are given as devices requiring high-resolution display devices and have been actively developed.

Light-emitting apparatuses including light-emitting devices (also referred to as light-emitting elements) using organic compounds have been developed as display devices. Light-emitting devices utilizing electroluminescence (hereinafter referred to as EL; such devices are also referred to as organic EL devices or light-emitting devices) have features such as ease of reduction in thickness and weight, high-speed response to input signals, and driving with a constant DC voltage power source, and have been used in display devices.

Displays or lighting devices including light-emitting devices are suitable for a variety of electronic devices, and research and development of materials and devices have progressed to obtain light-emitting devices with more favorable characteristics (see Patent Document 1, for example).

### REFERENCE

[Patent Document 1] PCT International Publication No. 2023/072977
[Patent Document 2] Patent Application Publication No. 2021-012526
[Non-Patent Document 1]Y. Noguchi et al., “Spontaneous Orientation Polarization of Polar Molecules and Interface Properties of Organic Electronic Devices”, Journal of the Vacuum Society of Japan, 2015, Vol. 58, No. 3.

### SUMMARY OF THE INVENTION

An object of one embodiment of the present invention is to provide a novel organic compound. Another object of one embodiment of the present invention is to provide a novel carrier-transport material. Another object of one embodiment of the present invention is to provide a novel hole-transport material. Another object of one embodiment of the present invention is to provide a carrier-transport material or a hole-transport material capable of forming a film with a low refractive index. Another object of one embodiment of the present invention is to provide an organic compound capable of forming a film whose slope of a giant surface potential (GSP) (hereinafter referred to as a GSP_slope) is large. Another object of one embodiment of the present invention is to provide a carrier-transport material or a hole-transport material capable of forming a film with a large GSP_slope. Another object of one embodiment of the present invention is to provide a carrier-transport material or a hole-transport material capable of forming a film with a low refractive index and a large GSP_slope.

Another object of one embodiment of the present invention is to provide a light-emitting device having high emission efficiency. An object of another embodiment of the present invention is to provide a light-emitting device having a low driving voltage. Another object of one embodiment of the present invention is to provide a light-emitting device, a light-emitting apparatus, an electronic device, and a display device each having low power consumption.

Note that the description of these objects does not preclude the existence of other objects. One embodiment of the present invention does not necessarily achieve all of these objects. Other objects can be derived from the description of the specification, the drawings, and the claims.

One embodiment of the present invention is an organic compound represented by General Formula (G1) below.

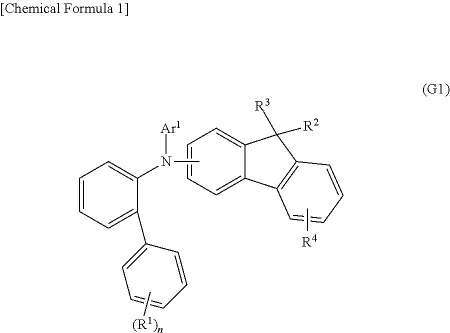

In the organic compound represented by General Formula (G1) above, Ar¹ represents a phenyl group or a biphenyl group having at least one alkyl group having 1 to 6 carbon atoms; when the phenyl group or the biphenyl group has two or more alkyl groups, the two or more alkyl groups may be the same or different from each other. Furthermore, R¹ represents an alkyl group having 3 to 7 carbon atoms, and n represents 2 or 3. A plurality of R¹s may be the same or different from each other. R² and R³ each independently represent any one of an alkyl group having 1 to 6 carbon atoms, an unsubstituted phenyl group, and a phenyl group having one or more alkyl groups each having 1 to 6 carbon atoms, and R² and R³ may be bonded to each other to form a ring. R⁴ represents hydrogen or an alkyl group having 1 to 6 carbon atoms.

Another embodiment of the present invention is an organic compound represented by General Formula (G2).

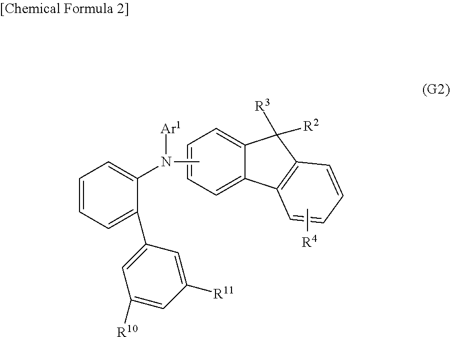

In the organic compound represented by General Formula (G2) above, Ar¹ represents a phenyl group or a biphenyl group having at least one alkyl group having 1 to 6 carbon atoms; when the phenyl group or the biphenyl group has two or more alkyl groups, the two or more alkyl groups may be the same or different from each other. In addition, R¹⁰ and R¹¹ each independently represent an alkyl group having 3 to 7 carbon atoms. R² and R³ each independently represent any one of an alkyl group having 1 to 6 carbon atoms, an unsubstituted phenyl group, and a phenyl group having one or more alkyl groups each having 1 to 6 carbon atoms, and R² and R³ may be bonded to each other to form a ring. R⁴ represents hydrogen or an alkyl group having 1 to 6 carbon atoms.

Another embodiment of the present invention is an organic compound represented by General Formula (G3) below.

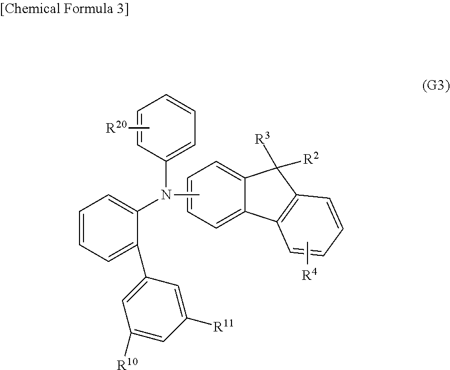

In the organic compound represented by General Formula (G3) above, R¹⁰ and R¹¹ each independently represent an alkyl group having 3 to 7 carbon atoms, and R²⁰ represents an alkyl group having 1 to 6 carbon atoms or a phenyl group having an alkyl group having 1 to 6 carbon atoms. R² and R³ each independently represent any one of an alkyl group having 1 to 6 carbon atoms, an unsubstituted phenyl group, and a phenyl group having one or more alkyl groups each having 1 to 6 carbon atoms, and R² and R³ may be bonded to each other to form a ring. R⁴ represents hydrogen or an alkyl group having 1 to 6 carbon atoms.

Another embodiment of the present invention is an organic compound represented by General Formula (G4).

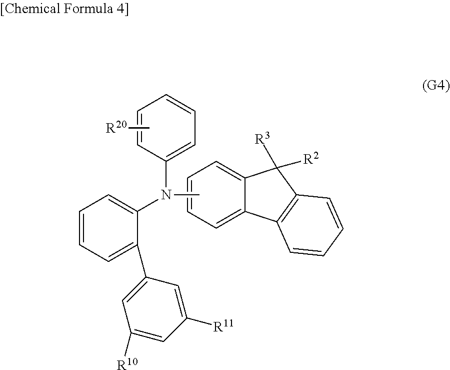

In the organic compound represented by General Formula (G4) above, R¹⁰ and R¹¹ each independently represent an alkyl group having 3 to 7 carbon atoms, and R²⁰ represents an alkyl group having 1 to 6 carbon atoms or a phenyl group having an alkyl group having 1 to 6 carbon atoms. R² and R³ each independently represent any one of an alkyl group having 1 to 6 carbon atoms, an unsubstituted phenyl group, and a phenyl group having one or more alkyl groups each having 1 to 6 carbon atoms, and R² and R³ may be bonded to each other to form a ring.

Another embodiment of the present invention is a film that contains any of the above-described organic compounds.

Another embodiment of the present invention is any of the above-described organic compounds capable of forming a film with a GSP_slope greater than or equal to 30 mV/nm.

Another embodiment of the present invention is the organic compound with any of the above structures, in which the film is evaporated at a rate greater than or equal to 3 nm/min and less than or equal to 600 nm/min.

Another embodiment of the present invention is the organic compound with any of the above structures, in which the thickness of the film is greater than or equal to 3 nm and less than or equal to 500 nm.

Another embodiment of the present invention is any of the above organic compounds with the above structure, in which the thickness of the film is greater than or equal to 50 nm and less than or equal to 300 nm.

Another embodiment of the present invention is the organic compound with any of the above structure, in which the film is evaporated at a rate greater than or equal to 3 nm/min and less than or equal to 600 nm/min and the thickness of the film is greater than or equal to 3 nm and less than or equal to 500 nm.

Another embodiment of the present invention is any of the above organic compounds a film of which is evaporated at a rate greater than or equal to 3 nm/min and less than or equal to 600 nm/min. The film has a thickness greater than or equal to 3 nm and less than or equal to 500 nm. A GSP_slope of the film is greater than or equal to 30 mV/nm.

Another embodiment of the present invention is any of the above organic compounds capable of forming a film whose ordinary refractive index at a wavelength of 450 nm to 460 nm is lower than or equal to 1.75.

Another embodiment of the present invention is any of the organic compounds capable of forming a film whose ordinary refractive index at a wavelength of 510 nm to 545 nm is lower than or equal to 1.70.

Another embodiment of the present invention is an electronic device including any of the above films.

Another embodiment of the present invention is a light-emitting device including a first electrode, a second electrode, and an organic compound layer. The organic compound layer is between the first electrode and the second electrode, and the organic compound layer contains any of the above organic compounds.

Another embodiment of the present invention is a light-emitting device including a first electrode, a second electrode, and an organic compound layer; the organic compound layer is positioned between the first electrode and the second electrode; the organic compound layer includes a light-emitting layer and a hole-transport layer; and the hole-transport layer contains any of the above organic compounds.

Another embodiment of the present invention is the light-emitting device having the above structure, in which the first electrode is formed over a substrate. The hole-transport layer includes a first hole-transport layer and a second hole-transport layer. The first hole-transport layer is between the second hole-transport layer and the first electrode. The second hole-transport layer contains any of the above organic compounds.

Another embodiment of the present invention is a light-emitting device including a first electrode, a second electrode, and an organic compound layer. The organic compound layer is positioned between the first electrode and the second electrode. The organic compound layer includes a light-emitting layer and a hole-transport layer. The hole-transport layer is positioned between the light-emitting layer and the first electrode. The hole-transport layer contains any of the above organic compounds.

Another embodiment of the present invention is the light-emitting device having the above structure. The hole-transport layer includes a first hole-transport layer and a second hole-transport layer. The second hole-transport layer is positioned between the first hole-transport layer and the light-emitting layer. The second hole-transport layer contains any of the above organic compounds.

Another embodiment of the present invention is the light emitting device having the above structure, in which the second hole-transport layer and the light-emitting layer are in contact with each other.

Another embodiment of the present invention is the light-emitting device having the above structure, in which the GSP_slope of the second hole-transport layer is greater than the GSP_slope of the first hole-transport layer.

Another embodiment of the present invention is an organic electronic device including any of the organic compounds described above.

Another embodiment of the present invention is a light-emitting device including any of the organic compounds described above.

Another embodiment of the present invention is a light-receiving device including any of the organic compounds described above.

Another embodiment of the present invention is an organic electronic device using any of the organic compounds described above for a cap layer.

Another embodiment of the present invention is an electronic device including the above organic electronic device.

According to one embodiment of the present invention, a novel organic compound can be provided. According to one embodiment of the present invention, a novel carrier-transport material can be provided. According to one embodiment of the present invention, a novel hole-transport material can be provided. According to one embodiment of the present invention, a carrier-transport material or a hole-transport material capable of forming a film with a low refractive index can be provided. According to one embodiment of the present invention, an organic compound capable of forming a film with a large GSP_slope can be provided. According to one embodiment of the present invention, a carrier-transport material or a hole-transport material capable of forming a film with a large GSP_slope can be provided. According to another embodiment of the present invention, a carrier-transport material or a hole-transport material capable of forming a film with a low refractive index and a large GSP_slope can be provided.

According to another embodiment of the present invention, a light-emitting device having high emission efficiency can be provided. According to another embodiment of the present invention, a light-emitting device having a low driving voltage can be provided. One embodiment of the present invention can provide a light-emitting device, a light-emitting apparatus, an electronic device, and a display device each having low power consumption.

One embodiment of the present invention can provide a novel light-emitting device, a novel display device, a novel display module, and a novel electronic device.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

FIGS. 1A to 1C illustrate light-emitting devices.

FIGS. 2A and 2B are a top view and a cross-sectional view of a light-emitting apparatus.

FIGS. 3A and 3B are perspective views illustrating a structure example of a display module.

FIGS. 4A and 4B are cross-sectional views illustrating structure examples of a display device.

FIG. 5 is a perspective view illustrating a structure example of a display device.

FIG. 6 is a cross-sectional view illustrating a structure example of a display device.

FIG. 7 is a cross-sectional view illustrating a structure example of a display device.

FIG. 8 is a cross-sectional view illustrating a structure example of a display device.

FIGS. 9A to 9D illustrate examples of electronic devices.

FIGS. 10A to 10F illustrate examples of electronic devices.

FIGS. 11A to 11G illustrate examples of electronic devices.

FIG. 12 shows capacity-voltage characteristics of a measurement device 1.

FIGS. 13A and 13B show a ¹H-NMR chart of mmtBuoBichPAF.

FIGS. 14A and 14B each show an absorption spectrum and a PL spectrum of mmtBuoBichPAF.

FIGS. 15A and 15B show a ¹H-NMR chart of dmmtBuopBBAF.

FIGS. 16A and 16B each show an absorption spectrum and a PL spectrum of dmmtBuopBBAF.

FIG. 17 shows luminance-current density characteristics of a light-emitting device 1 and a comparative light-emitting device 1.

FIG. 18 shows current efficiency-luminance characteristics of the light-emitting device 1 and the comparative light-emitting device 1.

FIG. 19 shows luminance-voltage characteristics of the light-emitting device 1 and the comparative light-emitting device 1.

FIG. 20 shows current density-voltage characteristics of the light-emitting device 1 and the comparative light-emitting device 1.

FIG. 21 shows external quantum efficiency-luminance characteristics of the light-emitting device 1 and the comparative light-emitting device 1.

FIG. 22 shows electroluminescence spectra of the light-emitting device 1 and the comparative light-emitting device 1.

FIG. 23 shows luminance-current density characteristics of a light-emitting device 2.

FIG. 24 shows current efficiency-luminance characteristics of the light-emitting device 2.

FIG. 25 shows luminance-voltage characteristics of the light-emitting device 2.

FIG. 26 shows current density-voltage characteristics of the light-emitting device 2.

FIG. 27 shows external quantum efficiency-luminance characteristics of the light-emitting device 2.

FIG. 28 shows an electroluminescence spectrum of the light-emitting device 2.

FIG. 29 shows luminance-current density characteristics of a light-emitting device 3 and comparative light-emitting devices 3-1 to 3-3.

FIG. 30 shows current efficiency-luminance characteristics of the light-emitting device 3 and the comparative light-emitting devices 3-1 to 3-3.

FIG. 31 shows luminance-voltage characteristics of the light-emitting device 3 and the comparative light-emitting devices 3-1 to 3-3.

FIG. 32 shows current density-voltage characteristics of the light-emitting device 3 and the comparative light-emitting devices 3-1 to 3-3.

FIG. 33 shows external quantum efficiency-luminance characteristics of the light-emitting device 3 and the comparative light-emitting devices 3-1 to 3-3.

FIG. 34 shows power efficiency-luminance characteristics of the light-emitting device 3 and the comparative light-emitting devices 3-1 to 3-3.

FIG. 35 shows electroluminescence spectra of the light-emitting device 3 and the comparative light-emitting devices 3-1 to 3-3.

FIG. 36 shows luminance-current density characteristics of a light-emitting device 4 and comparative light-emitting devices 4-1 to 4-3.

FIG. 37 shows current efficiency-luminance characteristics of the light-emitting device 4 and the comparative light-emitting devices 4-1 to 4-3.

FIG. 38 shows luminance-voltage characteristics of the light-emitting device 4 and the comparative light-emitting devices 4-1 to 4-3.

FIG. 39 shows current density-voltage characteristics of the light-emitting device 4 and the comparative light-emitting devices 4-1 to 4-3.

FIG. 40 shows external quantum efficiency-luminance characteristics of the light-emitting device 4 and the comparative light-emitting devices 4-1 to 4-3.

FIG. 41 shows power efficiency-luminance characteristics of the light-emitting device 4 and the comparative light-emitting devices 4-1 to 4-3.

FIG. 42 shows electroluminescence spectra of the light-emitting device 4 and the comparative light-emitting devices 4-1 to 4-3.

FIG. 43 shows current density-voltage characteristics of the measurement device 1.

FIG. 44 shows measurement results of refractive indices of mmtBuoBichPAF.

FIG. 45 shows measurement results of refractive indices of PCBBiF.

FIG. 46 shows measurement results of refractive indices of dmmtBuopBBAF.

FIG. 47 shows measurement results of refractive indices of mmtBuBichPAF.

FIG. 48 shows measurement results of refractive indices of oFBiSF(2).

FIG. 49 shows measurement results of refractive indices of mmtBuBioFBi.

### DETAILED DESCRIPTION OF THE INVENTION

Embodiments will be described in detail with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

Ordinal numbers such as “first” and “second” in this specification and the like are used in order to avoid confusion among components and do not denote the order such as the order of steps or the stacking order. A term without an ordinal number in this specification and the like might be provided with an ordinal number in a claim in order to avoid confusion among components. A term with an ordinal number in this specification and the like might be provided with a different ordinal number in a claim. A term with an ordinal number in this specification and the like might not be provided with an ordinal number in a claim.

Note that in this specification and the like, a photoluminescence (PL) spectrum refers to a spectrum obtained by measuring the wavelength of light emission while an excitation wavelength of excitation light is fixed in a fluorometry. Such a spectrum is also referred to as an emission spectrum in some cases. Note that an emission spectrum may include a fluorescent component and a phosphorescent component. In this specification and the like, an emission spectrum including a fluorescent component is particularly referred to as a fluorescent spectrum, and an emission spectrum including a phosphorescent component is particularly referred to as a phosphorescent spectrum in some cases.

### Embodiment 1

A light-emitting device including an organic thin-film (also referred to as a light-emitting device in this specification) is a kind of semiconductor element including an organic thin film (organic semiconductor element). Typical examples of the organic semiconductor element include a photodiode and an organic TFT. The light-emitting device including an organic thin-film has a structure where organic thin film layers which are functionally separated (also referred to as functional layers), such as a carrier-injection layer, a carrier-transport layer, and a light-emitting layer, are stacked. With the progress of research on the light-emitting device including an organic thin-film, the devices have higher performance owing to improvements in functions of functional layers typified by carrier-transport properties and emission quantum yields, and other characteristics.

An example of other characteristics is a refractive index of the organic thin film. When a carrier-transport layer, a carrier-injection layer, or the like is formed using a material capable of forming a film with a low refractive index, light extraction efficiency can be improved and a light-emitting device with high emission efficiency can be obtained.

Examples of the other characteristics include a giant surface potential (GSP) and a slope thereof (GSP_slope). GSP is a phenomenon due to spontaneous orientation polarization (SOP) caused by deviation of permanent dipole moment orientation of a film to the thickness direction. When GSP changes in proportion to the thickness of a film whose surface potential and thickness are represented by V (mV) and d (nm), respectively, a parameter represented by V/d is GSP_slope (mV/nm).

When functional layers each having an appropriate GSP_slope are stacked in a light-emitting device having a stacked-layer structure, the light-emitting device with low driving voltage can be obtained. Specifically, a light-emitting device with low driving voltage can be obtained when a value obtained by subtracting the GSP_slope of an organic thin film on the side of an electrode formed over the substrate from the GSP_slope of an organic thin film on the side of a counter electrode is large in a hole-transport region, and when a value obtained by subtracting the GSP_slope of the organic thin film on the side of the counter electrode from the GSP_slope of the organic thin film on the side of the electrode formed over the substrate is large in an electron-transport region. Alternatively, a light-emitting device with low driving voltage can be obtained when films are stacked in ascending order of GSP_slope from the side of the electrode formed over the substrate toward the counter electrode side in the hole-transport region, and when films are stacked in descending order of GSP_slope from the side of the electrode formed over the substrate toward the counter electrode side in the electron-transport region. That is, what is called an ordered stacked light-emitting device in which the electrode formed over the substrate functions as an anode can have low driving voltage when a value obtained by subtracting the GSP_slope of the organic thin film on the anode side from the GSP_slope of the organic thin film on the cathode side is large in the hole-transport region, and when a value obtained by subtracting the GSP_slope on the cathode side from the GSP_slope on the anode side is large in the electron-transport region. Alternatively, a light-emitting device with low driving voltage can be obtained when films are stacked in ascending order of GSP_slope from the anode side toward the cathode side in the hole-transport region, and when films are stacked in descending order of GSP_slope from the anode side toward the cathode side in the electron-transport region. What is called an inversed stacked light-emitting device in which an electrode formed over the substrate functions as the cathode can have low driving voltage when the value obtained by subtracting the GSP_slope of the organic thin film on the cathode side from the GSP_slope of the organic thin film on the anode side is large in the hole-transport region, and when the value obtained by subtracting the GSP_slope on the anode side from the GSP_slope on the cathode side is large in the electron-transport region. Alternatively, a light-emitting device with low driving voltage can be obtained when films are stacked in ascending order of GSP_slope from the cathode side toward the anode side in the hole-transport region, and when films are stacked in descending order of GSP_slope from the cathode side toward the anode side in the electron-transport region.

That is, when the carrier-transport layer and/or the carrier-injection layer having a low refractive index and an appropriate GSP_slope are/is provided, an extremely favorable light-emitting device with high emission efficiency and low driving voltage can be achieved.

However, in consideration of stacking films with different GSP_slopes, the number of organic compounds each capable of forming a film with a GSP_slope greatly different from that of films of conventionally used organic compounds is not so large partly because a GSP_slope is derived from the molecular structure of an organic compound.

Since a low refractive index and a high carrier-transport property are hardly compatible with each other, it is difficult to find an organic compound capable of forming a film having all of the following three properties: a low refractive index, a high carrier-transport property, and a large (or small) GSP_slope. Furthermore, the organic compound needs to have a highest occupied molecular orbital (HOMO) level or a lowest unoccupied molecular orbital (LUMO) level suitable for a carrier-transport layer or a carrier-injection layer.

Here, the present inventors have found that a film formed of an arylamine compound including an orthobiphenyl group having a plurality of alkyl groups has a low refractive index, a large GSP_slope (specifically, greater than or equal to 30 mV/nm), and a high hole-transport property, and the organic compound has an appropriate HOMO level.

That is, one embodiment of the present invention is an organic compound represented by General Formula (G1) below.

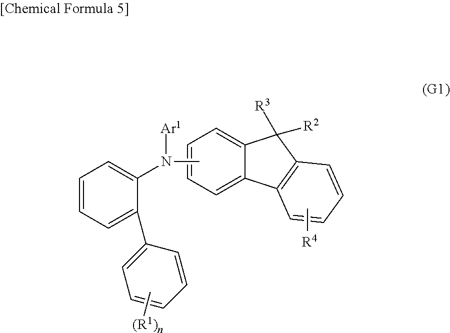

In the organic compound represented by General Formula (G1) above, Ar¹ represents a phenyl group or a biphenyl group having at least one alkyl group having 1 to 6 carbon atoms; when the phenyl group or the biphenyl group has two or more alkyl groups, the two or more alkyl groups may be the same or different from each other.

Examples of the alkyl group having 1 to 6 carbon atoms which is substituted for the phenyl group or the biphenyl group in Ar¹ include a methyl group, an ethyl group, a propyl group, an isopropyl group, an n-butyl group, a sec-butyl group, an isobutyl group, a tert-butyl group, an n-pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, a hexyl group, an isohexyl group, a cyclopropyl group, a cyclobutyl group, a cyclopentyl group, and a cyclohexyl group. Note that a tert-butyl group or a cyclohexyl group is particularly preferable to lower the refractive index. The alkyl group having 1 to 6 carbon atoms which is substituted for the phenyl group or the biphenyl group in Ar¹ includes a branched alkyl group or a cycloalkyl group.

It is preferable that the number of alkyl groups included in Ar¹ be one or two, in which case the hole-transport property is less likely to be inhibited. An alkyl group is necessary to reduce the refractive index of an organic compound; the larger the number of alkyl groups is, the lower the refractive index is and the higher the emission efficiency of the light-emitting device is. Meanwhile, it is known that the alkyl group tends to reduce the hole-transport property. Thus, the number of substituents to be introduced is preferably the number that does not degrade both properties. In the case where Ar¹ includes two alkyl groups, the two alkyl groups are preferably at the 3-position and 5-position of a phenyl group or a phenyl group at the end of a biphenyl group because of the easy availability of a material for synthesis.

Note that as Ar¹, a 4-cyclohexylphenyl group, a 3′,5′-ditertiarybutylbiphenyl group, a 3′,5′-dicyclohexylbiphenyl group, or the like is particularly preferable.

Furthermore, R¹ represents an alkyl group having 3 to 7 carbon atoms, and n represents 2 or 3. A plurality of R¹s may be the same or different from each other. Examples of the alkyl group having 3 to 7 carbon atoms include an isopropyl group, a sec-butyl group, an isobutyl group, a tert-butyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, an isohexyl group, a 3-methylpentyl group, a 2-methylpentyl group, a 2-ethylbutyl group, a 1,2-dimethylbutyl group, a 2,3-dimethylbutyl group, a 2-ethylhexyl group, a 1-ethylpropyl group, a cyclopropyl group, a cyclobutyl group, a cyclopentyl group, a cyclohexyl group, and a norbornyl group. Note that a tert-butyl group or a cyclohexyl group is particularly preferable because the refractive index can be reduced. In order to ensure a hole-transport property, n is preferably 2. Note that when n is 2, the two alkyl groups are preferably at the 3-position and 5-position of a phenyl group in terms of the high availability of a material for synthesis. The alkyl group having 3 to 7 carbon atoms in R¹ includes a branched alkyl group or a cycloalkyl group.

R² and R³ each independently represent any one of an alkyl group having 1 to 6 carbon atoms, an unsubstituted phenyl group, and a phenyl group having one or more alkyl groups each having 1 to 6 carbon atoms. Note that the alkyl group having 1 to 6 carbon atoms can be the same group as the alkyl group having 1 to 6 carbon atoms in Ar¹. Furthermore, R² and R³ may be bonded to each other to form a ring; in the case where R² and R³ are each a phenyl group and R² and R³ are bonded to each other, for example, the organic compound represented by General Formula (G1) above may be an organic compound including a spirofluorenyl group.

R⁴ represents hydrogen or an alkyl group having 1 to 6 carbon atoms. The alkyl group having 1 to 6 carbon atoms can be the same group as the alkyl group having 1 to 6 carbon atoms in Ar¹. R⁴ is preferably hydrogen to improve the hole-transport property. In terms of a reduction in refractive index, R⁴ is preferably an alkyl group having 1 to 6 carbon atoms.

Note that in the organic compound represented by General Formula (G1) above, the number of alkyl groups bonded to the phenyl group at the end of the o-biphenyl group is preferably 2 in order to increase a GSP_slope value. The two alkyl groups are preferably at the 3-position and 5-position of the phenyl group at the end of the o-biphenyl group, in which case the alkyl groups are aligned in the direction perpendicular to a molecular plane composed of Ar¹ and fluorenylamine.

That is, the organic compound represented by General Formula (G1) is preferably an organic compound represented by General Formula (G2) below or an organic compound represented by General Formula (G3) below.

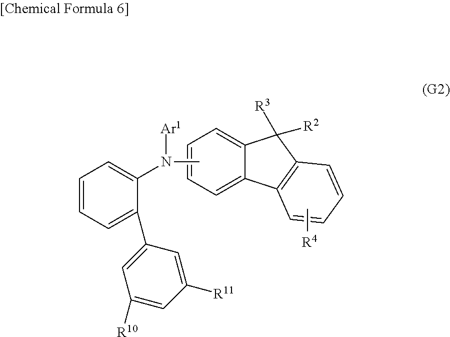

In the organic compound represented by General Formula (G2) above, Ar¹ represents a phenyl group or a biphenyl group having at least one alkyl group having 1 to 6 carbon atoms; when the phenyl group or the biphenyl group has two or more alkyl groups, the two or more alkyl groups may be the same or different from each other. For the description of Ar¹ in General Formula (G2), the description of Ar¹ in General Formula (G1) can be referred to.

In addition, R¹⁰ and R¹¹ each independently represent an alkyl group having 3 to 7 carbon atoms. For the description of the alkyl group having 3 to 7 carbon atoms in each of R¹⁰ and R¹¹, the description of the alkyl group having 3 to 7 carbon atoms in R¹ in General Formula (G1) can be referred to. Note that R¹⁰ and R¹¹ are preferably tert-butyl groups, in which case the phenyl group having a di-tert-butyl group is aligned in the direction perpendicular to the molecular plane composed of Ar¹ and fluorenylamine. In addition, the arrangement of the phenyl group having the di-tert-butyl group is less likely to change because the tert-butyl group is bulky, which is preferable.

R² and R³ each independently represent any one of an alkyl group having 1 to 6 carbon atoms, an unsubstituted phenyl group, and a phenyl group having an alkyl group having 1 to 6 carbon atoms, and R² and R³ may be bonded to each other to form a ring. For the description of R² and R³ in General Formula (G2), the description of R² and R³ in General Formula (G1) can be referred to.

R⁴ represents hydrogen or an alkyl group having 1 to 6 carbon atoms. For the description of R⁴ in General Formula (G2), the description of R⁴ in General Formula (G1) can be referred to.

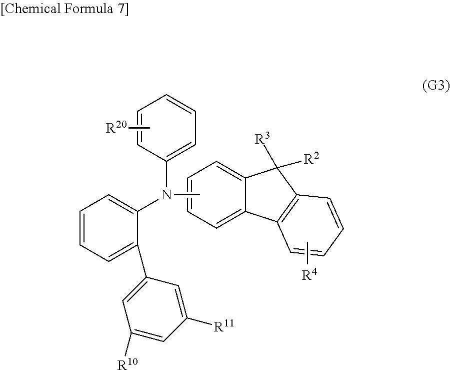

In the organic compound represented by General Formula (G3) above, R¹⁰ and R¹¹ each independently represent an alkyl group having 3 to 7 carbon atoms. For the description of R¹⁰ and R¹¹ in General Formula (G3), the description of R¹⁰ and R¹¹ in General Formula (G2) can be referred to.

Furthermore, R²⁰ represents an alkyl group having 1 to 6 carbon atoms or a phenyl group having an alkyl group having 1 to 6 carbon atoms. Note that the alkyl group having 1 to 6 carbon atoms can be the same group as the alkyl group having 1 to 6 carbon atoms in Ar¹. R²¹ is preferably a cyclohexyl group, a tert-butyl group, or a phenyl group having a cyclohexyl group or a tert-butyl group because the refractive index can be reduced.

R² and R³ each independently represent any one of an alkyl group having 1 to 6 carbon atoms, an unsubstituted phenyl group, and a phenyl group having an alkyl group having 1 to 6 carbon atoms, and R² and R³ may be bonded to each other to form a ring. For the description of R² and R³ in General Formula (G3), the description of R² and R³ in General Formula (G1) can be referred to.

R⁴ represents hydrogen or an alkyl group having 1 to 6 carbon atoms. For the description of R⁴ in General Formula (G3), the description of R⁴ in General Formula (G1) can be referred to.

In the organic compound represented by General Formula (G3) above, R⁴ is preferably hydrogen, in which case synthesis is facilitated. That is, the organic compound represented by General Formula (G3) above is preferably an organic compound represented by General Formula (G4) below.

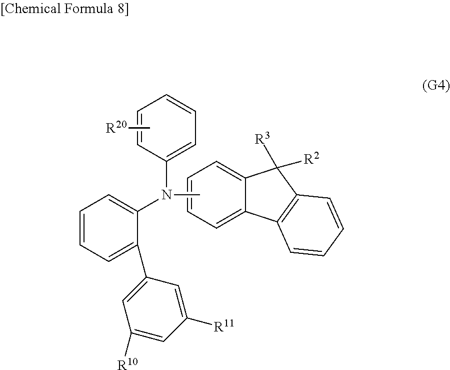

In the organic compound represented by General Formula (G4) above, R¹⁰ and R¹¹ each independently represent an alkyl group having 3 to 7 carbon atoms. For the description of R¹⁰ and R¹¹ in General Formula (G4), the description of R¹⁰ and R¹¹ in General Formula (G2) can be referred to.

Furthermore, R²⁰ represents an alkyl group having 1 to 6 carbon atoms or a phenyl group having an alkyl group having 1 to 6 carbon atoms. For the description of R²⁰ in General Formula (G4), the description of R²⁰ in General Formula (G3) can be referred to.

R² and R³ each independently represent any one of an alkyl group having 1 to 6 carbon atoms, an unsubstituted phenyl group, and a phenyl group having an alkyl group having 1 to 6 carbon atoms, and R² and R³ may be bonded to each other to form a ring. For the description of R² and R³ in General Formula (G4), the description of R² and R³ in General Formula (G1) can be referred to.

A film including the organic compound of one embodiment of the present invention having the above structure has a low refractive index, a large GSP_slope, and a high hole-transport property, and the organic compound of one embodiment of the present invention can have an appropriate HOMO level. A light-emitting device including the organic compound with such a structure can have high emission efficiency, low driving voltage, and extremely high power efficiency.

Next, a synthesis method of the organic compound represented by General Formula (G1) shown below is described.

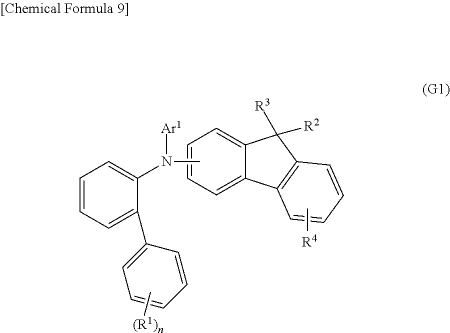

In General Formula (G1) above, R¹ to R⁴, n, and Ar¹ are as described above.

The organic compound represented by General Formula (G1) can be obtained by, as shown in the following synthesis scheme, coupling fluorenylamine and an organic halide using a metal catalyst, a metal, or a metal compound in the presence of a base.

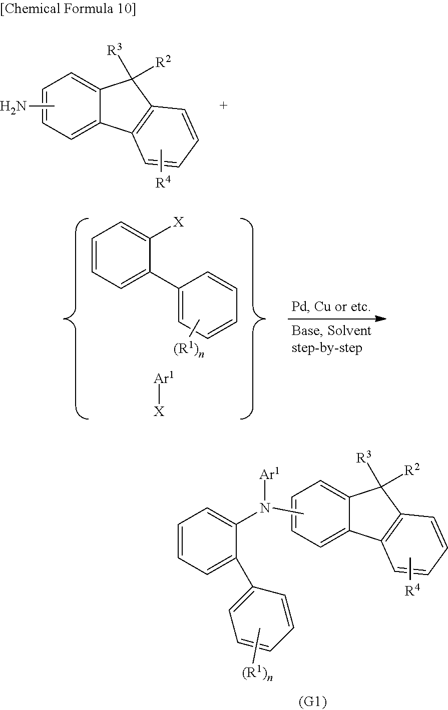

In the case where a Buchwald-Hartwig reaction is performed in the above synthesis scheme, X represents halogen or a trifluoromethanesulfonyl group. As the halogen, iodine, bromine, or chlorine is preferred.

In this reaction, a palladium catalyst including a palladium complex or a palladium compound such as bis(dibenzylideneacetone)palladium(0) or allylpalladium(II) chloride dimer and a ligand that is coordinated to the palladium complex or the palladium compound, such as tri(tert-butyl)phosphine, di(tert-butyl)(1-methyl-2,2-diphenylcyclopropyl)phosphine, or tricyclohexylphosphine, can be used.

In the case where the base is used for the above reaction, specific examples of the base include organic bases such as sodium-tert-butoxide and inorganic bases such as potassium carbonate. In the case where a solvent is used, toluene, xylene, 1,3,5-trimethylbenzene, or the like can be used.

In the case where Ullmann reaction is performed in the above synthesis scheme, X represents halogen. As the halogen, iodine, bromine, or chlorine is preferred. As a catalyst, copper or a copper compound can be used. Note that copper(I) iodide, copper(II) acetate, or the like is preferably used.

In the case where the base is used for the reaction, examples of the base include inorganic bases such as potassium carbonate and cesium carbonate. In the case where a solvent is used, 1,3-dimethyl-3,4,5,6-tetrahydro-2(1H)-pyrimidinone (DMPU), N-methyl-2-pyrrolidone (NMP), toluene, xylene, 1,3,5-trimethylbenzene, or the like can be used. In the Ullmann reaction, the target substance can be obtained in a shorter time and in a higher yield when the reaction temperature is 100° C. or higher; therefore, it is preferable to use DMPU, NMP, or 1,3,5-trimethylbenzene each having a high boiling point. In addition, the reaction temperature is further preferably 150° C. or higher; therefore, DMPU is further preferably used.

Since a variety of kinds of arylamine and organic halides used in the above synthesis scheme are commercially available or can be easily synthesized by known techniques, a great variety of the organic compounds represented by General Formula (G1) can be synthesized. Thus, the organic compound of one embodiment of the present invention is rich in variety. Note that the base, the catalyst, and the solvent which can be used are not limited thereto.

In the above manner, the organic compound of one embodiment of the present invention represented by General Formula (G1) can be obtained.

Specific examples of the organic compound of one embodiment of the present invention described in this embodiment include organic compounds represented by Structural Formulae (100) to (384) below.

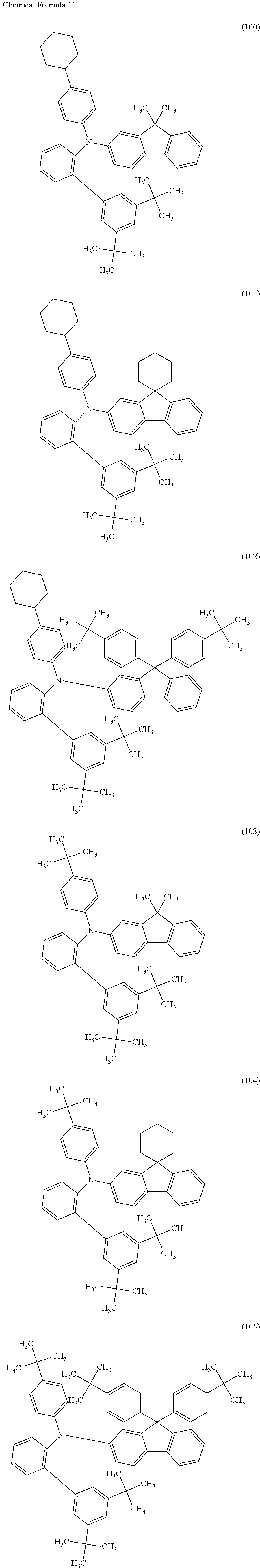

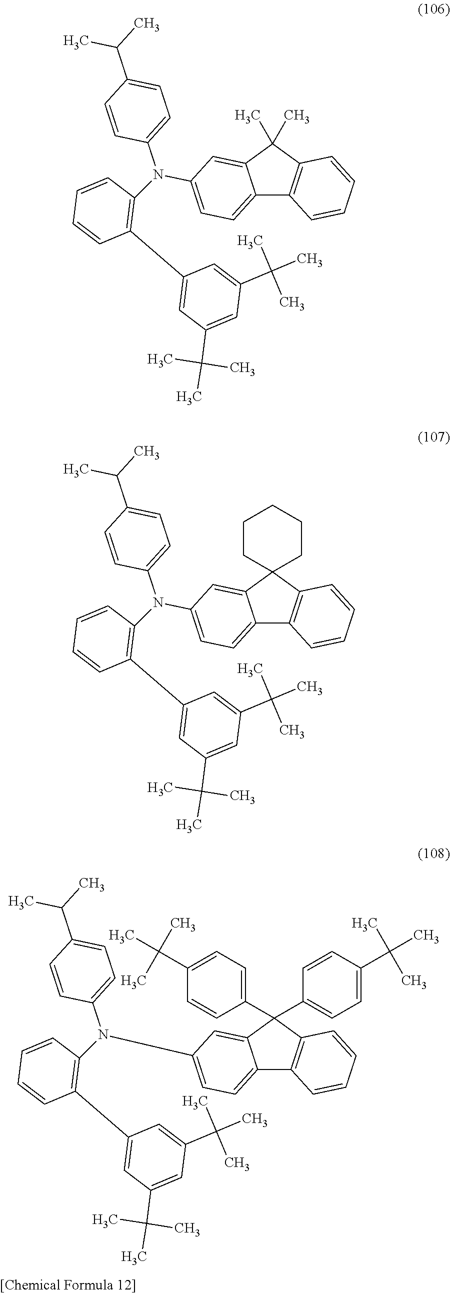 [SMILES: CC(C)c1ccc(N(c2ccc3c(c2)C(C)(C)c2ccccc2-3)c2ccccc2-c2cc(C(C)(C)C)cc(C(C)(C)C)c2)cc1 ; CC(C)c1ccc(N(c2ccc3c(c2)C2(CCCCC2)c2ccccc2-3)c2ccccc2-c2cc(C(C)(C)C)cc(C(C)(C)C)c2)cc1 ; CC(C)c1ccc(N(c2ccc3c(c2)C(c2ccc(C(C)(C)C)cc2)(c2ccc(C(C)(C)C)cc2)c2ccccc2-3)c2ccccc2-c2cc(C(C)(C)C)cc(C(C)(C)C)c2)cc1]

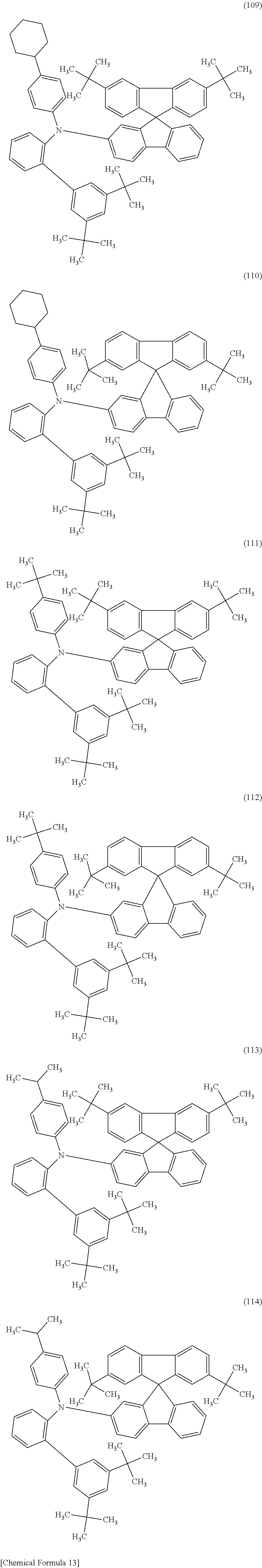

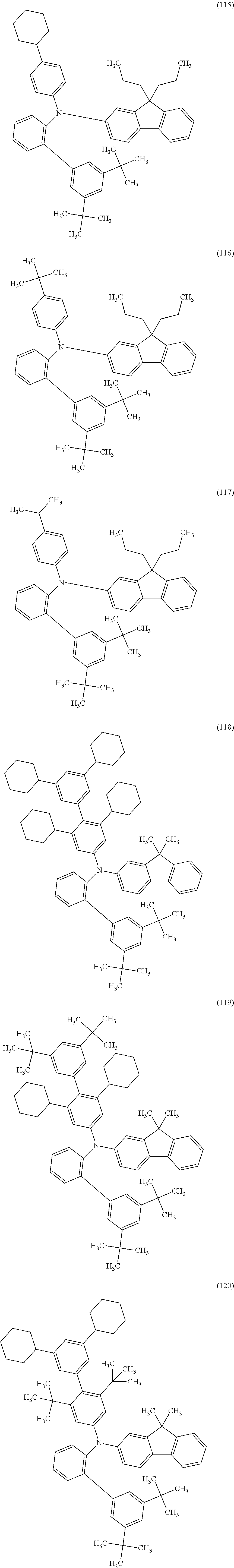

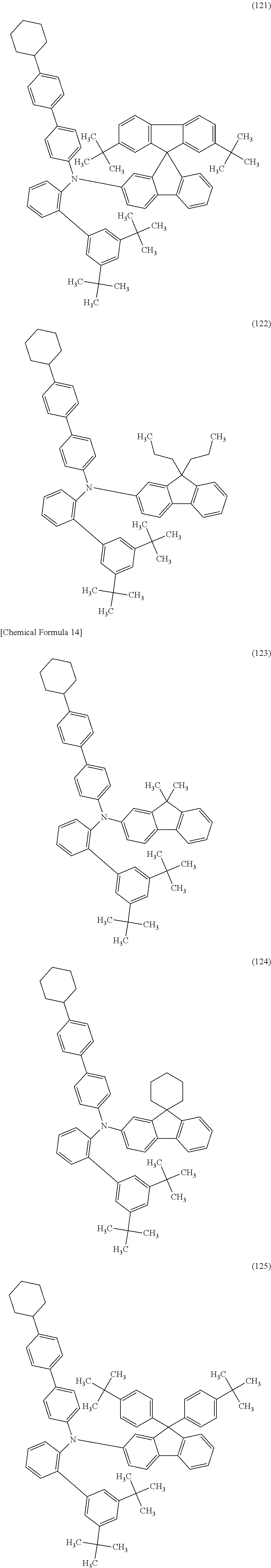

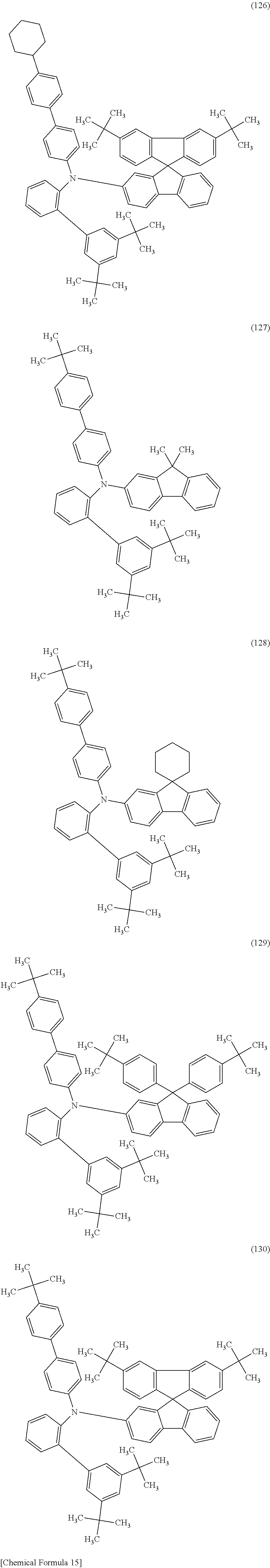

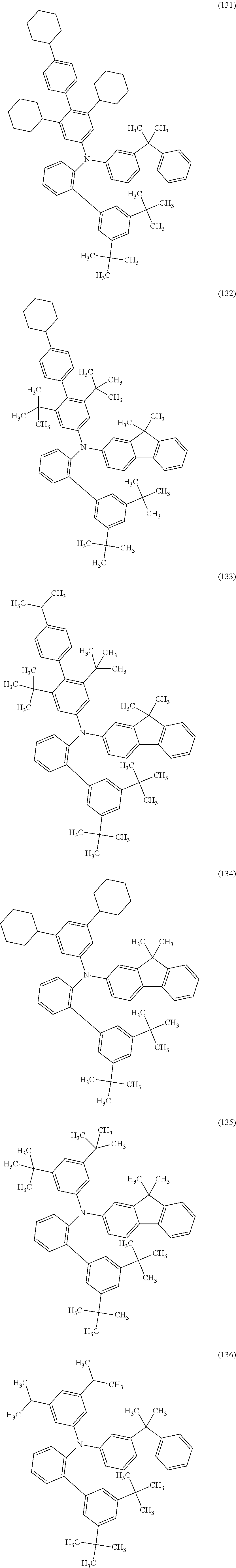

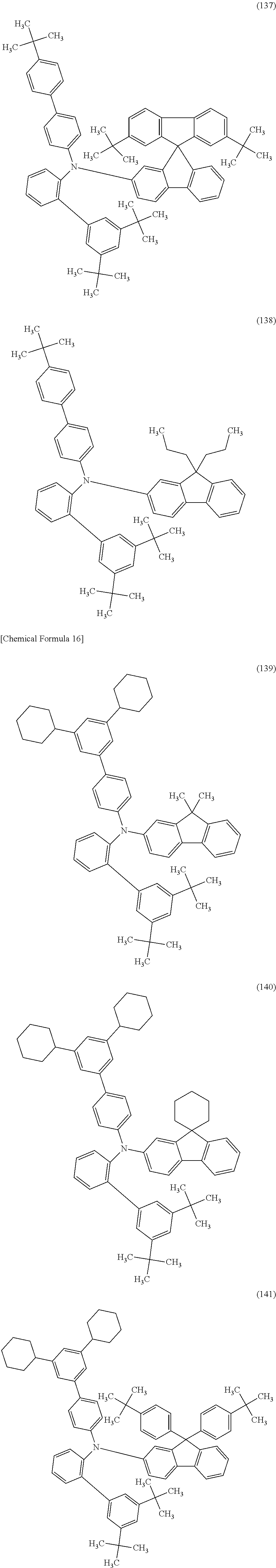

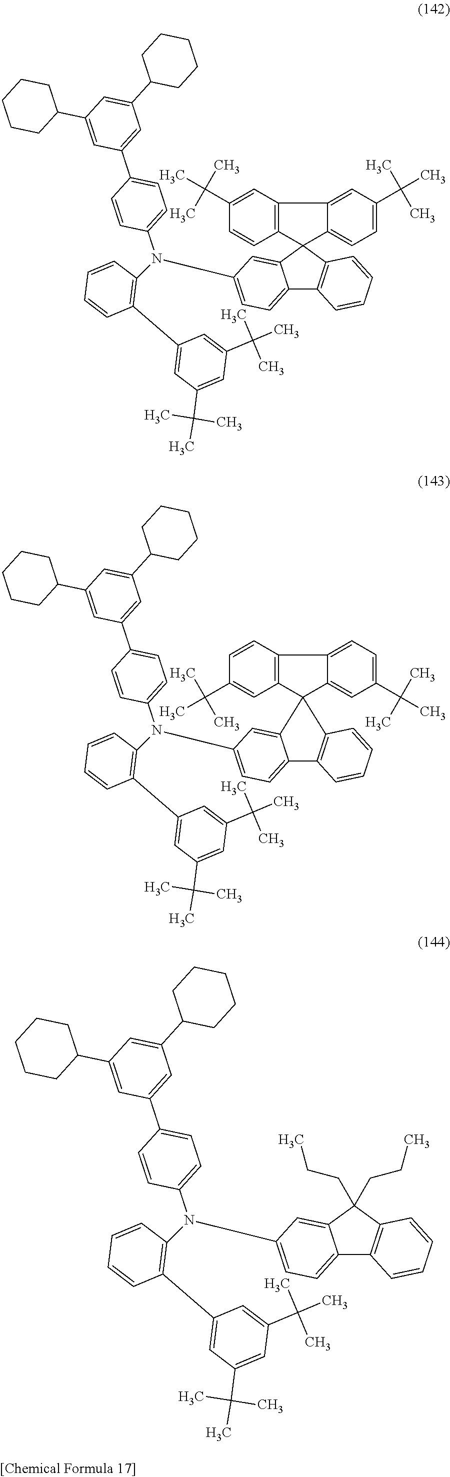 [SMILES: CC(C)(C)c1cc(-c2ccccc2N(c2ccc(-c3cc(C4CCCCC4)cc(C4CCCCC4)c3)cc2)c2ccc3c(c2)C2(c4ccccc4-3)c3ccc(C(C)(C)C)cc3-c3cc(C(C)(C)C)ccc32)cc(C(C)(C)C)c1 ; CC(C)(C)c1cc(-c2ccccc2N(c2ccc(-c3cc(C4CCCCC4)cc(C4CCCCC4)c3)cc2)c2ccc3c(c2)C2(c4ccccc4-3)c3cc(C(C)(C)C)ccc3-c3ccc(C(C)(C)C)cc32)cc(C(C)(C)C)c1 ; CCCC1(CCC)c2ccccc2-c2ccc(N(c3ccc(-c4cc(C5CCCCC5)cc(C5CCCCC5)c4)cc3)c3ccccc3-c3cc(C(C)(C)C)cc(C(C)(C)C)c3)cc21]

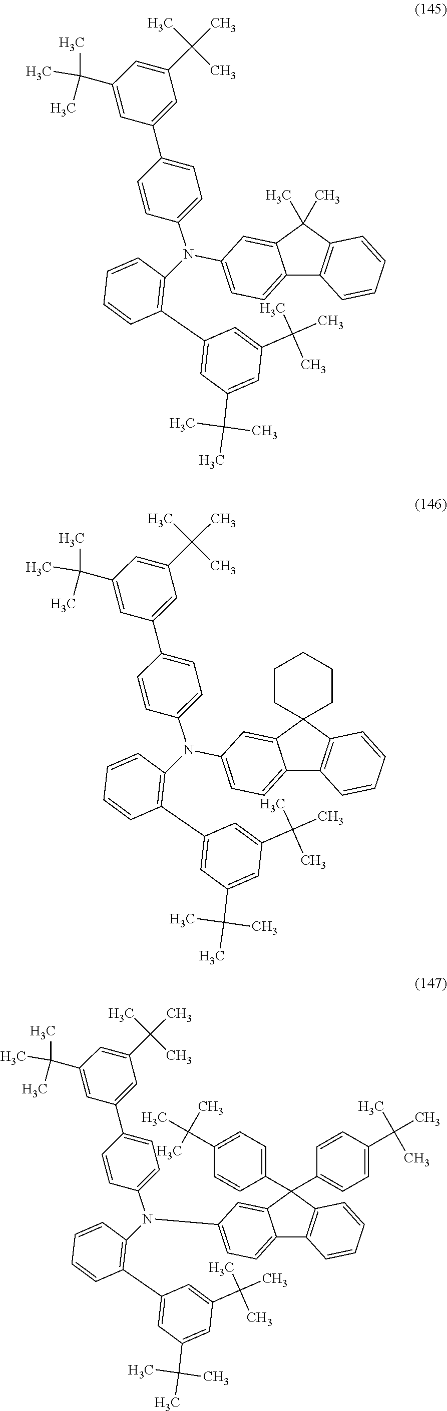 [SMILES: CC(C)(C)c1cc(-c2ccc(N(c3ccc4c(c3)C(C)(C)c3ccccc3-4)c3ccccc3-c3cc(C(C)(C)C)cc(C(C)(C)C)c3)cc2)cc(C(C)(C)C)c1 ; CC(C)(C)c1cc(-c2ccc(N(c3ccc4c(c3)C3(CCCCC3)c3ccccc3-4)c3ccccc3-c3cc(C(C)(C)C)cc(C(C)(C)C)c3)cc2)cc(C(C)(C)C)c1 ; CC(C)(C)c1ccc(C2(c3ccc(C(C)(C)C)cc3)c3ccccc3-c3ccc(N(c4ccc(-c5cc(C(C)(C)C)cc(C(C)(C)C)c5)cc4)c4ccccc4-c4cc(C(C)(C)C)cc(C(C)(C)C)c4)cc32)cc1]

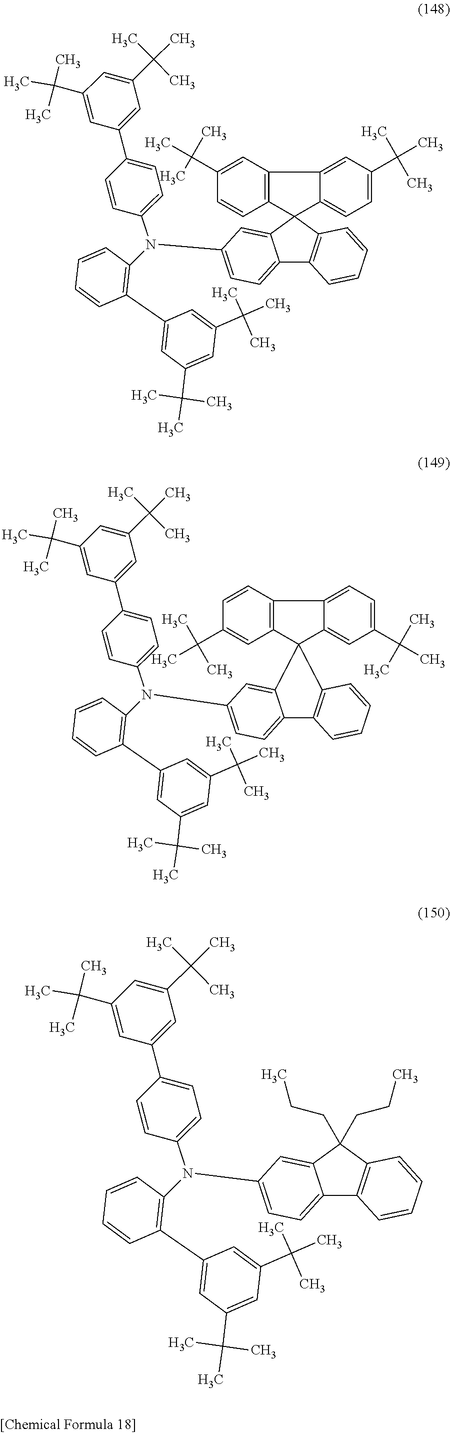

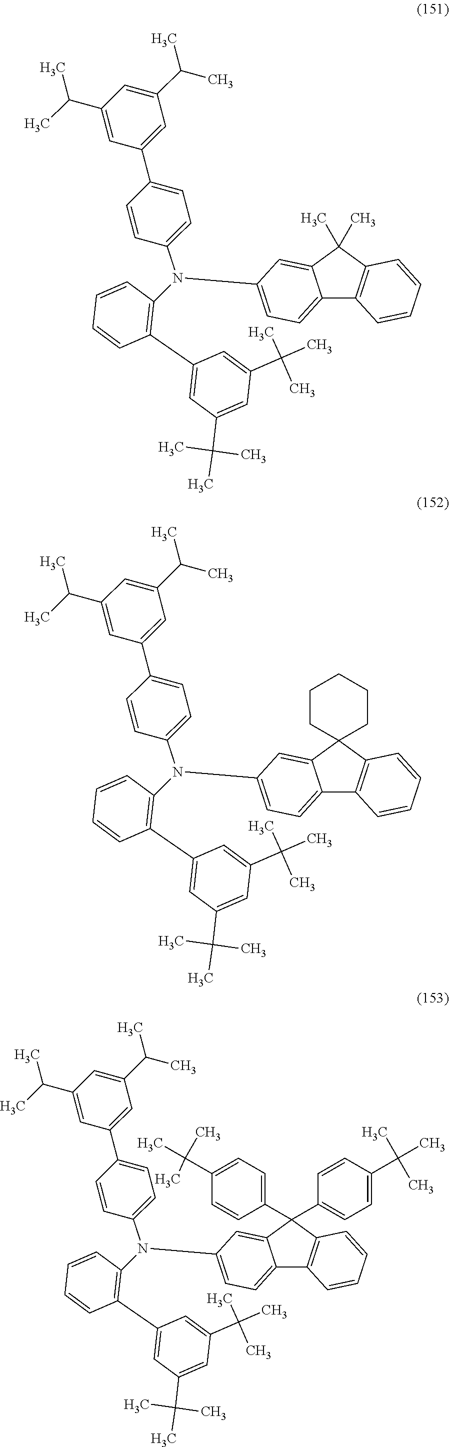 [SMILES: CC(C)c1cc(-c2ccc(N(c3ccc4c(c3)C(C)(C)c3ccccc3-4)c3ccccc3-c3cc(C(C)(C)C)cc(C(C)(C)C)c3)cc2)cc(C(C)C)c1 ; CC(C)c1cc(-c2ccc(N(c3ccc4c(c3)C3(CCCCC3)c3ccccc3-4)c3ccccc3-c3cc(C(C)(C)C)cc(C(C)(C)C)c3)cc2)cc(C(C)C)c1 ; CC(C)c1cc(-c2ccc(N(c3ccc4c(c3)C(c3ccc(C(C)(C)C)cc3)(c3ccc(C(C)(C)C)cc3)c3ccccc3-4)c3ccccc3-c3cc(C(C)(C)C)cc(C(C)(C)C)c3)cc2)cc(C(C)C)c1]

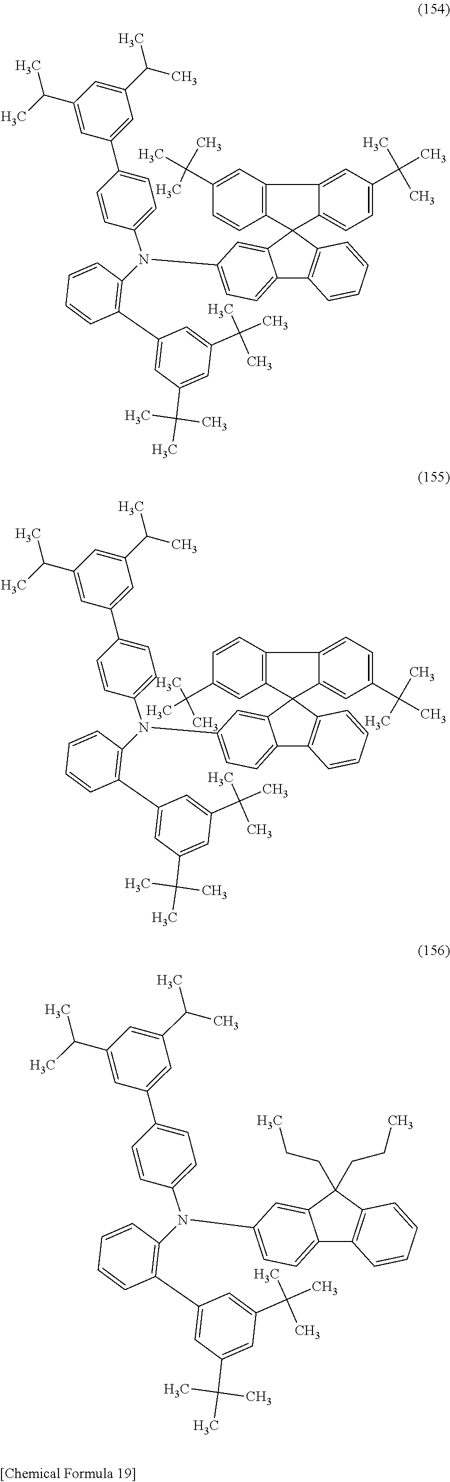

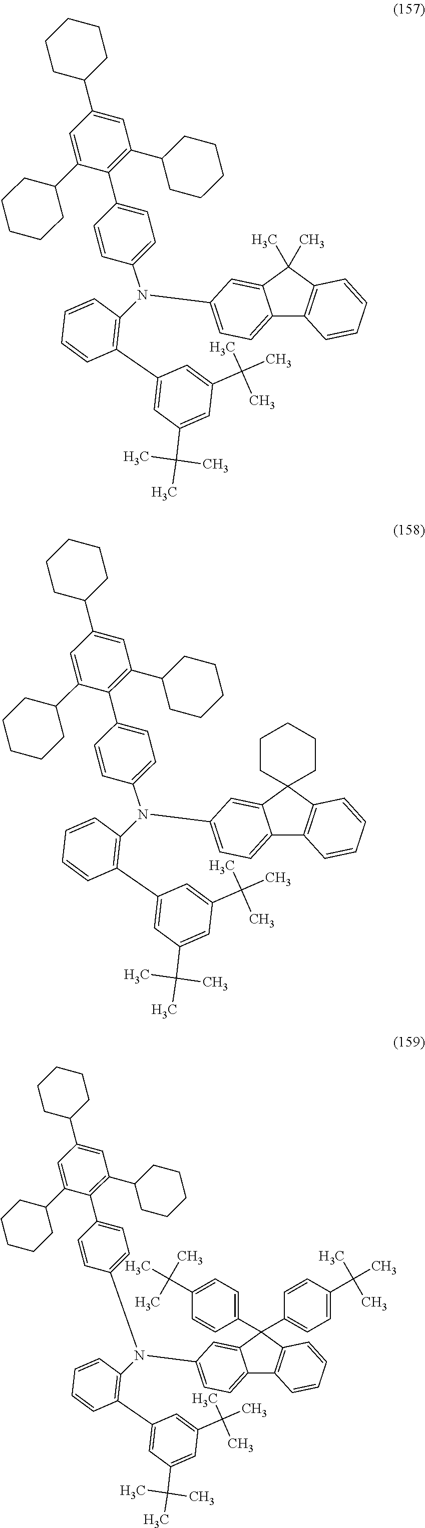 [SMILES: CC(C)(C)c1cc(-c2ccccc2N(c2ccc(-c3c(C4CCCCC4)cc(C4CCCCC4)cc3C3CCCCC3)cc2)c2ccc3c(c2)C(C)(C)c2ccccc2-3)cc(C(C)(C)C)c1 ; CC(C)(C)c1cc(-c2ccccc2N(c2ccc(-c3c(C4CCCCC4)cc(C4CCCCC4)cc3C3CCCCC3)cc2)c2ccc3c(c2)C2(CCCCC2)c2ccccc2-3)cc(C(C)(C)C)c1 ; CC(C)(C)c1ccc(C2(c3ccc(C(C)(C)C)cc3)c3ccccc3-c3ccc(N(c4ccc(-c5c(C6CCCCC6)cc(C6CCCCC6)cc5C5CCCCC5)cc4)c4ccccc4-c4cc(C(C)(C)C)cc(C(C)(C)C)c4)cc32)cc1]

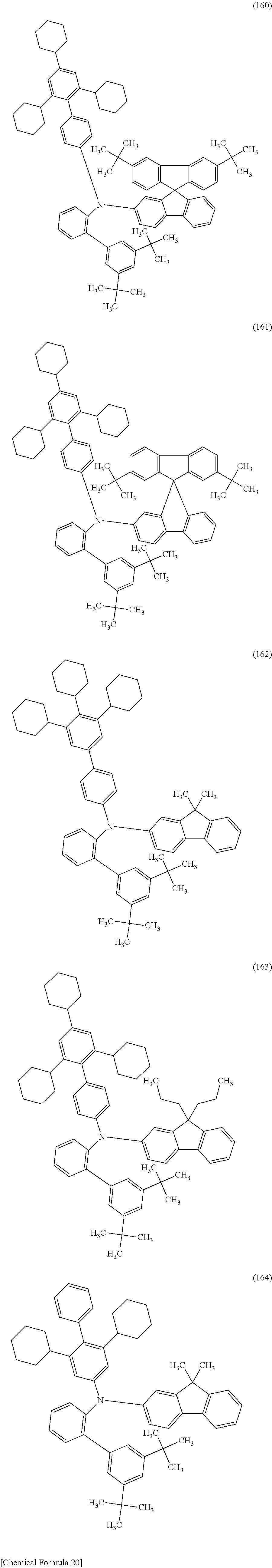

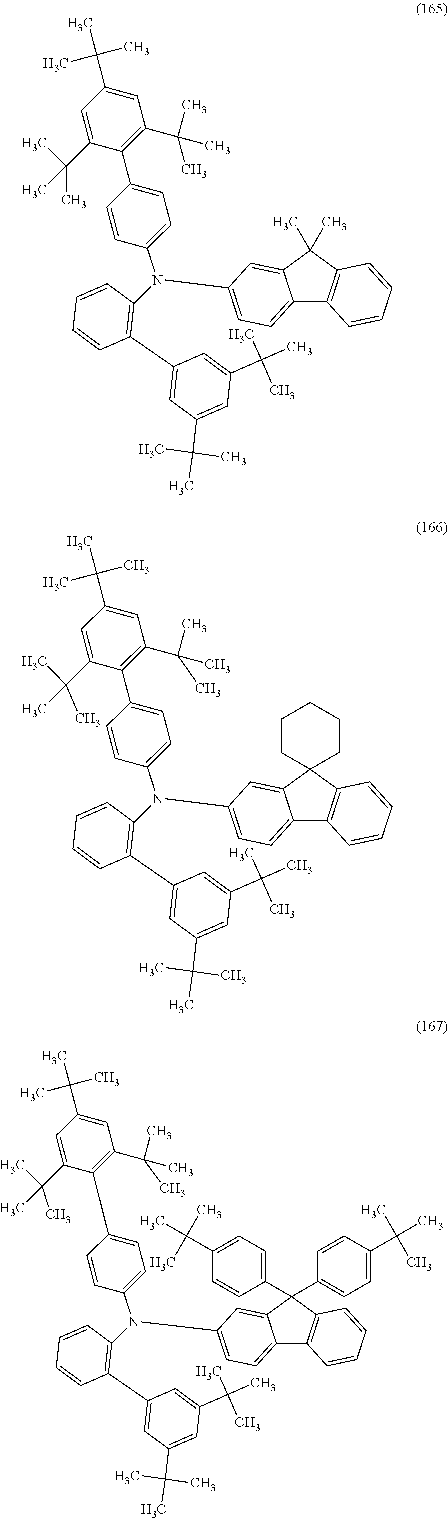 [SMILES: CC(C)(C)c1cc(-c2ccccc2N(c2ccc(-c3c(C(C)(C)C)cc(C(C)(C)C)cc3C(C)(C)C)cc2)c2ccc3c(c2)C(C)(C)c2ccccc2-3)cc(C(C)(C)C)c1 ; CC(C)(C)c1cc(-c2ccccc2N(c2ccc(-c3c(C(C)(C)C)cc(C(C)(C)C)cc3C(C)(C)C)cc2)c2ccc3c(c2)C2(CCCCC2)c2ccccc2-3)cc(C(C)(C)C)c1 ; CC(C)(C)c1ccc(C2(c3ccc(C(C)(C)C)cc3)c3ccccc3-c3ccc(N(c4ccc(-c5c(C(C)(C)C)cc(C(C)(C)C)cc5C(C)(C)C)cc4)c4ccccc4-c4cc(C(C)(C)C)cc(C(C)(C)C)c4)cc32)cc1]

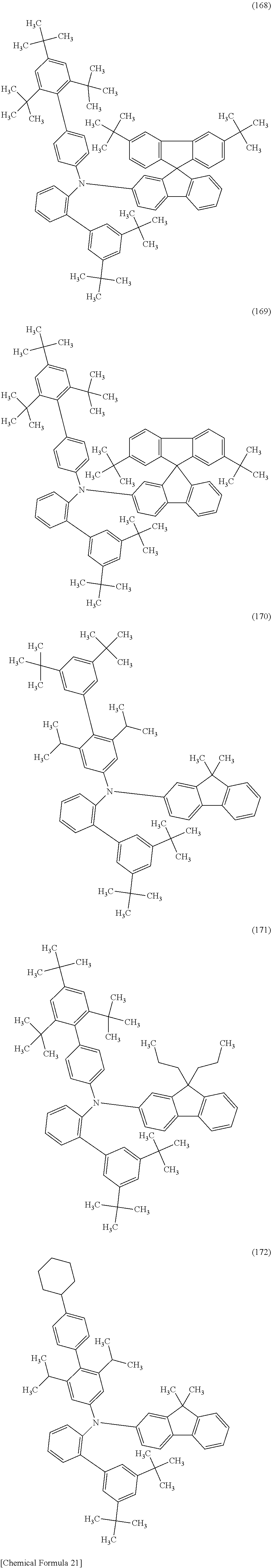

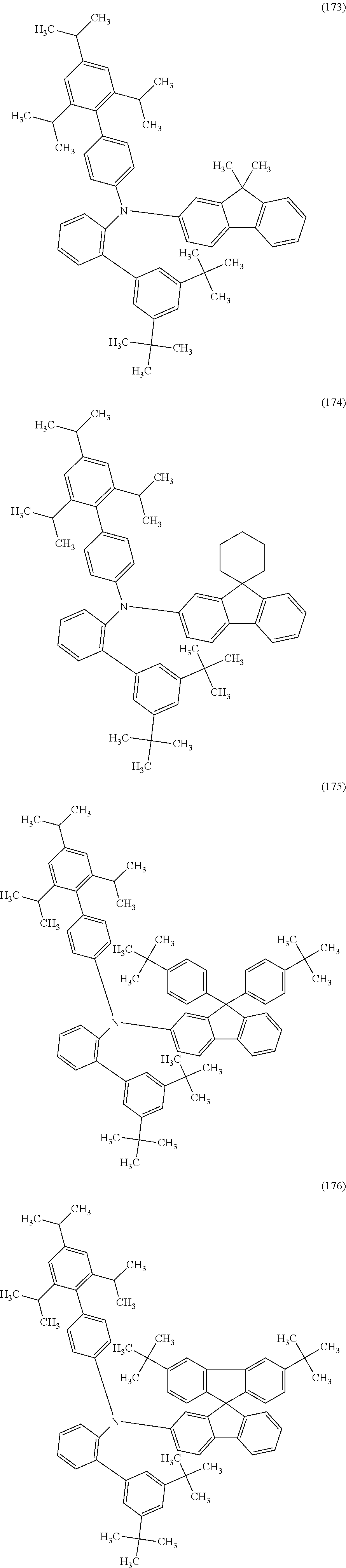

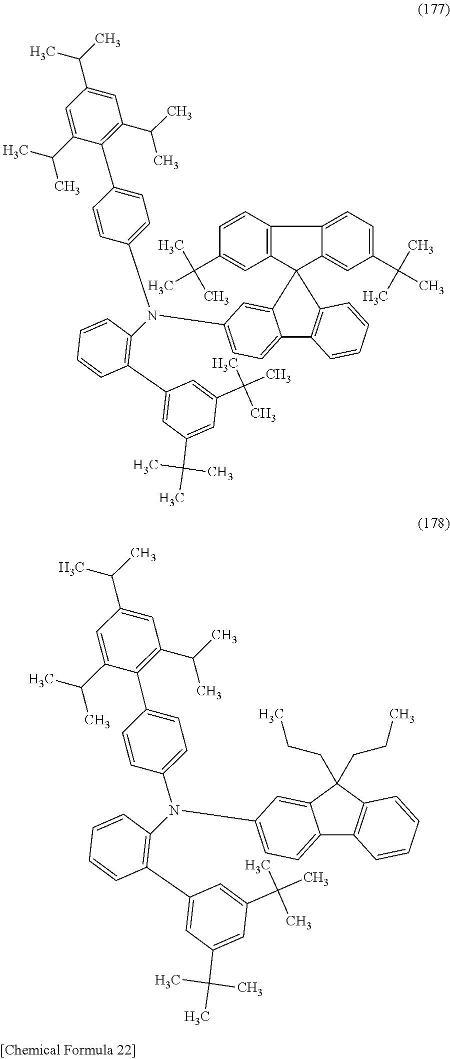

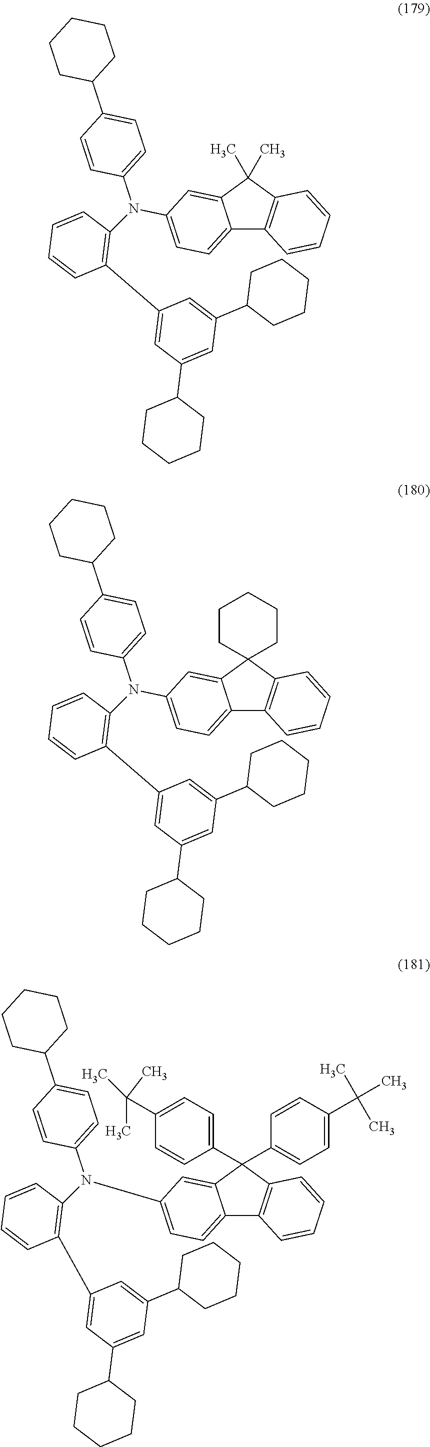 [SMILES: CC1(C)c2ccccc2-c2ccc(N(c3ccc(C4CCCCC4)cc3)c3ccccc3-c3cc(C4CCCCC4)cc(C4CCCCC4)c3)cc21 ; c1ccc(N(c2ccc(C3CCCCC3)cc2)c2ccc3c(c2)C2(CCCCC2)c2ccccc2-3)c(-c2cc(C3CCCCC3)cc(C3CCCCC3)c2)c1 ; CC(C)(C)c1ccc(C2(c3ccc(C(C)(C)C)cc3)c3ccccc3-c3ccc(N(c4ccc(C5CCCCC5)cc4)c4ccccc4-c4cc(C5CCCCC5)cc(C5CCCCC5)c4)cc32)cc1]

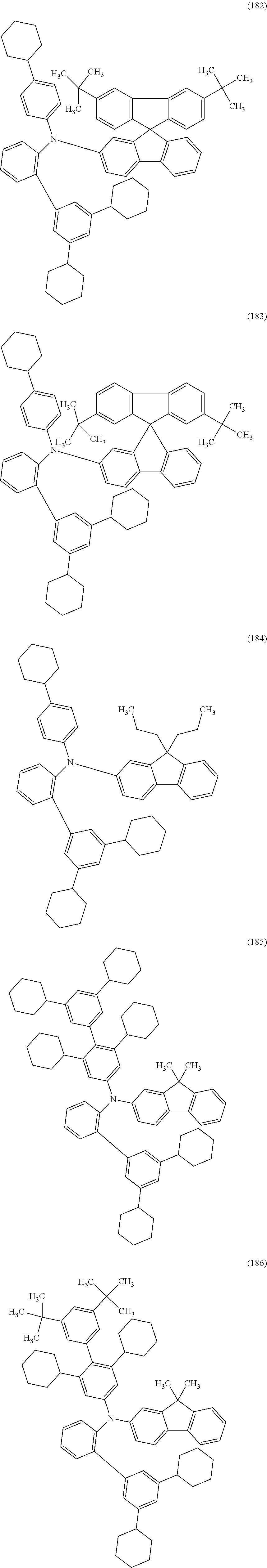

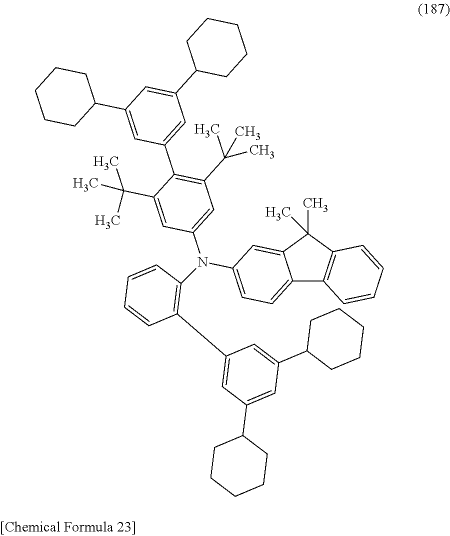 [SMILES: CC(C)(C)c1cc(N(c2ccc3c(c2)C(C)(C)c2ccccc2-3)c2ccccc2-c2cc(C3CCCCC3)cc(C3CCCCC3)c2)cc(C(C)(C)C)c1-c1cc(C2CCCCC2)cc(C2CCCCC2)c1]

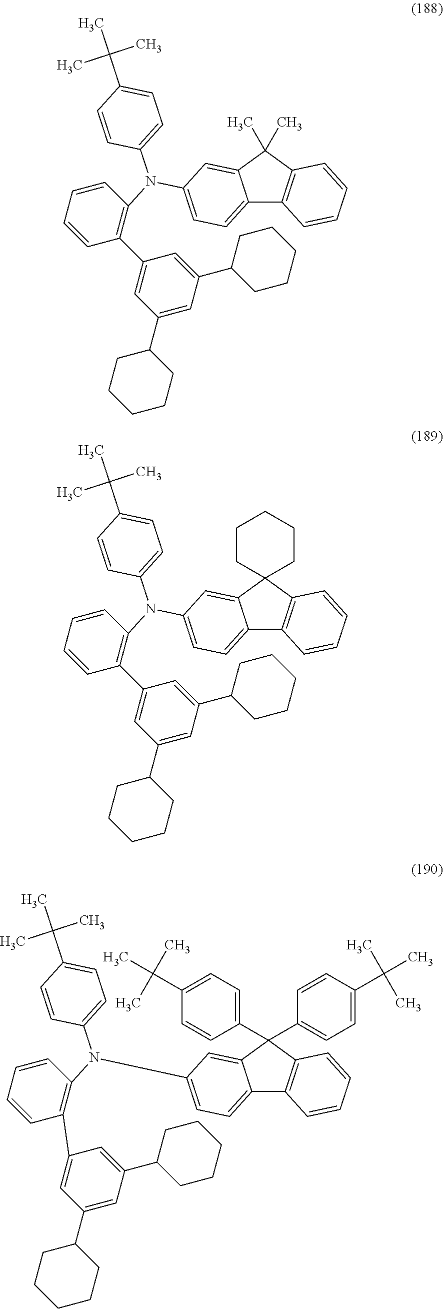 [SMILES: CC(C)(C)c1ccc(N(c2ccc3c(c2)C(C)(C)c2ccccc2-3)c2ccccc2-c2cc(C3CCCCC3)cc(C3CCCCC3)c2)cc1 ; CC(C)(C)c1ccc(N(c2ccc3c(c2)C2(CCCCC2)c2ccccc2-3)c2ccccc2-c2cc(C3CCCCC3)cc(C3CCCCC3)c2)cc1 ; CC(C)(C)c1ccc(N(c2ccc3c(c2)C(c2ccc(C(C)(C)C)cc2)(c2ccc(C(C)(C)C)cc2)c2ccccc2-3)c2ccccc2-c2cc(C3CCCCC3)cc(C3CCCCC3)c2)cc1]

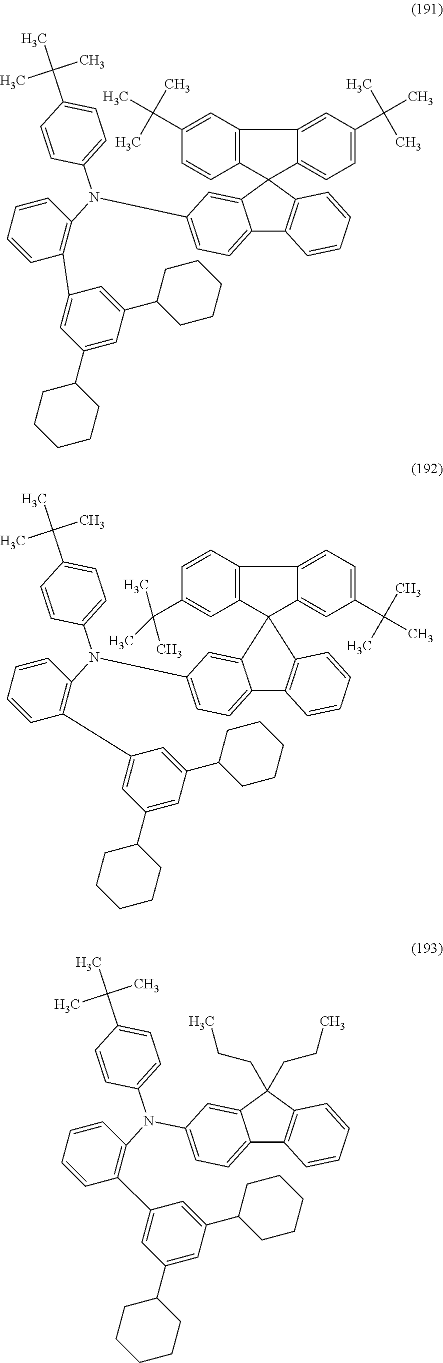 [SMILES: CC(C)(C)c1ccc(N(c2ccc3c(c2)C2(c4ccccc4-3)c3ccc(C(C)(C)C)cc3-c3cc(C(C)(C)C)ccc32)c2ccccc2-c2cc(C3CCCCC3)cc(C3CCCCC3)c2)cc1 ; CC(C)(C)c1ccc(N(c2ccc3c(c2)C2(c4ccccc4-3)c3cc(C(C)(C)C)ccc3-c3ccc(C(C)(C)C)cc32)c2ccccc2-c2cc(C3CCCCC3)cc(C3CCCCC3)c2)cc1 ; CCCC1(CCC)c2ccccc2-c2ccc(N(c3ccc(C(C)(C)C)cc3)c3ccccc3-c3cc(C4CCCCC4)cc(C4CCCCC4)c3)cc21]

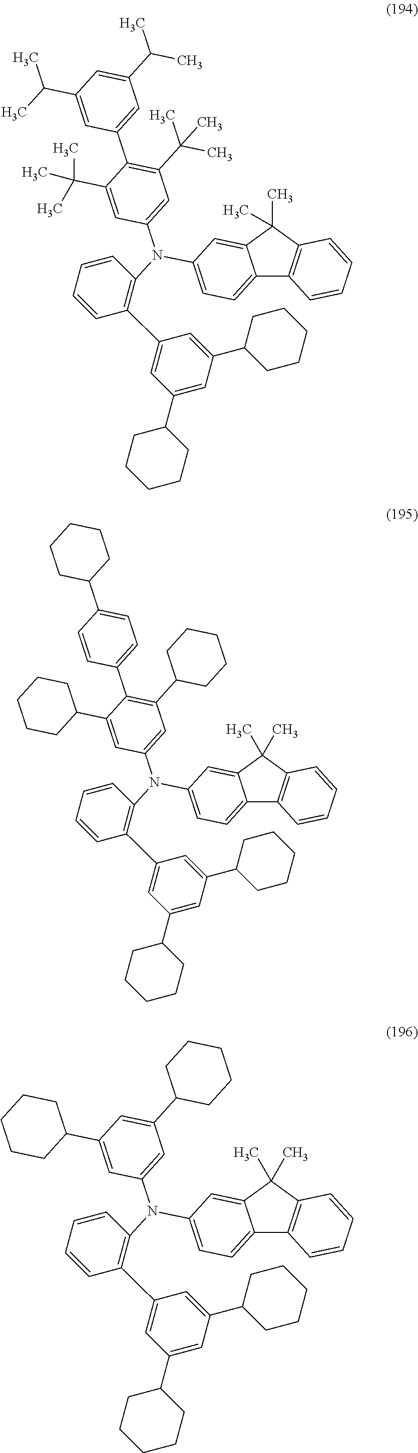 [SMILES: CC(C)c1cc(-c2c(C(C)(C)C)cc(N(c3ccc4c(c3)C(C)(C)c3ccccc3-4)c3ccccc3-c3cc(C4CCCCC4)cc(C4CCCCC4)c3)cc2C(C)(C)C)cc(C(C)C)c1 ; CC1(C)c2ccccc2-c2ccc(N(c3cc(C4CCCCC4)c(-c4ccc(C5CCCCC5)cc4)c(C4CCCCC4)c3)c3ccccc3-c3cc(C4CCCCC4)cc(C4CCCCC4)c3)cc21 ; CC1(C)c2ccccc2-c2ccc(N(c3cc(C4CCCCC4)cc(C4CCCCC4)c3)c3ccccc3-c3cc(C4CCCCC4)cc(C4CCCCC4)c3)cc21]

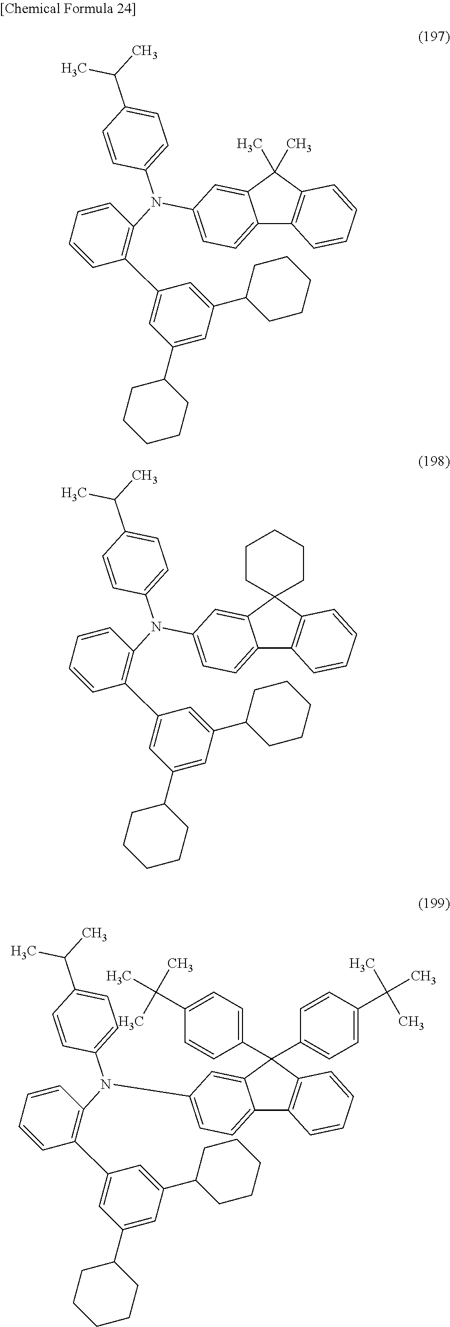

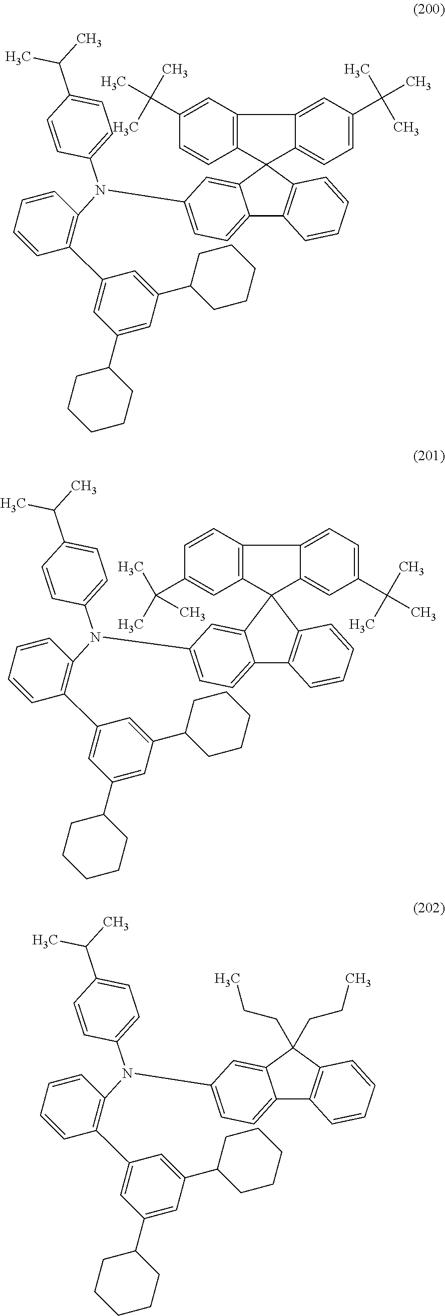 [SMILES: CC(C)c1ccc(N(c2ccc3c(c2)C2(c4ccccc4-3)c3ccc(C(C)(C)C)cc3-c3cc(C(C)(C)C)ccc32)c2ccccc2-c2cc(C3CCCCC3)cc(C3CCCCC3)c2)cc1 ; CC(C)c1ccc(N(c2ccc3c(c2)C2(c4ccccc4-3)c3cc(C(C)(C)C)ccc3-c3ccc(C(C)(C)C)cc32)c2ccccc2-c2cc(C3CCCCC3)cc(C3CCCCC3)c2)cc1 ; CCCC1(CCC)c2ccccc2-c2ccc(N(c3ccc(C(C)C)cc3)c3ccccc3-c3cc(C4CCCCC4)cc(C4CCCCC4)c3)cc21]

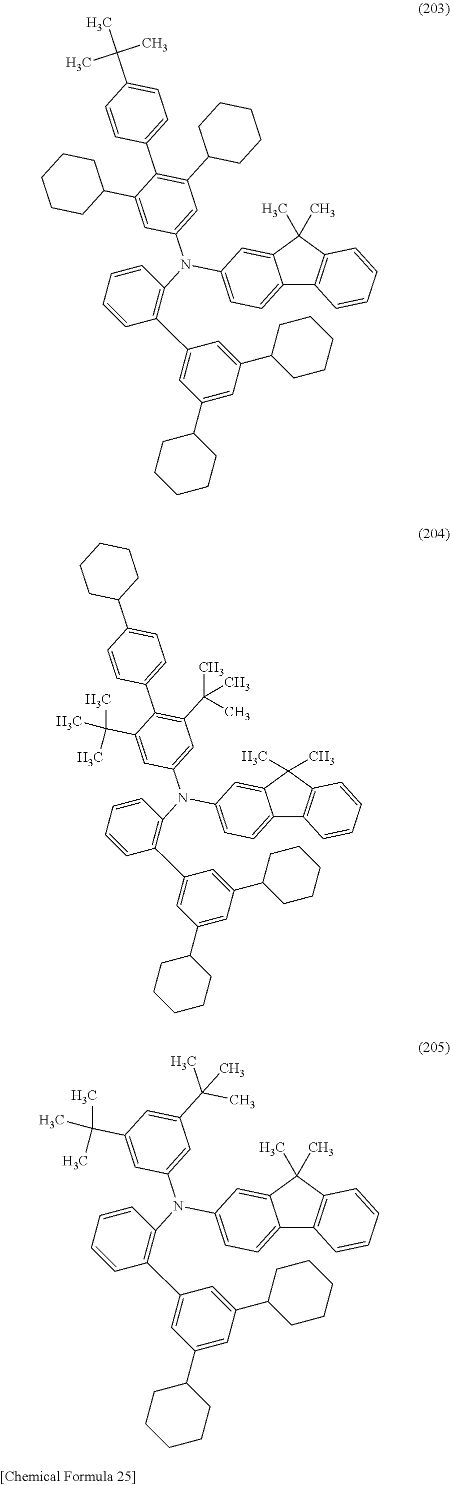 [SMILES: CC(C)(C)c1ccc(-c2c(C3CCCCC3)cc(N(c3ccc4c(c3)C(C)(C)c3ccccc3-4)c3ccccc3-c3cc(C4CCCCC4)cc(C4CCCCC4)c3)cc2C2CCCCC2)cc1 ; CC(C)(C)c1cc(N(c2ccc3c(c2)C(C)(C)c2ccccc2-3)c2ccccc2-c2cc(C3CCCCC3)cc(C3CCCCC3)c2)cc(C(C)(C)C)c1-c1ccc(C2CCCCC2)cc1 ; CC(C)(C)c1cc(N(c2ccc3c(c2)C(C)(C)c2ccccc2-3)c2ccccc2-c2cc(C3CCCCC3)cc(C3CCCCC3)c2)cc(C(C)(C)C)c1]

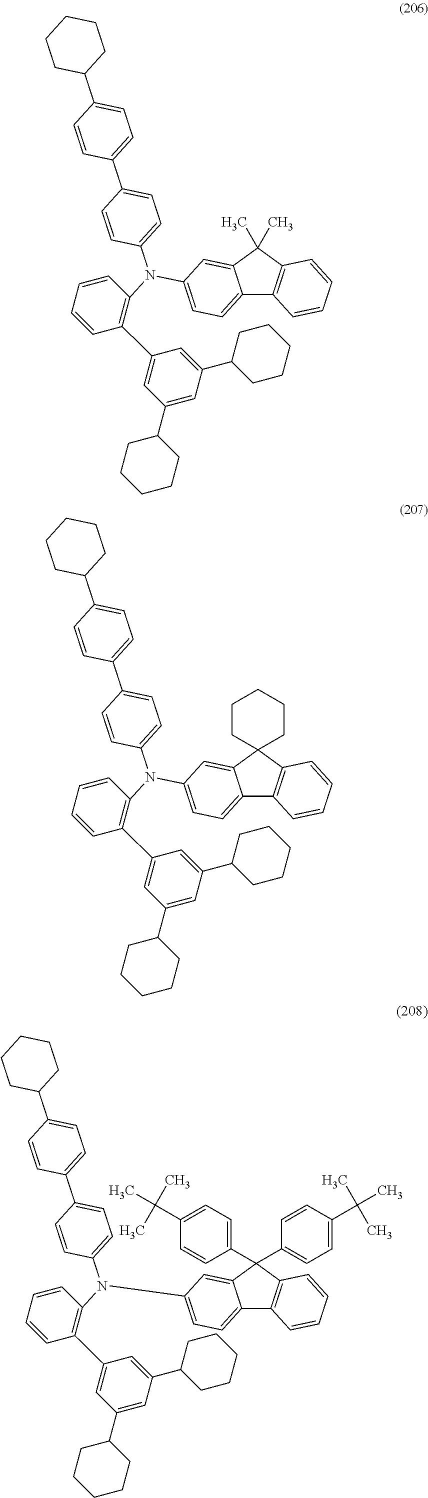 [SMILES: CC1(C)c2ccccc2-c2ccc(N(c3ccc(-c4ccc(C5CCCCC5)cc4)cc3)c3ccccc3-c3cc(C4CCCCC4)cc(C4CCCCC4)c3)cc21 ; c1ccc(N(c2ccc(-c3ccc(C4CCCCC4)cc3)cc2)c2ccc3c(c2)C2(CCCCC2)c2ccccc2-3)c(-c2cc(C3CCCCC3)cc(C3CCCCC3)c2)c1 ; CC(C)(C)c1ccc(C2(c3ccc(C(C)(C)C)cc3)c3ccccc3-c3ccc(N(c4ccc(-c5ccc(C6CCCCC6)cc5)cc4)c4ccccc4-c4cc(C5CCCCC5)cc(C5CCCCC5)c4)cc32)cc1]

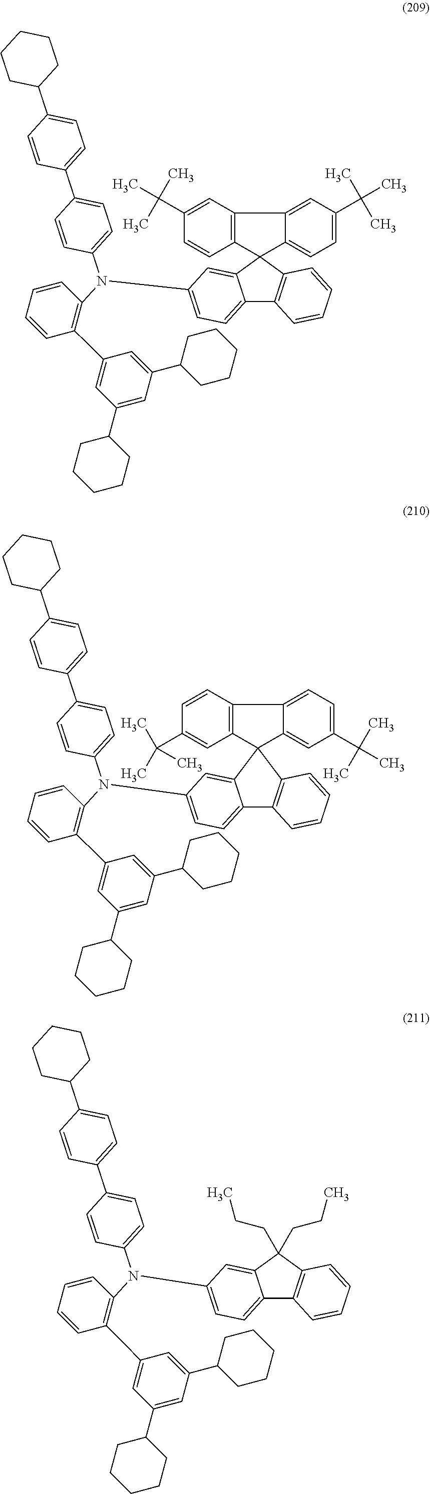

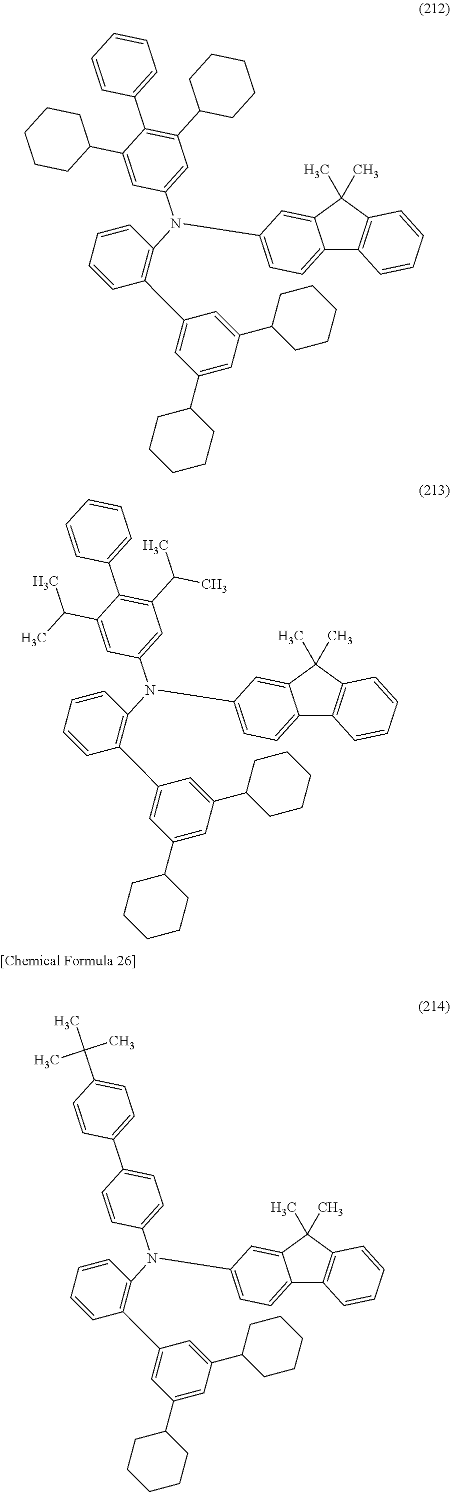 [SMILES: CC1(C)c2ccccc2-c2ccc(N(c3cc(C4CCCCC4)c(-c4ccccc4)c(C4CCCCC4)c3)c3ccccc3-c3cc(C4CCCCC4)cc(C4CCCCC4)c3)cc21 ; CC(C)c1cc(N(c2ccc3c(c2)C(C)(C)c2ccccc2-3)c2ccccc2-c2cc(C3CCCCC3)cc(C3CCCCC3)c2)cc(C(C)C)c1-c1ccccc1 ; CC(C)(C)c1ccc(-c2ccc(N(c3ccc4c(c3)C(C)(C)c3ccccc3-4)c3ccccc3-c3cc(C4CCCCC4)cc(C4CCCCC4)c3)cc2)cc1]

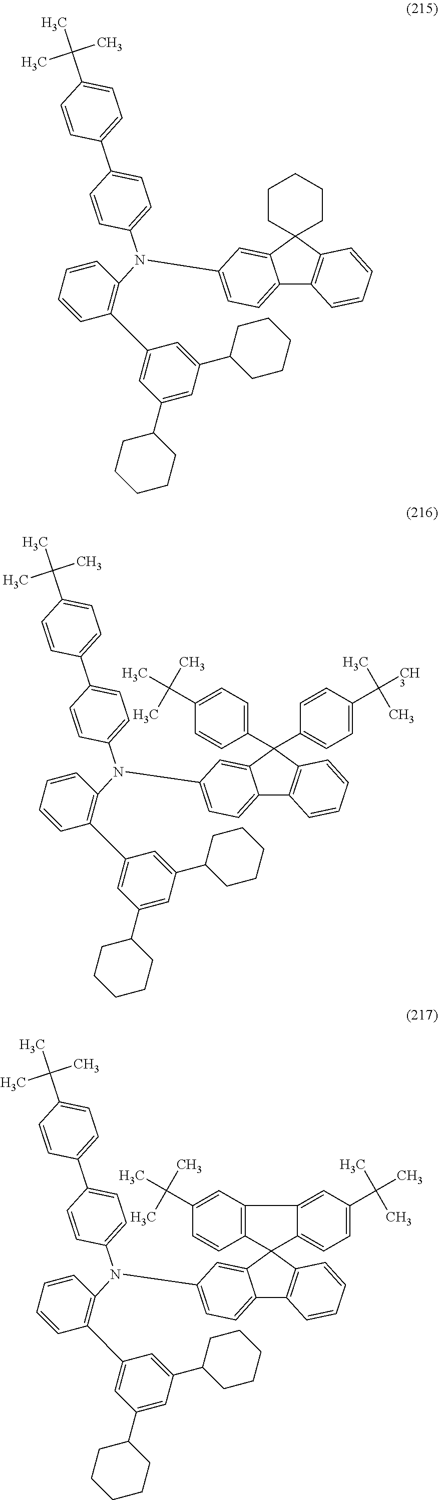

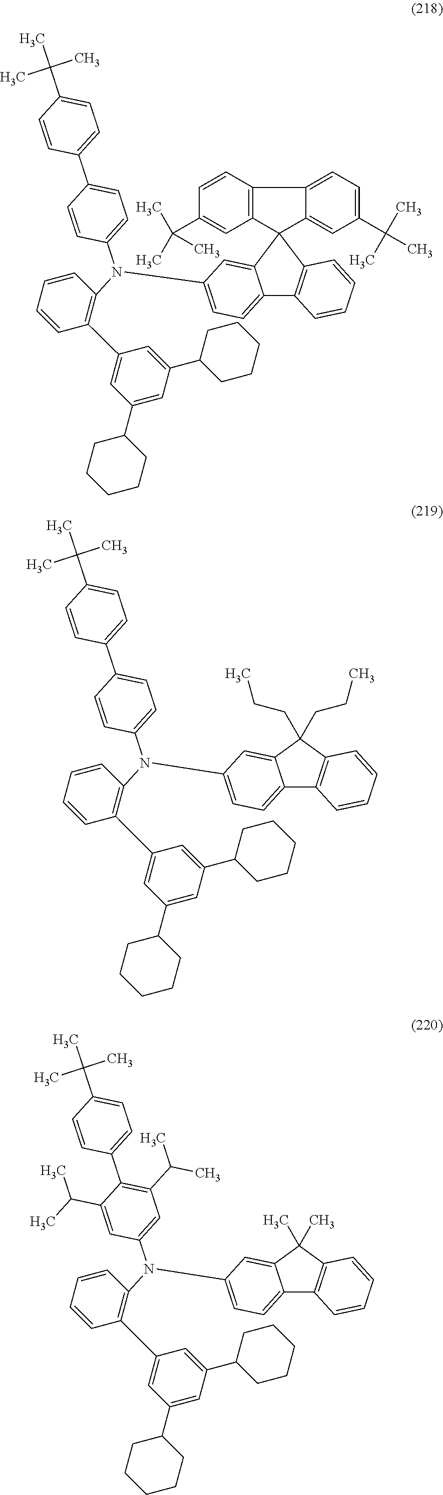

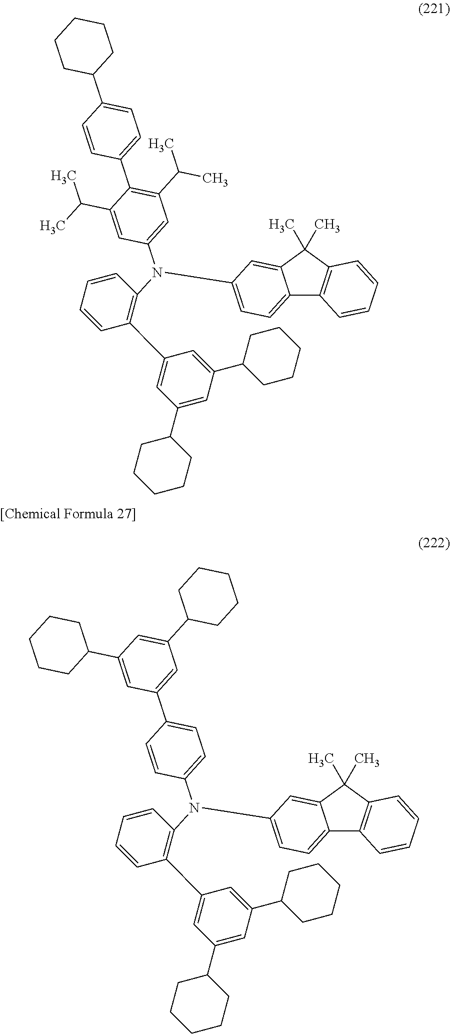 [SMILES: CC(C)c1cc(N(c2ccc3c(c2)C(C)(C)c2ccccc2-3)c2ccccc2-c2cc(C3CCCCC3)cc(C3CCCCC3)c2)cc(C(C)C)c1-c1ccc(C2CCCCC2)cc1 ; CC1(C)c2ccccc2-c2ccc(N(c3ccc(-c4cc(C5CCCCC5)cc(C5CCCCC5)c4)cc3)c3ccccc3-c3cc(C4CCCCC4)cc(C4CCCCC4)c3)cc21]

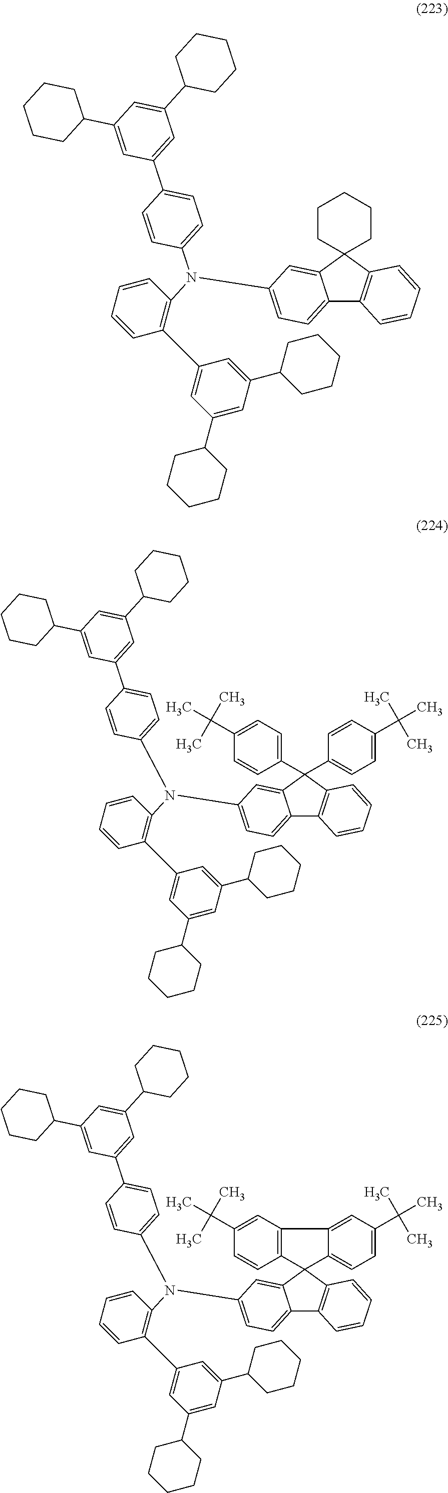 [SMILES: c1ccc(N(c2ccc(-c3cc(C4CCCCC4)cc(C4CCCCC4)c3)cc2)c2ccc3c(c2)C2(CCCCC2)c2ccccc2-3)c(-c2cc(C3CCCCC3)cc(C3CCCCC3)c2)c1 ; CC(C)(C)c1ccc(C2(c3ccc(C(C)(C)C)cc3)c3ccccc3-c3ccc(N(c4ccc(-c5cc(C6CCCCC6)cc(C6CCCCC6)c5)cc4)c4ccccc4-c4cc(C5CCCCC5)cc(C5CCCCC5)c4)cc32)cc1 ; CC(C)(C)c1ccc2c(c1)-c1cc(C(C)(C)C)ccc1C21c2ccccc2-c2ccc(N(c3ccc(-c4cc(C5CCCCC5)cc(C5CCCCC5)c4)cc3)c3ccccc3-c3cc(C4CCCCC4)cc(C4CCCCC4)c3)cc21]

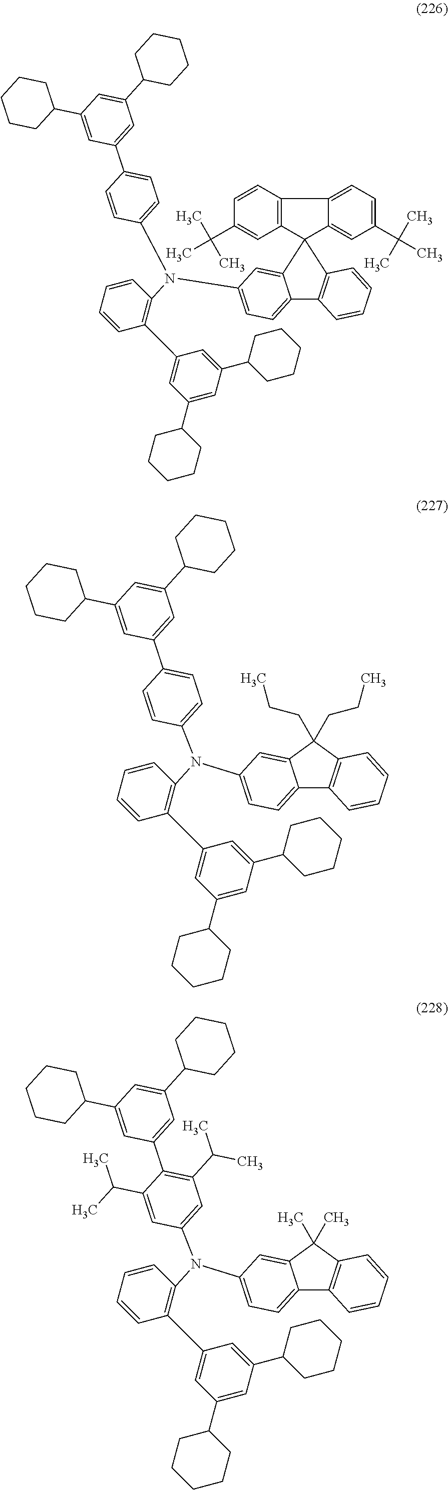

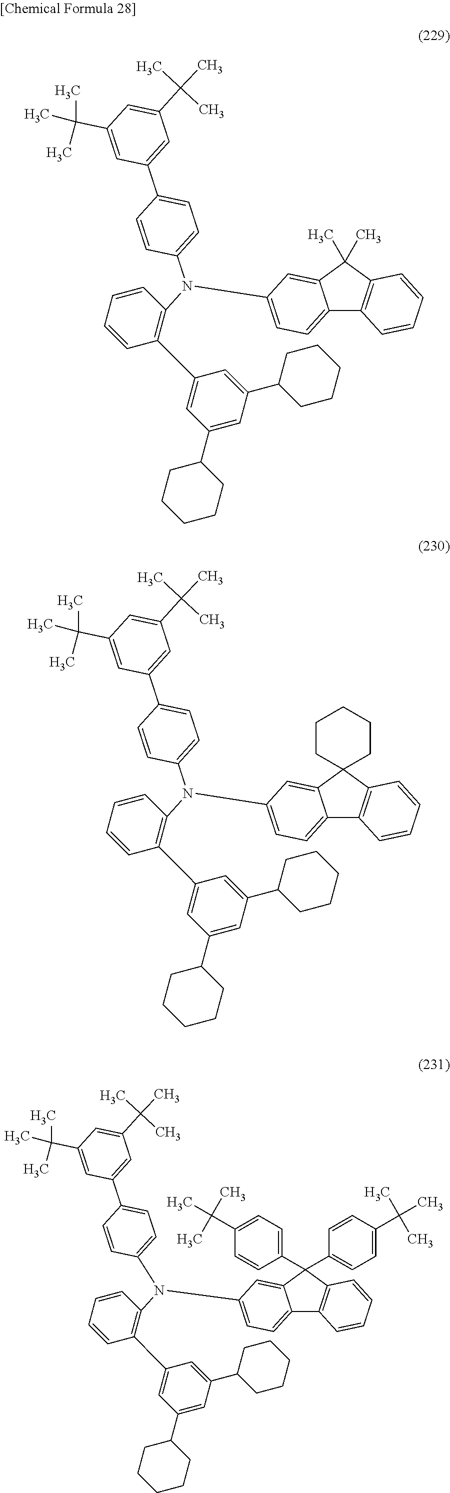 [SMILES: CC(C)(C)c1cc(-c2ccc(N(c3ccc4c(c3)C(C)(C)c3ccccc3-4)c3ccccc3-c3cc(C4CCCCC4)cc(C4CCCCC4)c3)cc2)cc(C(C)(C)C)c1 ; CC(C)(C)c1cc(-c2ccc(N(c3ccc4c(c3)C3(CCCCC3)c3ccccc3-4)c3ccccc3-c3cc(C4CCCCC4)cc(C4CCCCC4)c3)cc2)cc(C(C)(C)C)c1 ; CC(C)(C)c1ccc(C2(c3ccc(C(C)(C)C)cc3)c3ccccc3-c3ccc(N(c4ccc(-c5cc(C(C)(C)C)cc(C(C)(C)C)c5)cc4)c4ccccc4-c4cc(C5CCCCC5)cc(C5CCCCC5)c4)cc32)cc1]

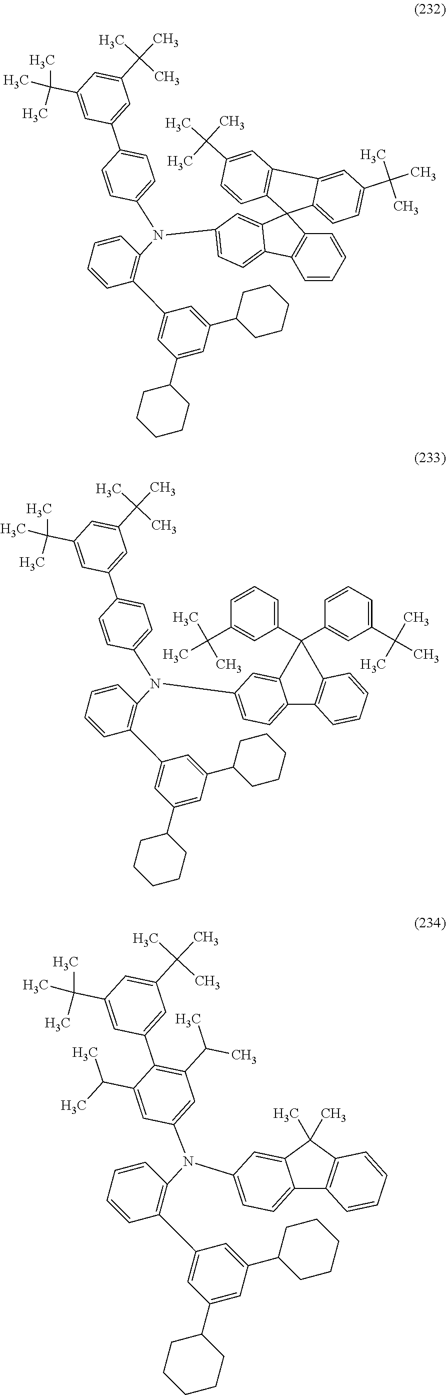

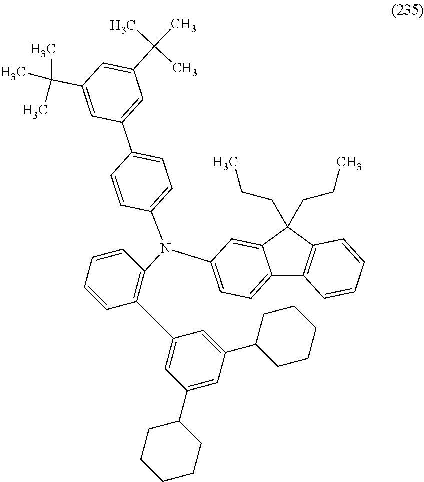 [SMILES: CCCC1(CCC)c2ccccc2-c2ccc(N(c3ccc(-c4cc(C(C)(C)C)cc(C(C)(C)C)c4)cc3)c3ccccc3-c3cc(C4CCCCC4)cc(C4CCCCC4)c3)cc21]

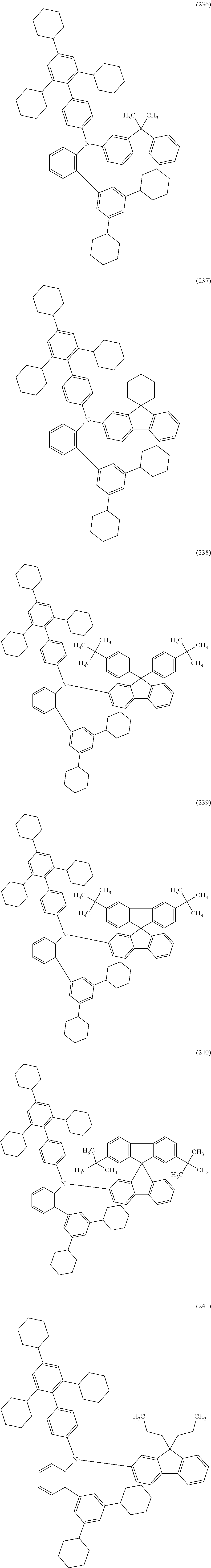

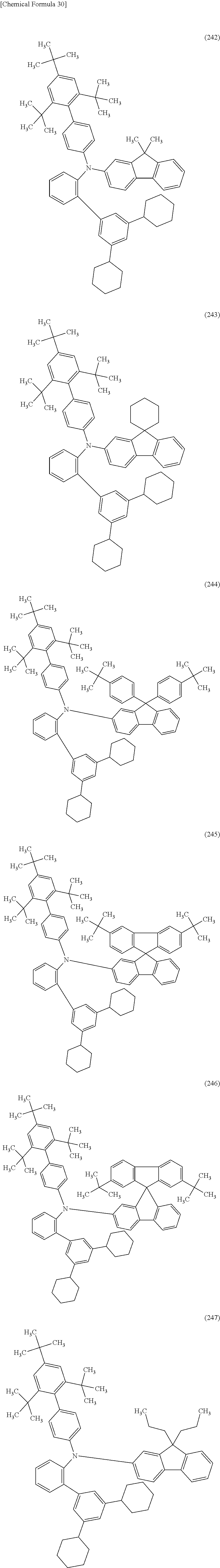

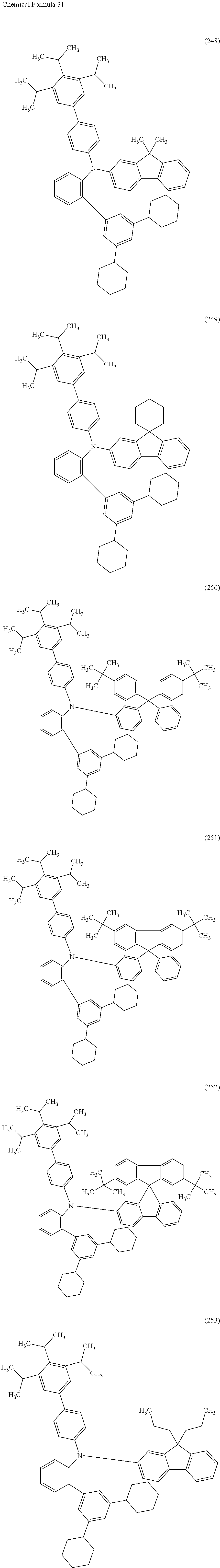

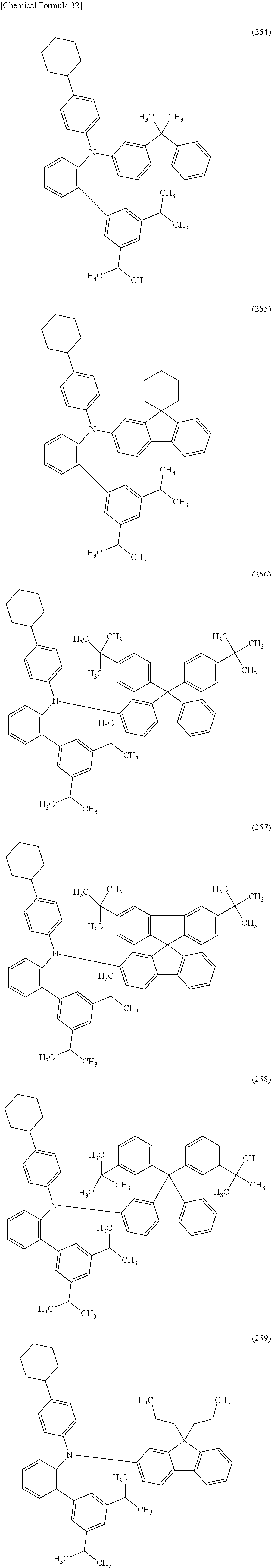

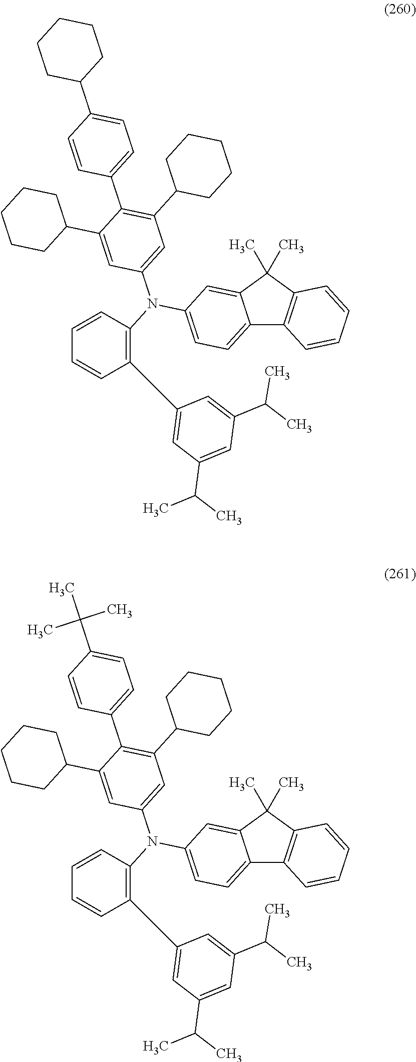 [SMILES: CC(C)c1cc(-c2ccccc2N(c2cc(C3CCCCC3)c(-c3ccc(C4CCCCC4)cc3)c(C3CCCCC3)c2)c2ccc3c(c2)C(C)(C)c2ccccc2-3)cc(C(C)C)c1 ; CC(C)c1cc(-c2ccccc2N(c2cc(C3CCCCC3)c(-c3ccc(C(C)(C)C)cc3)c(C3CCCCC3)c2)c2ccc3c(c2)C(C)(C)c2ccccc2-3)cc(C(C)C)c1]

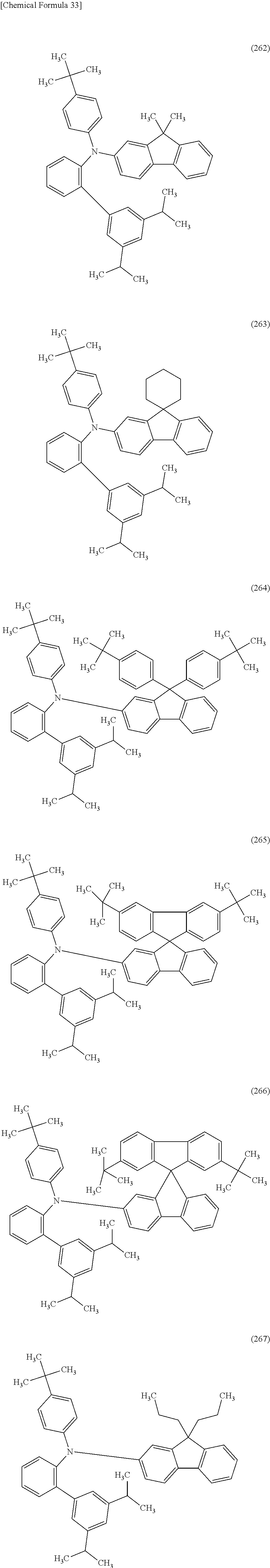

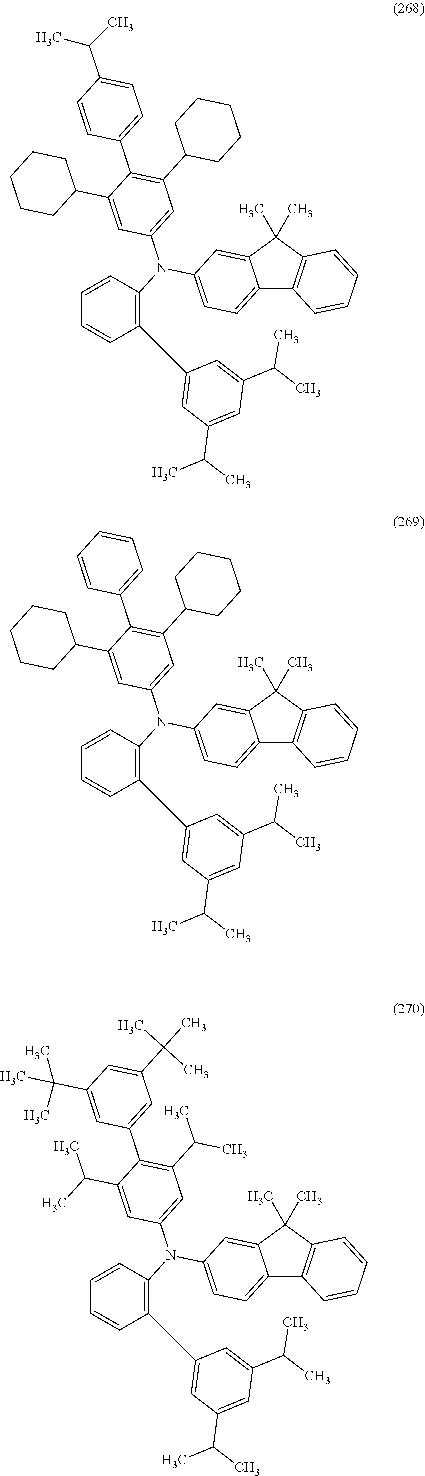 [SMILES: CC(C)c1ccc(-c2c(C3CCCCC3)cc(N(c3ccc4c(c3)C(C)(C)c3ccccc3-4)c3ccccc3-c3cc(C(C)C)cc(C(C)C)c3)cc2C2CCCCC2)cc1 ; CC(C)c1cc(-c2ccccc2N(c2cc(C3CCCCC3)c(-c3ccccc3)c(C3CCCCC3)c2)c2ccc3c(c2)C(C)(C)c2ccccc2-3)cc(C(C)C)c1 ; CC(C)c1cc(-c2ccccc2N(c2cc(C(C)C)c(-c3cc(C(C)(C)C)cc(C(C)(C)C)c3)c(C(C)C)c2)c2ccc3c(c2)C(C)(C)c2ccccc2-3)cc(C(C)C)c1]

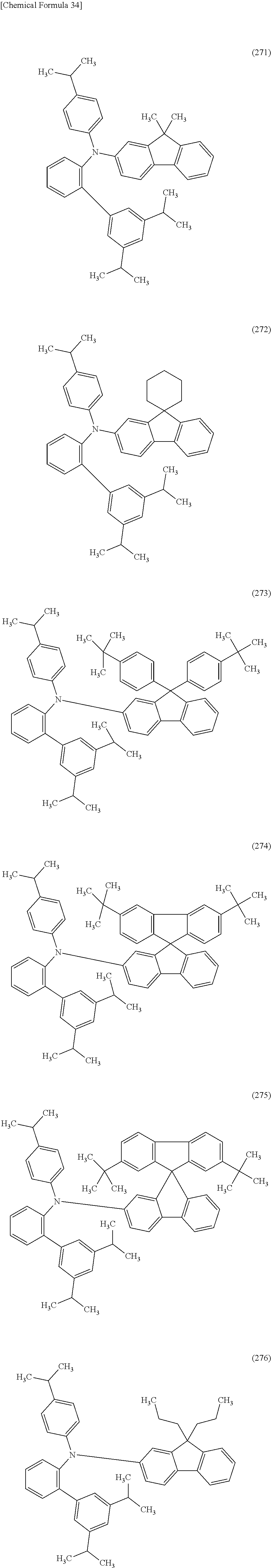

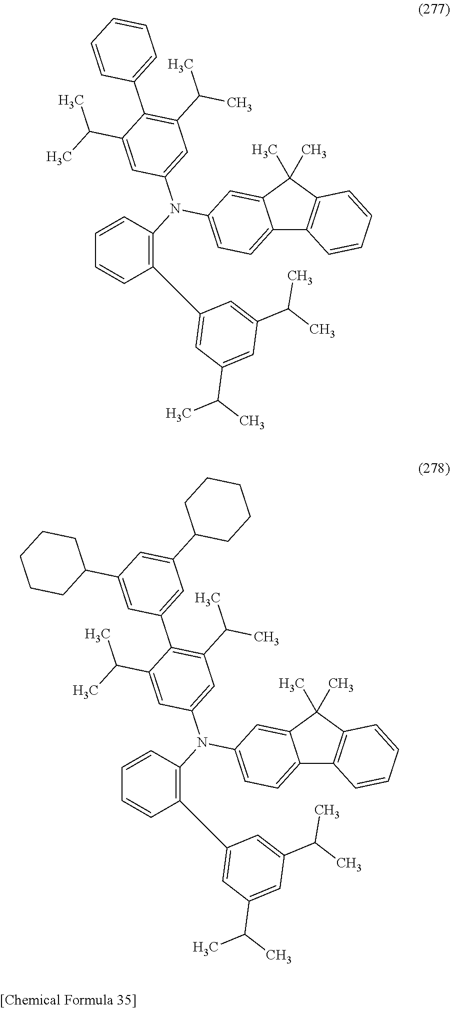 [SMILES: CC(C)c1cc(-c2ccccc2N(c2cc(C(C)C)c(-c3ccccc3)c(C(C)C)c2)c2ccc3c(c2)C(C)(C)c2ccccc2-3)cc(C(C)C)c1 ; CC(C)c1cc(-c2ccccc2N(c2cc(C(C)C)c(-c3cc(C4CCCCC4)cc(C4CCCCC4)c3)c(C(C)C)c2)c2ccc3c(c2)C(C)(C)c2ccccc2-3)cc(C(C)C)c1]

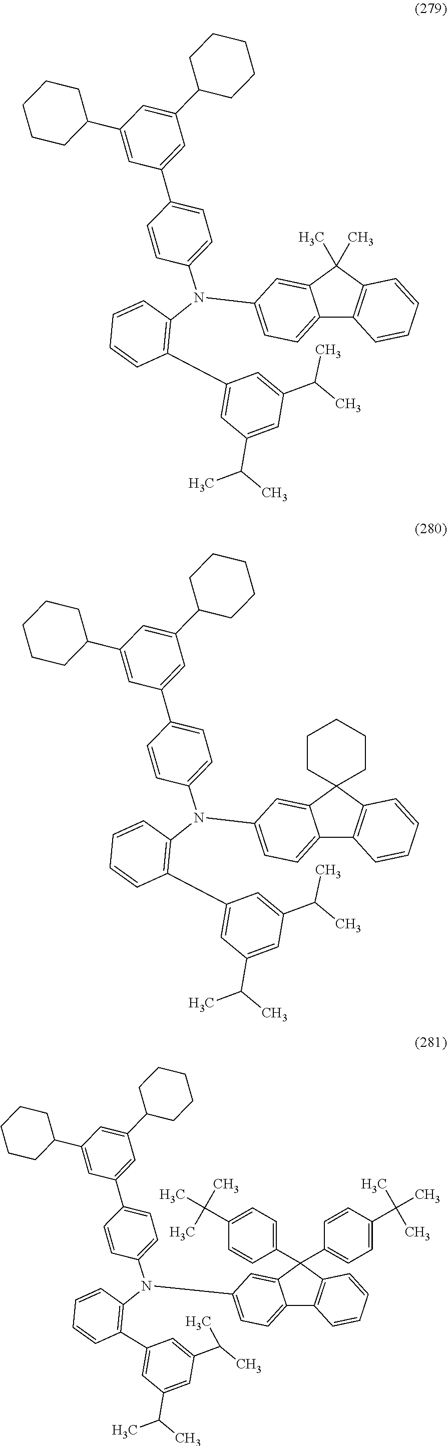 [SMILES: CC(C)c1cc(-c2ccccc2N(c2ccc(-c3cc(C4CCCCC4)cc(C4CCCCC4)c3)cc2)c2ccc3c(c2)C(C)(C)c2ccccc2-3)cc(C(C)C)c1 ; CC(C)c1cc(-c2ccccc2N(c2ccc(-c3cc(C4CCCCC4)cc(C4CCCCC4)c3)cc2)c2ccc3c(c2)C2(CCCCC2)c2ccccc2-3)cc(C(C)C)c1 ; CC(C)c1cc(-c2ccccc2N(c2ccc(-c3cc(C4CCCCC4)cc(C4CCCCC4)c3)cc2)c2ccc3c(c2)C(c2ccc(C(C)(C)C)cc2)(c2ccc(C(C)(C)C)cc2)c2ccccc2-3)cc(C(C)C)c1]

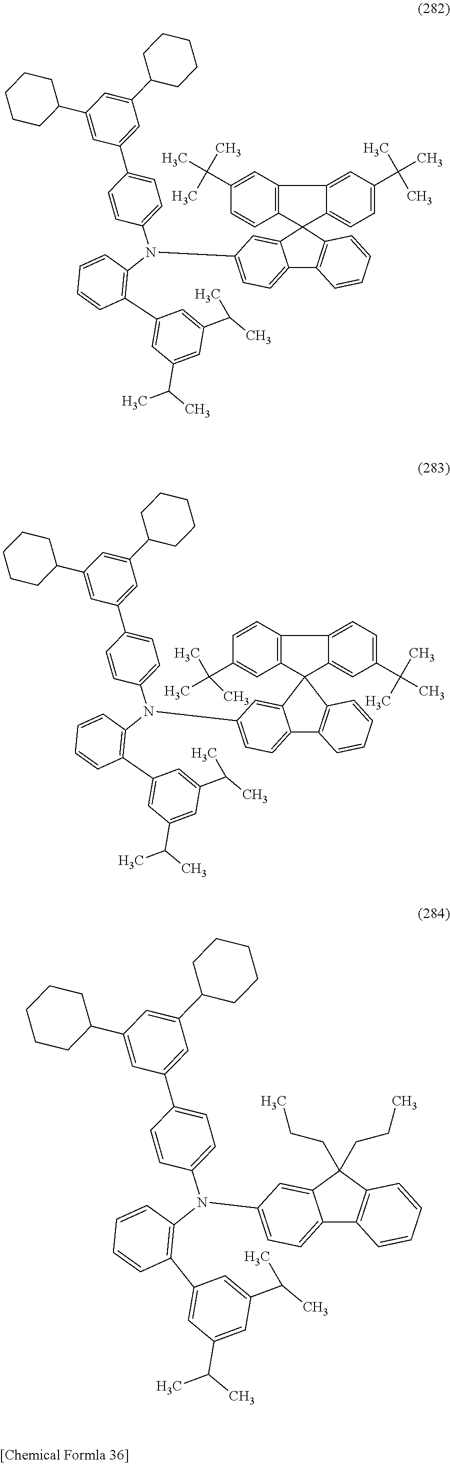 [SMILES: CC(C)c1cc(-c2ccccc2N(c2ccc(-c3cc(C4CCCCC4)cc(C4CCCCC4)c3)cc2)c2ccc3c(c2)C2(c4ccccc4-3)c3ccc(C(C)(C)C)cc3-c3cc(C(C)(C)C)ccc32)cc(C(C)C)c1 ; CC(C)c1cc(-c2ccccc2N(c2ccc(-c3cc(C4CCCCC4)cc(C4CCCCC4)c3)cc2)c2ccc3c(c2)C2(c4ccccc4-3)c3cc(C(C)(C)C)ccc3-c3ccc(C(C)(C)C)cc32)cc(C(C)C)c1 ; CCCC1(CCC)c2ccccc2-c2ccc(N(c3ccc(-c4cc(C5CCCCC5)cc(C5CCCCC5)c4)cc3)c3ccccc3-c3cc(C(C)C)cc(C(C)C)c3)cc21]

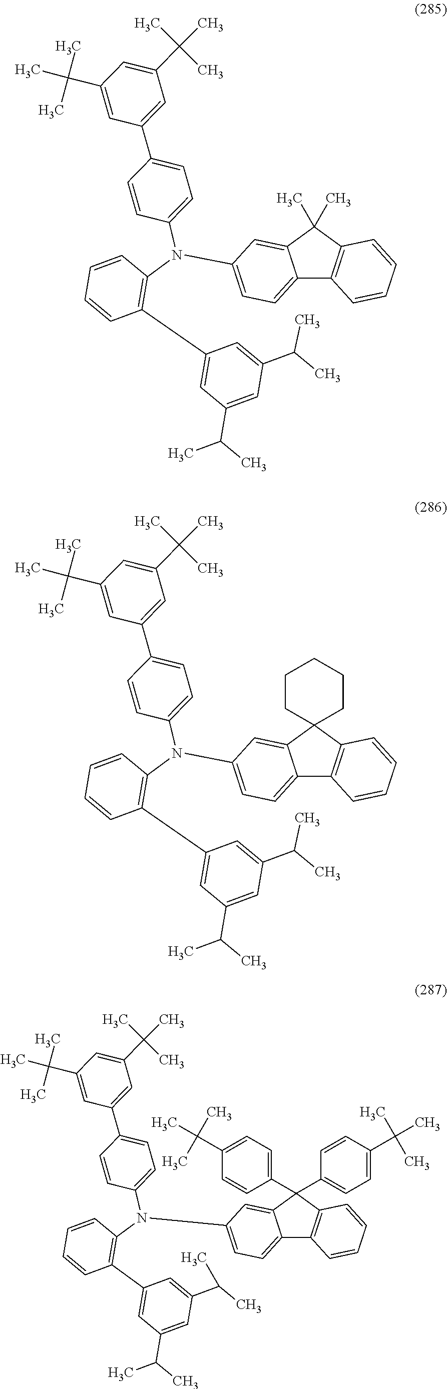 [SMILES: CC(C)c1cc(-c2ccccc2N(c2ccc(-c3cc(C(C)(C)C)cc(C(C)(C)C)c3)cc2)c2ccc3c(c2)C(C)(C)c2ccccc2-3)cc(C(C)C)c1 ; CC(C)c1cc(-c2ccccc2N(c2ccc(-c3cc(C(C)(C)C)cc(C(C)(C)C)c3)cc2)c2ccc3c(c2)C2(CCCCC2)c2ccccc2-3)cc(C(C)C)c1 ; CC(C)c1cc(-c2ccccc2N(c2ccc(-c3cc(C(C)(C)C)cc(C(C)(C)C)c3)cc2)c2ccc3c(c2)C(c2ccc(C(C)(C)C)cc2)(c2ccc(C(C)(C)C)cc2)c2ccccc2-3)cc(C(C)C)c1]

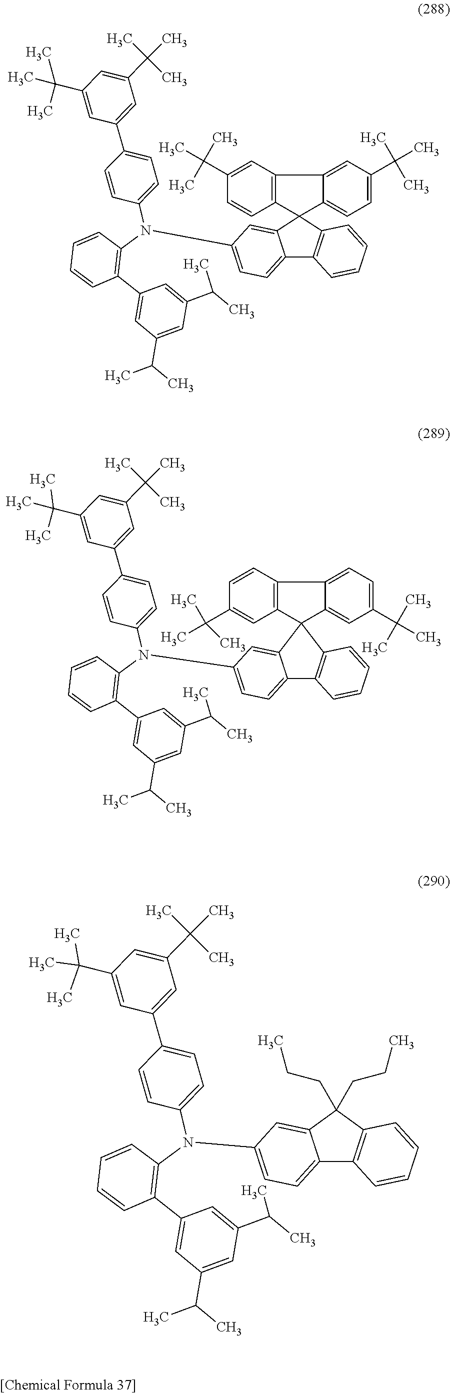

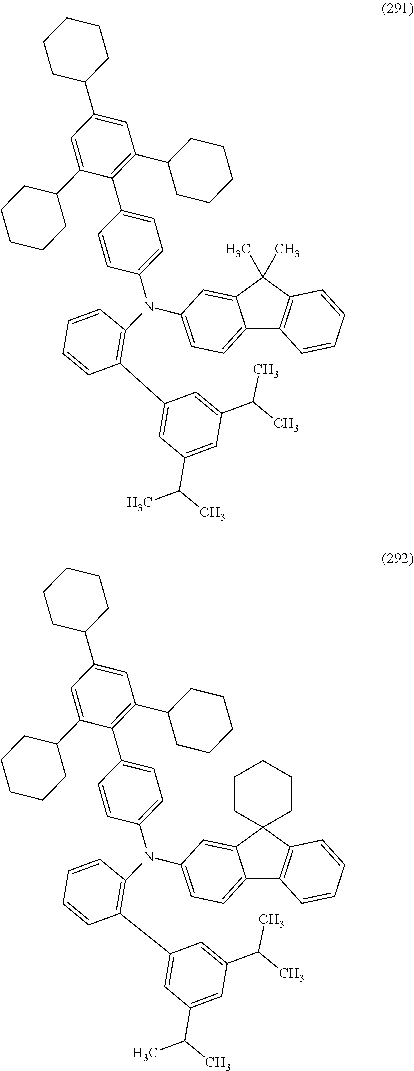 [SMILES: CC(C)c1cc(-c2ccccc2N(c2ccc(-c3c(C4CCCCC4)cc(C4CCCCC4)cc3C3CCCCC3)cc2)c2ccc3c(c2)C(C)(C)c2ccccc2-3)cc(C(C)C)c1 ; CC(C)c1cc(-c2ccccc2N(c2ccc(-c3c(C4CCCCC4)cc(C4CCCCC4)cc3C3CCCCC3)cc2)c2ccc3c(c2)C2(CCCCC2)c2ccccc2-3)cc(C(C)C)c1]

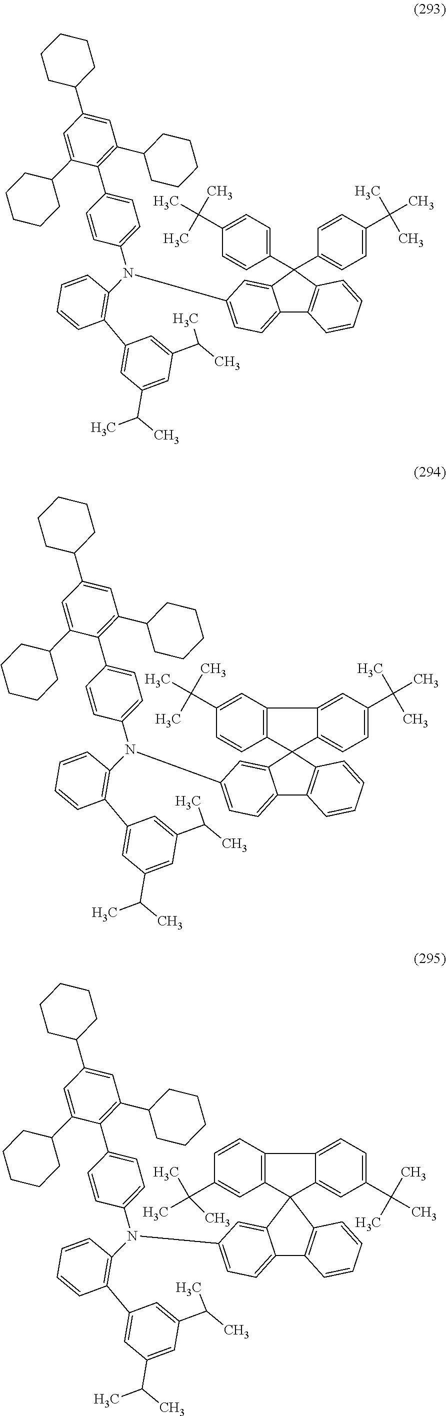 [SMILES: CC(C)c1cc(-c2ccccc2N(c2ccc(-c3c(C4CCCCC4)cc(C4CCCCC4)cc3C3CCCCC3)cc2)c2ccc3c(c2)C(c2ccc(C(C)(C)C)cc2)(c2ccc(C(C)(C)C)cc2)c2ccccc2-3)cc(C(C)C)c1 ; CC(C)c1cc(-c2ccccc2N(c2ccc(-c3c(C4CCCCC4)cc(C4CCCCC4)cc3C3CCCCC3)cc2)c2ccc3c(c2)C2(c4ccccc4-3)c3ccc(C(C)(C)C)cc3-c3cc(C(C)(C)C)ccc32)cc(C(C)C)c1 ; CC(C)c1cc(-c2ccccc2N(c2ccc(-c3c(C4CCCCC4)cc(C4CCCCC4)cc3C3CCCCC3)cc2)c2ccc3c(c2)C2(c4ccccc4-3)c3cc(C(C)(C)C)ccc3-c3ccc(C(C)(C)C)cc32)cc(C(C)C)c1]

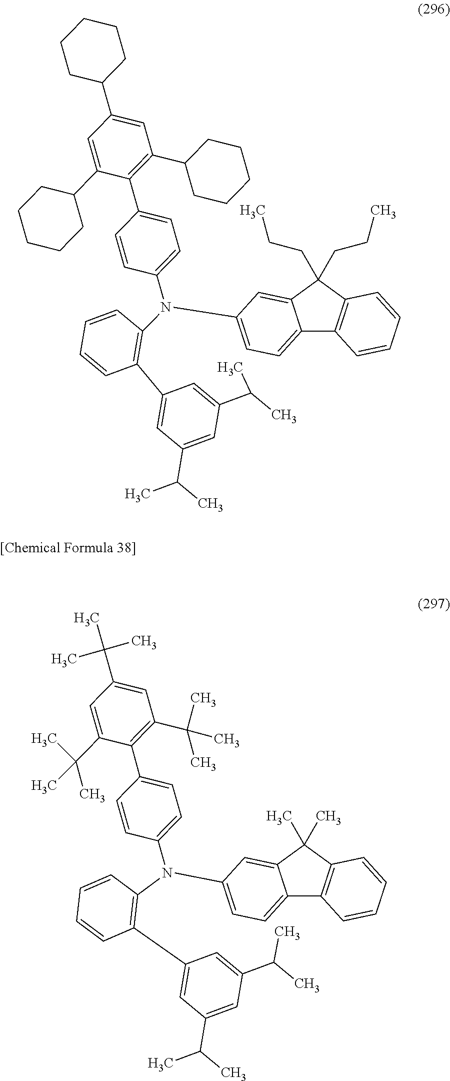 [SMILES: CCCC1(CCC)c2ccccc2-c2ccc(N(c3ccc(-c4c(C5CCCCC5)cc(C5CCCCC5)cc4C4CCCCC4)cc3)c3ccccc3-c3cc(C(C)C)cc(C(C)C)c3)cc21 ; CC(C)c1cc(-c2ccccc2N(c2ccc(-c3c(C(C)(C)C)cc(C(C)(C)C)cc3C(C)(C)C)cc2)c2ccc3c(c2)C(C)(C)c2ccccc2-3)cc(C(C)C)c1]

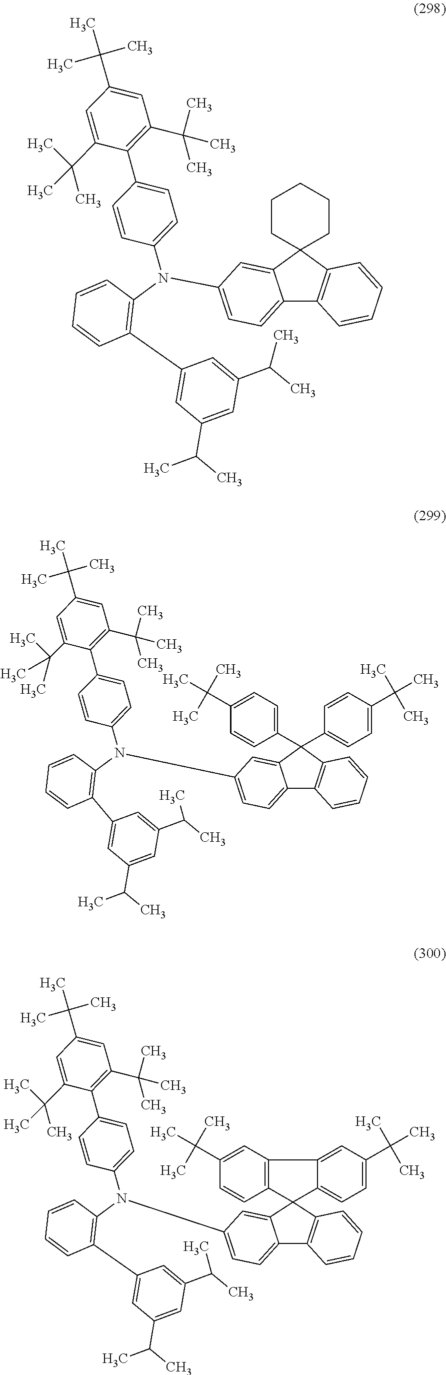

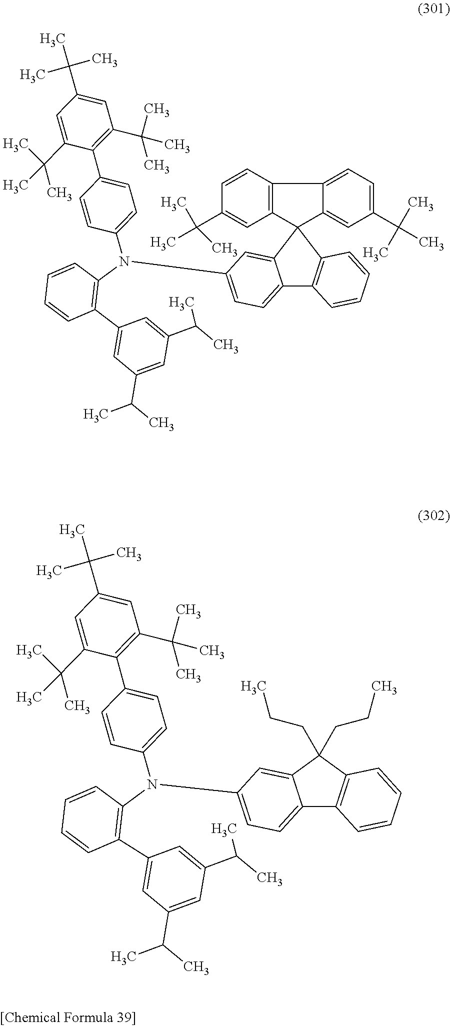

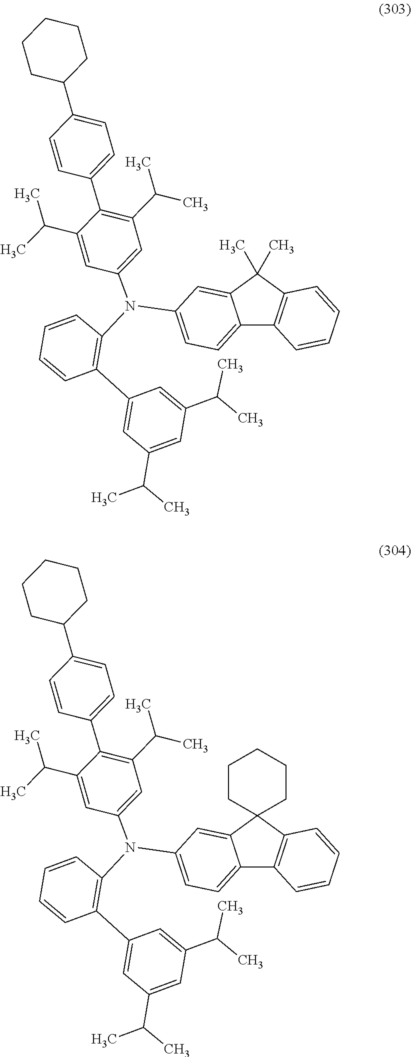 [SMILES: CC(C)c1cc(-c2ccccc2N(c2cc(C(C)C)c(-c3ccc(C4CCCCC4)cc3)c(C(C)C)c2)c2ccc3c(c2)C(C)(C)c2ccccc2-3)cc(C(C)C)c1 ; CC(C)c1cc(-c2ccccc2N(c2cc(C(C)C)c(-c3ccc(C4CCCCC4)cc3)c(C(C)C)c2)c2ccc3c(c2)C2(CCCCC2)c2ccccc2-3)cc(C(C)C)c1]

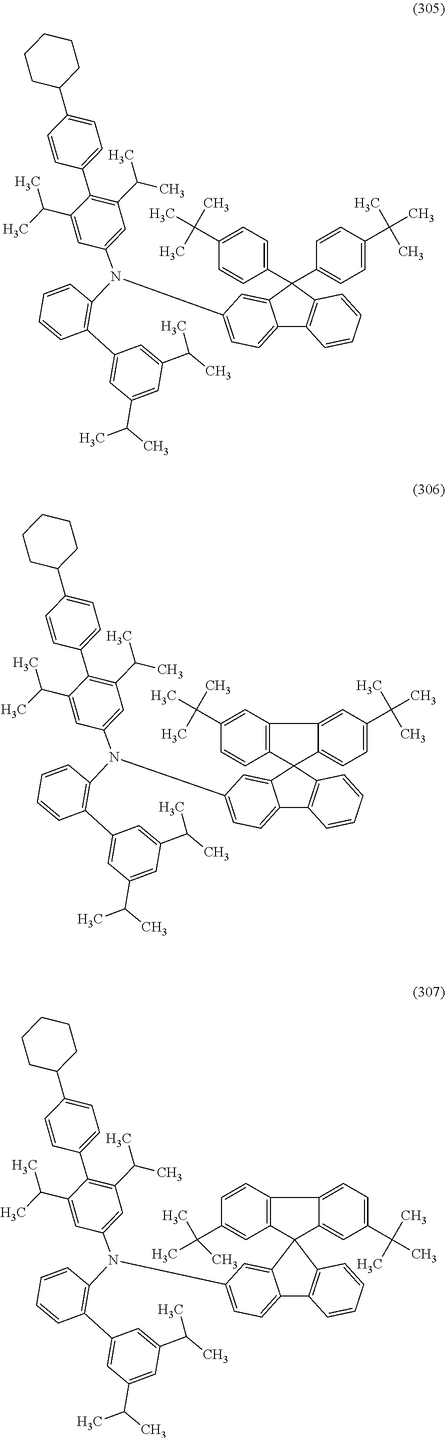

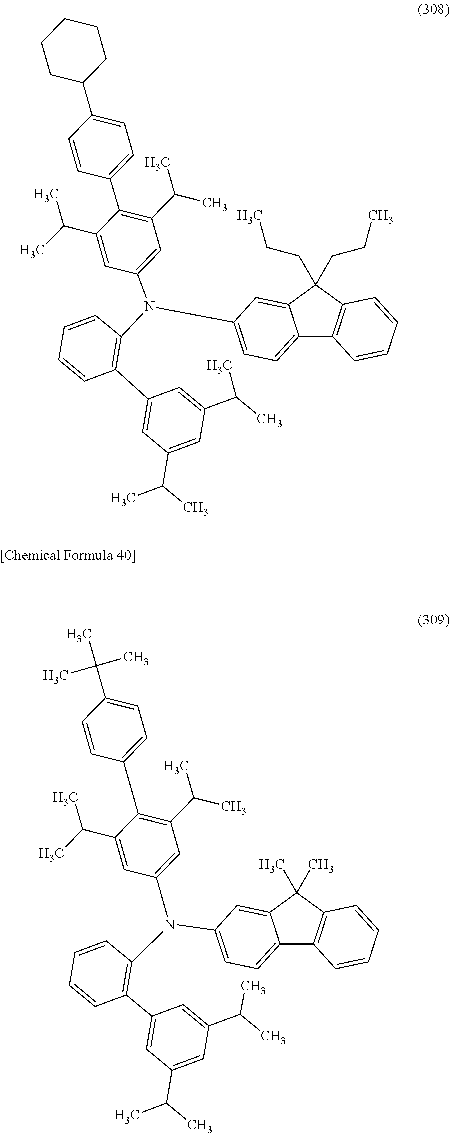 [SMILES: CCCC1(CCC)c2ccccc2-c2ccc(N(c3cc(C(C)C)c(-c4ccc(C5CCCCC5)cc4)c(C(C)C)c3)c3ccccc3-c3cc(C(C)C)cc(C(C)C)c3)cc21 ; CC(C)c1cc(-c2ccccc2N(c2cc(C(C)C)c(-c3ccc(C(C)(C)C)cc3)c(C(C)C)c2)c2ccc3c(c2)C(C)(C)c2ccccc2-3)cc(C(C)C)c1]

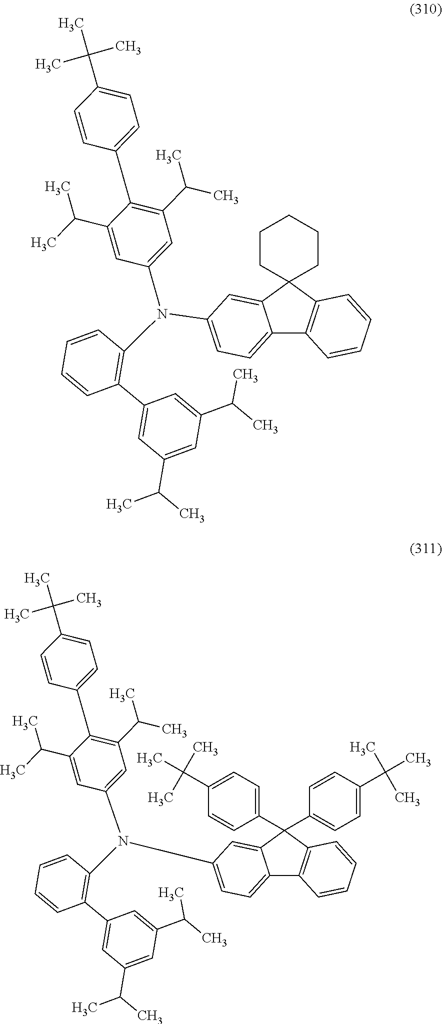 [SMILES: CC(C)c1cc(-c2ccccc2N(c2cc(C(C)C)c(-c3ccc(C(C)(C)C)cc3)c(C(C)C)c2)c2ccc3c(c2)C2(CCCCC2)c2ccccc2-3)cc(C(C)C)c1 ; CC(C)c1cc(-c2ccccc2N(c2cc(C(C)C)c(-c3ccc(C(C)(C)C)cc3)c(C(C)C)c2)c2ccc3c(c2)C(c2ccc(C(C)(C)C)cc2)(c2ccc(C(C)(C)C)cc2)c2ccccc2-3)cc(C(C)C)c1]

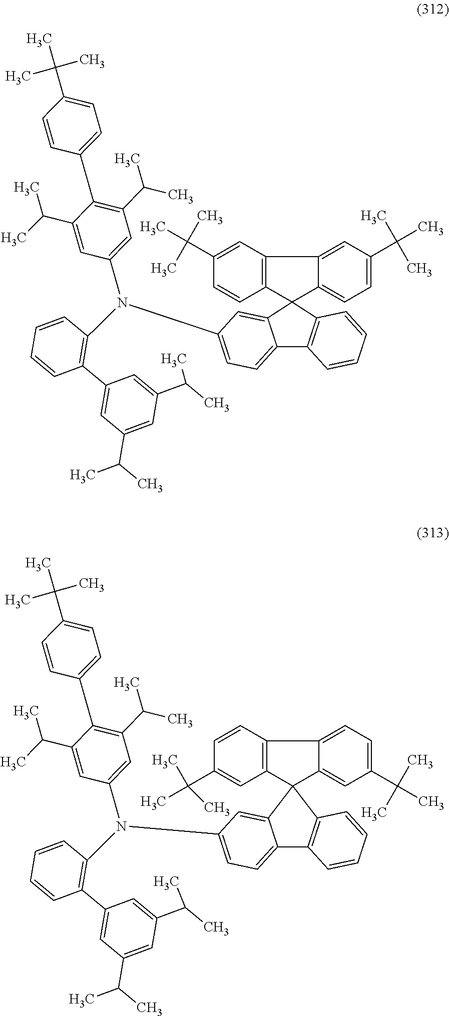

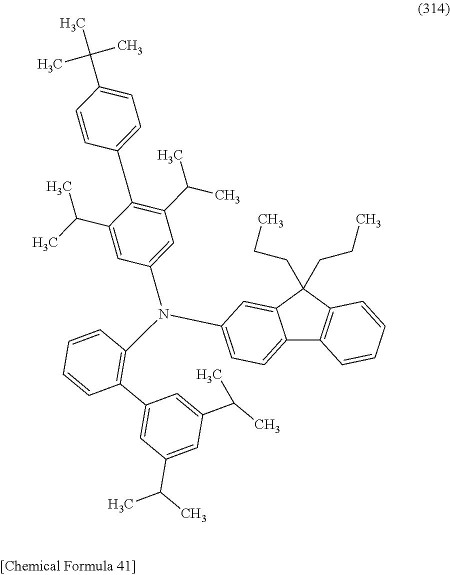 [SMILES: CCCC1(CCC)c2ccccc2-c2ccc(N(c3cc(C(C)C)c(-c4ccc(C(C)(C)C)cc4)c(C(C)C)c3)c3ccccc3-c3cc(C(C)C)cc(C(C)C)c3)cc21]

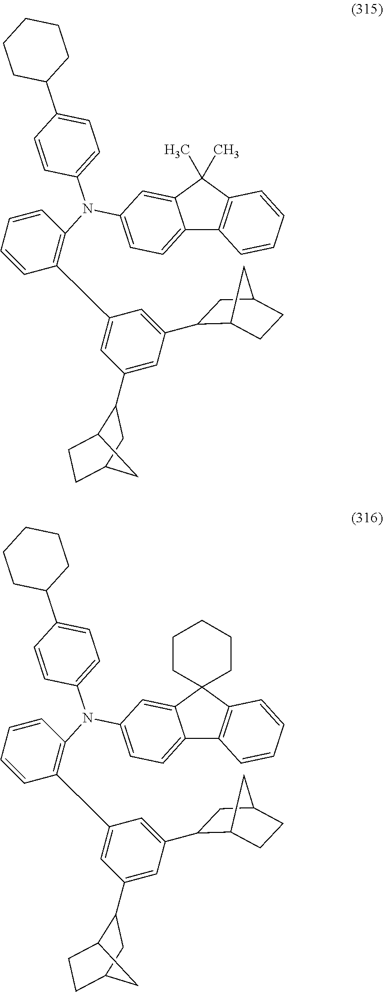 [SMILES: CC1(C)c2ccccc2-c2ccc(N(c3ccc(C4CCCCC4)cc3)c3ccccc3-c3cc(C4CC5CCC4C5)cc(C4CC5CCC4C5)c3)cc21 ; c1ccc(N(c2ccc(C3CCCCC3)cc2)c2ccc3c(c2)C2(CCCCC2)c2ccccc2-3)c(-c2cc(C3CC4CCC3C4)cc(C3CC4CCC3C4)c2)c1]

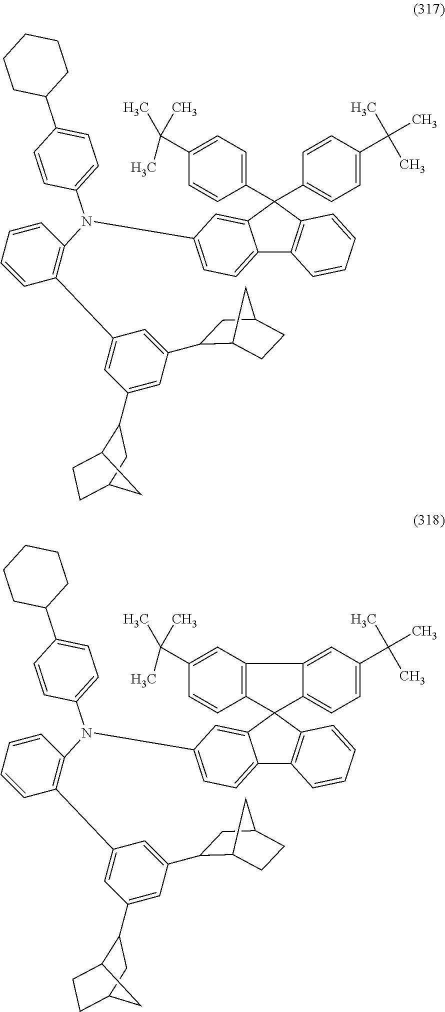 [SMILES: CC(C)(C)c1ccc(C2(c3ccc(C(C)(C)C)cc3)c3ccccc3-c3ccc(N(c4ccc(C5CCCCC5)cc4)c4ccccc4-c4cc(C5CC6CCC5C6)cc(C5CC6CCC5C6)c4)cc32)cc1 ; CC(C)(C)c1ccc2c(c1)-c1cc(C(C)(C)C)ccc1C21c2ccccc2-c2ccc(N(c3ccc(C4CCCCC4)cc3)c3ccccc3-c3cc(C4CC5CCC4C5)cc(C4CC5CCC4C5)c3)cc21]

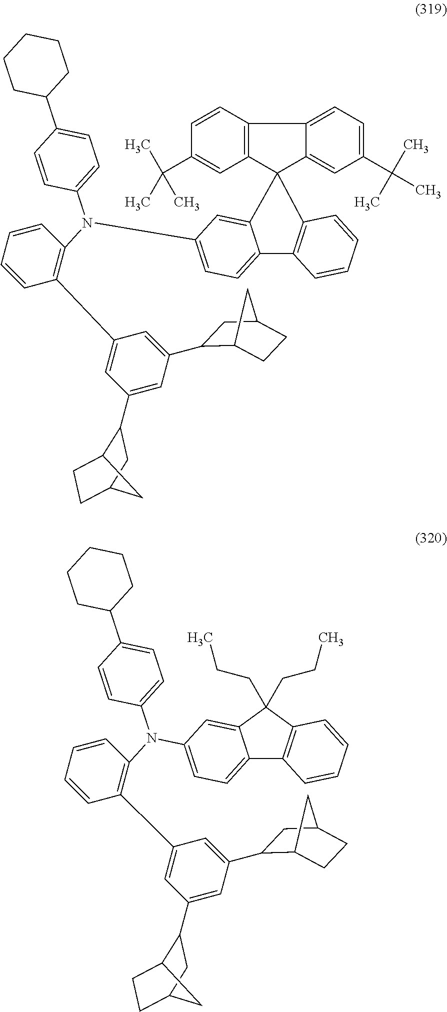

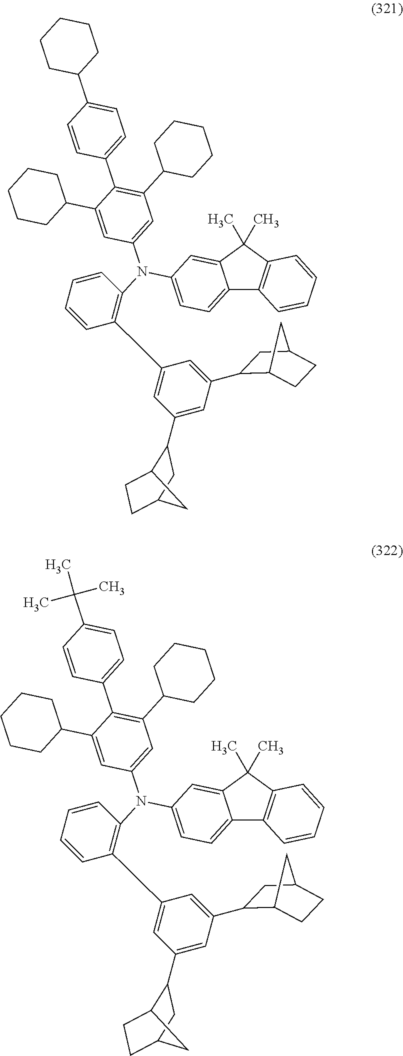 [SMILES: CC1(C)c2ccccc2-c2ccc(N(c3cc(C4CCCCC4)c(-c4ccc(C5CCCCC5)cc4)c(C4CCCCC4)c3)c3ccccc3-c3cc(C4CC5CCC4C5)cc(C4CC5CCC4C5)c3)cc21 ; CC(C)(C)c1ccc(-c2c(C3CCCCC3)cc(N(c3ccc4c(c3)C(C)(C)c3ccccc3-4)c3ccccc3-c3cc(C4CC5CCC4C5)cc(C4CC5CCC4C5)c3)cc2C2CCCCC2)cc1]

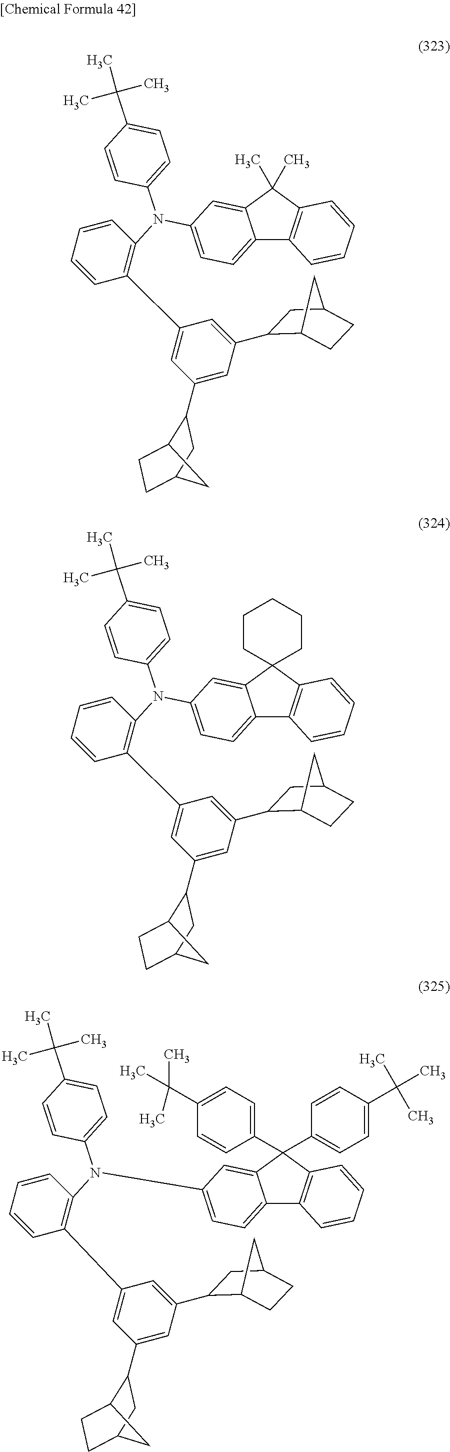 [SMILES: CC(C)(C)c1ccc(N(c2ccc3c(c2)C(C)(C)c2ccccc2-3)c2ccccc2-c2cc(C3CC4CCC3C4)cc(C3CC4CCC3C4)c2)cc1 ; CC(C)(C)c1ccc(N(c2ccc3c(c2)C2(CCCCC2)c2ccccc2-3)c2ccccc2-c2cc(C3CC4CCC3C4)cc(C3CC4CCC3C4)c2)cc1 ; CC(C)(C)c1ccc(N(c2ccc3c(c2)C(c2ccc(C(C)(C)C)cc2)(c2ccc(C(C)(C)C)cc2)c2ccccc2-3)c2ccccc2-c2cc(C3CC4CCC3C4)cc(C3CC4CCC3C4)c2)cc1]

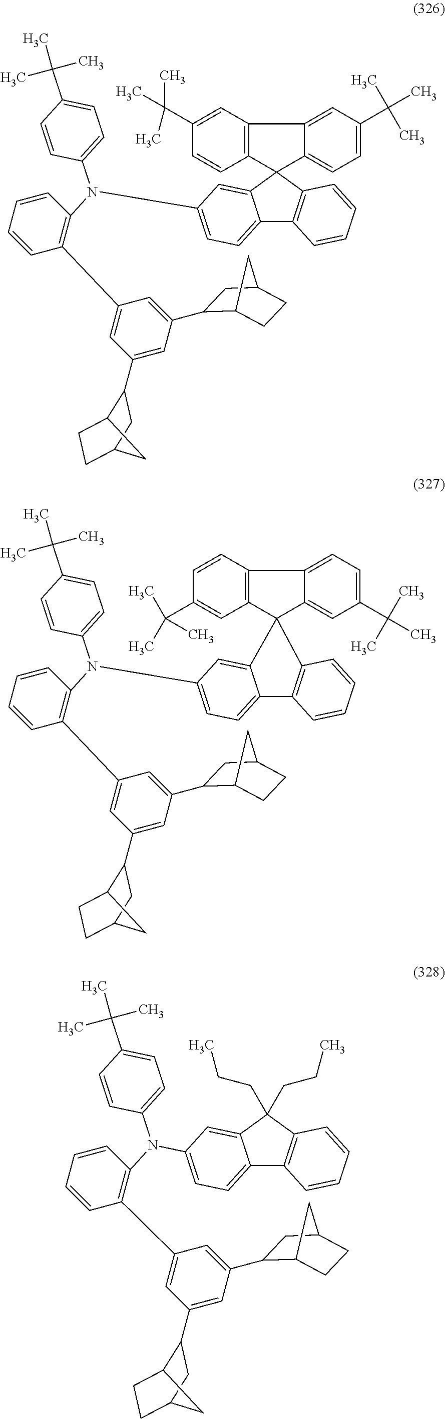 [SMILES: CC(C)(C)c1ccc(N(c2ccc3c(c2)C2(c4ccccc4-3)c3ccc(C(C)(C)C)cc3-c3cc(C(C)(C)C)ccc32)c2ccccc2-c2cc(C3CC4CCC3C4)cc(C3CC4CCC3C4)c2)cc1 ; CC(C)(C)c1ccc(N(c2ccc3c(c2)C2(c4ccccc4-3)c3cc(C(C)(C)C)ccc3-c3ccc(C(C)(C)C)cc32)c2ccccc2-c2cc(C3CC4CCC3C4)cc(C3CC4CCC3C4)c2)cc1 ; CCCC1(CCC)c2ccccc2-c2ccc(N(c3ccc(C(C)(C)C)cc3)c3ccccc3-c3cc(C4CC5CCC4C5)cc(C4CC5CCC4C5)c3)cc21]

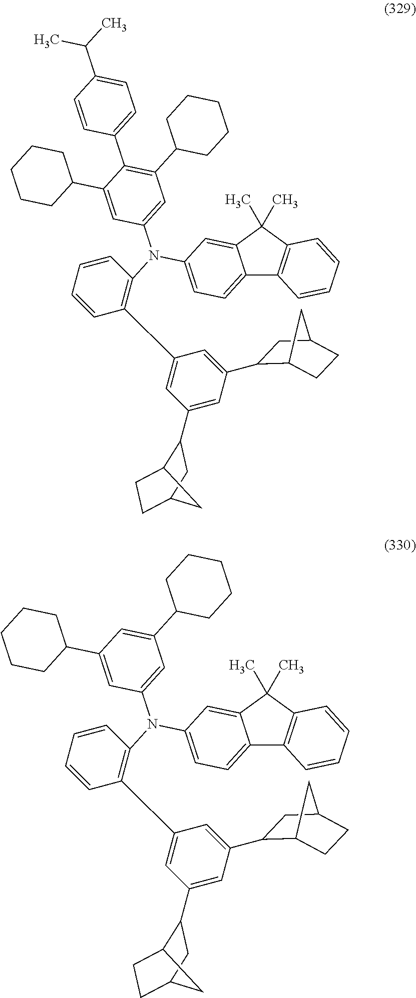 [SMILES: CC(C)c1ccc(-c2c(C3CCCCC3)cc(N(c3ccc4c(c3)C(C)(C)c3ccccc3-4)c3ccccc3-c3cc(C4CC5CCC4C5)cc(C4CC5CCC4C5)c3)cc2C2CCCCC2)cc1 ; CC1(C)c2ccccc2-c2ccc(N(c3cc(C4CCCCC4)cc(C4CCCCC4)c3)c3ccccc3-c3cc(C4CC5CCC4C5)cc(C4CC5CCC4C5)c3)cc21]

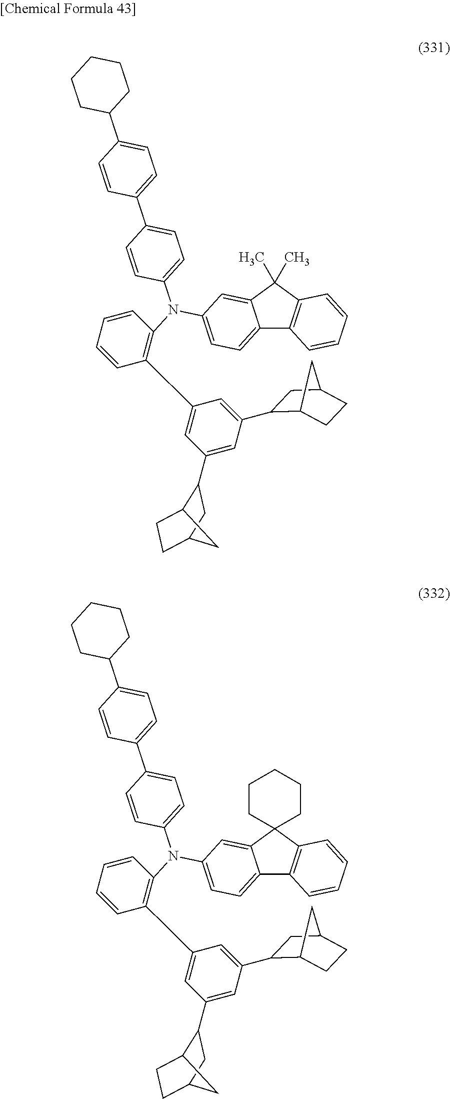 [SMILES: CC1(C)c2ccccc2-c2ccc(N(c3ccc(-c4ccc(C5CCCCC5)cc4)cc3)c3ccccc3-c3cc(C4CC5CCC4C5)cc(C4CC5CCC4C5)c3)cc21 ; c1ccc(N(c2ccc(-c3ccc(C4CCCCC4)cc3)cc2)c2ccc3c(c2)C2(CCCCC2)c2ccccc2-3)c(-c2cc(C3CC4CCC3C4)cc(C3CC4CCC3C4)c2)c1]

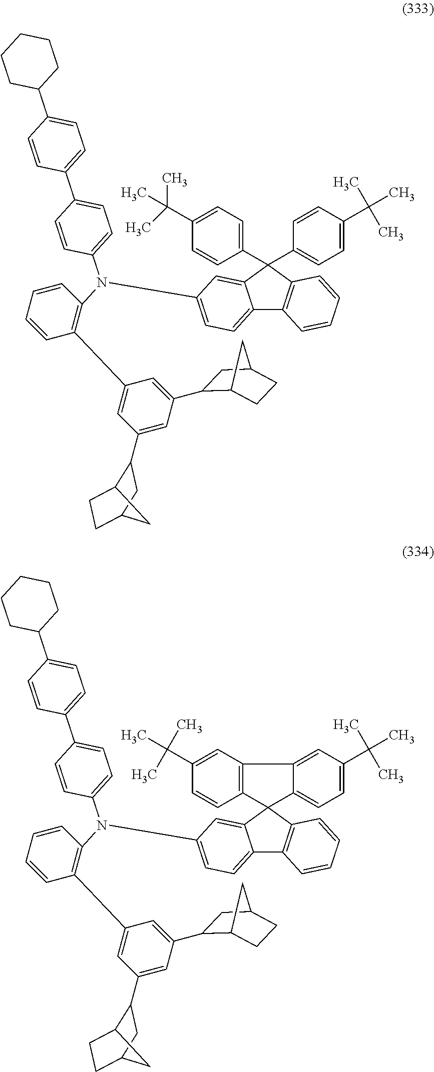 [SMILES: CC(C)(C)c1ccc(C2(c3ccc(C(C)(C)C)cc3)c3ccccc3-c3ccc(N(c4ccc(-c5ccc(C6CCCCC6)cc5)cc4)c4ccccc4-c4cc(C5CC6CCC5C6)cc(C5CC6CCC5C6)c4)cc32)cc1 ; CC(C)(C)c1ccc2c(c1)-c1cc(C(C)(C)C)ccc1C21c2ccccc2-c2ccc(N(c3ccc(-c4ccc(C5CCCCC5)cc4)cc3)c3ccccc3-c3cc(C4CC5CCC4C5)cc(C4CC5CCC4C5)c3)cc21]

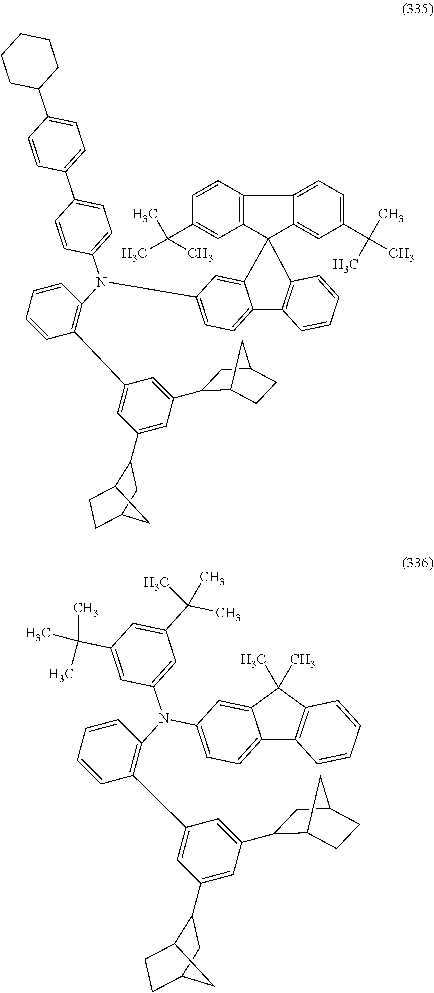

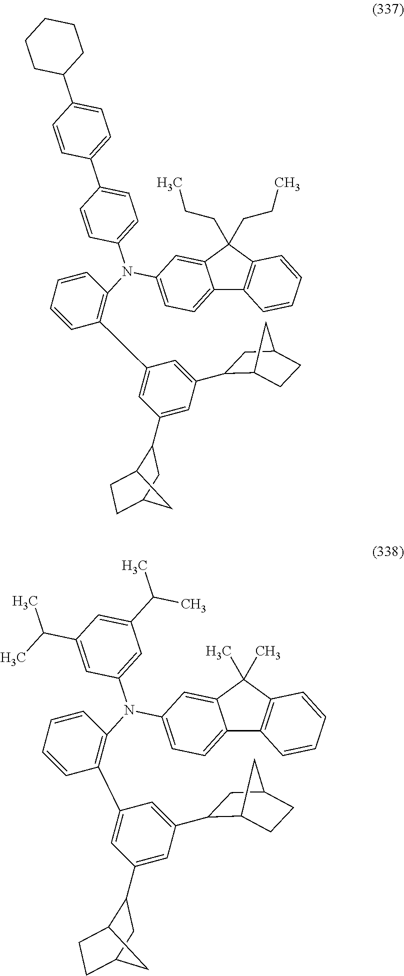 [SMILES: CCCC1(CCC)c2ccccc2-c2ccc(N(c3ccc(-c4ccc(C5CCCCC5)cc4)cc3)c3ccccc3-c3cc(C4CC5CCC4C5)cc(C4CC5CCC4C5)c3)cc21 ; CC(C)c1cc(C(C)C)cc(N(c2ccc3c(c2)C(C)(C)c2ccccc2-3)c2ccccc2-c2cc(C3CC4CCC3C4)cc(C3CC4CCC3C4)c2)c1]

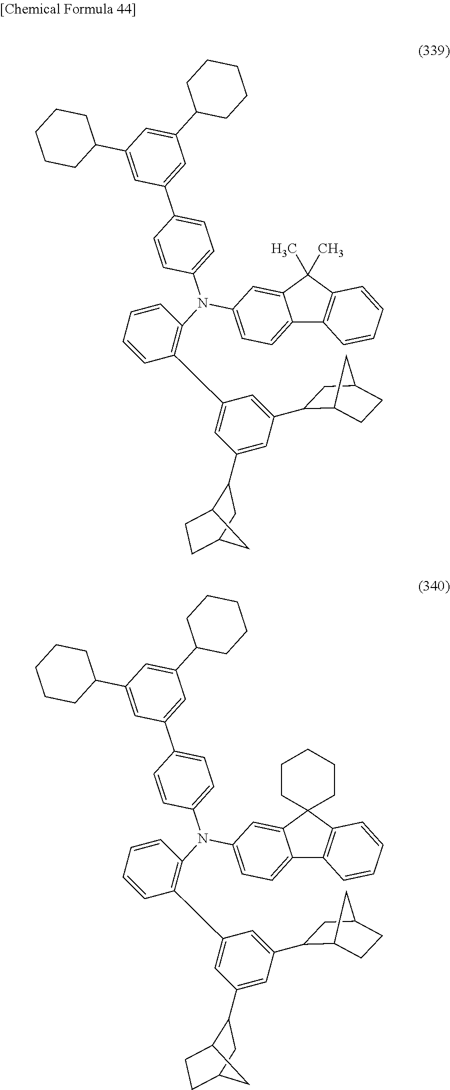 [SMILES: CC1(C)c2ccccc2-c2ccc(N(c3ccc(-c4cc(C5CCCCC5)cc(C5CCCCC5)c4)cc3)c3ccccc3-c3cc(C4CC5CCC4C5)cc(C4CC5CCC4C5)c3)cc21 ; c1ccc(N(c2ccc(-c3cc(C4CCCCC4)cc(C4CCCCC4)c3)cc2)c2ccc3c(c2)C2(CCCCC2)c2ccccc2-3)c(-c2cc(C3CC4CCC3C4)cc(C3CC4CCC3C4)c2)c1]

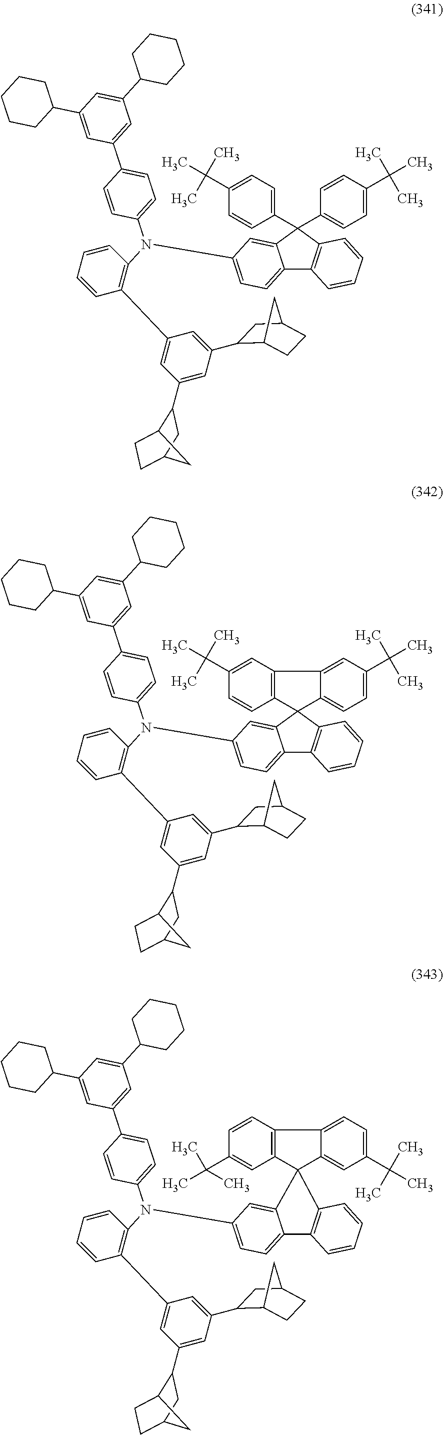 [SMILES: CC(C)(C)c1ccc(C2(c3ccc(C(C)(C)C)cc3)c3ccccc3-c3ccc(N(c4ccc(-c5cc(C6CCCCC6)cc(C6CCCCC6)c5)cc4)c4ccccc4-c4cc(C5CC6CCC5C6)cc(C5CC6CCC5C6)c4)cc32)cc1 ; CC(C)(C)c1ccc2c(c1)-c1cc(C(C)(C)C)ccc1C21c2ccccc2-c2ccc(N(c3ccc(-c4cc(C5CCCCC5)cc(C5CCCCC5)c4)cc3)c3ccccc3-c3cc(C4CC5CCC4C5)cc(C4CC5CCC4C5)c3)cc21 ; CC(C)(C)c1ccc2c(c1)C1(c3ccccc3-c3ccc(N(c4ccc(-c5cc(C6CCCCC6)cc(C6CCCCC6)c5)cc4)c4ccccc4-c4cc(C5CC6CCC5C6)cc(C5CC6CCC5C6)c4)cc31)c1cc(C(C)(C)C)ccc1-2]

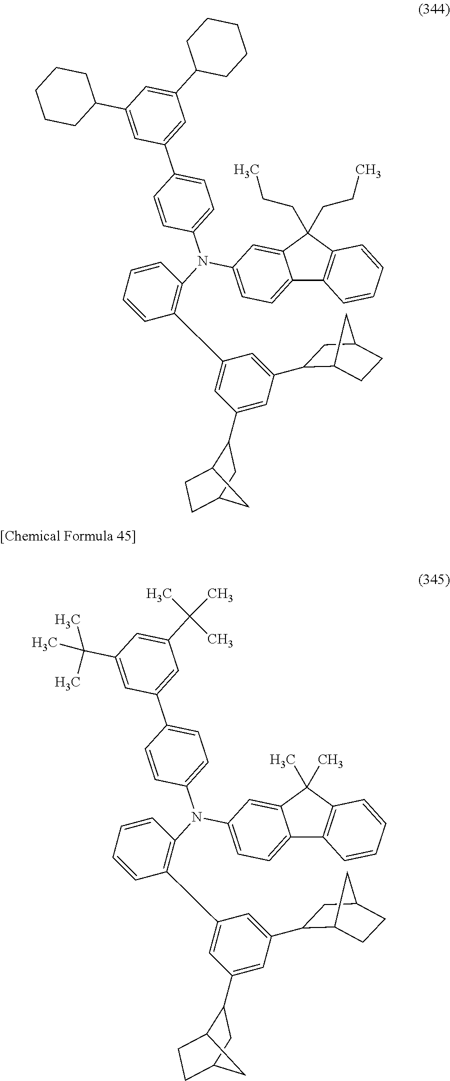 [SMILES: CCCC1(CCC)c2ccccc2-c2ccc(N(c3ccc(-c4cc(C5CCCCC5)cc(C5CCCCC5)c4)cc3)c3ccccc3-c3cc(C4CC5CCC4C5)cc(C4CC5CCC4C5)c3)cc21 ; CC(C)(C)c1cc(-c2ccc(N(c3ccc4c(c3)C(C)(C)c3ccccc3-4)c3ccccc3-c3cc(C4CC5CCC4C5)cc(C4CC5CCC4C5)c3)cc2)cc(C(C)(C)C)c1]

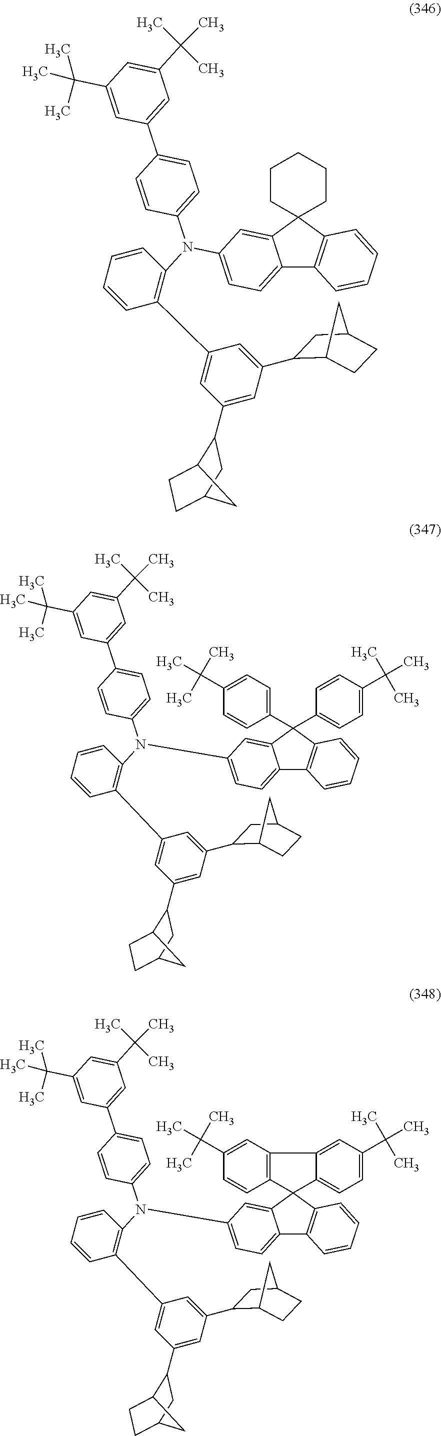

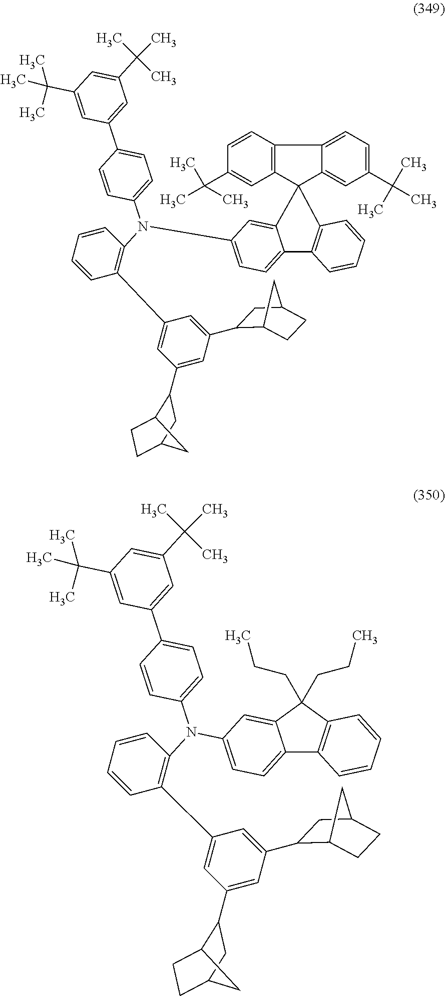

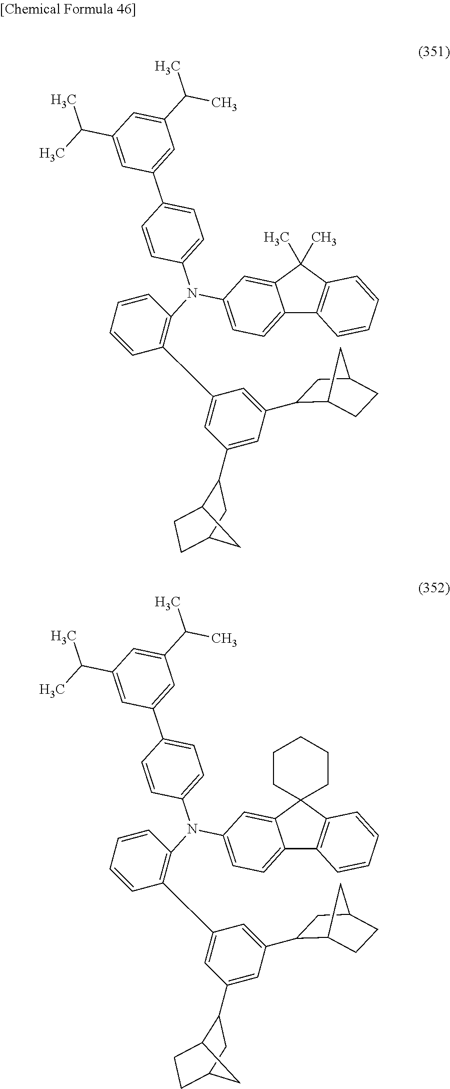 [SMILES: CC(C)c1cc(-c2ccc(N(c3ccc4c(c3)C(C)(C)c3ccccc3-4)c3ccccc3-c3cc(C4CC5CCC4C5)cc(C4CC5CCC4C5)c3)cc2)cc(C(C)C)c1 ; CC(C)c1cc(-c2ccc(N(c3ccc4c(c3)C3(CCCCC3)c3ccccc3-4)c3ccccc3-c3cc(C4CC5CCC4C5)cc(C4CC5CCC4C5)c3)cc2)cc(C(C)C)c1]

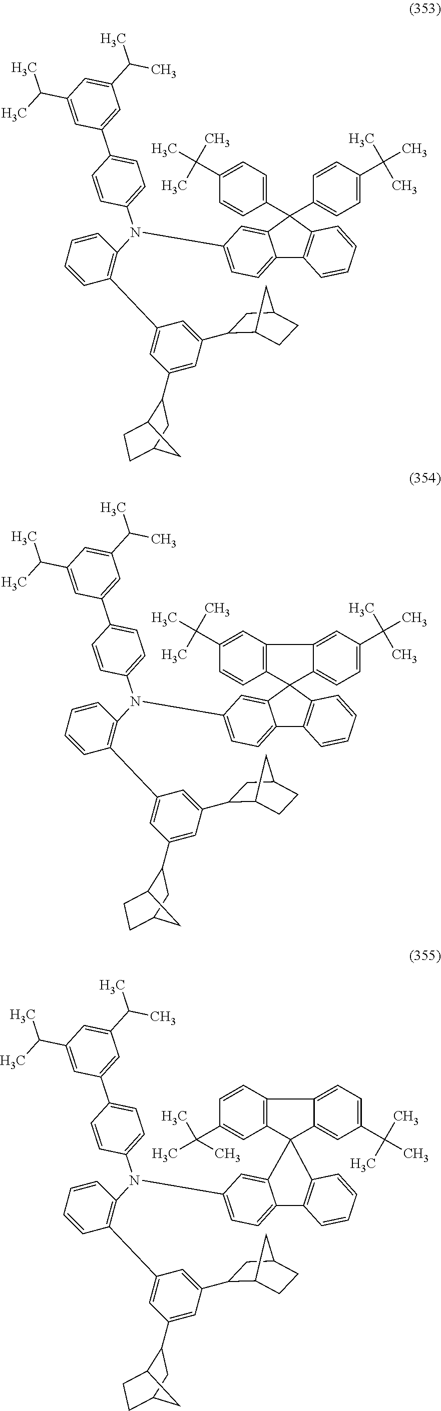

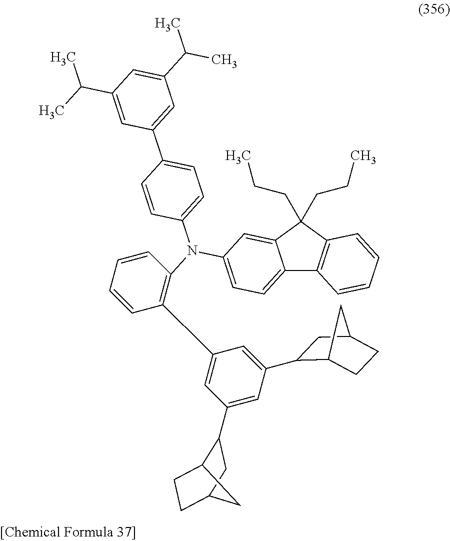 [SMILES: CCCC1(CCC)c2ccccc2-c2ccc(N(c3ccc(-c4cc(C(C)C)cc(C(C)C)c4)cc3)c3ccccc3-c3cc(C4CC5CCC4C5)cc(C4CC5CCC4C5)c3)cc21]

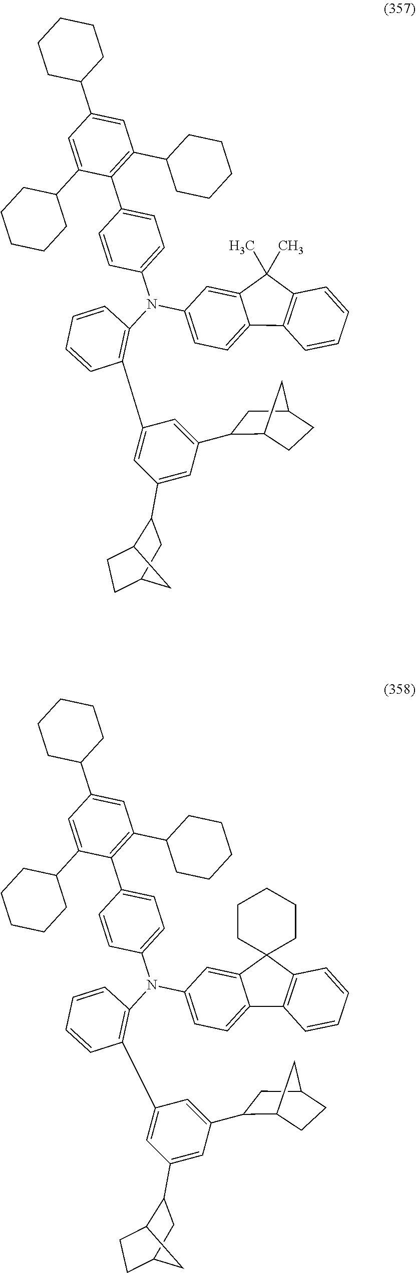 [SMILES: CC1(C)c2ccccc2-c2ccc(N(c3ccc(-c4c(C5CCCCC5)cc(C5CCCCC5)cc4C4CCCCC4)cc3)c3ccccc3-c3cc(C4CC5CCC4C5)cc(C4CC5CCC4C5)c3)cc21 ; c1ccc(N(c2ccc(-c3c(C4CCCCC4)cc(C4CCCCC4)cc3C3CCCCC3)cc2)c2ccc3c(c2)C2(CCCCC2)c2ccccc2-3)c(-c2cc(C3CC4CCC3C4)cc(C3CC4CCC3C4)c2)c1]

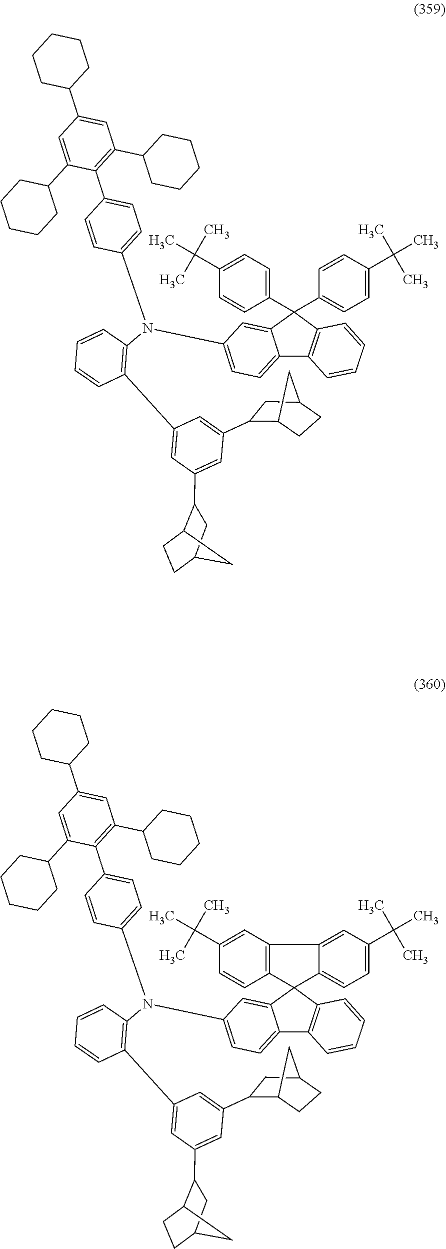 [SMILES: CC(C)(C)c1ccc(C2(c3ccc(C(C)(C)C)cc3)c3ccccc3-c3ccc(N(c4ccc(-c5c(C6CCCCC6)cc(C6CCCCC6)cc5C5CCCCC5)cc4)c4ccccc4-c4cc(C5CC6CCC5C6)cc(C5CC6CCC5C6)c4)cc32)cc1 ; CC(C)(C)c1ccc2c(c1)-c1cc(C(C)(C)C)ccc1C21c2ccccc2-c2ccc(N(c3ccc(-c4c(C5CCCCC5)cc(C5CCCCC5)cc4C4CCCCC4)cc3)c3ccccc3-c3cc(C4CC5CCC4C5)cc(C4CC5CCC4C5)c3)cc21]

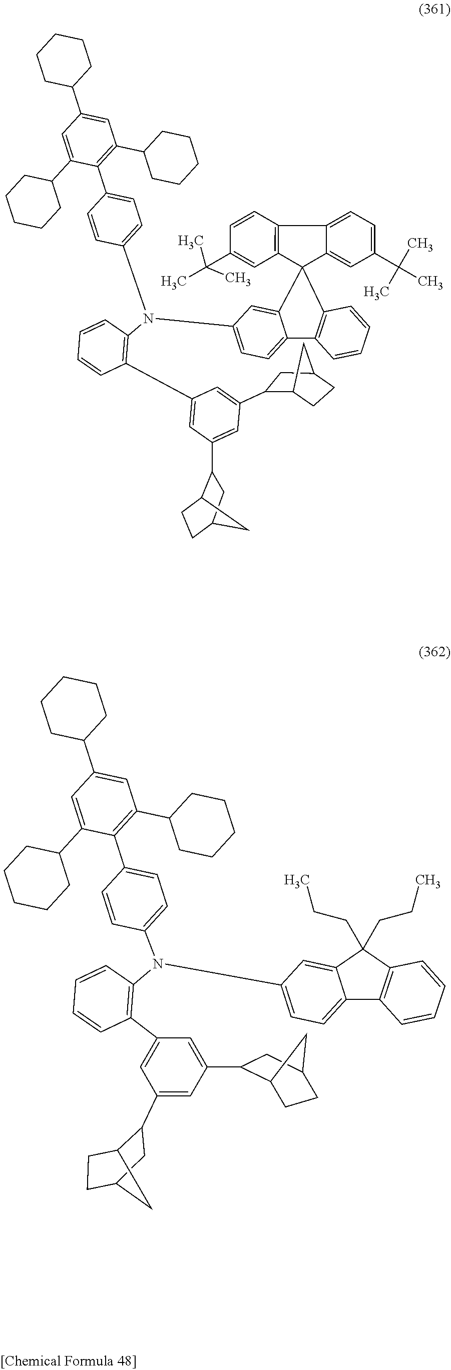 [SMILES: CC(C)(C)c1ccc2c(c1)C1(c3ccccc3-c3ccc(N(c4ccc(-c5c(C6CCCCC6)cc(C6CCCCC6)cc5C5CCCCC5)cc4)c4ccccc4-c4cc(C5CC6CCC5C6)cc(C5CC6CCC5C6)c4)cc31)c1cc(C(C)(C)C)ccc1-2 ; CCCC1(CCC)c2ccccc2-c2ccc(N(c3ccc(-c4c(C5CCCCC5)cc(C5CCCCC5)cc4C4CCCCC4)cc3)c3ccccc3-c3cc(C4CC5CCC4C5)cc(C4CC5CCC4C5)c3)cc21]

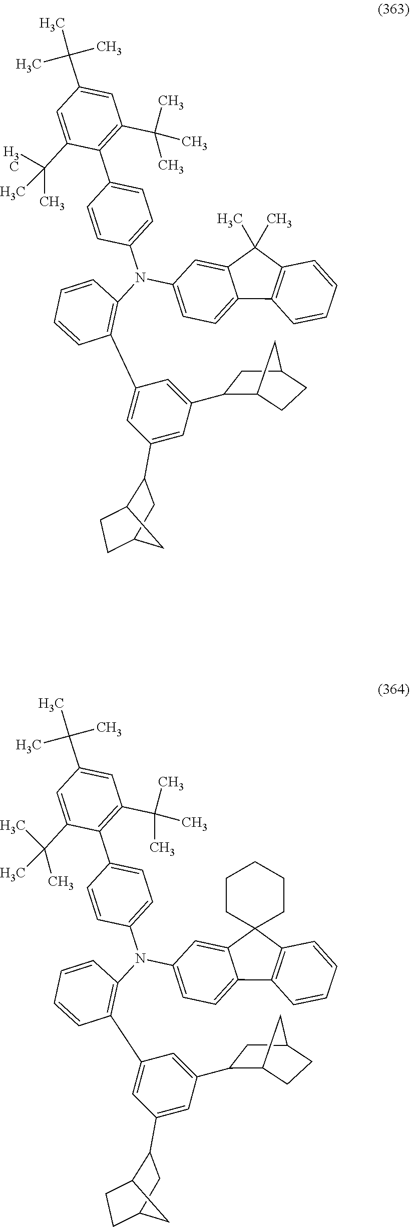 [SMILES: CC(C)(C)c1cc(C(C)(C)C)c(-c2ccc(N(c3ccc4c(c3)C(C)(C)c3ccccc3-4)c3ccccc3-c3cc(C4CC5CCC4C5)cc(C4CC5CCC4C5)c3)cc2)c(C(C)(C)C)c1 ; CC(C)(C)c1cc(C(C)(C)C)c(-c2ccc(N(c3ccc4c(c3)C3(CCCCC3)c3ccccc3-4)c3ccccc3-c3cc(C4CC5CCC4C5)cc(C4CC5CCC4C5)c3)cc2)c(C(C)(C)C)c1]

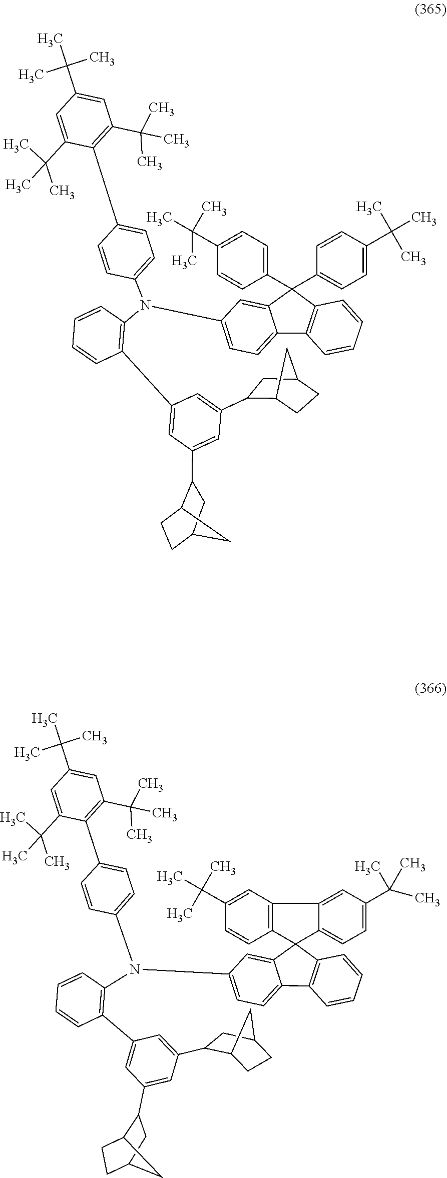

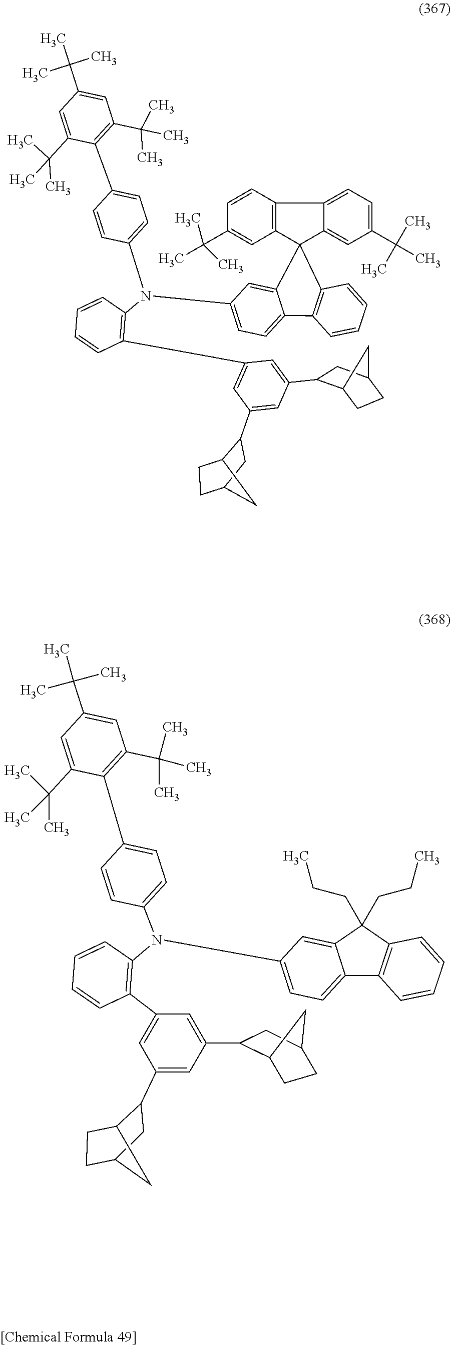

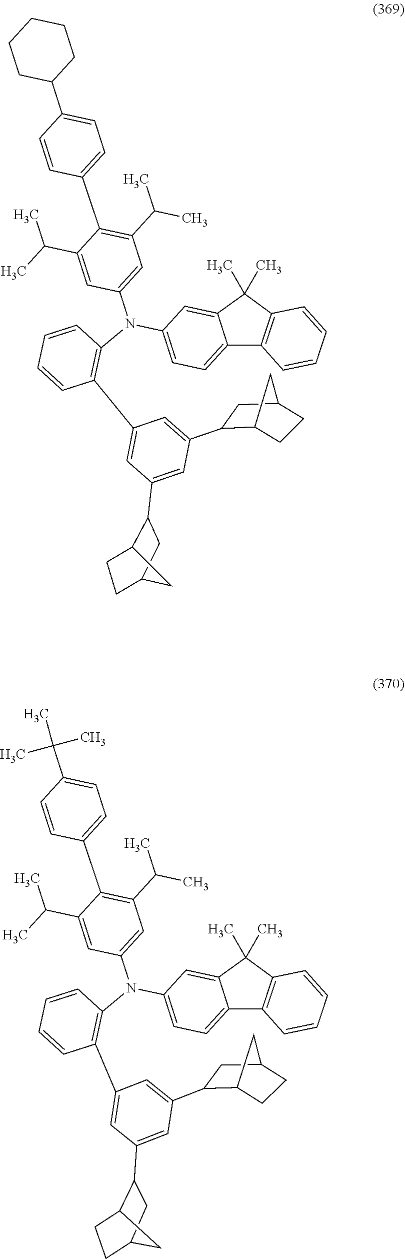 [SMILES: CC(C)c1cc(N(c2ccc3c(c2)C(C)(C)c2ccccc2-3)c2ccccc2-c2cc(C3CC4CCC3C4)cc(C3CC4CCC3C4)c2)cc(C(C)C)c1-c1ccc(C2CCCCC2)cc1 ; CC(C)c1cc(N(c2ccc3c(c2)C(C)(C)c2ccccc2-3)c2ccccc2-c2cc(C3CC4CCC3C4)cc(C3CC4CCC3C4)c2)cc(C(C)C)c1-c1ccc(C(C)(C)C)cc1]

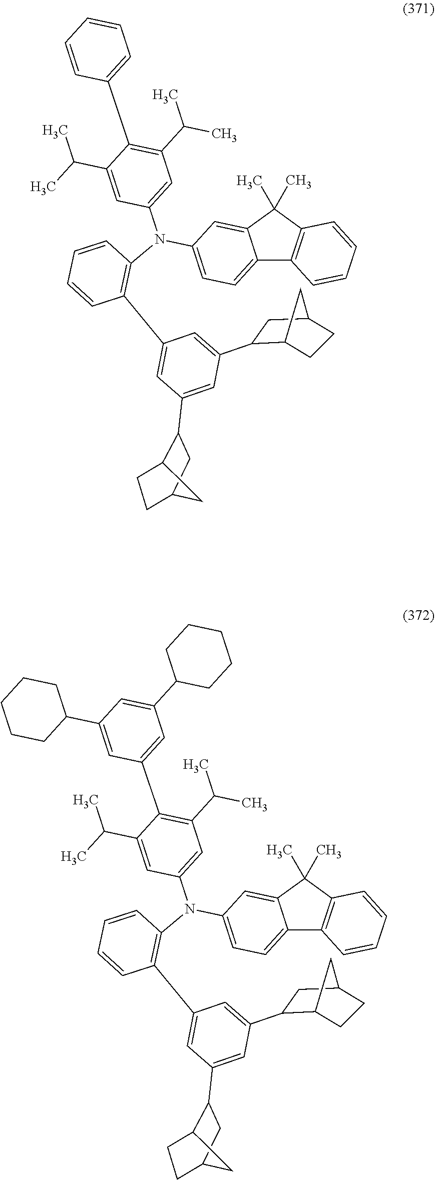 [SMILES: CC(C)c1cc(N(c2ccc3c(c2)C(C)(C)c2ccccc2-3)c2ccccc2-c2cc(C3CC4CCC3C4)cc(C3CC4CCC3C4)c2)cc(C(C)C)c1-c1ccccc1 ; CC(C)c1cc(N(c2ccc3c(c2)C(C)(C)c2ccccc2-3)c2ccccc2-c2cc(C3CC4CCC3C4)cc(C3CC4CCC3C4)c2)cc(C(C)C)c1-c1cc(C2CCCCC2)cc(C2CCCCC2)c1]

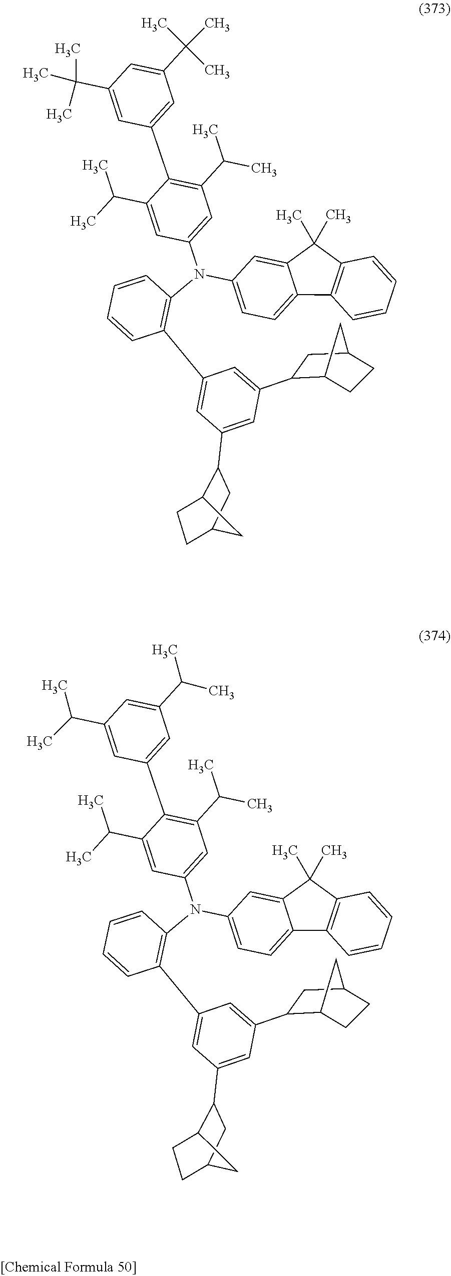 [SMILES: CC(C)c1cc(N(c2ccc3c(c2)C(C)(C)c2ccccc2-3)c2ccccc2-c2cc(C3CC4CCC3C4)cc(C3CC4CCC3C4)c2)cc(C(C)C)c1-c1cc(C(C)(C)C)cc(C(C)(C)C)c1 ; CC(C)c1cc(-c2c(C(C)C)cc(N(c3ccc4c(c3)C(C)(C)c3ccccc3-4)c3ccccc3-c3cc(C4CC5CCC4C5)cc(C4CC5CCC4C5)c3)cc2C(C)C)cc(C(C)C)c1]

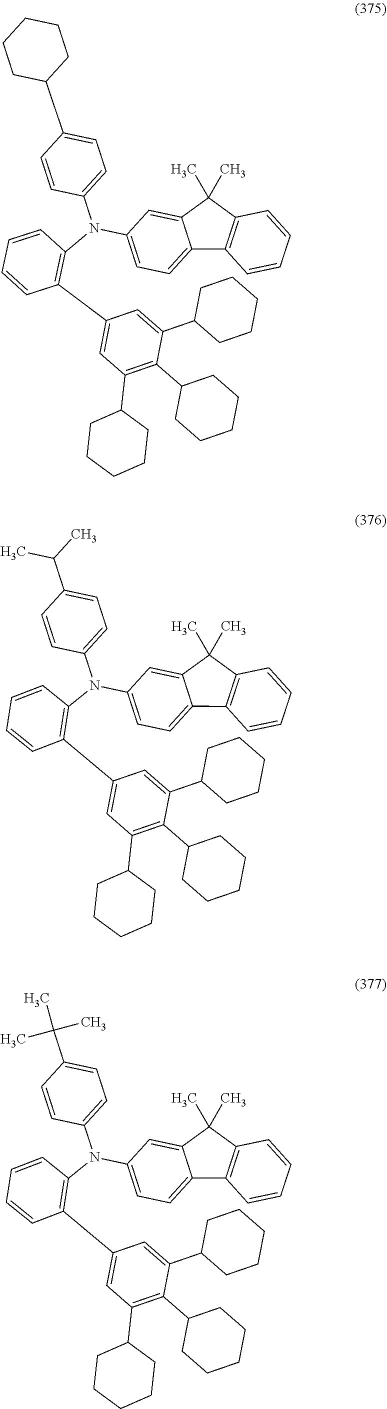 [SMILES: CC1(C)c2ccccc2-c2ccc(N(c3ccc(C4CCCCC4)cc3)c3ccccc3-c3cc(C4CCCCC4)c(C4CCCCC4)c(C4CCCCC4)c3)cc21 ; CC(C)c1ccc(N(c2ccc3c(c2)C(C)(C)c2ccccc2-3)c2ccccc2-c2cc(C3CCCCC3)c(C3CCCCC3)c(C3CCCCC3)c2)cc1 ; CC(C)(C)c1ccc(N(c2ccc3c(c2)C(C)(C)c2ccccc2-3)c2ccccc2-c2cc(C3CCCCC3)c(C3CCCCC3)c(C3CCCCC3)c2)cc1]

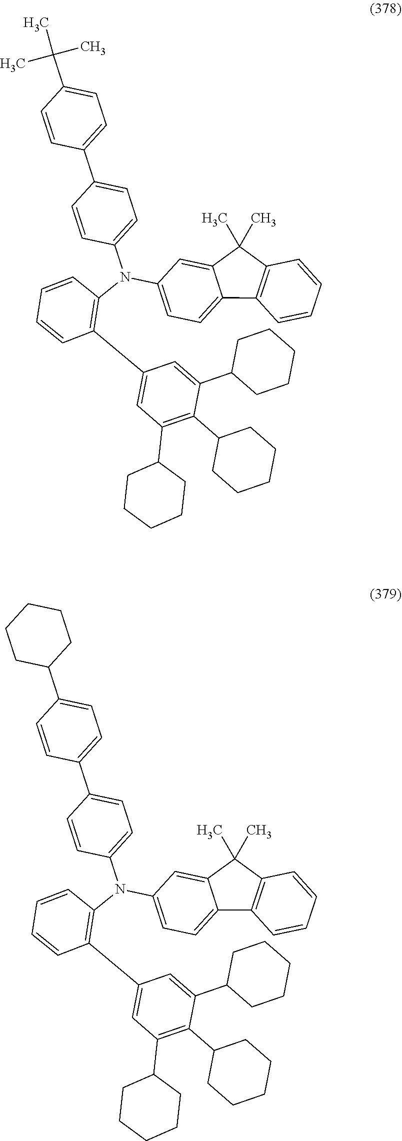 [SMILES: CC(C)(C)c1ccc(-c2ccc(N(c3ccc4c(c3)C(C)(C)c3ccccc3-4)c3ccccc3-c3cc(C4CCCCC4)c(C4CCCCC4)c(C4CCCCC4)c3)cc2)cc1 ; CC1(C)c2ccccc2-c2ccc(N(c3ccc(-c4ccc(C5CCCCC5)cc4)cc3)c3ccccc3-c3cc(C4CCCCC4)c(C4CCCCC4)c(C4CCCCC4)c3)cc21]

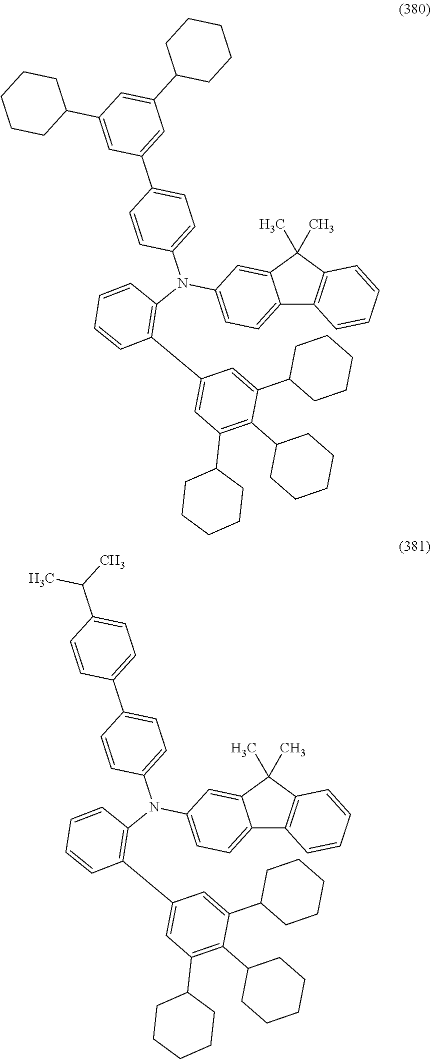 [SMILES: CC1(C)c2ccccc2-c2ccc(N(c3ccc(-c4cc(C5CCCCC5)cc(C5CCCCC5)c4)cc3)c3ccccc3-c3cc(C4CCCCC4)c(C4CCCCC4)c(C4CCCCC4)c3)cc21 ; CC(C)c1ccc(-c2ccc(N(c3ccc4c(c3)C(C)(C)c3ccccc3-4)c3ccccc3-c3cc(C4CCCCC4)c(C4CCCCC4)c(C4CCCCC4)c3)cc2)cc1]

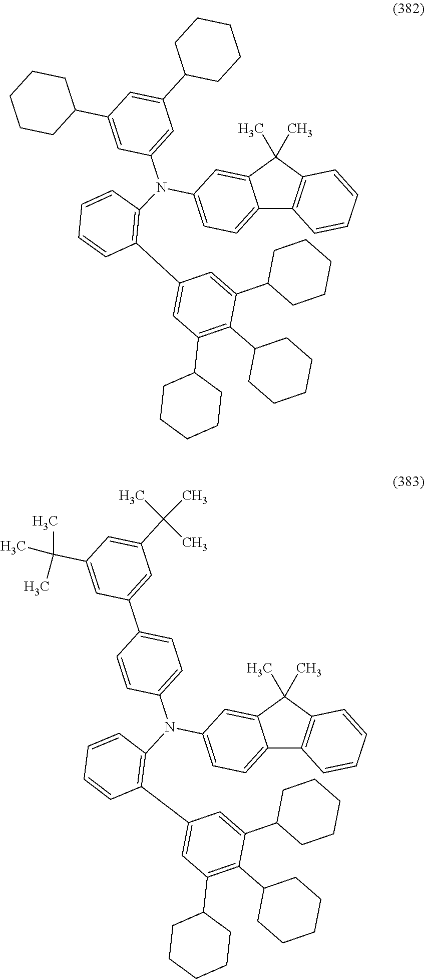 [SMILES: CC1(C)c2ccccc2-c2ccc(N(c3cc(C4CCCCC4)cc(C4CCCCC4)c3)c3ccccc3-c3cc(C4CCCCC4)c(C4CCCCC4)c(C4CCCCC4)c3)cc21 ; CC(C)(C)c1cc(-c2ccc(N(c3ccc4c(c3)C(C)(C)c3ccccc3-4)c3ccccc3-c3cc(C4CCCCC4)c(C4CCCCC4)c(C4CCCCC4)c3)cc2)cc(C(C)(C)C)c1]

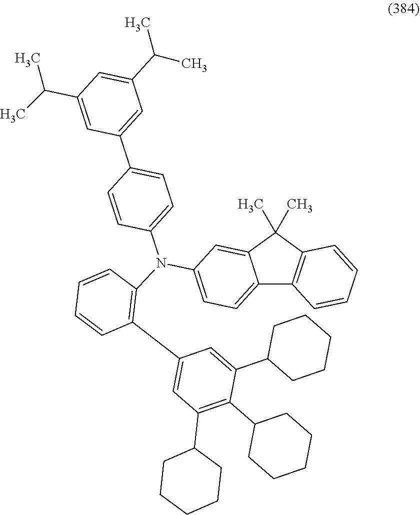 [SMILES: CC(C)c1cc(-c2ccc(N(c3ccc4c(c3)C(C)(C)c3ccccc3-4)c3ccccc3-c3cc(C4CCCCC4)c(C4CCCCC4)c(C4CCCCC4)c3)cc2)cc(C(C)C)c1]

Here, a method for obtaining GSP_slope of an organic compound film formed by a vacuum evaporation method is described.

A phenomenon in which a surface potential of an evaporated film increases in proportion to a film thickness is called the giant surface potential as described above. In general, a slope of a plot of a surface potential of an evaporated film in the thickness direction by Kelvin probe measurement is assumed as the level of the giant surface potential, that is, GSP_slope (mV/nm); in the case where two different layers are stacked, a change in the density of charges (mC/m²) accumulated at the interface, which is in association with GSP, can be utilized to estimate a GSP_slope.

Non-Patent Document 1 discloses that the following formulae hold when current is made to flow through a stack of organic thin films (a thin film 1 and a thin film 2; note that the thin film 1 is positioned on the anode side and the thin film 2 is positioned on the cathode side) with different kinds of spontaneous polarization.

$$\begin{matrix}[ Formula 1 ] & \\ {σ}_{a c c} = ( {V}_{t h} - {V}_{i n j} ) \frac{{ε}_{2}}{{d}_{2}} = - {σ}_{i n t} & ( 1 )\end{matrix}$$

$$\begin{matrix}[ Formula 2 ] & \\ {σ}_{int} = {P}_{1} - {P}_{2} = \frac{{ε}_{1} {V}_{1}}{{d}_{1}} - \frac{{ε}_{2} {V}_{2}}{{d}_{2}} & ( 2 )\end{matrix}$$

In Formula (1), σ_{acc} is an accumulated charge density, σᵢₙₜ is an interface charge density, Vᵢₙⱼ is a hole-injection voltage, Vₜₕ is a threshold voltage, d₂ is a thickness of the thin film 2, and ε₂ is a dielectric constant of the thin film 2. Note that Vᵢₙⱼ and Vₜₕ can be estimated from the capacity-voltage characteristics of a device. The square of an ordinary refractive index nₒ(633 nm) can be used as the dielectric constant. As described above, according to Formula (1), the interface charge density σᵢₙₜ can be calculated using Vᵢₙⱼ and Vₜₕ estimated from the capacity-voltage characteristics, the dielectric constant ε₂ of the thin film 2 calculated from the refractive index, and the thickness d₂ of the thin film 2.

Next, in Formula (2), Pₙ is spontaneous polarization of a thin film n in the substrate normal direction, εₙ is a dielectric constant of the thin film n, Vₙ is a potential of the surface of the film, and dₙ is a thickness of the thin film n. Then, a GSP_slope can be calculated from a value obtained by dividing the potential (Vₙ) of the surface of the film by the thickness (dₙ). Since the interface charge density σᵢₙₜ can be obtained from Formula (1) above, the use of a substance with known GSP_slope for the thin film 2 and an appropriate dielectric constant enables the GSP_slope of the thin film 1 to be estimated.

In view of this, an example in which a measurement device 1 using tris(8-quinolinolato)aluminum (abbreviation: Alq₃) with known GSP_slope (48 (mV/nm)) as the thin film 2 is fabricated, and the GSP_slope of 4,4′-bis[N-(1-naphthyl)-N-phenylamino]biphenyl (abbreviation: NPB) is obtained is shown below.

The following table shows the device structure of the measurement device 1. Note that layers 1_1 to 4_1 and a cathode in the measurement device 1 are formed from an anode side by a vacuum evaporation method under the conditions where the substrate temperature is set to room temperature and the deposition rate ranges from 0.2 nm/s to 0.4 nm/s. One layer is formed without interruption of evaporation. In the measurement device 1, the layer 2_1 corresponds to the thin film 1 and the layer 3_1 corresponds to the thin film 2. OCHD-003 is an organic compound having an electron-acceptor property.

Note that in fabricating the measurement device, the deposition rate of each layer is preferably greater than or equal to 3 nm/min and less than or equal to 600 nm/min. The thickness of each layer in the measurement device is preferably greater than or equal to 3 nm and less than or equal to 500 nm, further preferably greater than or equal to 50 nm and less than or equal to 300 nm.

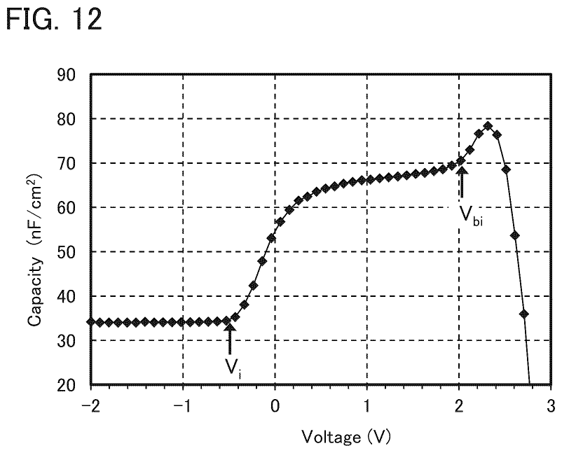

FIG. 12 shows the capacity-voltage characteristics of the measurement device 1.

TABLE 1

| | Film thickness | Measurement device 1 |
|---|---|---|
| Cathode | 200 nm | Al |
| Layer 4_1 | 1 nm | LiF |
| Layer 3_1 | 60 nm | Alq₃ |
| Layer 2_1 | 80 nm | NPB |
| Layer 1_1 | 10 nm | NPB:OCHD-003 |
| | | (1:0.1) |
| Anode | 70 nm | ITSO |

Table 2 shows the hole-injection voltage Vᵢₙⱼ, the threshold voltage Vₜₕ, the interface charge density σᵢₙₜ, and the GSP_slope of the measurement device 1 that are obtained from FIG. 12 and Formulae (1) and (2) and the ordinary refractive index nₒ of the thin film 2 used in the calculation. The refractive indices are measured with a spectroscopic ellipsometer (M-2000U, produced by J.A. Woollam Japan Corp.).

TABLE 2

| | Measurement device 1 |
|---|---|
| Hole-injection voltage Vᵢₙⱼ | −0.53 |
| (V) | |
| Threshold voltage Vₜₕ | 2.02 |
| (V) | |
| Polarization charge density σᵢₙₜ | −1.1 |
| (mC/m²) | |
| Ordinary refractive index nₒ | 1.77 |
| (@633 nm) | |
| GSP_slope | 5.2 |
| (mV/nm) | |

Note that a measurement device 2 having substantially the same structures as the measurement device 1 except that the thickness of Alq₃ is 80 nm is fabricated. It is confirmed that the hole-injection voltages of the measurement device 2 shift to a lower voltage side than that of the measurement device 1. That is, it is presumed that holes are injected first and charges are accumulated at the interface with Alq₃ in such devices. Furthermore, a GSP_slope is estimated for the measurement device 2 in a manner similar to that for the measurement device 1, and the same results as those of the measurement device 1 are obtained.

In the case where the threshold voltage Vₜₕ is difficult to determine from the capacity-voltage characteristics, the threshold voltage may be determined from the current density-voltage characteristics.

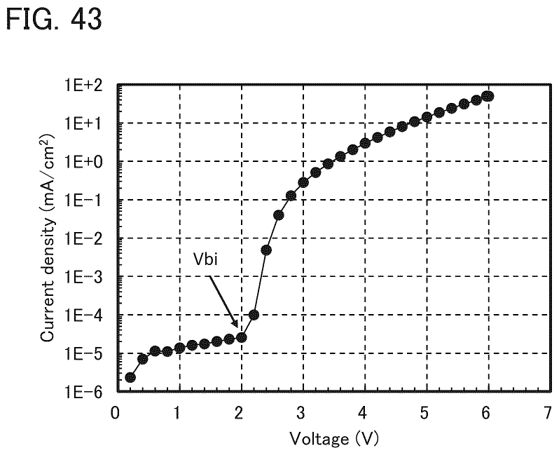

FIG. 43 shows the current density-voltage characteristics of the measurement device 1.

Vₜₕ calculated from the current density-voltage characteristics is 2.0 V, which is the same as the value calculated from the capacity-voltage characteristics.

In this manner, a device in which Alq₃ with known GSP_slope and an organic compound film whose GSP_slope is to be obtained are stacked is fabricated and the capacity-voltage characteristics are measured, so that the GSP_slope of the organic compound can be estimated.

The table below shows the GSP_slope, which is obtained by the above method, of films of compounds 1 and 2 of one embodiment of the present invention and comparative compounds 1 and 2 having similar structures to the compounds 1 and 2. The films are formed by a vacuum evaporation method. The compound 1 is N-(3′,5′-ditertiarybutylbiphenyl-2-yl)-N-(4-cyclohexylphenyl)-9,9-dimethyl-9H-fluoren-2-amine (abbreviation: mmtBuoBichPAF), the compound 2 is N-(3′,5′-ditertiarybutylbiphenyl-4-yl)-N-(3′,5′-ditertiarybutylbiphenyl-2-yl)-9,9-dimethyl-9H-fluoren-2-amine (abbreviation: dmmtBuopBBAF), the comparative compound 1 is N-[(3′,5′-ditertiarybutyl)biphenyl-4-yl]-N-(4-cyclohexylphenyl)-9,9-dimethyl-9H-fluoren-2-amine (abbreviation: mmtBuBichPAF), and the comparative compound 2 is N-(3′,5′-ditertiarybutylbiphenyl-4-yl)-N-(biphenyl-2-yl)-9,9-dimethyl-9H-fluoren-2-amine (abbreviation: mmtBuBioFBi). The molecular structures of the compound 1, the compound 2, the comparative compound 1, and the comparative compound 2 are shown below.

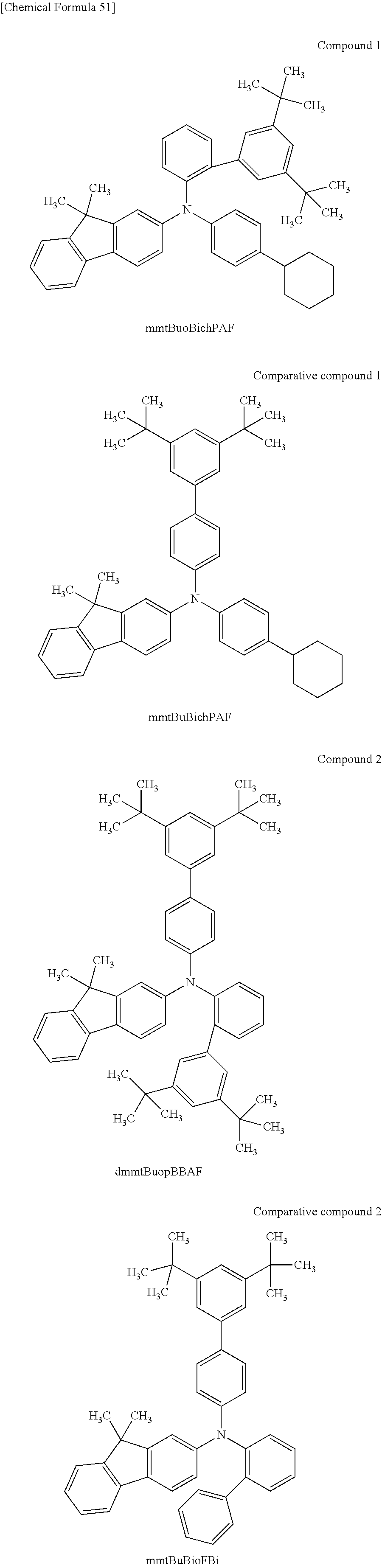 [SMILES: CC(C)(C)c1cc(-c2ccccc2N(c2ccc(C3CCCCC3)cc2)c2ccc3c(c2)C(C)(C)c2ccccc2-3)cc(C(C)(C)C)c1 ; CC(C)(C)c1cc(-c2ccc(N(c3ccc(C4CCCCC4)cc3)c3ccc4c(c3)C(C)(C)c3ccccc3-4)cc2)cc(C(C)(C)C)c1 ; CC(C)(C)c1cc(-c2ccc(N(c3ccc4c(c3)C(C)(C)c3ccccc3-4)c3ccccc3-c3cc(C(C)(C)C)cc(C(C)(C)C)c3)cc2)cc(C(C)(C)C)c1 ; CC(C)(C)c1cc(-c2ccc(N(c3ccc4c(c3)C(C)(C)c3ccccc3-4)c3ccccc3-c3ccccc3)cc2)cc(C(C)(C)C)c1]

TABLE 3

| | | GSP slope |
|---|---|---|
| | Abbreviation | (mV/nm) |
| Compound 1 | mmtBuoBichPAF | 37.5 |
| Comparative compound 1 | mmtBuBichPAF | 31.6 |
| Compound 2 | dmmtBuopBBAF | 46.2 |
| Comparative compound 2 | mmtBuBioFBi | 25.5 |

It is found that the GSP_slope of the film of the compound 1 (mmtBuoBichPAF) is greatly different from that of the film of the comparative compound 1 (mmtBuBichPAF) although the compound 1 (mmtBuoBichPAF) and the comparative compound 1 (mmtBuBichPAF) have the same molecular structure except a biphenyl group to which two alkyl groups are bonded at the end as described above. The biphenyl group is an orthobiphenyl group in the compound 1 and a parabiphenyl group in the comparative compound 1. The film of the compound 1 (mmtBuoBichPAF) has a large GSP_slope by including an orthobiphenyl group in which two alkyl groups are bonded to its end; as a result, the compound 1 is found to be an organic compound which can provide a light-emitting device with high emission efficiency, low driving voltage, and extremely high power efficiency.

It is found that the GSP_slope of the film of the compound 2 (dmmtBuopBBAF) is greatly different from that of the film of the comparative compound 2 (mmtBuBioFBi) although the compound 2 (dmmtBuopBBAF) and the comparative compound 2 (mmtBuBioFBi) have the same molecular structure except the presence of two alkyl groups at the end of the orthobiphenyl group as described above. The film of the compound 2 (dmmtBuopBBAF) has a large GSP_slope by including an orthobiphenyl group in which two alkyl groups are bonded to its end; as a result, the compound 2 is found to be an organic compound which can provide a light-emitting device with high emission efficiency, low driving voltage, and extremely high power efficiency.

The compounds 1 and 2 are each an organic compound capable of forming a film with a low refractive index by including a plurality of alkyl groups, and are each an organic compound capable of forming a film with a high hole-transport property and an appropriate HOMO level by including an amine skeleton.

As described above, the organic compound of one embodiment of the present invention is an organic compound capable of forming a film with a low refractive index, a large GSP_slope, a high hole-transport property, and an appropriate HOMO level because the organic compound is an arylamine compound including an orthobiphenyl group including a plurality of alkyl groups.

### Embodiment 2

In this embodiment, a light-emitting device of one embodiment of the present invention will be described in detail.

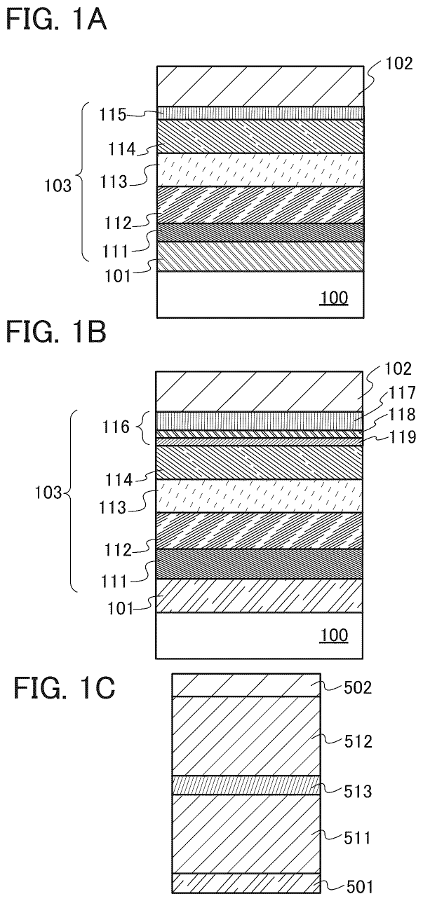

FIGS. 1A to 1C are schematic diagrams of light-emitting devices of embodiments of the present invention. Each of the light-emitting devices includes a first electrode 101 over an insulator 100, and an organic compound layer 103 between the first electrode 101 and a second electrode 102. The organic compound layer 103 includes at least one of the organic compounds represented by General Formulae (G1) to (G4) described in Embodiment 1. The organic semiconductor device of one embodiment of the present invention includes an active layer (e.g., a light-emitting layer 113 in a light-emitting device or a photoelectric conversion layer in a photosensor). The light-emitting layer 113 in the light-emitting device contains an emission center substance that emits light when voltage is applied between the first electrode 101 and the second electrode 102.

The organic compound layer 103 preferably includes, besides the light-emitting layer 113, functional layers such as a hole-injection layer 111, a hole-transport layer 112, an electron-transport layer 114, and an electron-injection layer 115, as shown in FIG. 1A. Note that the organic compound layer 103 may include functional layers other than the above functional layers, such as a hole-blocking layer, an electron-blocking layer, an exciton-blocking layer, and a charge-generation layer. Alternatively, any of the above layers may be omitted.

Since the organic compounds represented by General Formulae (G1) to (G4) in Embodiment 1 have high hole-transport properties, the organic compounds are preferably contained in a layer where holes are moved. Examples of the layer where holes are moved include a hole-injection layer, a hole-transport layer, an electron-blocking layer, and a light-emitting layer; the hole-injection layer, the hole-transport layer, and the electron-blocking layer are preferable. Since the organic compounds are each an organic compound capable of forming a film with a low refractive index, a light-emitting device with high emission efficiency can be obtained.

The organic compounds represented by General Formulae (G1) to (G4) in Embodiment 1 are each an organic compound capable of forming a film with a large GSP_slope; thus, particularly in the hole-transport layer having a stacked-layer structure, the organic compounds are preferably included in a layer closer to an electrode facing an electrode formed over a substrate. That is, in the ordered stacked light-emitting device, it is preferable that the organic compound be included in the layer closest to the cathode of the stacked hole-transport layers or in the electron-blocking layer when the electron-blocking layer is provided. In the inversed stacked light-emitting device, it is preferable that the organic compound be included in the layer closest to the anode of the stacked hole-transport layers. Accordingly, with the organic compound capable of forming a film with a large GSP_slope, the light-emitting device having a low driving voltage can be obtained. In addition, since the organic compound is capable of forming a film with a low refractive index, the light-emitting device can have high emission efficiency, a low driving voltage, and extremely favorable characteristics.

Although the first electrode 101 includes an anode and the second electrode 102 includes a cathode in this embodiment, the first electrode 101 may include a cathode and the second electrode 102 may include an anode. The first electrode 101 and the second electrode 102 each have a single-layer structure or a stacked-layer structure. In the case of the stacked-layer structure, a layer in contact with the organic compound layer 103 serves as an anode or a cathode. In the case where the electrodes each have the stacked-layer structure, there is no limitation on work functions of materials for layers other than the layer in contact with the organic compound layer 103, and the materials can be selected in accordance with required properties such as a resistance value, processing easiness, reflectivity, light-transmitting property, and stability.

The anode is preferably formed using any of metals, alloys, and conductive compounds with a high work function (specifically, higher than or equal to 4.0 eV), mixtures thereof, and the like. Specific examples include indium oxide-tin oxide (ITO: indium tin oxide), indium oxide-tin oxide containing silicon or silicon oxide (ITSO: indium tin silicon oxide), indium oxide-zinc oxide, and indium oxide containing tungsten oxide and zinc oxide (IWZO). Films of such conductive metal oxides are usually formed by a sputtering method, but may be formed by application of a sol-gel method or the like. For example, a film of indium oxide-zinc oxide is formed by a sputtering method using a target in which 1 wt % to 20 wt % zinc oxide is added to indium oxide. Furthermore, a film of indium oxide containing tungsten oxide and zinc oxide (IWZO) can be formed by a sputtering method using a target in which 0.5 wt % to 5 wt % tungsten oxide and 0.1 wt % to 1 wt % zinc oxide are added to indium oxide. Alternatively, gold (Au), platinum (Pt), nickel (Ni), tungsten (W), chromium (Cr), molybdenum (Mo), iron (Fe), cobalt (Co), copper (Cu), palladium (Pd), titanium (Ti), aluminum (Al), nitride of a metal material (e.g., titanium nitride), or the like can be used for the anode. The anode may be a stack of layers formed of any of these materials. For example, a film in which Al, Ti, and ITSO are stacked in this order over Ti is preferable because the film has high efficiency owing to high reflectivity and enables high resolution of several thousand ppi. Graphene can also be used for the anode. When a composite material that can be included in the hole-injection layer 111 described later is used for a layer (typically, the hole-injection layer) in contact with the anode, an electrode material can be selected regardless of its work function.

The hole-injection layer 111 is provided in contact with the anode and has a function of facilitating injection of holes into the organic compound layer 103. The hole-injection layer 111 can be formed using phthalocyanine (abbreviation: H₂Pc), a phthalocyanine compound or a phthalocyanine-based complex compound such as copper phthalocyanine (abbreviation: CuPc), an aromatic amine compound such as 4,4′-bis[N-(4-diphenylaminophenyl)-N-phenylamino]biphenyl (abbreviation: DPAB) or N,N′-bis[4-bis(3-methylphenyl)aminophenyl]-N,N′-diphenyl-4,4′-diaminobiphenyl (abbreviation: DNTPD), or a high molecular compound such as poly(3,4-ethylenedioxythiophene)/poly(styrenesulfonic acid) (abbreviation: PEDOT/PSS).

The hole-injection layer 111 may be formed using a substance having an electron-accepting property. Examples of the substance having an acceptor property include organic compounds having an electron-withdrawing group (a halogen group or a cyano group), such as 7,7,8,8-tetracyano-2,3,5,6-tetrafluoroquinodimethane (abbreviation: F4-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylene-1,3,4,5,6,8,9,10-octafluoro-7H-pyren-2-ylidene)malononitrile. A compound in which electron-withdrawing groups are bonded to a fused aromatic ring having a plurality of heteroatoms, such as HAT-CN, is particularly preferable because it is thermally stable. A [3]radialene derivative having an electron-withdrawing group (in particular, a cyano group, a halogen group such as a fluoro group, or the like) has a significantly high electron-accepting property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a transition metal oxide such as molybdenum oxide, vanadium oxide, ruthenium oxide, tungsten oxide, or manganese oxide can be used, other than the above-described organic compounds.

The hole-injection layer 111 is preferably formed using a composite material containing any of the aforementioned materials having an acceptor property and an organic compound having a hole-transport property.

As the organic compound having a hole-transport property used in the composite material, any of a variety of organic compounds such as aromatic amine compounds, heteroaromatic compounds, aromatic hydrocarbons, and high molecular compounds (e.g., oligomers, dendrimers, and polymers) can be used. Note that the organic compound having a hole-transport property used in the composite material preferably has a hole mobility higher than or equal to 1×10⁻⁶ cm²/Vs. The organic compound having a hole-transport property used in the composite material preferably has a fused aromatic hydrocarbon ring or a π-electron rich heteroaromatic ring. As the fused aromatic hydrocarbon ring, an anthracene ring, a naphthalene ring, or the like is preferable. As the π-electron rich heteroaromatic ring, a fused aromatic ring having at least one of a pyrrole skeleton, a furan skeleton, and a thiophene skeleton is preferable; specifically, a carbazole ring, a dibenzothiophene ring, or a ring in which an aromatic ring or a heteroaromatic ring is further fused to a carbazole ring or a dibenzothiophene ring is preferable.

Such an organic compound having a hole-transport property further preferably has at least one of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that has a naphthalene ring, or an aromatic monoamine in which a 9-fluorenyl group is bonded to nitrogen of an amine through an arylene group may be used. Note that the organic compound having a hole-transport property preferably has an N,N-bis(4-biphenyl)amino group to enable fabricating a light-emitting device with a long lifetime.

Specific examples of the organic compound having a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Examples of the aromatic amine compounds that can be used as the material having a hole-transport property include N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), 4,4′-bis(N-{4-[N-(3-methylphenyl)-N-phenylamino]phenyl}-N-phenylamino)biphenyl (abbreviation: DNTPD), and 1,3,5-tris[N-(4-diphenylaminophenyl)-N-phenylamino]benzene (abbreviation: DPA3B). The organic compounds represented by General Formulae (G1) to (G4) in Embodiment 1 can also be suitably used. In the case where the organic compounds represented by General Formulae (G1) to (G4) in Embodiment 1 are used, a light-emitting device with high emission efficiency can be obtained because the organic compounds are each a material capable of forming a film with a low refractive index.

The formation of the hole-injection layer 111 can improve the hole-injection property, which allows the light-emitting device to be driven at a low voltage.

Among substances having an acceptor property, the organic compound having an acceptor property is easy to use because it is easily evaporated.

The hole-transport layer 112 is formed using an organic compound having a hole-transport property. The organic compound having a hole-transport property preferably has a hole mobility higher than or equal to 1×10⁻⁶ cm²/Vs.

Examples of the aforementioned organic compound with a hole-transport property include the following compounds: compounds having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-3,3′-bi-9H-carbazole (abbreviation: PNCCP), 9-(3-biphenyl)-9′-(2-naphthyl)-3,3′-bi-9H-carbazole (abbreviation: PNCCmBP), 9-(4-biphenyl)-9′-(2-naphthyl)-3,3′-bi-9H-carbazole (abbreviation: βNCCBP), 9,9′-di-2-naphthyl-3,3′-9H,9′H-bicarbazole (abbreviation: BisβNCz), 9-(2-naphthyl)-9′-[1,1′: 4′,1″-terphenyl]-3-yl-3,3′-9H,9′H-bicarbazole, 9-(2-naphthyl)-9′-[1,1′: 3′,1″-terphenyl]-3-yl-3,3′-9H,9′H-bicarbazole, 9-(2-naphthyl)-9′-[1,1′: 3′,1″-terphenyl]-5′-yl-3,3′-9H,9′H-bicarbazole, 9-(2-naphthyl)-9′-[1,1′: 4′,1″-terphenyl]-4-yl-3,3′-9H,9′H-bicarbazole, 9-(2-naphthyl)-9′-[1,1′: 3′,1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-9H,9′H-bicarbazole, and 9-(triphenylen-2-yl)-9′-[1,1′: 3′,1″-terphenyl]-4-yl-3,3′-9H,9′H-bicarbazole; compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton or the compound having a carbazole skeleton is preferable because the compound is highly reliable and has a high hole-transport property to contribute to a reduction in driving voltage. Note that any of the substances given as examples of the material having a hole-transport property used in the composite material for the hole-injection layer 111 can also be suitably used as the material contained in the hole-transport layer 112.

The organic compounds represented by General Formulae (G1) to (G4) in Embodiment 1 can also be suitably used as materials included in the above-described hole-transport layer. In the case where the organic compounds represented by General Formulae (G1) to (G4) in Embodiment 1 are used, a light-emitting device with high emission efficiency can be provided because the organic compounds are each an organic compound capable of forming a film with a low refractive index. In the case where the hole-transport layer is formed to have a stacked-layer structure, it is particularly preferable that the organic compounds represented by General Formulae (G1) to (G4) in Embodiment 1 be used for the layer closest to the cathode in the stacked-layer structure. This is because the organic compounds are each an organic compound capable of forming a film with a large GSP_slope and thus a light-emitting device with a low driving voltage can be provided. Note that the organic compounds represented by General Formulae (G1) to (G4) in Embodiment 1 are further preferably contained in a layer which is in contact with the light-emitting layer in the ordered stacked light-emitting device.

As described above, with the use of the organic compounds represented by General Formulae (G1) to (G4) in Embodiment 1, a light-emitting device with high emission efficiency and a low driving voltage, i.e., a light-emitting device with particularly high power efficiency, can be provided.

The emission center substance may be a fluorescent substance, a phosphorescent substance, a substance exhibiting thermally activated delayed fluorescence (TADF), or any other light-emitting substance.

Examples of the material that can be used as a fluorescent substance in the light-emitting layer are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPrn), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N″′,N″′-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine](abbreviation: 1,6BnfAPrn-03), N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Fused aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, or high reliability.

A fused heteroaromatic compound containing nitrogen and boron, especially a compound having a diaza-boranaphtho-anthracene skeleton, exhibits a narrow emission spectrum, emits blue light with high color purity, and can thus be suitably used. Examples of the compound include 5,9-diphenyl-5,9-diaza-13b-boranaphtho[3,2,1-de]anthracene (abbreviation: DABNA1), 9-(diphenyl-3-yl)-N,N,5,11-tetraphenyl-5,9-dihydro-5,9-diaza-13b-boranaphtho[3,2,1-de]anthracen-3-amine (abbreviation: DABNA2), 2,12-di(tert-butyl)-5,9-di(4-tert-butylphenyl)-N,N-diphenyl-5H,9H-[1,4]benzazaborino[2,3,4-kl]phenazaborin-7-amine (abbreviation: DPhA-tBu4DABNA), 2,12-di(tert-butyl)-N,N,5,9-tetra(4-tert-butylphenyl)-5H,9H-[1,4]benzazaborino[2,3,4-kl]phenazaborin-7-amine (abbreviation: tBuDPhA-tBu4DABNA), 2,12-di(tert-butyl)-5,9-di(4-tert-butylphenyl)-7-methyl-5H,9H-[1,4]benzazaborino[2,3,4-kl]phenazaborine (abbreviation: Me-tBu4DABNA), N⁷,N⁷,N¹³,N¹³,5,9,11,15-octaphenyl-5H,9H,11H,15H-[1,4]benzazaborino[2,3,4-kl][1,4]benzazaborino[4′,3′,2′: 4,5][1,4]benzazaborino[3,2-b]phenazaborine-7,13-diamine (abbreviation: v-DABNA), and 2-(4-tert-butylphenyl)benz[5,6]indolo[3,2,1-jk]benzo[b]carbazole (abbreviation: tBuPBibc).

Besides the above compounds, 9,10,11-tris[3,6-bis(1,1-dimethylethyl)-9H-carbazolyl-9-yl]-2,5,15,18-tetrakis(1,1-dimethylethyl)indolo[3,2,1-de]indolo[3′,2′,1′: 8,1][1,4]benzazaborino[2,3,4-kl]phenazaborine (abbreviation: BBCz-G), 9,11-bis[3,6-bis(1,1-dimethylethyl)-9H-carbazolyl-9-yl]-2,5,15,18-tetrakis(1,1-dimethylethyl)indolo[3,2,1-de]indolo[3′,2′,1′: 8,1][1,4]benzazaborino[2,3,4-kl]phenazaborine (abbreviation: BBCz-Y), or the like can be suitably used.

In the case where a phosphorescent substance is used as the light-emitting device in the light-emitting layer, a metal complex, in particular, an iridium complex or a platinum complex is preferable as the phosphorescent substance; examples of the materials are as follows.

The examples include an organometallic iridium complex having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), and tris(5-methyl-3,4-diphenyl-4H-1,2,4-triazolato)iridium(III) (abbreviation: [Ir(Mptz)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); an organometallic iridium complex having an imidazole skeleton, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-{1-[2,6-bis(1-methylethyl)phenyl]-1H-imidazol-2-yl-κN³}-4-cyanophenyl-κC)iridium(III) (abbreviation: CNImIr); an organometallic complex having a benzimidazolidene skeleton, such as tris[(6-tert-butyl-3-phenyl-2H-imidazo[4,5-b]pyrazin-1-yl-κC²)phenyl-κC]iridium(III) (abbreviation: [Ir(cb)₃]); and an organometallic iridium complex in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIracac). These compounds emit blue phosphorescent light and have an emission peak in the wavelength range from 450 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: [Ir(5mppy-d₃)₂(mbfpypy-d₃)]), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d₆)₂(mbfpypy-iPr-d₄)), [2-d₃-methyl-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy-d₃)]), [2-(4-methyl-5-phenyl-2-pyridinyl-κN)phenyl-κC]bis[2-(2-pyridinyl-κN)phenyl-κC]iridium(III) (abbreviation: [Ir(ppy)₂(mdppy)]), [2-(4-d₃-methyl-5-phenyl-2-pyridinyl-κN²)phenyl-κC]bis[2-(5-d₃-methyl-2-pyridinyl-κN²)phenyl-κC]iridium(III) (abbreviation: [Ir(5mppy-d₃)₂(mdppy-d₃)]), and [2-methyl-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy)]); organometallic platinum complexes such as (2-{1-(5-tert-butylbiphenyl-2-yl)-4-[3-tert-butyl-5-(4-phenyl-2-pyridinyl-κN)phenyl-κC⁶]-2-benzimidazolyl-κN³}-4,6-di-tert-butylphenolato-κO)platinum(II) (abbreviation: Pt(tBudppymmtBubiz-tBubp)) and [2-(4-(3,5-di-tert-butylphenyl)-6-{3-[4-(5′-tert-butyl[1,1′:3′,1″-terphenyl]-2′-yl)-2-pyridinyl-N]phenyl-κC²}-2-pyridinyl-κN)phenolato-κO]platinum(II) (abbreviation: Pt(4tButpppypyp-mmtBup)); and rare earth metal complexes such as tris(acetylacetonato) (monophenanthroline)terbium(III) (abbreviation: [Tb(acac)₃(Phen)]). These are mainly compounds that emit green phosphorescent light and have an emission peak in the wavelength range from 500 nm to 600 nm. Note that organometallic iridium complexes including a pyrimidine skeleton have distinctively high reliability or emission efficiency and thus are particularly preferable.

Other examples include an organometallic iridium complex having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); an organometallic iridium complex having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); an organometallic iridium complex having a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), (3,7-diethyl-4,6-nonanedionato-κO⁴,κO⁶)bis[2,4-dimethyl-6-[7-(1-methylethyl)-1-isoquinolinyl-κN]phenyl-κC]iridium(III), and (3,7-diethyl-4,6-nonanedionato-κO⁴,κO⁶)bis[2,4-dimethyl-6-[5-(1-methylethyl)-2-quinolinyl-κN]phenyl-κC]iridium(III); a platinum complex such as 2,3,7,8,12,13,17,18-octaethyl-21H,23H-porphyrinplatinum(II) (abbreviation: PtOEP); and a rare earth metal complex such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit red phosphorescent light and have an emission peak in the wavelength range from 600 nm to 700 nm. Furthermore, the organometallic iridium complexes having a pyrazine skeleton can provide red light emission with favorable chromaticity.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

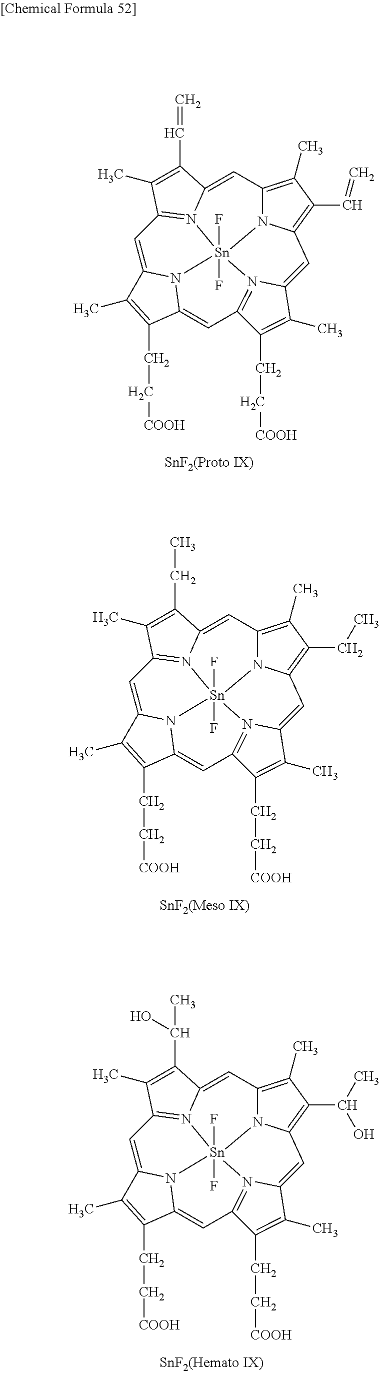

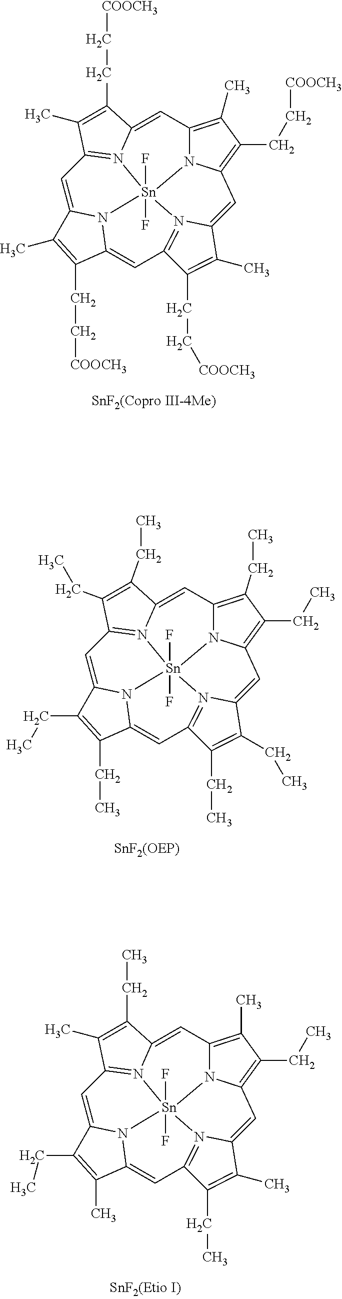

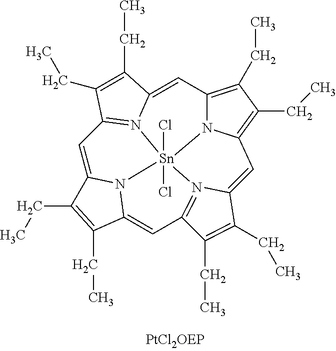

Alternatively, it is possible to use a heterocyclic compound having one or both of a π-electron rich heteroaromatic ring and a π-electron deficient heteroaromatic ring that is represented by the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA). Such a heterocyclic compound is preferable because of having high electron-transport and hole-transport properties owing to a π-electron rich heteroaromatic ring and a π-electron deficient heteroaromatic ring. Among skeletons having the π-electron deficient heteroaromatic ring, a pyridine skeleton, a diazine skeleton (a pyrimidine skeleton, a pyrazine skeleton, and a pyridazine skeleton), and a triazine skeleton are preferable because of their high stability and reliability. In particular, a benzofuropyrimidine skeleton, a benzothienopyrimidine skeleton, a benzofuropyrazine skeleton, and a benzothienopyrazine skeleton are preferable because of their high acceptor properties and high reliability. Among skeletons having the π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which the π-electron rich heteroaromatic ring is directly bonded to the π-electron deficient heteroaromatic ring is particularly preferable because the electron-donating property of the π-electron rich heteroaromatic ring and the electron-accepting property of the π-electron deficient heteroaromatic ring are both improved, the energy difference between the S₁ level and the T₁ level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

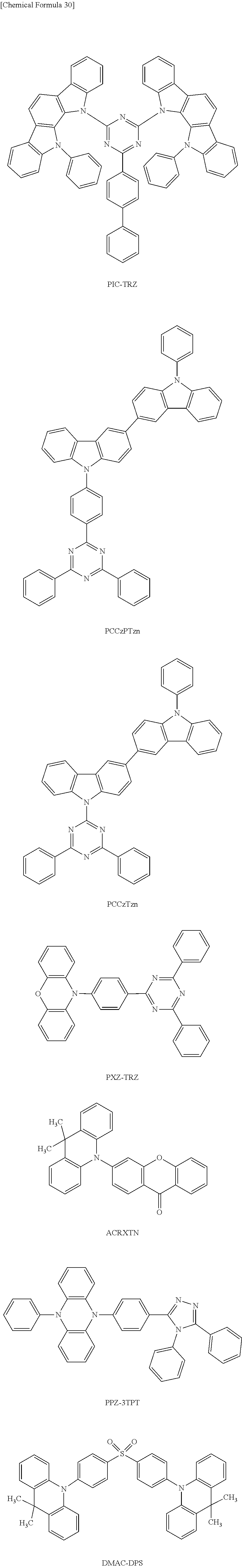

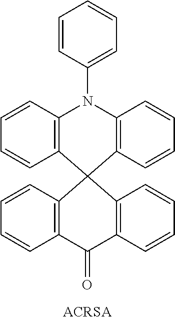 [SMILES: O=C1c2ccccc2C2(c3ccccc31)c1ccccc1N(c1ccccc1)c1ccccc12]

Note that a TADF material is a material having a small difference between the S₁ level and the T₁ level and a function of converting triplet excitation energy into singlet excitation energy by reverse intersystem crossing. Thus, a TADF material can upconvert triplet excitation energy into singlet excitation energy (i.e., reverse intersystem crossing) using a small amount of thermal energy and efficiently generate a singlet excited state. In addition, the triplet excitation energy can be converted into light emission.

An exciplex whose excited state is formed of two kinds of substances has an extremely small difference between the S₁ level and the T₁ level and functions as a TADF material capable of converting triplet excitation energy into singlet excitation energy.

A phosphorescent spectrum observed at a low temperature (e.g., 77 K to 10 K) is used for an index of the T₁ level. When the level of energy with a wavelength of the line obtained by extrapolating a tangent to the fluorescent spectrum at a tail on the short wavelength side is the S₁ level and the level of energy with a wavelength of the line obtained by extrapolating a tangent to the phosphorescent spectrum at a tail on the short wavelength side is the T₁ level, the difference between the S₁ level and the T₁ level of the TADF material is preferably smaller than or equal to 0.3 eV, further preferably smaller than or equal to 0.2 eV.

When a TADF material is used as the light-emitting substance, the S₁ level of the host material is preferably higher than that of the TADF material. In addition, the T₁ level of the host material is preferably higher than that of the TADF material.

As the host material in the light-emitting layer, various carrier-transport materials such as materials having an electron-transport property and/or materials having a hole-transport property, and the TADF materials can be used.

The material with a hole-transport property is preferably an organic compound having an amine skeleton or a π-electron rich heteroaromatic ring skeleton, for example. As the π-electron rich heteroaromatic ring, a fused aromatic ring having at least one of an acridine skeleton, a phenoxazine skeleton, a phenothiazine skeleton, a furan skeleton, a thiophene skeleton, and a pyrrole skeleton is preferable; specifically, a carbazole ring, a dibenzothiophene ring, or a ring in which an aromatic ring or a heteroaromatic ring is further fused to a carbazole ring or a dibenzothiophene ring is preferable.

Such an organic compound having a hole-transport property further preferably has at least one of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that has a naphthalene ring, or an aromatic monoamine in which a 9-fluorenyl group is bonded to nitrogen of an amine through an arylene group may be used. Note that the organic compound having a hole-transport property preferably has an N,N-bis(4-biphenyl)amino group to enable fabricating a light-emitting device with a long lifetime.

Examples of such an organic compound include a compound having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), and 3,3′-bis(9-phenyl-9H-carbazole) (abbreviation: PCCP); a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and a compound having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. In addition, the organic compounds given as examples of the material having a hole-transport property that can be used for the hole-transport layer can also be used. The organic compounds represented by General Formulae (G1) to (G4) in Embodiment 1 can also be suitably used.

The material having an electron-transport property preferably has an electron mobility higher than or equal to 1×10⁻⁷ cm²/Vs, further preferably higher than or equal to 1×10⁻⁶ cm²/Vs in the case where the square root of the electric field strength [V/cm] is 600. Note that any other substance can also be used as long as the substance has an electron-transport property higher than a hole-transport property.

As the material having an electron-transport property, for example, a metal complex such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis[2-(2-benzothiazolyl)phenolato]zinc(II) (abbreviation: ZnBTZ); or an organic compound having a π-electron deficient heteroaromatic ring is preferably used. Examples of the organic compound having a π-electron deficient heteroaromatic ring skeleton include an organic compound including a heteroaromatic ring having an azole skeleton, an organic compound including a heteroaromatic ring having a pyridine skeleton, an organic compound including a heteroaromatic ring having a diazine skeleton, and an organic compound including a heteroaromatic ring having a triazine skeleton.

Among the above materials, the organic compound that includes a heteroaromatic ring having a diazine skeleton (a pyrimidine skeleton, a pyrazine skeleton, or a pyridazine skeleton), the organic compound that includes a heteroaromatic ring having a pyridine skeleton, and the organic compound that includes a heteroaromatic ring having a triazine skeleton have high reliability and thus are preferable. In particular, the organic compound that includes a heteroaromatic ring having a diazine (pyrimidine or pyrazine) skeleton and the organic compound that includes a heteroaromatic ring having a triazine skeleton have a high electron-transport property to contribute to a reduction in driving voltage. A benzofuropyrimidine skeleton, a benzothienopyrimidine skeleton, a benzofuropyrazine skeleton, and a benzothienopyrazine skeleton are preferable because of their high acceptor property and high reliability.

Examples of the organic compound having a π-electron deficient heteroaromatic ring skeleton include an organic compound having an azole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOS); an organic compound having a heteroaromatic ring having a pyridine skeleton, such as 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathophenanthroline (abbreviation: BPhen), bathocuproine (abbreviation: BCP), 2,9-di(naphthalen-2-yl)-4,7-diphenyl-1,10-phenanthroline (abbreviation: NBPhen), 2,2′-(1,3-phenylene)bis(9-phenyl-1,10-phenanthroline) (abbreviation: mPPhen2P), 2-[3-(2-triphenylenyl)phenyl]-1,10-phenanthroline (abbreviation: mTpPPhen), 2-phenyl-9-(2-triphenylenyl)-1,10-phenanthroline (abbreviation: Ph-TpPhen), 2-[4-(9-phenanthryl)-1-naphthyl]-1,10-phenanthroline (abbreviation: PnNPhen), or 2-[4-(2-triphenylenyl)phenyl]-1,10-phenanthroline (abbreviation: pTpPPhen); an organic compound having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)biphenyl-3-yl]naphtho[1′,2′:4,5]furo[3,2-d]pyrimidine (abbreviation: 8mDBtBPNfpm), 8-[(2,2′-binaphthalen)-6-yl]-4-[3-(dibenzothiophen-4-yl)phenyl]-[1]benzofuro[3,2-d]pyrimidine (abbreviation: 8(βN2)-4mDBtPBfpm), 2,2′-(pyridine-2,6-diyl)bis(4-phenylbenzo[h]quinazoline) (abbreviation: 2,6(P-Bqn)2Py), 2,2′-(pyridine-2,6-diyl)bis{4-[4-(2-naphthyl)phenyl]-6-phenylpyrimidine}(abbreviation: 2,6(NP-PPm)2Py), 6-(biphenyl-3-yl)-4-[3,5-bis(9H-carbazol-9-yl)phenyl]-2-phenylpyrimidine (abbreviation: 6mBP-4Cz2PPm), 2,6-bis(4-naphthalen-1-ylphenyl)-4-[4-(3-pyridyl)phenyl]pyrimidine (abbreviation: 2,4NP-6PyPPm), 4-[3,5-bis(9H-carbazol-9-yl)phenyl]-2-phenyl-6-(biphenyl-4-yl)pyrimidine (abbreviation: 6BP-4Cz2PPm), 7-[4-(9-phenyl-9H-carbazol-2-yl)quinazolin-2-yl]-7H-dibenzo[c,g]carbazole (abbreviation: PC-cgDBCzQz), or 8-(p-terphenyl-3-yl)-4-[3-(dibenzothiophen-4-yl)phenyl]-[1]benzofuro[3,2-d]pyrimidine (abbreviation: 8mpTP-4mDBtPBfpm); and an organic compound having a heteroaromatic ring having a triazine skeleton, such as 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 2-[3′-(9,9-dimethyl-9H-fluoren-2-yl)biphenyl-3-yl]-4,6-diphenyl-1,3,5-triazine (abbreviation: mFBPTzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris(3′-(pyridin-3-yl)biphenyl-3-yl)-1,3,5-triazine (abbreviation: TmPPPyTz), 2-[3-(2,6-dimethyl-3-pyridinyl)-5-(9-phenanthryl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), or 2-(biphenyl-3-yl)-4-phenyl-6-{8-[(1,1′: 4′,1″-terphenyl)-4-yl]-1-dibenzofuranyl}-1,3,5-triazine (abbreviation: mBP-TPDBfTzn). The organic compound that includes a heteroaromatic ring having a diazine skeleton, the organic compound that includes a heteroaromatic ring having a pyridine skeleton, and the organic compound that includes a heteroaromatic ring having a triazine skeleton are preferable because of having high reliability. In particular, the organic compound that includes a heteroaromatic ring having a diazine (pyrimidine or pyrazine) skeleton and the organic compound that includes a heteroaromatic ring having a triazine skeleton have a high electron-transport property to contribute to a reduction in driving voltage.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S₁ level of the TADF material is preferably higher than that of the fluorescent substance in order that high emission efficiency can be achieved. Furthermore, the T₁ level of the TADF material is preferably higher than the S₁ level of the fluorescent substance. Therefore, the T₁ level of the TADF material is preferably higher than that of the fluorescent substance.

It is also preferable to use a TADF material that emits light whose wavelength overlaps with the wavelength of the lowest-energy absorption band of the fluorescent substance. This enables smooth transfer of excitation energy from the TADF material to the fluorescent substance and accordingly enables efficient light emission, which is preferable.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton which causes light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transportation or carrier recombination. Here, the luminophore refers to an atomic group (skeleton) that causes light emission in a fluorescent substance. The luminophore is preferably a skeleton having a π bond, further preferably includes an aromatic ring, and still further preferably includes a fused aromatic ring or a fused heteroaromatic ring. Examples of such a luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a material having an acene skeleton, especially an anthracene skeleton is suitably used as the host material. The use of a substance having an anthracene skeleton as the host material for the fluorescent substance makes it possible to obtain a light-emitting layer with high emission efficiency and high durability. Among the substances having an anthracene skeleton, a substance having a diphenylanthracene skeleton, in particular, a substance having a 9,10-diphenylanthracene skeleton, is chemically stable and thus is preferably used as the host material. The host material preferably has a carbazole skeleton to have higher hole-injection and hole-transport properties; further preferably, the host material has a benzocarbazole skeleton in which a benzene ring is further condensed to a carbazole skeleton, because the HOMO level of the host material having a benzocarbazole skeleton is higher than that of the host material having a carbazole skeleton by approximately 0.1 eV and the host material having a benzocarbazole skeleton is thus easier for holes to enter than the host material having a carbazole skeleton. In particular, the host material preferably has a dibenzocarbazole skeleton, because the HOMO level of the host material having a dibenzocarbazole skeleton is higher than that of the host material having a carbazole skeleton by approximately 0.1 eV, the host material having a dibenzocarbazole skeleton is thus easier for holes to enter than the host material having a carbazole skeleton, and the host material having a dibenzocarbazole skeleton has a higher hole-transport property and higher heat resistance than the host material having a carbazole skeleton. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Furthermore, a dibenzofuran skeleton is preferably included, in which case the reliability can be ensured without a reduction in the T₁ level.

Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthryl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4-(9-phenyl-9H-fluoren-9-yl)biphenyl-4′-yl]anthracene (abbreviation: FLPPA), 9-(1-naphthyl)-10-[4-(2-naphthyl)phenyl]anthracene (abbreviation: αN-βNPAnth), 9-(1-naphthyl)-10-(2-naphthyl)anthracene (abbreviation: α,βADN), 2-(10-phenylanthracen-9-yl)dibenzofuran, 2-(10-phenyl-9-anthryl)benzo[b]naphtho[2,3-d]furan (abbreviation: Bnf(II)PhA), 9-(2-naphthyl)-10-[3-(2-naphthyl)phenyl]anthracene (abbreviation: βN-mβNPAnth), and 1-{4-[10-(biphenyl-4-yl)-9-anthryl]phenyl}-2-ethyl-1H-benzimidazole (abbreviation: EtBImPBPhA). In particular, CzPA, cgDBCzPA, 2mBnfPPA, and PCzPA exhibit excellent properties and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material with a hole-transport property to the content of the material with an electron-transport property may be 1:19 to 19:1.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed of these mixed materials. These mixed materials are preferably selected so as to form an exciplex that exhibits light emission whose wavelength overlaps with the wavelength on a lowest-energy-side absorption band of the light-emitting substance, in which case energy can be transferred smoothly and light emission can be obtained efficiently. The use of such a structure is preferable because the driving voltage can also be reduced.

Note that at least one of the materials forming an exciplex may be a phosphorescent substance. In this case, triplet excitation energy can be efficiently converted into singlet excitation energy by reverse intersystem crossing.

In order to form an exciplex efficiently, a material having an electron-transport property is preferably combined with a material having a hole-transport property and a HOMO level higher than or equal to that of the material having an electron-transport property. In addition, the LUMO level of the hole-transport material is preferably higher than or equal to that of the electron-transport material. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength side than the emission spectrum of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient photoluminescence (PL) lifetime of the mixed film has a longer lifetime component or has a larger proportion of delayed component than that of each of the materials, observed by comparison of transient PL of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

The electron-transport layer 114 contains a material having an electron-transport property. The material having an electron-transport property preferably has an electron mobility higher than or equal to 1×10⁻⁷ cm²/Vs, further preferably higher than or equal to 1×10⁻⁶ cm²/Vs in the case where the square root of the electric field strength [V/cm] is 600. Note that any other substance can also be used as long as the substance has an electron-transport property higher than a hole-transport property. An organic compound including a π-electron deficient heteroaromatic ring is preferable as the above organic compound. The organic compound including a π-electron deficient heteroaromatic ring is preferably one or more of an organic compound including a heteroaromatic ring having an azole skeleton, an organic compound including a heteroaromatic ring having a pyridine skeleton, an organic compound including a heteroaromatic ring having a diazine skeleton, and an organic compound including a heteroaromatic ring having a triazine skeleton.

As the organic compound having an electron-transport property that can be used for the electron-transport layer 114, any of the aforementioned organic compounds that can be given as the organic compound having an electron-transport property in the light-emitting layer 113 can be used. Among the above materials, the organic compound that includes a heteroaromatic ring having a diazine skeleton, the organic compound that includes a heteroaromatic ring having a pyridine skeleton, and the organic compound that includes a heteroaromatic ring having a triazine skeleton are preferable because of having high reliability. In particular, the organic compound that includes a heteroaromatic ring having a diazine (pyrimidine or pyrazine) skeleton and the organic compound that includes a heteroaromatic ring having a triazine skeleton have a high electron-transport property to contribute to a reduction in driving voltage. In particular, an organic compound having a phenanthroline skeleton such as mTpPPhen, PnNPhen, or mPPhen2P is preferable, and an organic compound having a phenanthroline dimer structure such as mPPhen2P is further preferable because of high stability.

Note that the electron-transport layer 114 may have a stacked-layer structure. A layer in the stacked-layer structure of the electron-transport layer 114, which is in contact with the light-emitting layer 113, may function as a hole-blocking layer. In the case where the electron-transport layer in contact with the light-emitting layer functions as a hole-blocking layer, the electron-transport layer is preferably formed using a material having a lower HOMO level than a material contained in the light-emitting layer 113 by greater than or equal to 0.5 eV.

A layer that contains a compound or a complex of an alkali metal or an alkaline earth metal such as 8-hydroxyquinolinato-lithium (abbreviation: Liq), 1,1′-pyridine-2,6-diyl-bis(1,3,4,6,7,8-hexahydro-2H-pyrimido[1,2-a]pyrimidine) (abbreviation: hpp2Py), or the like may be provided as the electron-injection layer 115. As the electron-injection layer 115, an alkali metal, an alkaline earth metal, or a compound thereof may be contained in a layer formed using a substance having an electron-transport property.

Instead of the electron-injection layer 115, a charge-generation layer 116 may be provided (FIG. 1B). The charge-generation layer 116 refers to a layer capable of injecting holes into a layer in contact with the cathode side of the charge-generation layer 116 and electrons into a layer in contact with the anode side thereof when a potential is applied. The charge-generation layer 116 includes at least a p-type layer 117. The p-type layer 117 is preferably formed using any of the composite materials given above as examples of materials that can be used for the hole-injection layer 111. The p-type layer 117 may be formed by stacking a film containing the above-described acceptor material as a material included in the composite material and a film containing a hole-transport material. When a potential is applied to the p-type layer 117, electrons are injected into the electron-transport layer 114 and holes are injected into the cathode; thus, the light-emitting device operates. Since the organic compound of one embodiment of the present invention is an organic compound capable of forming a film with a low refractive index, using the organic compound for the p-type layer 117 enables the light-emitting device to have high external quantum efficiency.

Note that the charge-generation layer 116 preferably includes one or both of an electron-relay layer 118 and an electron-injection buffer layer 119 in addition to the p-type layer 117.

The electron-relay layer 118 includes at least the substance having an electron-transport property and has a function of preventing an interaction between the electron-injection buffer layer 119 and the p-type layer 117 and smoothly transferring electrons. The LUMO level of the substance having an electron-transport property included in the electron-relay layer 118 is preferably between the LUMO level of the acceptor substance in the p-type layer 117 and the LUMO level of a substance included in a layer of the electron-transport layer 114 that is in contact with the charge-generation layer 116. As a specific value of the energy level, the LUMO level of the substance having an electron-transport property in the electron-relay layer 118 is preferably higher than or equal to −5.0 eV, further preferably higher than or equal to −5.0 eV and lower than or equal to −3.0 eV. Note that as the substance having an electron-transport property in the electron-relay layer 118, a phthalocyanine-based material or a metal complex having a metal-oxygen bond and an aromatic ligand is preferably used.

The electron-injection buffer layer 119 can be formed using a substance having a high electron-injection property, e.g., an alkali metal, an alkaline earth metal, a rare earth metal, or a compound thereof (an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)).

In the case where the electron-injection buffer layer 119 contains a substance having an electron-transport property and a donor substance, the donor substance can be an organic compound such as tetrathianaphthacene (abbreviation: TTN), nickelocene, or decamethylnickelocene, as well as an alkali metal, an alkaline earth metal, a rare earth metal, or a compound thereof (e.g., an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)). As the substance having an electron-transport property, a material similar to the above-described material for the electron-transport layer 114 can be used.

The second electrode 102 is an electrode including a cathode. The second electrode 102 may have a stacked-layer structure, in which case a layer in contact with the organic compound layer 103 functions as a cathode. For the cathode, a metal, an alloy, an electrically conductive compound, or a mixture thereof each having a low work function (specifically, lower than or equal to 3.8 eV) can be used, for example. Specific examples of such a cathode material include elements belonging to Group 1 or 2 of the periodic table, such as alkali metals (e.g., lithium (Li) or cesium (Cs)), magnesium (Mg), calcium (Ca), and strontium (Sr), alloys containing these elements (e.g., MgAg and AlLi), compounds containing these elements (e.g., lithium fluoride (LiF), cesium fluoride (CsF), and calcium fluoride (CaF₂)), rare earth metals such as europium (Eu) and ytterbium (Yb), and alloys containing these rare earth metals. However, when the electron-injection layer 115 or a thin film formed using any of the above materials having a low work function is provided between the second electrode 102 and the electron-transport layer, a variety of conductive materials such as Al, Ag, ITO, or indium oxide-tin oxide containing silicon or silicon oxide can be used for the cathode regardless of the work function.

When the second electrode 102 is formed using a material that transmits visible light, the light-emitting device can emit light from the second electrode 102 side.

Films of these conductive materials can be deposited by a dry process such as a vacuum evaporation method or a sputtering method, an ink-jet method, a spin coating method, or the like. Alternatively, a wet process using a sol-gel method or a wet process using a paste of a metal material may be employed.

The organic compound layer 103 can be formed by any of a variety of methods, including a dry process and a wet process. For example, a vacuum evaporation method, a gravure printing method, an offset printing method, a screen printing method, an ink-jet method, a spin coating method, or the like may be used.

Different deposition methods may be used to form the electrodes or the layers described above.

Next, an embodiment of a light-emitting device with a structure in which a plurality of light-emitting units are stacked (this type of light-emitting device is also referred to as a stacked or tandem device) is described with reference to FIG. 1C. This light-emitting device includes a plurality of light-emitting units between an anode and a cathode. One light-emitting unit has substantially the same structure as the organic compound layer 103 illustrated in FIG. 1A. In other words, the light-emitting device illustrated in FIG. 1C includes a plurality of light-emitting units, and the light-emitting device illustrated in FIG. 1A or 1B includes a single light-emitting unit.

In FIG. 1C, a first light-emitting unit 511 and a second light-emitting unit 512 are stacked between a first electrode 501 and a second electrode 502, and a charge-generation layer 513 is provided between the first light-emitting unit 511 and the second light-emitting unit 512. The first electrode 501 and the second electrode 502 correspond, respectively, to the first electrode 101 and the second electrode 102 illustrated in FIG. 1A, and the materials given in the description for FIG. 1A can be used. Furthermore, the first light-emitting unit 511 and the second light-emitting unit 512 may have the same structure or different structures.

The charge-generation layer 513 has a function of injecting electrons into one of the light-emitting units and injecting holes into the other of the light-emitting units when voltage is applied between the first electrode 501 and the second electrode 502. That is, in FIG. 1C, the charge-generation layer 513 injects electrons into the first light-emitting unit 511 and holes into the second light-emitting unit 512 when voltage is applied such that the potential of the anode becomes higher than the potential of the cathode.

The charge-generation layer 513 preferably has a structure similar to that of the charge-generation layer 116 described with reference to FIG. 1B. A composite material of an organic compound and a metal oxide enables low-voltage driving and low-current driving because of having an excellent carrier-injection property and an excellent carrier-transport property. In the case where the anode-side surface of a light-emitting unit is in contact with the charge-generation layer 513, the charge-generation layer 513 can also function as a hole-injection layer of the light-emitting unit; therefore, a hole-injection layer is not necessarily provided in the light-emitting unit.

In the case where the electron-injection buffer layer 119 is provided in the charge-generation layer 513, the electron-injection buffer layer 119 functions as the electron-injection layer in the light-emitting unit on the anode side; thus, an electron-injection layer is not necessarily formed in the light-emitting unit on the anode side.

The light-emitting device having two light-emitting units is described with reference to FIG. 1C; however, one embodiment of the present invention can also be applied to a light-emitting device in which three or more light-emitting units are stacked. With a plurality of light-emitting units partitioned by the charge-generation layer 513 between a pair of electrodes as in the light-emitting device of this embodiment, it is possible to provide a long-life device that can emit light with high luminance at a low current density. A light-emitting apparatus that can be driven at a low voltage and has low power consumption can also be provided.

When the emission colors of the light-emitting units are different, light emission of a desired color can be obtained from the light-emitting device as a whole. For example, in a light-emitting device having two light-emitting units, the emission colors of the first light-emitting unit may be red and green and the emission color of the second light-emitting unit may be blue, so that the light-emitting device can emit white light as the whole.

The organic compound layer 103, the first light-emitting unit 511, the second light-emitting unit 512, the layers such as the charge-generation layer, and the electrodes that are described above can be formed by a method such as an evaporation method (including a vacuum evaporation method), a droplet discharge method (also referred to as an ink-jet method), a coating method, or a gravure printing method. A low molecular material, a middle molecular material (including an oligomer and a dendrimer), or a high molecular material may be included in the above components.

### Embodiment 3

Described in this embodiment is an example in which the light-emitting device of one embodiment of the present invention is used as a display element of a display device. Note that although a light-emitting device shown in this embodiment is formed by a photolithography method, the light-emitting device may be formed by a method using a fine metal mask or the like.

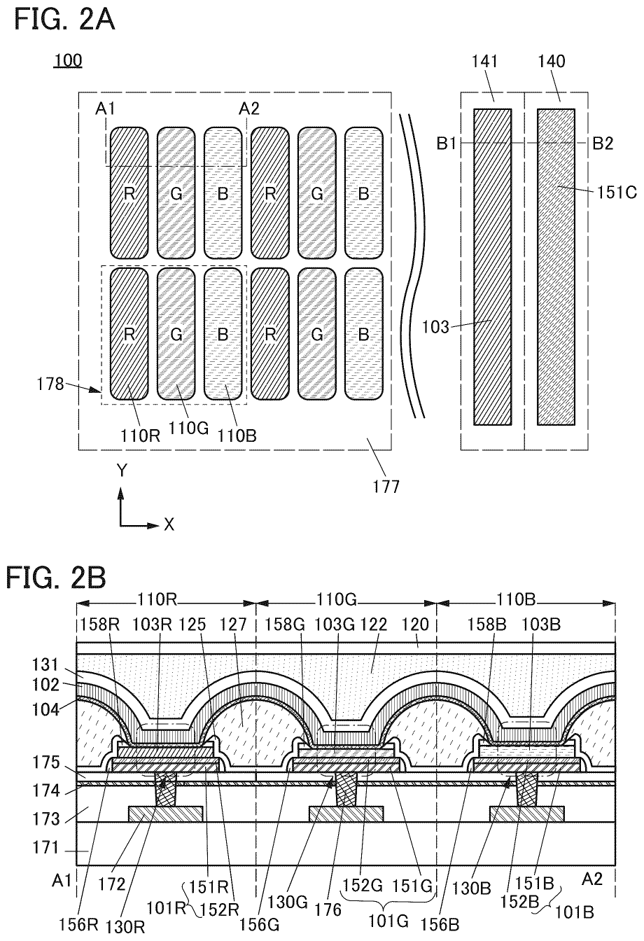

As illustrated in FIGS. 2A and 2B, a plurality of light-emitting devices 130 are formed over an insulating layer 175 to constitute a display device.

A display device includes a pixel portion 177 in which a plurality of pixels 178 are arranged in matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and yellow (Y), and four subpixels emitting light of R, G, and B and infrared light (IR).

In this specification and the like, the row direction and the column direction are sometimes referred to as the X direction and the Y direction, respectively. The X direction and the Y direction intersect with each other and are perpendicular to each other, for example.

FIG. 2A illustrates an example where subpixels of different colors are arranged in the X direction and subpixels of the same color are arranged in the Y direction. Note that subpixels of different colors may be arranged in the Y direction, and subpixels of the same color may be arranged in the X direction.

Outside the pixel portion 177, a connection portion 140 is provided and a region 141 may also be provided. The region 141 is provided between the pixel portion 177 and the connection portion 140. The organic compound layer 103 is provided in the region 141. A conductive layer 151C is provided in the connection portion 140.

Although FIG. 2A illustrates an example where the region 141 and the connection portion 140 are positioned on the right side of the pixel portion 177, the positions of the region 141 and the connection portion 140 are not particularly limited. The number of regions 141 and the number of connection portions 140 can each be one or more.

FIG. 2B is an example of a cross-sectional view along the dashed-dotted line A1-A2 in FIG. 2A. As illustrated in FIG. 2B, the display device includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 131 is provided to cover the light-emitting device 130. A substrate 120 is bonded to the protective layer 131 with a resin layer 122. An inorganic insulating layer 125 and an insulating layer 127 over the inorganic insulating layer 125 are preferably provided between the adjacent light-emitting devices 130.

Although FIG. 2B shows cross sections of a plurality of the inorganic insulating layers 125 and a plurality of the insulating layers 127, it is preferable that the inorganic insulating layers 125 be connected to each other and the insulating layers 127 be connected to each other when the display device is seen from above. In other words, the inorganic insulating layer 125 and the insulating layer 127 each preferably has an opening over a first electrode.

In FIG. 2B, a light-emitting device 130R, a light-emitting device 130G, and a light-emitting device 130B are shown as the light-emitting devices 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, 130G, or 130B may emit visible light of another color or infrared light. It can be said that in FIG. 2B, the light-emitting devices 130R and 130G are adjacent light-emitting devices and the light-emitting devices 130G and 130B are adjacent light-emitting devices.

The display device of one embodiment of the present invention can be, for example, a top-emission display device where light is emitted in the direction opposite to a substrate over which light-emitting devices are formed. Note that the display device of one embodiment of the present invention may be of a bottom emission type.

The light-emitting device 130R emits red light (preferably emits phosphorescent light), and preferably has the structure shown in Embodiment 2. The light-emitting device 130R includes a first electrode (pixel electrode) including a conductive layer 151R and a conductive layer 152R, a first layer 103R over the first electrode, a common layer 104 over the first layer 103R, and the second electrode (common electrode) 102 over the common layer 104. The common layer 104 is preferably an electron-injection layer.

The light-emitting device 130G emits green light (preferably emits phosphorescent light), and preferably has the structure shown in Embodiment 2. The light-emitting device 130G includes a first electrode (pixel electrode) including a conductive layer 151G and a conductive layer 152G, a first layer 103G over the first electrode, the common layer 104 over the first layer 103G, and the second electrode (common electrode) 102 over the common layer 104. The common layer 104 is preferably an electron-injection layer.

The light-emitting device 130B emits blue light (preferably emits fluorescent light), and preferably has the structure shown in Embodiment 2. The light-emitting device 130B includes a first electrode (pixel electrode) including a conductive layer 151B and a conductive layer 152B, a first layer 103B over the first electrode, the common layer 104 over the first layer 103B, and the second electrode (common electrode) 102 over the common layer 104. The common layer 104 is preferably an electron-injection layer.

In the light-emitting device, one of the pixel electrode (first electrode) and the common electrode (second electrode) functions as an anode and the other functions as a cathode. In this embodiment, description is made on the assumption that the pixel electrode functions as the anode and the common electrode functions as the cathode unless otherwise specified.

The first layers 103R, 103G, and 103B are island-shaped layers that are independent of each other on a light-emitting device basis or on an emission color basis. It is preferable that the first layers 103R, 103G, and 103B not overlap with one another. The first layers included in the plurality of light-emitting devices 130 formed in the light-emitting apparatus, such as the first layers 103R, 103G, and 103B, are collectively referred to as a first layer group 103 in some cases. Providing the island-shaped first layer group 103 in each of the light-emitting devices 130 can suppress leakage current between the adjacent light-emitting devices 130 even in a high-resolution display device. This can prevent crosstalk, so that a display device with extremely high contrast can be obtained. Specifically, a display device having high current efficiency at low luminance can be obtained.

The island-shaped first layer group 103 is formed by forming an EL film for each emission color and processing the EL film by a photolithography technique.

The first layer 103 is preferably provided to cover the top surface and the side surface of the first electrode (pixel electrode) 101 of the light-emitting device 130. In this case, the aperture ratio of the display device can be easily increased as compared to the structure where an end portion of the first layer 103 is positioned inward from an end portion of the pixel electrode. Covering the side surface of the pixel electrode of the light-emitting device 130 with the first layer 103 can inhibit the first electrode 101 from being in contact with the second electrode 102; hence, a short circuit of the light-emitting device 130 can be inhibited.

In the display device of one embodiment of the present invention, the first electrode (pixel electrode) 101 of the light-emitting device preferably has a stacked-layer structure. For example, in the example illustrated in FIG. 2B, the first electrode 101 of the light-emitting device 130 is a stack of the conductive layer 151 on the insulating layer 171 side and the conductive layer 152 on the organic compound layer side.

A metal material can be used for the conductive layer 151, for example. Specifically, it is possible to use a metal such as aluminum (Al), titanium (T₁), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, indium tin oxide containing silicon can be suitably used for the conductive layer 152 because of having a high work function, for example, a work function higher than or equal to 4.0 eV.

The conductive layer 151 and the conductive layer 152 may each be a stack of a plurality of layers containing different materials. In that case, the conductive layer 151 may include a layer formed using a material that can be used for the conductive layer 152, such as a conductive oxide, and the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

The conductive layer 151 preferably has a tapered end portion. Specifically, the conductive layer 151 preferably has a tapered end portion with a taper angle of less than 90°. In that case, the conductive layer 152 provided along the side surface of the conductive layer 151 also has a tapered shape. When the side surface of the conductive layer 152 has a tapered shape, coverage with the first layer 103 provided along the side surface of the conductive layer 152 can be improved.

### Embodiment 4

In this embodiment, a display device of one embodiment of the present invention will be described.

The display device in this embodiment can be a high-resolution display device. Thus, the display device in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The display device in this embodiment can be a high-definition display device or a large-sized display device. Accordingly, the display device in this embodiment can be used for display portions of a digital camera, a digital video camera, a digital photo frame, a mobile phone, a portable game console, a portable information terminal, and an audio reproducing device, in addition to display portions of electronic devices with a relatively large screen, such as a television device, desktop and laptop personal computers, a monitor of a computer and the like, digital signage, and a large game machine such as a pachinko machine.

### [Display Module]

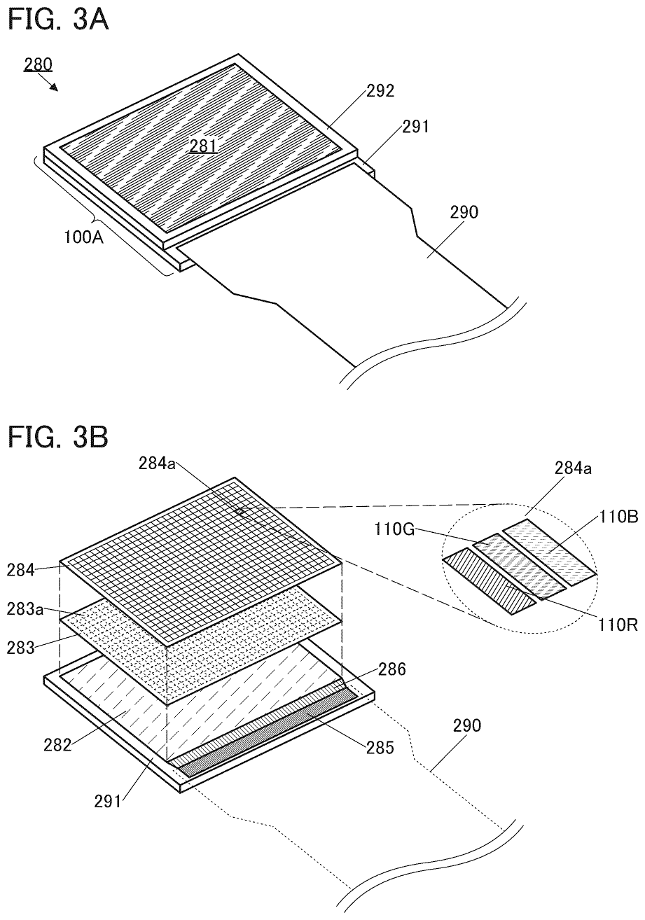

FIG. 3A is a perspective view of a display module 280. The display module 280 includes a display device 100A and an FPC 290. Note that the display device included in the display module 280 is not limited to the display device 100A and may be any of display devices 100B to 100E described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 3B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion over the substrate 291 that does not overlap with the pixel portion 284. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 3B. The pixels 284a can employ any of the structures described in the above embodiments. FIG. 3B illustrates an example where the pixel 284a has a structure similar to that of the pixel 178 illustrated in FIGS. 2A and 2B.

The pixel circuit portion 283 includes a plurality of pixel circuits 283a arranged periodically.

Each of the pixel circuits 283a is a circuit that controls driving of a plurality of elements included in one pixel 284a.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high.

Such a display module 280 has extremely high resolution, and thus can be suitably used for a VR device such as an HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic devices including a relatively small display portion.

### [Display Device 100A]

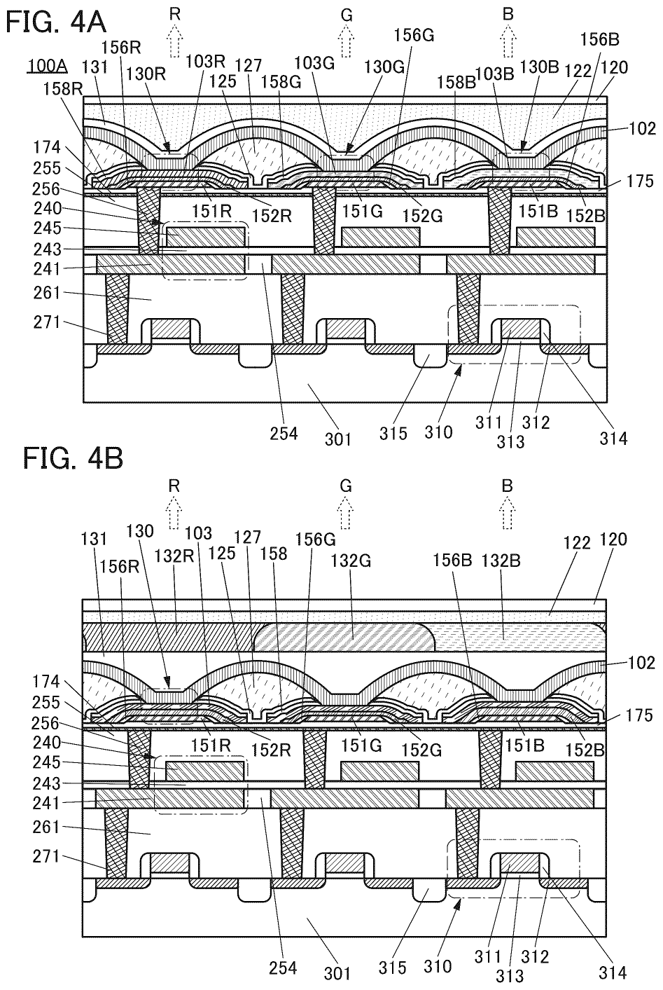

The display device 100A illustrated in FIG. 4A includes a substrate 301, the light-emitting devices 130R, 130G, and 130B, a capacitor 240, and a transistor 310.

The substrate 301 corresponds to the substrate 291 in FIGS. 3A and 3B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 so as to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. An insulator is provided in regions between adjacent light-emitting devices.

An insulating layer 156R is provided to include a region overlapping with the side surface of the conductive layer 151R. An insulating layer 156G is provided to include a region overlapping with the side surface of the conductive layer 151G. An insulating layer 156B is provided to include a region overlapping with the side surface of the conductive layer 151B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. A sacrificial layer 158R is positioned over the first layer 103R. A sacrificial layer 158G is positioned over the first layer 103G. A sacrificial layer 158B is positioned over the first layer 103B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. Any of a variety of conductive materials can be used for the plugs.

The protective layer 131 is provided over the light-emitting devices 130R, 130G, and 130B. The substrate 120 is bonded to the protective layer 131 with the resin layer 122. Embodiment 3 can be referred to for the details of the light-emitting device 130 and the components thereover up to the substrate 120. The substrate 120 corresponds to the substrate 292 in FIG. 3A.

FIG. 4B illustrates a variation example of the display device 100A illustrated in FIG. 4A. The display device illustrated in FIG. 4B includes a coloring layer 132R, a coloring layer 132G, and a coloring layer 132B, and each of the light-emitting devices 130 includes a region overlapping with one of the coloring layers 132R, 132G, and 132B. In the display device illustrated in FIG. 4B, the light-emitting device 130 can emit white light, for example. The coloring layer 132R, the coloring layer 132G, and the coloring layer 132B can transmit red light, green light, and blue light, respectively, for example.

### [Display Device 100B]

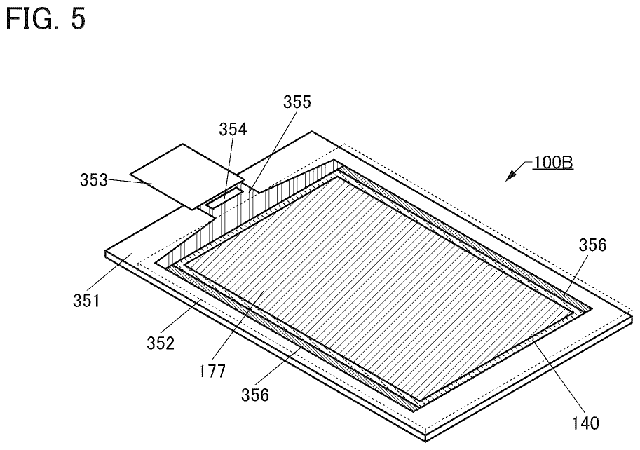

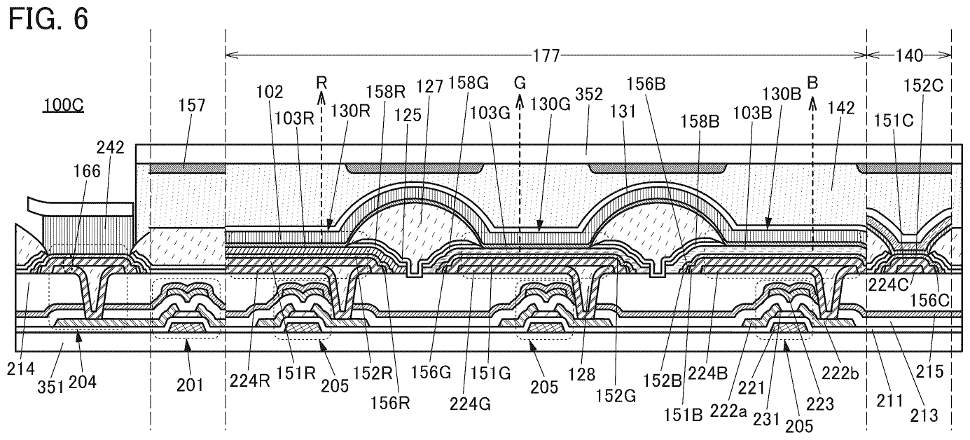

FIG. 5 is a perspective view of the display device 100B, and FIG. 6 is a cross-sectional view of the display device 100C.

In the display device 100B, a substrate 352 and a substrate 351 are bonded to each other. In FIG. 5, the substrate 352 is denoted by a dashed line.

The display device 100B includes the pixel portion 177, the connection portion 140, a circuit 356, a wiring 355, and the like. FIG. 5 illustrates an example in which an IC 354 and an FPC 353 are mounted on the display device 100B. Thus, the structure illustrated in FIG. 5 can be regarded as a display module including the display device 100B, the integrated circuit (IC), and the FPC. Here, a display device in which a substrate is equipped with a connector such as an FPC or mounted with an IC is referred to as a display module.

The connection portion 140 is provided outside the pixel portion 177. The number of connection portions 140 may be one or more. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 5 illustrates an example in which the IC 354 is provided over the substrate 351 by a chip on glass (COG) method, a chip on film (COF) method, or the like. An IC including a scan line driver circuit, a signal line driver circuit, or the like can be used as the IC 354, for example. Note that the display device 100B and the display module are not necessarily provided with an IC. Alternatively, the IC may be mounted on the FPC by a COF method, for example.

FIG. 6 illustrates an example of cross sections of part of a region including the FPC 353, part of the circuit 356, part of the pixel portion 177, part of the connection portion 140, and part of a region including an end portion of the display device 100B.

### [Display Device 100C]

The display device 100C illustrated in FIG. 6 includes a transistor 201, a transistor 205, the light-emitting device 130R that emits red light, the light-emitting device 130G that emits green light, the light-emitting device 130B that emits blue light, and the like between the substrate 351 and the substrate 352.

Embodiment 1 can be referred to for the details of the light-emitting devices 130R, 130G, and 130B.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B.

The conductive layer 224R is connected to a conductive layer 222b included in the transistor 205 through an opening provided in an insulating layer 214. An end portion of the conductive layer 151R is positioned outward from an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G and the insulating layer 156G in the light-emitting device 130G are not described in detail because they are respectively similar to the conductive layers 224R, 151R, and 152R and the insulating layer 156R in the light-emitting device 130R; the same applies to the conductive layers 224B, 151B, and 152B and the insulating layer 156B in the light-emitting device 130B.

The conductive layers 224R, 224G, and 224B each have a depression portion covering the opening provided in the insulating layer 214. A layer 128 is embedded in the depression portion.

The layer 128 has a function of filling the depression portions of the conductive layers 224R, 224G, and 224B to obtain planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depression portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 131 is provided over the light-emitting devices 130R, 130G, and 130B. The protective layer 131 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 6, a solid sealing structure is employed, in which a space between the substrate 352 and the substrate 351 is filled with the adhesive layer 142. Alternatively, the space may be filled with an inert gas (e.g., nitrogen or argon), that is, a hollow sealing structure may be employed. In that case, the adhesive layer 142 may be provided so as not to overlap with the light-emitting device. Furthermore, the space may be filled with a resin other than the frame-like adhesive layer 142.

FIG. 6 illustrates an example in which the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and a conductive layer 152C obtained by processing the same conductive film as the conductive layers 152R, 152G, and 152B. In the example illustrated in FIG. 6, an insulating layer 156C is provided to include a region overlapping with the side surface of the conductive layer 151C.

The display device 100C has a top-emission structure. Light from the light-emitting device is emitted toward the substrate 352. For the substrate 352, a material having a high visible-light-transmitting property is preferably used. In the case where the light-emitting device emits infrared or near-infrared light, a material having a high transmitting property with respect to infrared or near-infrared light is preferably used. The first electrode (pixel electrode) contains a material that reflects visible light, and the second electrode (counter electrode) contains a material that transmits visible light.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

An inorganic insulating film is preferably used as each of the insulating layers 211, 213, and 215.

An organic insulating layer is suitable for the insulating layer 214 functioning as a planarization layer.

Each of the transistors 201 and 205 includes a conductive layer 221 functioning as a gate, the insulating layer 211 functioning as the gate insulating layer, a conductive layer 222a and the conductive layer 222b functioning as a source and a drain, a semiconductor layer 231, the insulating layer 213 functioning as the gate insulating layer, and a conductive layer 223 functioning as a gate.

A connection portion 204 is provided in a region of the substrate 351 that does not overlap with the substrate 352. In the connection portion 204, the wiring 355 is electrically connected to the FPC 353 through a conductive layer 166 and a connection layer 242. As an example, the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

A light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### [Display Device 100D]

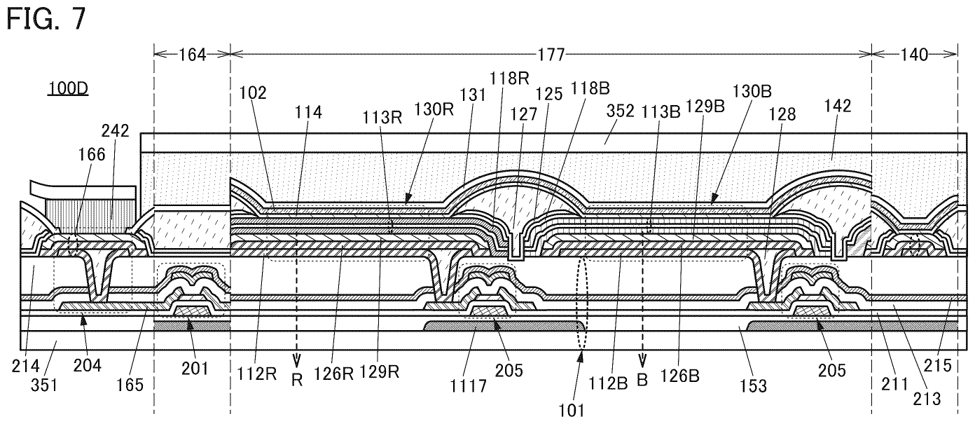

The display device 100D in FIG. 7 differs from the display device 100C in FIG. 6 mainly in having a bottom-emission structure.

Light from the light-emitting device is emitted toward the substrate 351. For the substrate 351, a material having a high visible-light-transmitting property is preferably used. By contrast, there is no limitation on the light-transmitting property of a material used for the substrate 352.

A light-blocking layer 1117 is preferably formed between the substrate 351 and the transistor 201 and between the substrate 351 and the transistor 205. FIG. 7 illustrates an example in which the light-blocking layer 1117 is provided over the substrate 351, an insulating layer 153 is provided over the light-blocking layer 1117, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material having a high visible-light-transmitting property is used for each of the conductive layers 112R, 112B, 126R, 126B, 129R, and 129B. A material that reflects visible light is preferably used for the second electrode 102.

Although not illustrated in FIG. 7, the light-emitting device 130G is also provided.

Although FIG. 7 and the like illustrate an example in which the top surface of the layer 128 includes a flat portion, the shape of the layer 128 is not particularly limited.

### [Display Device 100E]

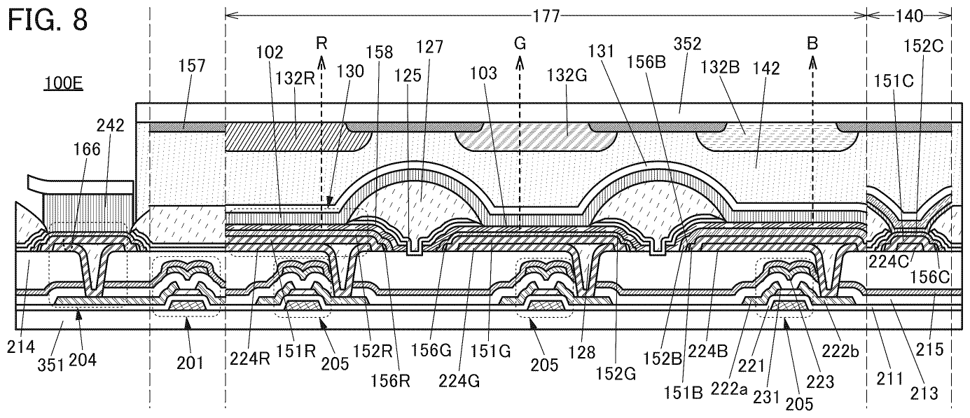

The display device 100E illustrated in FIG. 8 is a variation example of the display device 100C illustrated in FIG. 6 and differs from the display device 100C mainly in including the coloring layers 132R, 132G, and 132B.

In the display device 100E, the light-emitting device 130 includes a region overlapping with one of the coloring layers 132R, 132G, and 132B. The coloring layers 132R, 132G, and 132B can be provided on a surface of the substrate 352 on the substrate 351 side. End portions of the coloring layers 132R, 132G, and 132B can overlap with the light-blocking layer 157.

In the display device 100E, the light-emitting device 130 can emit white light, for example. The coloring layer 132R, the coloring layer 132G, and the coloring layer 132B can transmit red light, green light, and blue light, respectively, for example. Note that in the display device 100E, the coloring layers 132R, 132G, and 132B may be provided between the protective layer 131 and the adhesive layer 142.

Although FIGS. 6 and 8 and the like illustrate an example in which the top surface of the layer 128 includes a flat portion, the shape of the layer 128 is not particularly limited.

This embodiment can be combined as appropriate with the other embodiments or the examples. In this specification, in the case where a plurality of structure examples are shown in one embodiment, the structure examples can be combined as appropriate.

### Embodiment 5

In this embodiment, electronic devices of embodiments of the present invention will be described.

Electronic devices of this embodiment include the display device of one embodiment of the present invention in their display portions. The display device of one embodiment of the present invention has low power consumption. Thus, the display device of one embodiment of the present invention can be used for display portions of a variety of electronic devices.

Examples of the electronic devices include a digital camera, a digital video camera, a digital photo frame, a mobile phone, a portable game console, a portable information terminal, and an audio reproducing device, in addition to electronic devices with a relatively large screen, such as a television device, desktop and laptop personal computers, a monitor of a computer and the like, digital signage, and a large game machine such as a pachinko machine.

In particular, the display device of one embodiment of the present invention has low power consumption, and thus can be suitably used for a relatively small electronic device. Examples of such an electronic device include watch-type and bracelet-type information terminal devices (wearable devices) and wearable devices worn on the head, such as a VR device like a head-mounted display, a glasses-type AR device, and an MR device.

The electronic device in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

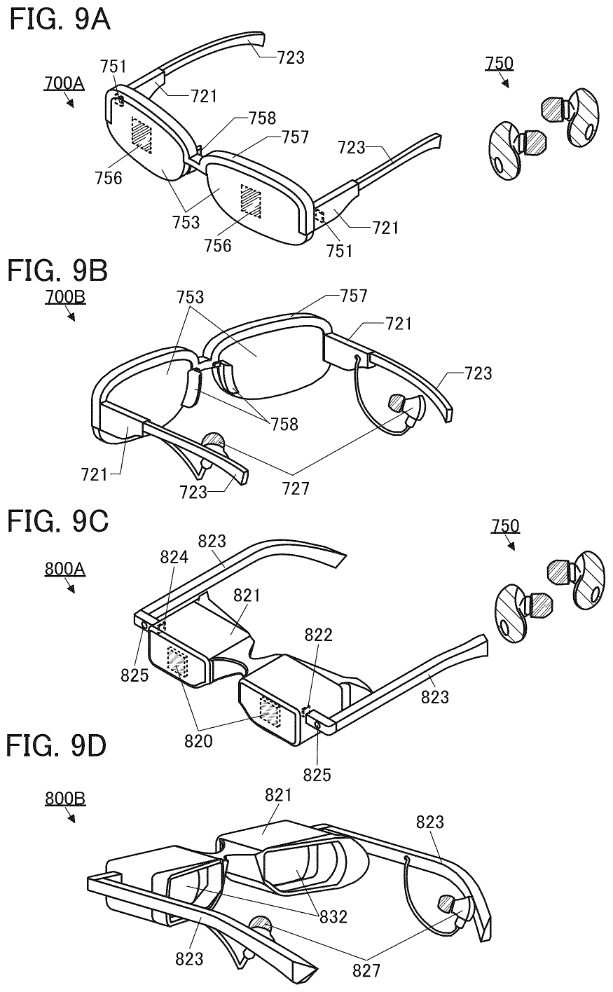

Examples of head-mounted wearable devices are described with reference to FIGS. 9A to 9D.

An electronic device 700A illustrated in FIG. 9A and an electronic device 700B illustrated in FIG. 9B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display device of one embodiment of the present invention can be used for the display panels 751. Thus, the electronic device can have low power consumption and be driven for a long time.

The electronic devices 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753.

In the electronic devices 700A and 700B, a camera capable of capturing images of the front side may be provided as the image capturing portion. Furthermore, when the electronic devices 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic devices 700A and 700B are provided with a battery, so that they can be charged wirelessly and/or by wire.

A touch sensor module may be provided in the housing 721.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

An electronic device 800A illustrated in FIG. 9C and an electronic device 800B illustrated in FIG. 9D each include a pair of display portions 820, a housing 821, a communication portion 822, a pair of wearing portions 823, a control portion 824, a pair of image capturing portions 825, and a pair of lenses 832.

The display device of one embodiment of the present invention can be used for the display portions 820. Thus, the electronic device can have low power consumption and be driven for a long time.

The display portions 820 are positioned inside the housing 821 so as to be seen through the lenses 832. When the pair of display portions 820 display different images, three-dimensional display using parallax can be performed.

The electronic devices 800A and 800B preferably include a mechanism for adjusting the lateral positions of the lenses 832 and the display portions 820 so that the lenses 832 and the display portions 820 are positioned optimally in accordance with the positions of the user's eyes.

The electronic device 800A or the electronic device 800B can be mounted on the user's head with the wearing portions 823.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of fields of view, such as a telescope field of view and a wide field of view.

The electronic device 800A may include a vibration mechanism that functions as bone-conduction earphones.

The electronic devices 800A and 800B may each include an input terminal. To the input terminal, a cable for supplying a video signal from a video output device or the like, power for charging a battery provided in the electronic device, and the like can be connected.

The electronic device of one embodiment of the present invention may have a function of performing wireless communication with earphones 750.

The electronic device may include an earphone portion. The electronic device 700B in FIG. 9B includes earphone portions 727. Part of a wiring that connects the earphone portion 727 and the control portion may be positioned inside the housing 721 or the wearing portion 723.

Similarly, the electronic device 800B in FIG. 9D includes earphone portions 827. For example, the earphone portion 827 can be connected to the control portion 824 by wire.

As described above, both the glasses-type device (e.g., the electronic devices 700A and 700B) and the goggles-type device (e.g., the electronic devices 800A and 800B) are preferable as the electronic device of one embodiment of the present invention.

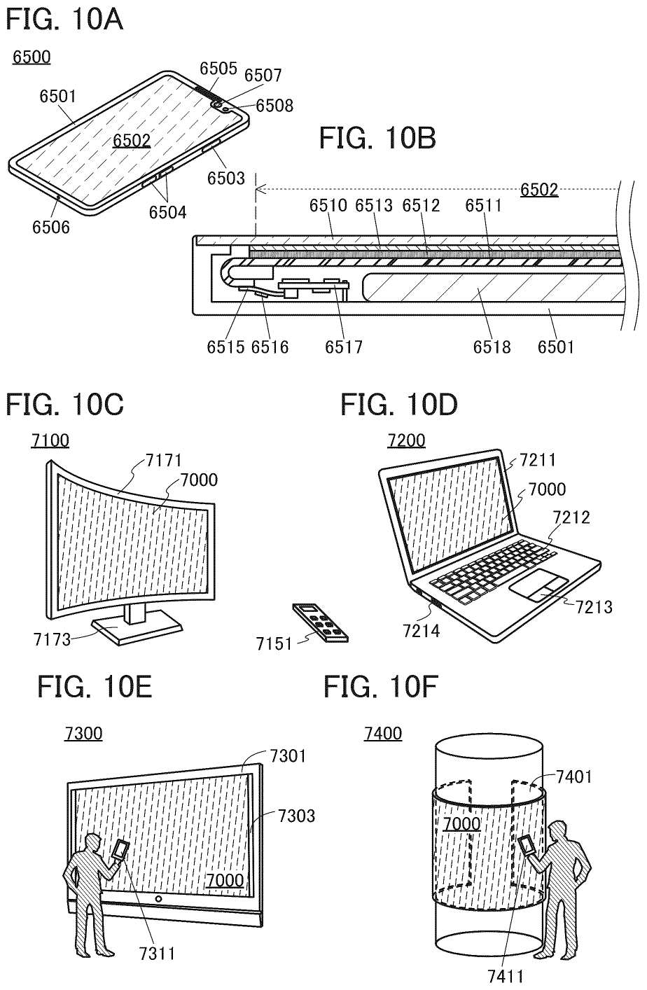

An electronic device 6500 in FIG. 10A is a portable information terminal that can be used as a smartphone.

The electronic device 6500 includes a housing 6501, a display portion 6502, a power button 6503, buttons 6504, a speaker 6505, a microphone 6506, a camera 6507, a light source 6508, and the like. The display portion 6502 has a touch panel function.

The display device of one embodiment of the present invention can be used for the display portion 6502. Thus, the electronic device can have low power consumption and be driven for a long time.

FIG. 10B is a schematic cross-sectional view including an end portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The display device of one embodiment of the present invention can be used in the display panel 6511. Thus, the electronic device can be extremely lightweight. Since the display panel 6511 is extremely thin, the battery 6518 with high capacity can be mounted without an increase in the thickness of the electronic device. Moreover, part of the display panel 6511 is folded back so that a connection portion with the FPC 6515 is provided on the back side of the pixel portion, whereby the electronic device can have a narrow bezel.

FIG. 10C illustrates an example of a television device. In a television device 7100, a display portion 7000 is incorporated in a housing 7171. Here, the housing 7171 is supported by a stand 7173.

The display device of one embodiment of the present invention can be used for the display portion 7000. Thus, the electronic device can have low power consumption and be driven for a long time.

Operation of the television device 7100 illustrated in FIG. 10C can be performed with an operation switch provided in the housing 7171 and a separate remote control 7151.

FIG. 10D illustrates an example of a laptop personal computer. A laptop personal computer 7200 includes a housing 7211, a keyboard 7212, a pointing device 7213, an external connection port 7214, and the like. The display portion 7000 is incorporated in the housing 7211.

The display device of one embodiment of the present invention can be used for the display portion 7000. Thus, the electronic device can have low power consumption and be driven for a long time.

FIGS. 10E and 10F illustrate examples of digital signage.

Digital signage 7300 illustrated in FIG. 10E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 10F illustrates digital signage 7400 attached to a cylindrical pillar 7401. The digital signage 7400 includes the display portion 7000 provided along a curved surface of the pillar 7401.

In FIGS. 10E and 10F, the display device of one embodiment of the present invention can be used in the display portion 7000. Thus, the electronic devices can be highly reliable.

A larger area of the display portion 7000 can increase the amount of information that can be provided at a time. The display portion 7000 having a larger area attracts more attention, so that the effectiveness of the advertisement can be increased, for example.

As illustrated in FIGS. 10E and 10F, it is preferable that the digital signage 7300 or the digital signage 7400 can work with an information terminal 7311 or an information terminal 7411, such as a smartphone that a user has, through wireless communication.

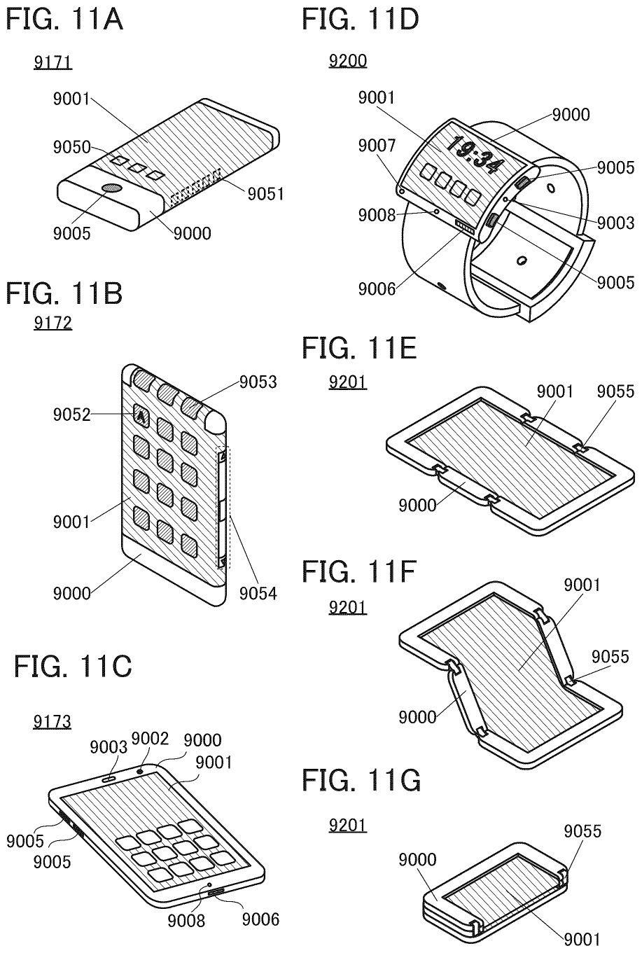

Electronic devices illustrated in FIGS. 11A to 11G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic devices illustrated in FIGS. 11A to 11G have a variety of functions. For example, the electronic devices can have a function of displaying a variety of information (e.g., a still image, a moving image, and a text image) on the display portion, a touch panel function, a function of displaying a calendar, date, time, and the like, a function of controlling processing with the use of a variety of software (programs), a wireless communication function, and a function of reading out and processing a program or data stored in a recording medium.

The electronic devices in FIGS. 11A to 11G will be described in detail below.

FIG. 11A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 11A illustrates an example in which three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, the title and sender of an e-mail, an SNS message, or the like, the date, the time, remaining battery, and the radio field intensity. Alternatively, the icon 9050 or the like may be displayed at the position where the information 9051 is displayed.

FIG. 11B is a perspective view of a portable information terminal 9172. The portable information terminal 9172 has a function of displaying information on three or more surfaces of the display portion 9001. Here, information 9052, information 9053, and information 9054 are displayed on the respective surfaces. For example, the user of the portable information terminal 9172 can check the information 9053 displayed such that it can be seen from above the portable information terminal 9172, with the portable information terminal 9172 put in a breast pocket of his/her clothes.

FIG. 11C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, a camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 11D is a perspective view of a watch-type portable information terminal 9200. The portable information terminal 9200 can be used as a Smartwatch (registered trademark), for example. The display surface of the display portion 9001 is curved, and an image can be displayed on the curved display surface. Furthermore, for example, mutual communication between the portable information terminal 9200 and a headset capable of wireless communication can be performed, and thus hands-free calling is possible. With the connection terminal 9006, the portable information terminal 9200 can perform mutual data transmission with another information terminal and charging. Note that the charging operation may be performed by wireless power feeding.

FIGS. 11E to 11G are perspective views of a foldable portable information terminal 9201. FIG. 11E is a perspective view showing the portable information terminal 9201 that is opened. FIG. 11G is a perspective view showing the portable information terminal 9201 that is folded. FIG. 11F is a perspective view showing the portable information terminal 9201 that is shifted from one of the states in FIGS. 11E and 11G to the other. The portable information terminal 9201 is highly portable when folded. When the portable information terminal 9201 is opened, a seamless large display region is highly browsable. The display portion 9001 of the portable information terminal 9201 is supported by three housings 9000 joined together by hinges 9055. The display portion 9001 can be folded with a radius of curvature of greater than or equal to 0.1 mm and less than or equal to 150 mm, for example.

This embodiment can be combined as appropriate with the other embodiments or the examples. In this specification, in the case where a plurality of structure examples are shown in one embodiment, the structure examples can be combined as appropriate.

### Example 1

### Synthesis Example 1

In this synthesis example, a synthesis method of N-(3′,5′-ditertiarybutylbiphenyl-2-yl)-N-(4-cyclohexylphenyl)-9,9-dimethyl-9H-fluoren-2-amine (abbreviation: mmtBuoBichPAF), which is represented by Structural Formula (100) in Embodiments, is described. The structural formula of mmtBuoBichPAF is shown below.

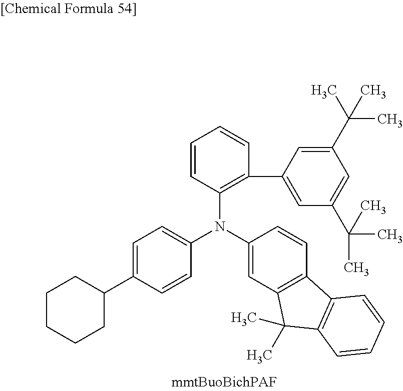 [SMILES: CC(C)(C)c1cc(-c2ccccc2N(c2ccc(C3CCCCC3)cc2)c2ccc3c(c2)C(C)(C)c2ccccc2-3)cc(C(C)(C)C)c1]

### Step 1: Synthesis of 2-bromo-3′,5′-di-tert-butylbiphenyl

Into a 500-mL three-neck flask were put 22 g (94 mmol) of (3,5-di-tert-butylphenyl)boronic acid, 23 g (81 mmol) of 1-bromo-2-iodobenzene, 42 g (0.30 mol) of potassium carbonate, 300 mL of toluene, 75 mL of ethanol, and 75 mL of water, and the flask was degassed under reduced pressure while the mixture was stirred, and then the air in the flask was replaced with nitrogen. After that, 91 g (0.79 mmol) of tetrakis(triphenylphosphine)palladium was added, and this mixture was stirred while being heated at 90° C. for approximately 7 hours. After that, 0.43 g (1.4 mmol) of tris(2-methylphenyl)phosphine and 0.19 g (0.85 mmol) of palladium acetate were added, and the mixture was stirred while being heated for 4 hours. After that, the temperature of the flask was lowered to room temperature, the mixture was separated, and the organic layer was washed with a saturated solution of sodium carbonate and saturated saline. The obtained organic layer was separated by filtration after being dried with magnesium sulfate. The filtrate was concentrated, and the obtained solution was purified by silica gel column chromatography. The solution was concentrated and dried under reduced pressure at room temperature, whereby 27 g of a target colorless oily substance was obtained in a yield of 96%. The synthesis scheme of Step 1 (s1-1) is shown below.

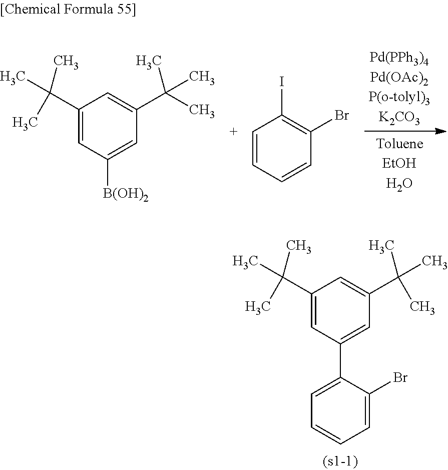 [SMILES: CC(C)(C)c1cc(B(O)O)cc(C(C)(C)C)c1 ; Brc1ccccc1I ; CC(C)(C)c1cc(-c2ccccc2Br)cc(C(C)(C)C)c1]

### Step 2: Synthesis of N-(4-cyclohexylphenyl)-N-(9,9-dimethyl-9H-fluoren-2yl)amine

Into a 1000 mL three-neck flask were put 25 g (0.12 mol) of 9,9-dimethyl-9H-fluoren-2-amine, 28 g (0.11 mol) of 4-cyclohexyl-1-bromobenzene, 34 g (0.35 mol) of sodium-tert-butoxide, and 600 mL of xylene, and the flask was degassed under reduced pressure while the mixture was stirred, and then the air in the flask was replaced with nitrogen. After that, 0.44 g (1.2 mmol) of allylpalladium chloride dimer (II) (abbreviation: (AllylPdCl)₂) and 1.5 g (2.0 mmol) of tris(2-methylphenyl)phosphine were added, and the mixture was heated at 120° C. for approximately 2 hours. After that, 0.74 g (1.8 mmol) of 2-dicyclohexylphosphino-2′,6′-dimethoxybiphenyl (abbreviation: SPhos (registered trademark)) was added, and the mixture was stirred while being heated for 30 minutes. After that, the temperature of the flask was lowered to approximately 60° C., approximately 4 mL of water was added to the mixture, and a precipitated solid was separated by filtration. The filtrate was concentrated, and the obtained solution was purified by silica gel column chromatography. The obtained solution was concentrated to give a condensed toluene solution. This toluene solution was concentrated and dried under reduced pressure at approximately 60° C., whereby a 39 g of a target brown oily substance was obtained in a yield of 91%. The synthesis scheme of Step 2 (s2-1) is shown below.

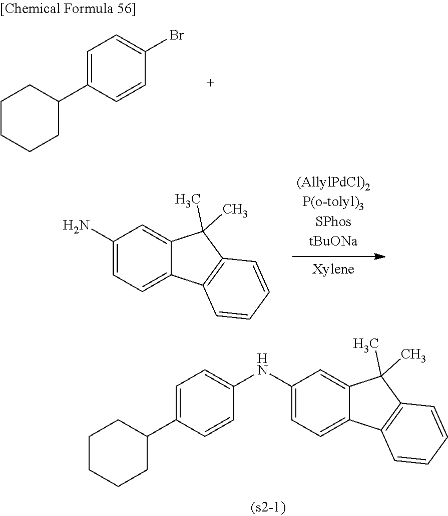 [SMILES: Brc1ccc(C2CCCCC2)cc1 ; CC1(C)c2ccccc2-c2ccc(N)cc21 ; CC1(C)c2ccccc2-c2ccc(Nc3ccc(C4CCCCC4)cc3)cc21]

### Step 3: Synthesis of mmtBuoBichPAF

Into a 100-mL three-neck flask were put 4.1 g (12 mmol) of 2-bromo-3′,5′-di-tert-butylbiphenyl, 5.2 g (14 mmol) of N-(4-cyclohexylphenyl)-N-(9,9-dimethyl-9H-fluoren-2yl)amine, 3.7 g (39 mmol) of sodium tert-butoxide, and 58 mL of xylene, and the flask was degassed under reduced pressure while the mixture was stirred, and then the air in the container was replaced with nitrogen. After the mixture was stirred while being heated at 50° C. for ten minutes, 60 mg (0.16 mmol) of allylpalladium(II) chloride dimer (abbreviation: (AllylPdCl)₂) and 0.17 g (0.48 mmol) of di(tert-butyl)(1-methyl-2,2-diphenylcyclopropyl)phosphine (abbreviation: cBRIDP (registered trademark)) were added, and the mixture was stirred while being heated at 120° C. for approximately 6 hours. After that, the temperature of the flask was lowered to approximately room temperature, approximately 2 mL of water was added to the mixture, and a precipitated solid was separated by filtration. The filtrate was concentrated, and the obtained condensed solution was purified by silica gel column chromatography. The obtained solution was concentrated to give a condensed toluene solution. The toluene solution was dropped into ethanol for reprecipitation. This suspension was cooled, and the precipitate was filtrated at approximately 10° C. and the obtained solid was dried at approximately 130° C. under reduced pressure, whereby 3.5 g of a target white solid was obtained in a yield of 47%. The synthesis scheme of Step 3 (s3-1) is shown below.

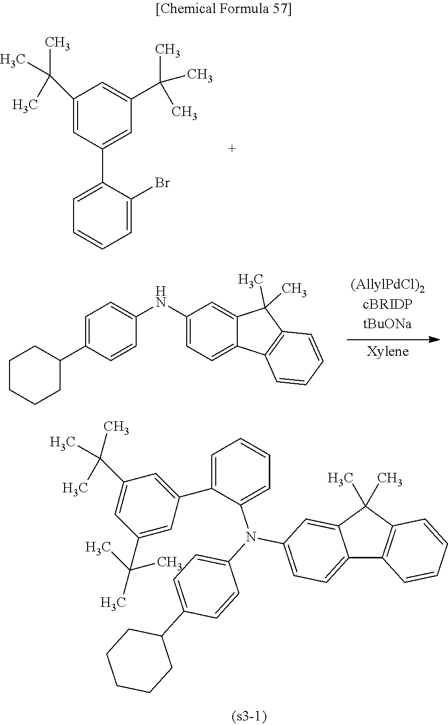 [SMILES: CC(C)(C)c1cc(-c2ccccc2Br)cc(C(C)(C)C)c1 ; CC1(C)c2ccccc2-c2ccc(Nc3ccc(C4CCCCC4)cc3)cc21 ; CC(C)(C)c1cc(-c2ccccc2N(c2ccc(C3CCCCC3)cc2)c2ccc3c(c2)C(C)(C)c2ccccc2-3)cc(C(C)(C)C)c1]

Next, 1.9 g of the obtained white solid was purified by a train sublimation method. In the purification by sublimation, a boat in which a material was put was heated under conditions where the argon flow rate was 10 mL/min and the pressure was 2.6 Pa. The boat was sandwiched between two heating bands, and the heating temperature of one of the heating bands was set to 210° C. and the heating temperature of the other heating band was set to 190° C. The heating temperature in a portion where the material was collected was set to 150° C., and the heating was performed for approximately 17 hours. After the purification by sublimation, 1.7 g of a pale yellow glassy solid was obtained at a collection rate of 89%.

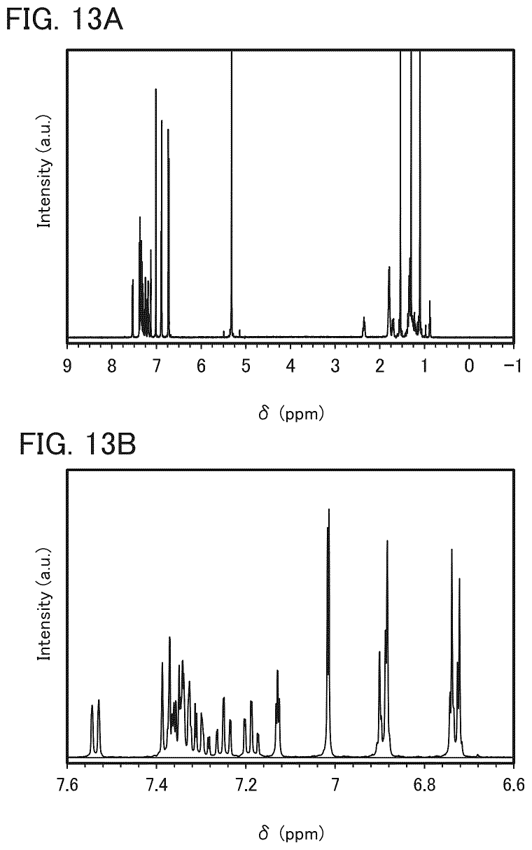

Measurement results by nuclear magnetic resonance (¹H-NMR) spectroscopy of the obtained white solid are shown below and in FIGS. 13A and 13B. FIG. 13B is a chart where the range from 6.6 ppm to 7.6 ppm in FIG. 13A is enlarged. The results show that mmtBuoBichPAF was obtained in this synthesis example.

¹H-NMR.δ (CD₂Cl₂): 7.54 (d, 1H, J=7.5 Hz), 7.39-7.28 (m, 6H), 7.25 (td, 1H, J1=7.4 Hz, J2=1.5 Hz), 7.19 (td, 1H, J1=7.5 Hz, J2=1.0 Hz), 7.13 (t, 1H, J=2.0 Hz), 7.02 (d, 2H, J=1.5 Hz), 6.91-6.88 (m, 3H), 6.74-6.72 (m, 3H), 2.38-2.33 (brm, 1H), 1.79 (d, 4H, J=6.5 Hz), 1.71 (d, 1H, J=13 Hz), 1.38-1.23 (m, 11H), 1.10 (s, 18H).

### <Measurement of Physical Property>

Next, the ultraviolet-visible absorption spectra (hereinafter, simply referred to as “absorption spectra”) and photoluminescence (PL) spectra of a toluene solution of mmtBuoBichPAF and a thin film of mmtBuoBichPAF were measured.

An ultraviolet-visible light spectrophotometer (V-550DS, manufactured by JASCO Corporation) was used for the measurement of the absorption spectrum. The PL spectrum was measured with a fluorescence spectrophotometer (FP-8600, JASCO Corporation).

To calculate the absorption spectrum of mmtBuoBichPAF in a toluene solution, the absorption spectrum of toluene put in a quartz cell was measured and then subtracted from the absorption spectrum of the toluene solution of mmtBuoBichPAF put in a quartz cell.

To obtain the absorption spectrum and the PL spectrum of the thin film, a measurement sample was measured. The measurement sample was fabricated in the following manner: mmtBuoBichPAF was deposited over a quartz substrate by a vacuum evaporation method and sealed using another quartz substrate as a counter substrate. Note that the PL spectrum was obtained by measuring the sealed sample, and the absorption spectrum was obtained by measuring the sample from which the sealing was removed and the counter substrate was detached. The absorption spectrum was obtained by subtraction of the absorption spectrum of the quartz substrate from the absorption spectrum of mmtBuoBichPAF deposited over the quartz substrate.

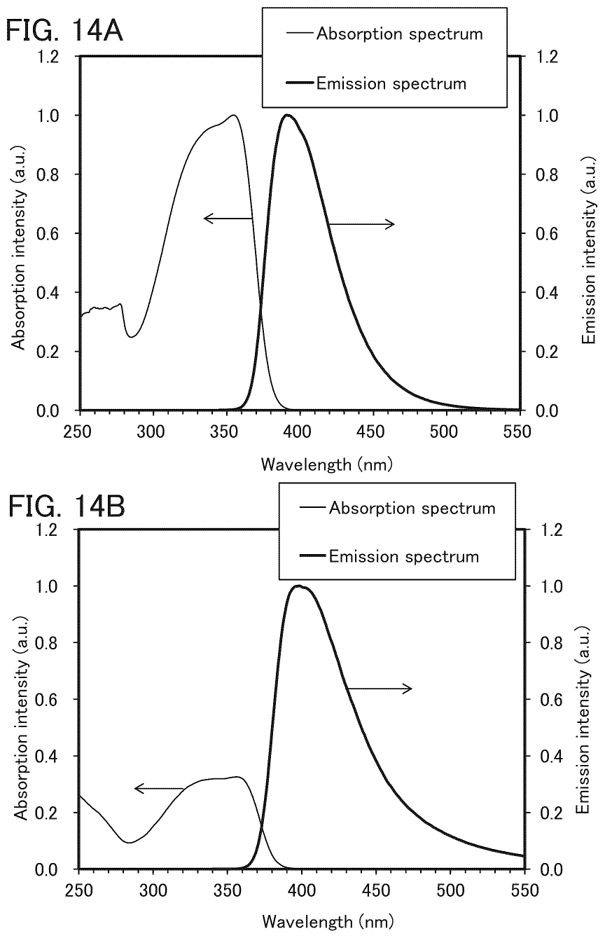

FIG. 14A shows the measurement results of the toluene solution and FIG. 14B shows the measurement results of the thin film. The measurement results show that the toluene solution of mmtBuoBichPAF has an absorption peak at around 355 nm, and the thin film of mmtBuoBichPAF has an absorption peak at around 356 nm. Furthermore, the toluene solution of mmtBuoBichPAF exhibited an emission wavelength peak at around 391 nm (excitation wavelength: 335 nm), and the thin film of mmtBuoBichPAF exhibited an emission wavelength peak at around 398 nm (excitation wavelength: 330 nm).

Next, the HOMO level and the LUMO level of mmtBuoBichPAF were obtained through a cyclic voltammetry (CV) measurement. The calculation method is described below.

An electrochemical analyzer (ALS model 600A or 600C, BAS Inc.) was used as a measurement apparatus. To prepare a solution for the CV measurement, dehydrated dimethylformamide (DMF; Sigma-Aldrich Inc., 99.8%, catalog No. 22705-6) was used as a solvent, and tetra-n-butylammonium perchlorate (n-Bu₄NClO₄; Tokyo Chemical Industry Co., Ltd., catalog No. T0836) as a supporting electrolyte was dissolved at a concentration of 100 mmol/L. Furthermore, the measurement target was also dissolved at a concentration of 2 mmol/L.

A platinum electrode (PTE platinum electrode, BAS Inc.) was used as a working electrode, a platinum electrode (Pt counter electrode for VC-3 (5 cm), BAS Inc.) was used as an auxiliary electrode, and an Ag/Ag⁺ electrode (RE-7 nonaqueous reference electrode, BAS Inc.) was used as a reference electrode. Note that the measurement was performed at room temperature (20° C. to 25° C.). The scan speed in the CV measurement was fixed to 0.1 V/s, and an oxidation potential Ea [V] and a reduction potential Ec [V] with respect to the reference electrode were measured. The potential Ea is an intermediate potential of an oxidation-reduction wave, and the potential Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Ea and LUMO level [eV]=−4.94−Ec.

As a result, in the measurement of an oxidation potential Ea [V] of mmtBuoBichPAF, the HOMO level was found to be −5.39 eV. In contrast, the LUMO level was found to be −1.9 eV in the measurement of the reduction potential Ec [V].

Next, the GSP_slope of an evaporated film of mmtBuoBichPAF was calculated. The GSP_slope of the evaporated film of mmtBuoBichPAF can be obtained by the method described in Embodiment 1. The results show that the GSP_slope of the evaporated film of mmtBuoBichPAF has a high value of 37.5 (mV/nm).

### Example 2

### Synthesis Example 2

In this synthesis example, a synthesis method of N-(3′,5′-ditertiarybutylbiphenyl-4-yl)-N-(3′,5′-ditertiarybutylbiphenyl-2-yl)-9,9-dimethyl-9H-fluoren-2-amine (abbreviation: dmmtBuopBBAF), which is represented by Structural Formula (145) in Embodiments, is described. The structural formula of dmmtBuopBBAF is shown below.

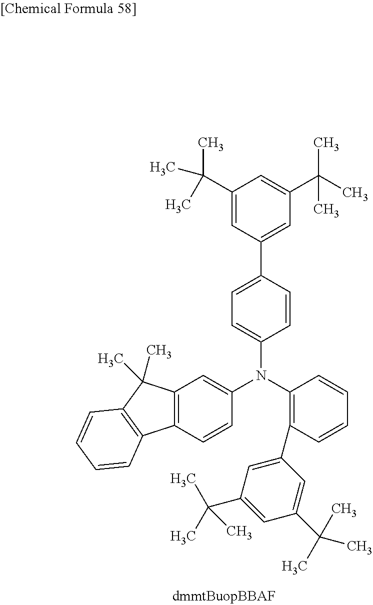 [SMILES: CC(C)(C)c1cc(-c2ccc(N(c3ccc4c(c3)C(C)(C)c3ccccc3-4)c3ccccc3-c3cc(C(C)(C)C)cc(C(C)(C)C)c3)cc2)cc(C(C)(C)C)c1]

### Step 1: Synthesis of 3′,5′-di-tert-butyl-4-chlorobiphenyl

Into a 2000 mL three-neck flask were put 30 g (0.11 mol) of 3,5-di-tert-butyl-1-bromobenzene, 19 g (0.12 mmol) of 4-chlorophenylboronic acid, 46 g (0.33 mol) of potassium carbonate, 550 mL of toluene, 140 mL of ethanol, and 160 mL of water, and the flask was degassed under reduced pressure while the mixture was stirred, and then the air in the flask was replaced with nitrogen. After that, 0.25 g (1.1 mmol) of palladium acetate and 0.70 g (2.3 mmol) of tris(2-methylphenyl)phosphine were added, and this mixture was stirred while being heated at 90° C. for approximately 5 hours. After that, the temperature of the flask was lowered to room temperature, the mixture was separated, and the organic layer was washed with a saturated aqueous solution of sodium carbonate and saturated saline. The obtained organic layer was separated by filtration after being dried with magnesium sulfate. The filtrate was concentrated, and the obtained solution was purified by silica gel column chromatography. The obtained solution was concentrated to give a condensed toluene solution. The toluene solution was dropped into ethanol for reprecipitation. This suspension was cooled, and the precipitate was filtrated at approximately 10° C., and the obtained solid was dried at approximately 60° C. under reduced pressure, whereby 30 g of a target white solid was obtained in a yield of 89%. The synthesis scheme of Step 1 (s1-2) is shown below.

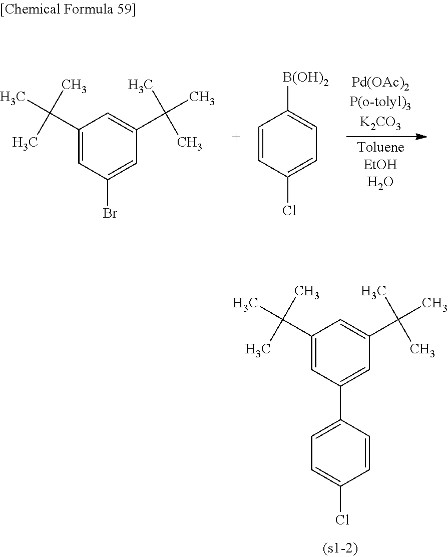 [SMILES: CC(C)(C)c1cc(Br)cc(C(C)(C)C)c1 ; OB(O)c1ccc(Cl)cc1 ; CC(C)(C)c1cc(-c2ccc(Cl)cc2)cc(C(C)(C)C)c1]

### Step 2: Synthesis of N-(3′,5′-di-tert-butylbiphenyl-2-yl)-9,9-dimethyl-9H-fluoren-2-amine

Into a 50-mL three-neck flask were put 3.6 g (10 mmol) of 2-bromo-3′,5′-di-tert-butylbiphenyl, 1.1 g (5.3 mmol) of 9,9-dimethyl-9H-fluoren-2-amine, 1.7 g (18 mmol) of sodium tert-butoxide, and 26 mL of xylene, and the flask was degassed under reduced pressure while the mixture was stirred, and then the air in the flask was replaced with nitrogen. After that, 40 mg (0.11 mmol) of allylpalladium chloride dimer (II) (abbreviation: (AllylPdCl)₂) and 0.10 mL of tri-tert-butylphosphine in a 10% hexane solution were added, and the mixture was stirred while being heated at approximately 140° C. for approximately 2 hours. After that, the temperature of the flask was lowered to room temperature, approximately 2 mL of water was added to the mixture, and a precipitated solid was separated by filtration. The filtrate was concentrated, and the obtained condensed solution was purified by silica gel column chromatography. The obtained fraction was concentrated and dried at room temperature under reduced pressure, whereby 4.4 g of a target brown oily substance was obtained in a yield of 89%. The synthesis scheme of Step 2 (s2-2) is shown below.

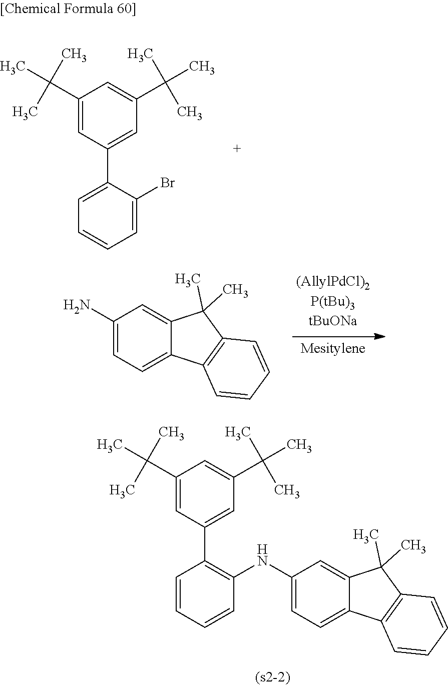

### Step 3: Synthesis of dmmtBuopBBAF

Into a 200-mL three-neck flask were put 3.2 g (6.8 mmol) of N-(3′,5′-di-tert-butylbiphenyl-2-yl)-9,9-dimethyl-9H-fluoren-2-amine, 2.2 g (7.3 mmol) of 3′,5′-di-tert-butyl-4-chlorobiphenyl, 2.0 g (21 mmol) of sodium tert-butoxide, and 38 mL of xylene, and the flask was degassed under reduced pressure while the mixture was stirred, and then the air in the container was replaced with nitrogen. After that, 29 mg (79 μmol) of allylpalladium(II) chloride dimer (abbreviation: (AllylPdCl)₂) and 0.10 g (0.28 mmol) of di(tert-butyl)(1-methyl-2,2-diphenylcyclopropyl)phosphine (abbreviation: cBRIDP (registered trademark)) were added, and the mixture was stirred while being heated at 160° C. for approximately 12 hours. After that, the temperature of the flask was lowered to approximately 70° C., approximately 2 mL of water was added to the mixture, and a precipitated solid was separated by filtration. The filtrate was concentrated, and the obtained condensed solution was purified by silica gel column chromatography. The obtained solution was concentrated to give a condensed toluene solution. The toluene solution was dropped into ethanol for reprecipitation. The precipitate was filtrated at approximately 10° C., and the obtained solid was dried at approximately 130° C. under reduced pressure, whereby 2.5 g of a target white solid was obtained in a yield of 50%. The synthesis scheme of Step 3 (s3-2) is shown below.

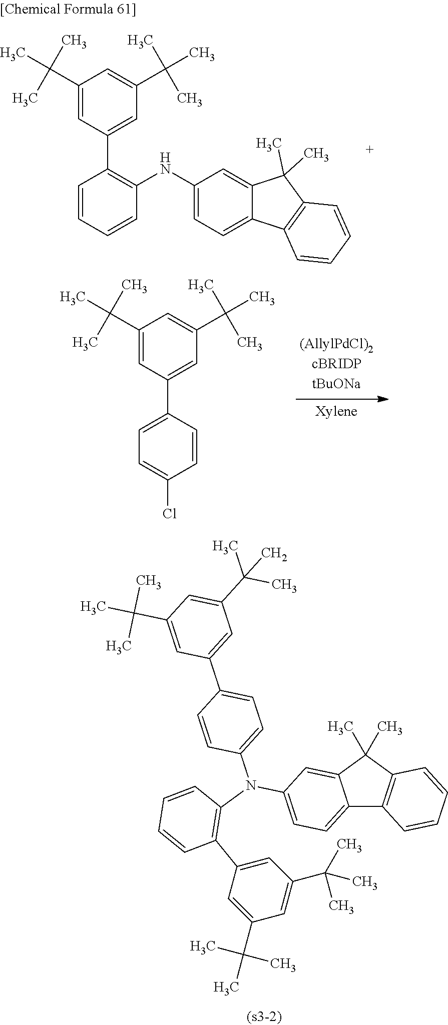 [SMILES: CC(C)(C)c1cc(-c2ccccc2Nc2ccc3c(c2)C(C)(C)c2ccccc2-3)cc(C(C)(C)C)c1 ; CC(C)(C)c1cc(-c2ccc(Cl)cc2)cc(C(C)(C)C)c1 ; CC(C)(C)c1cc(-c2ccc(N(c3ccc4c(c3)C(C)(C)c3ccccc3-4)c3ccccc3-c3cc(C(C)(C)C)cc(C(C)(C)C)c3)cc2)cc(C(C)(C)C)c1]

Then, 2.5 g of the obtained white solid was purified by a train sublimation method. In the purification by sublimation, a boat in which a material was put was heated under conditions where the argon flow rate was 10 mL/min and the pressure was 2.5 Pa. The boat was sandwiched between two heating bands, and the heating temperature of one of the heating bands was set to 222° C. and the heating temperature of the other heating band was set to 217° C. The heating temperature in a portion where the material was collected was set to 185° C., and the heating was performed for approximately 29 hours. After the purification by sublimation, 2.2 g of a pale yellow glassy solid was obtained at a collection rate of 88%.

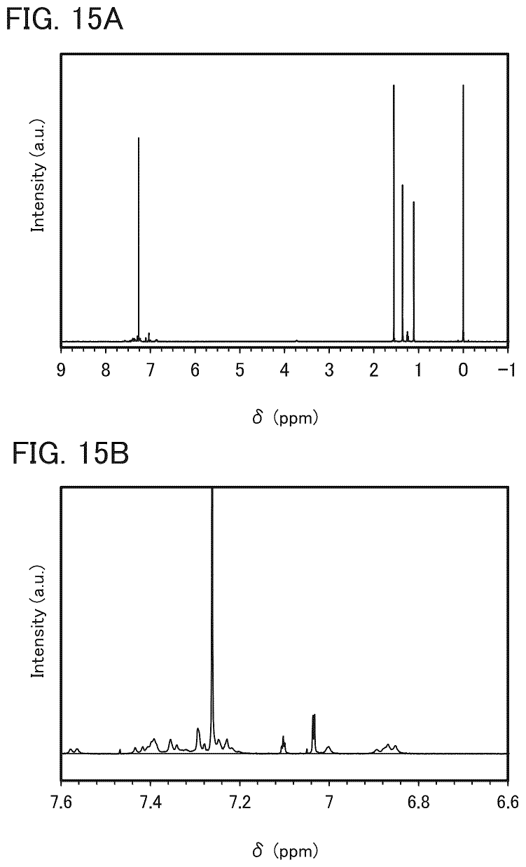

The ¹H-NMR measurement results of the obtained white solid are shown below and in FIGS. 15A and 15B. FIG. 15B is a chart where the range of from 6.6 ppm to 7.6 ppm in FIG. 15A is enlarged. The results show that dmmtBuopBBAF was obtained in this synthesis example.

¹H NMR.δ (CDCl₃): 7.57 (d, 1H, J=7.0 Hz), 7.47-7.28 (m, 10H), 7.25-7.20 (m, 3H), 7.10 (t, 1H, 1.8 Hz), 7.05-7.00 (m, 3H), 6.89-6.85 (m, 3H), 1.36 (s, 18H), 1.35 (s, 6H), 1.11 (s, 18H).

### <Measurement of Physical Property>

Next, the absorption spectra and the PL spectra of a toluene solution of dmmtBuopBBAF and a thin film of dmmtBuopBBAF were measured. The measurement was performed by a method similar to that in Example 1.

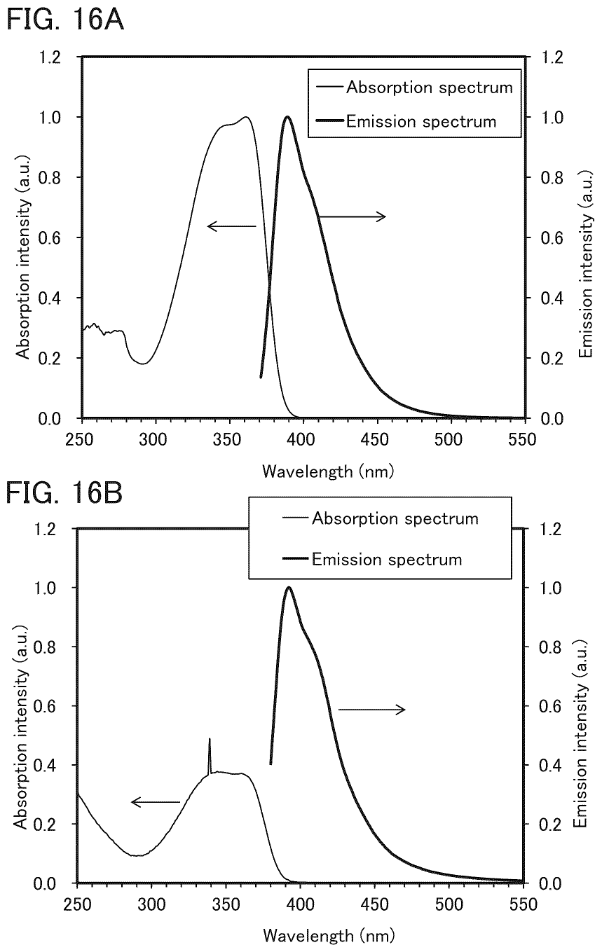

FIG. 16A shows the measurement results of the toluene solution and FIG. 16B shows the measurement results of the thin film. The measurement results show that the toluene solution of dmmtBuopBBAF has an absorption peak at around 361 nm, and the film of dmmtBuopBBAF has an absorption peak at around 367 nm. Furthermore, the toluene solution of dmmtBuopBBAF exhibited an emission wavelength peak at around 389 nm (excitation wavelength: 361 nm), and the thin film of dmmtBuopBBAF exhibited an emission wavelength peak at around 392 nm (excitation wavelength: 364 nm).

The HOMO level and the LUMO level of dmmtBuopBBAF were obtained through a cyclic voltammetry (CV) measurement. A calculation method is similar to that described in Example 1. According to the results, dmmtBuopBBAF has a HOMO level of −5.40 eV and a LUMO level of −2.0 eV.

Next, the GSP_slope of an evaporated film of dmmtBuopBBAF was calculated. The GSP_slope of the evaporated film of dmmtBuopBBAF can be obtained by the method described in Embodiment 1. The results show that the GSP_slope of the evaporated film of dmmtBuopBBAF has a high value of 46.2 (mV/nm).

### Example 3

In this example, a light-emitting device of one embodiment of the present invention will be described in detail. Structural formulae of typical organic compounds used in this example are shown below.

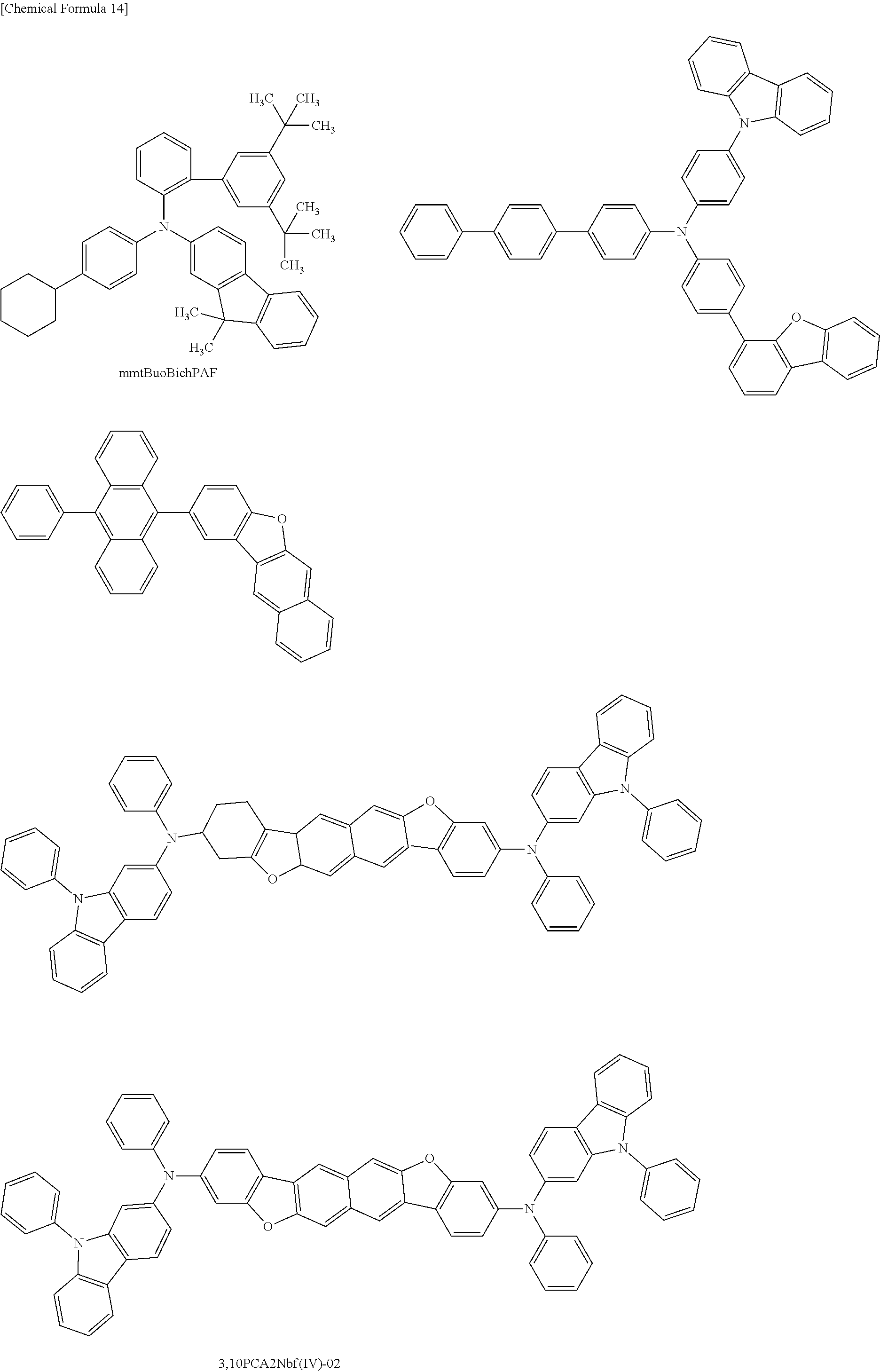

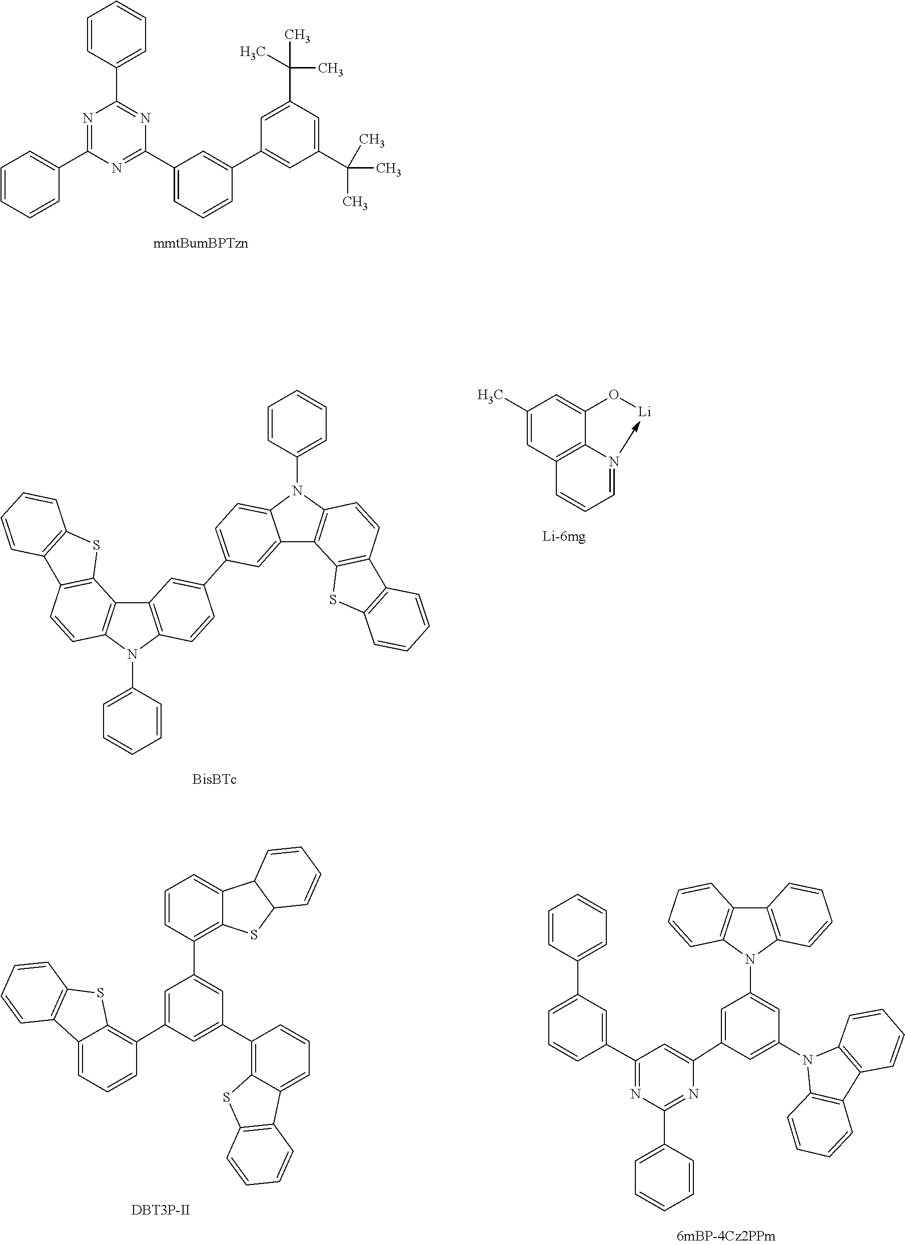

### (Method for Fabricating Light-Emitting Device 1)

First, indium tin oxide containing silicon oxide (ITSO) was deposited over a glass substrate to a thickness of 55 nm by a sputtering method to form the first electrode 101. Note that the electrode area was 2 mm×2 mm.

Next, in pretreatment for fabricating the light-emitting element over the substrate, the surface of the substrate was washed with water, and baking was performed at 200° C. for one hour.

After that, the substrate was transferred into a vacuum evaporation apparatus where the pressure had been reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Next, the substrate provided with the first electrode 101 was fixed to a substrate holder provided in the vacuum evaporation apparatus such that the surface on which the first electrode 101 was formed faced downward. Then, N-(3′,5′-ditertiarybutylbiphenyl-2-yl)-N-(4-cyclohexylphenyl)-9,9-dimethyl-9H-fluoren-2-amine (abbreviation: mmtBuoBichPAF) represented by Structural Formula (i) above and a fluorine-containing electron acceptor material with a molecular weight of 672 (OCHD-003) were deposited on the first electrode 101 (anode) to a thickness of 10 nm by co-evaporation using resistance heating such that the weight ratio of mmtBuoBichPAF to OCHD-003 was 1:0.1, whereby the hole-injection layer 111 was formed.

Subsequently, over the hole-injection layer 111, mmtBuoBichPAF was deposited to a thickness of 30 nm by evaporation to form a first hole-transport layer, and then N-[4-(dibenzofuran-4-yl)phenyl]-N-[4-(9H-carbazol-9-yl)phenyl]-p-terphenyl-4-amine (abbreviation: YGTPDBfB) represented by Structural Formula (ii) above was deposited to a thickness of 10 nm to form a second hole-transport layer, whereby the hole-transport layer 112 was formed. Note that the second hole-transport layer also functions as an electron-blocking layer.

Subsequently, 2-(10-phenyl-9-anthryl)benzo[b]naphtho[2,3-d]furan (abbreviation: Bnf(II)PhA) represented by Structural Formula (iii) above and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) represented by Structural Formula (iv) above were formed over the hole-transport layer 112 to a thickness of 25 nm by co-evaporation such that the weight ratio of Bnf(II)PhA to 3,10PCA2Nbf(IV)-02 was 1:0.015, whereby the light-emitting layer 113 was formed.

After that, over the light-emitting layer 113, 2-[3′-(9,9-dimethyl-9H-fluoren-2-yl)biphenyl-3-yl]-4,6-diphenyl-1,3,5-triazine (abbreviation: mFBPTzn) represented by Structure Formula (v) was deposited to a thickness of 10 nm, whereby a first electron-transport layer was formed. Sequentially, 2-[3-(2,6-dimethyl-3-pyridinyl)-5-(9-phenanthryl)phenyl]-4,6-diphenyl-1,3,5-triazine (abbreviation: mPn-mDMePyPTzn) represented by Structure Formula (vi) and 8-hydroxyquinolinato-lithium (abbreviation: Liq) were deposited by co-evaporation to a thickness of 20 nm at a weight ratio of 1:1, whereby a second electron-transport layer was formed. Accordingly, the electron-transport layer 114 was formed.

Over the electron-transport layer 114, Liq was deposited to a thickness of 1 nm to form the electron-injection layer 115.

Lastly, aluminum was deposited by evaporation to a thickness of 200 nm to form the second electrode 102, whereby the light-emitting device of one embodiment of the present invention was fabricated.

### (Method for Fabricating Comparative Light-Emitting Device 1)

A comparative light-emitting device 1 was fabricated in a manner similar to that for the light-emitting device 1 except that mmtBuoBichPAF in the light-emitting device 1 was replaced with N-(biphenyl-4-yl)-N-[4-(9-phenyl-9H-carbazol-3-yl)phenyl]-9,9-dimethyl-9H-fluoren-2-amine (abbreviation: PCBBiF) represented by Structural Formula (viii) above.

The structures of the light-emitting device 1 and the comparative light-emitting device 1 are listed in the following table.

TABLE 4

| | | Film | | Comparative |
|---|---|---|---|---|
| | | thickness | Light-emitting | light-emitting |
| | | (nm) | device 1 | device 1 |
| Second electrode | | 200 | Al | |
| Electron-injection layer | | 1 | Liq | |
| Electron-transport | 2 | 20 | mPn-mDMePyPTzn:Liq | |
| layer | | | (1:1) | |
| | 1 | 10 | mFBPTzn | |
| Light-emitting layer | | 25 | Bnf(II)PhA:3,10PCA2Nbf(IV)-02 | |
| | | | (1:0.015) | |
| Hole-transport | 2 | 10 | YGTPDBfB | |
| layer | 1 | 30 | mmtBuoBichPAF | PCBBiF |

| | | | |
|---|---|---|---|
| Hole-injection layer | 10 | mmtBuoBichPAF:OCHD-003 | PCBBiF:OCHD-003 |

| | |
|---|---|
| (1:0.1) | (1:0.1) |

| | | |
|---|---|---|
| First electrode | 55 | ITSO |

Each of the light-emitting devices was sealed using a glass substrate in a glove box including a nitrogen atmosphere so as not to be exposed to the air (a sealing material was applied to surround an element and UV treatment and heat treatment at 80° C. for an hour were performed at the time of sealing). Then, the initial characteristics of the light-emitting devices were measured.

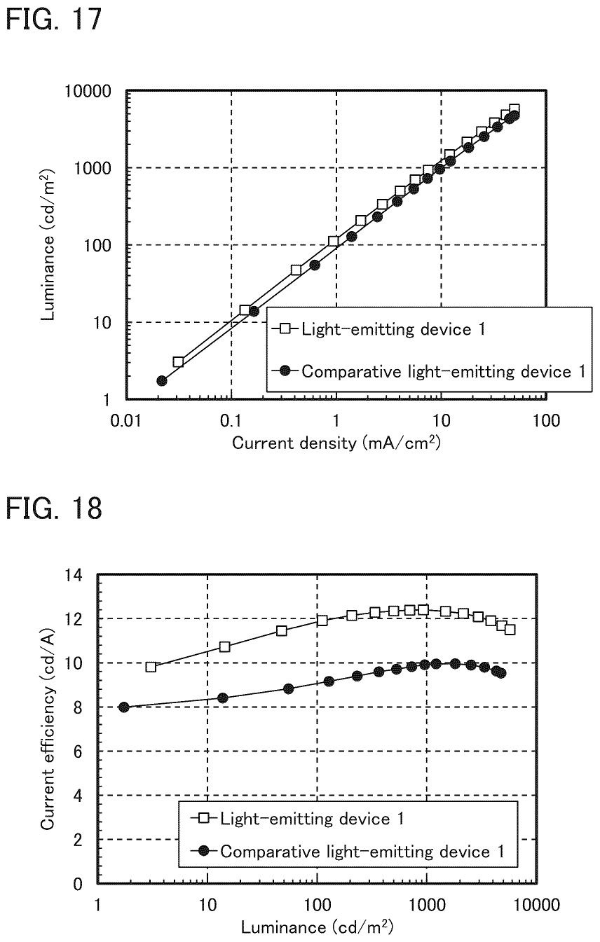

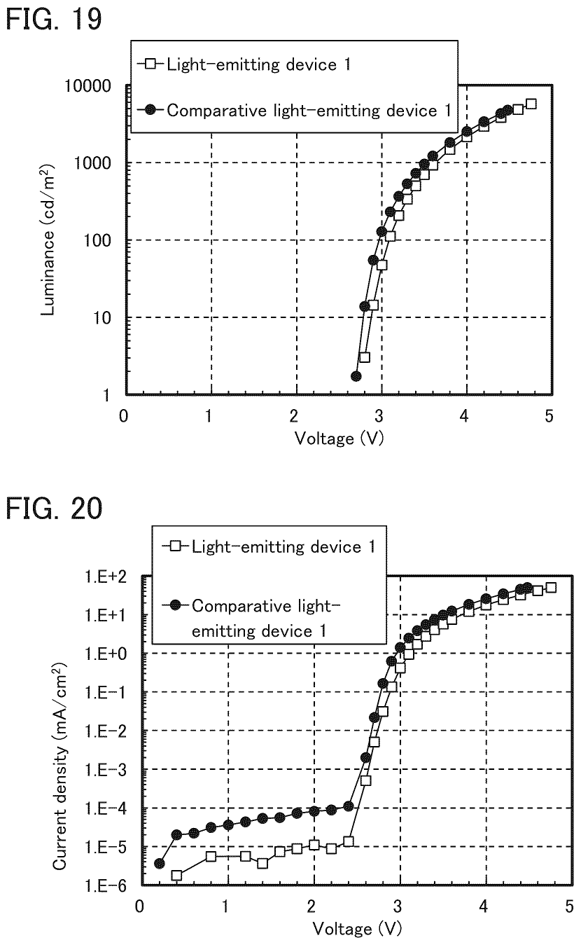

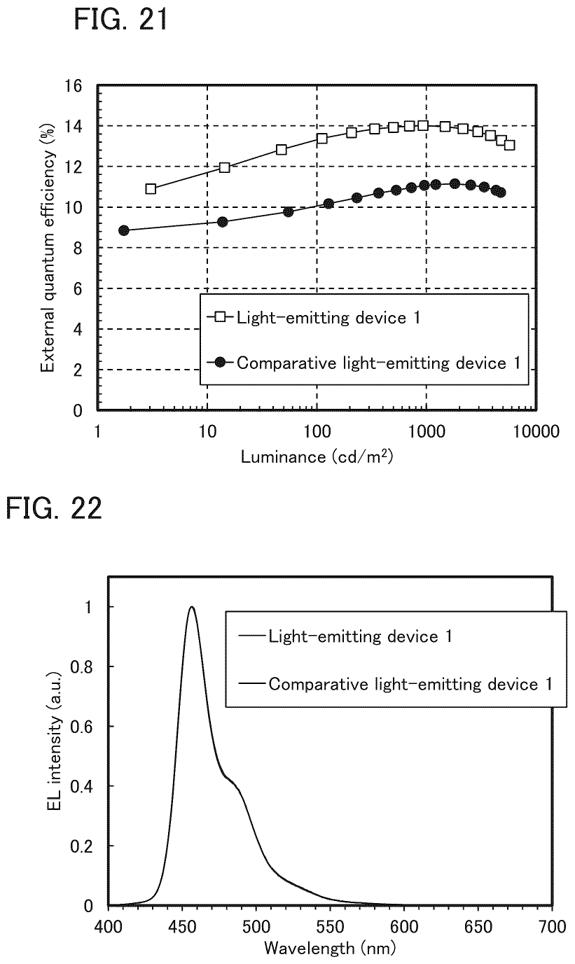

FIG. 17 shows the luminance-current density characteristics of the light-emitting device 1 and the comparative light-emitting device 1. FIG. 18 shows the current efficiency-luminance characteristics thereof. FIG. 19 shows the luminance-voltage characteristics thereof. FIG. 20 shows the current density-voltage characteristics thereof. FIG. 21 shows the external quantum efficiency-luminance characteristics thereof. FIG. 22 shows the electroluminescence spectra thereof. Table 5 shows main characteristics of the light-emitting devices at approximately 1000 cd/m². Note that the luminance, CIE chromaticity, and emission spectrum were measured at normal temperature with a spectroradiometer (SR-UL1R produced by TOPCON TECHNOHOUSE CORPORATION). The external quantum efficiency was calculated from the measured luminance and emission spectrum, on the assumption that the light-emitting device had Lambertian light-distribution characteristics.

TABLE 5

| | | | Current | | | Current | |
|---|---|---|---|---|---|---|---|
| | Voltage | Current | density | Chromaticity | Chromaticity | efficiency | External quantum |
| | (V) | (mA) | (mA/cm²) | x | y | (cd/A) | efficiency (%) |
| Light-emitting device 1 | 3.60 | 0.300 | 7.51 | 0.138 | 0.0992 | 12.4 | 14.0 |
| Comparative light- | 3.50 | 0.387 | 9.68 | 0.138 | 0.100 | 9.91 | 11.1 |
| emitting device 1 | | | | | | | |

FIG. 17 to FIG. 22 and Table 5 show that the light-emitting device 1 using mmtBuoBichPAF, which is the organic compound of one embodiment of the present invention, for a hole-injection layer and a hole-transport layer has high emission efficiency (current efficiency and external quantum efficiency).

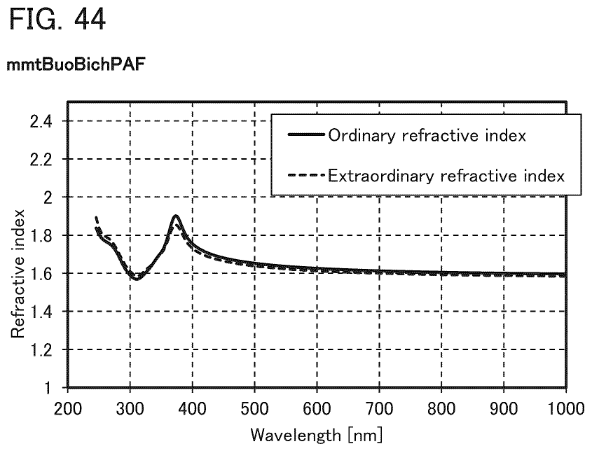

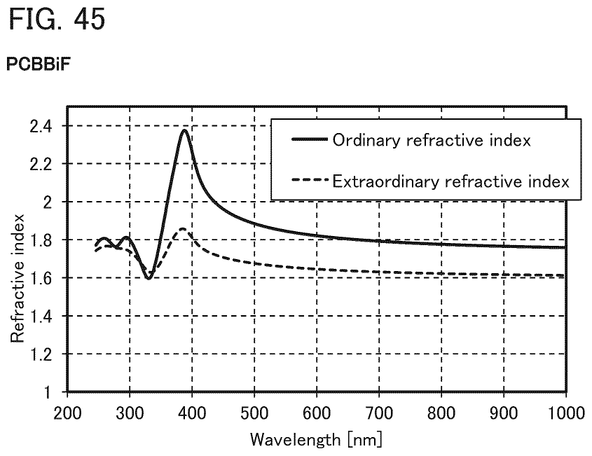

FIG. 44 and FIG. 45 show the measurement results of the refractive indices of mmtBuoBichPAF and PCBBiF. The measurement was performed with a spectroscopic ellipsometer (M-2000U, produced by J.A. Woollam Japan Corp.). To obtain films used as measurement samples, the material for each layer was deposited to a thickness of approximately 50 nm over a quartz substrate by a vacuum evaporation method. As a result, the film of mmtBuoBichPAF had an ordinary refractive index of 1.68 at a wavelength of 455 nm and an ordinary refractive index lower than or equal to 1.70 in the entire wavelength region of 450 nm to 460 nm, and the film of PCBBiF had an ordinary refractive index of 1.95 at a wavelength of 455 nm.

Accordingly, the light-emitting device 1 can have high emission efficiency when the light extraction efficiency is improved by including mmtBuoBichPAF capable of forming a film with a low refractive index.

### Example 4

In this example, a light-emitting device 2 of one embodiment of the present invention will be described in detail. Structural formulae of typical organic compounds used in this example are shown below.

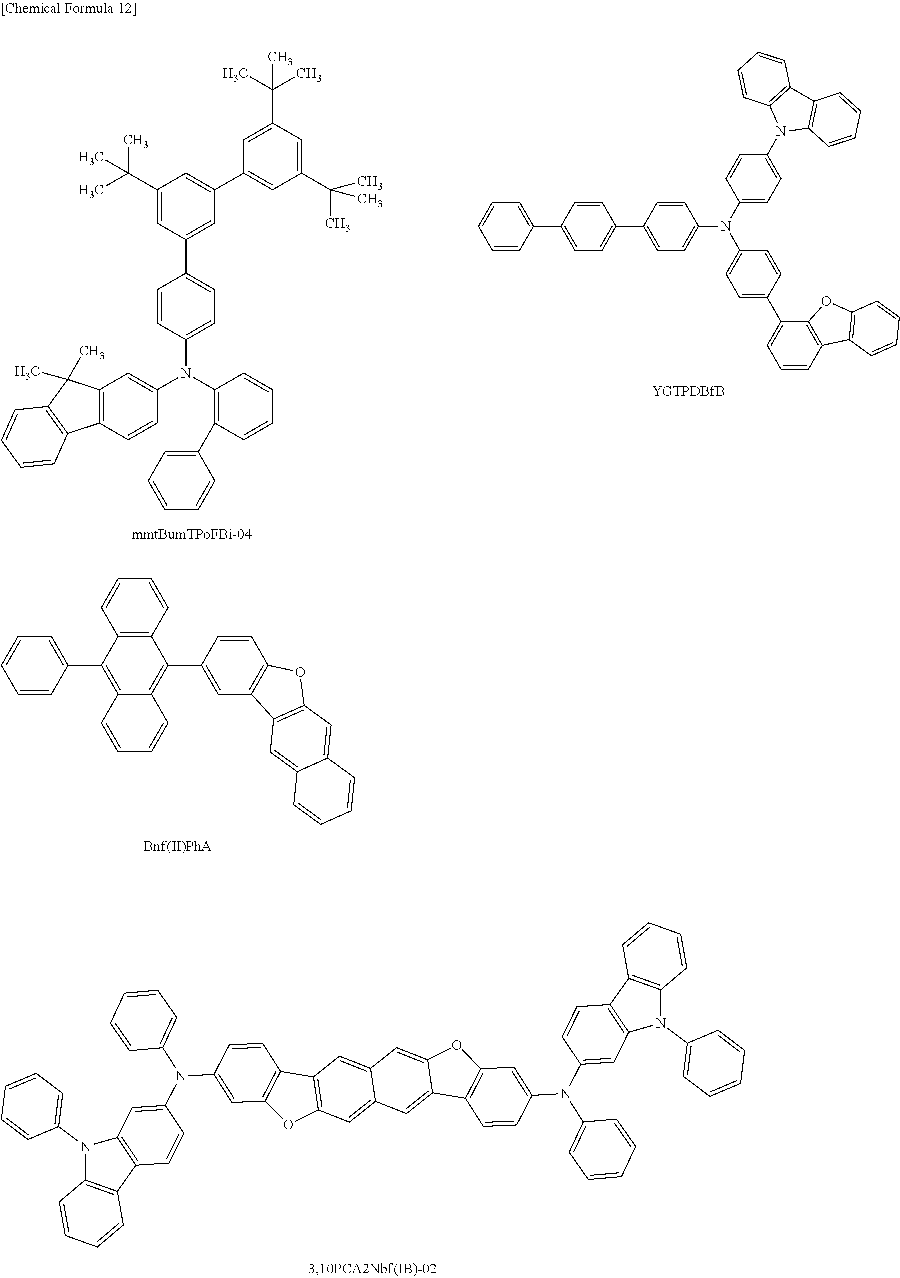 [SMILES: CC(C)(C)c1cc(-c2ccc(N(c3ccc4c(c3)C(C)(C)c3ccccc3-4)c3ccccc3-c3ccccc3)cc2)cc(-c2cc(C(C)(C)C)cc(C(C)(C)C)c2)c1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2c3ccccc3c(-c3ccc4oc5cc6ccccc6cc5c4c3)c3ccccc23)cc1 ; c1ccc(N(c2ccc3c(c2)oc2cc4cc5c(cc4cc23)oc2cc(N(c3ccccc3)c3ccc4c6ccccc6n(-c6ccccc6)c4c3)ccc25)c2ccc3c4ccccc4n(-c4ccccc4)c3c2)cc1]

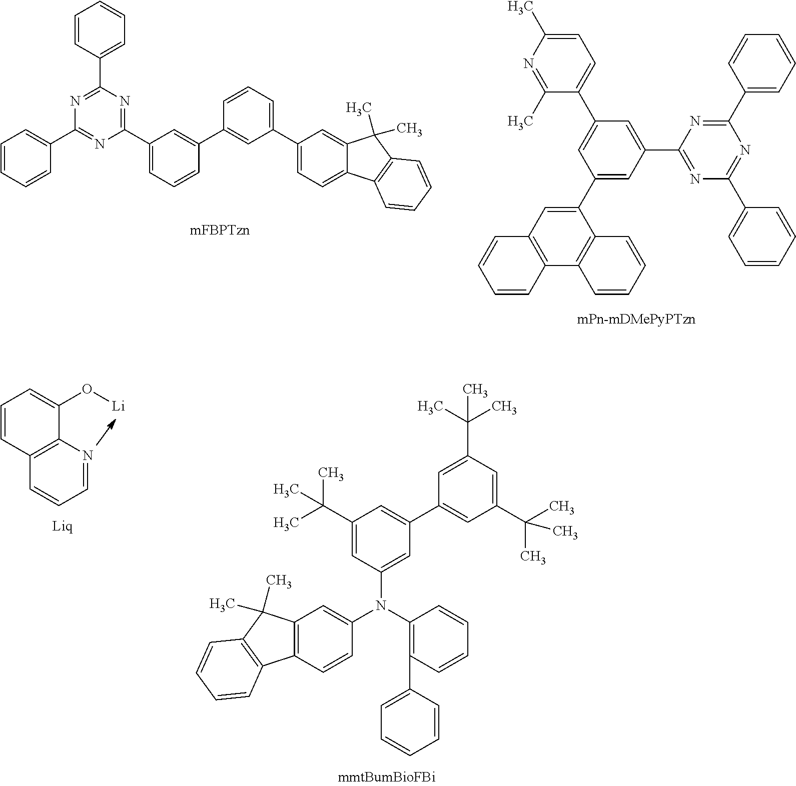

### (Method for Fabricating Light-Emitting Device 2)

First, indium tin oxide containing silicon oxide (ITSO) was deposited over a glass substrate to a thickness of 55 nm by a sputtering method to form the first electrode 101. Note that the electrode area was 2 mm×2 mm.

Next, in pretreatment for fabricating the light-emitting element over the substrate, the surface of the substrate was washed with water, and baking was performed at 200° C. for one hour.

After that, the substrate was transferred into a vacuum evaporation apparatus where the pressure had been reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Next, the substrate provided with the first electrode 101 was fixed to a substrate holder provided in the vacuum evaporation apparatus such that the surface on which the first electrode 101 was formed faced downward. Then, N-(3′,5′-ditertiarybutylbiphenyl-4-yl)-N-(3′,5′-ditertiarybutylbiphenyl-2-yl)-9,9-dimethyl-9H-fluoren-2-amine (abbreviation: dmmtBuopBBAF) represented by Structural Formula (ix) above and a fluorine-containing electron acceptor material with a molecular weight of 672 (OCHD-003) were deposited on the first electrode 101 (anode) to a thickness of 10 nm by co-evaporation using resistance heating such that the weight ratio of dmmtBuopBBAF to OCHD-003 was 1:0.1, whereby the hole-injection layer 111 was formed.

Subsequently, over the hole-injection layer 111, dmmtBuopBBAF was deposited to a thickness of 30 nm by evaporation to form a first hole-transport layer, and then N-[4-(dibenzofuran-4-yl)phenyl]-N-[4-(9H-carbazol-9-yl)phenyl]-p-terphenyl-4-amine (abbreviation: YGTPDBfB) represented by Structural Formula (ii) above was deposited to a thickness of 10 nm to form a second hole-transport layer, whereby the hole-transport layer 112 was formed. Note that the second hole-transport layer also functions as an electron-blocking layer.

Subsequently, 2-(10-phenyl-9-anthryl)benzo[b]naphtho[2,3-d]furan (abbreviation: Bnf(II)PhA) represented by Structural Formula (iii) above and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) represented by Structural Formula (iv) above were formed over the hole-transport layer 112 to a thickness of 25 nm by co-evaporation such that the weight ratio of Bnf(II)PhA to 3,10PCA2Nbf(IV)-02 was 1:0.015, whereby the light-emitting layer 113 was formed.

After that, over the light-emitting layer 113, 2-[3′-(9,9-dimethyl-9H-fluoren-2-yl)biphenyl-3-yl]-4,6-diphenyl-1,3,5-triazine (abbreviation: mFBPTzn) represented by Structure Formula (v) was deposited to a thickness of 10 nm, whereby a first electron-transport layer was formed. Sequentially, 2-[3-(2,6-dimethyl-3-pyridinyl)-5-(9-phenanthryl)phenyl]-4,6-diphenyl-1,3,5-triazine (abbreviation: mPn-mDMePyPTzn) represented by Structure Formula (vii) and 8-hydroxyquinolinato-lithium (abbreviation: Liq) were deposited by co-evaporation to a thickness of 20 nm at a weight ratio of 1:1, whereby a second electron-transport layer was formed. Accordingly, the electron-transport layer 114 was formed.

Over the electron-transport layer 114, Liq was deposited to a thickness of 1 nm to form the electron-injection layer 115.

Lastly, aluminum was deposited by evaporation to a thickness of 200 nm to form the second electrode 102, whereby the light-emitting device of one embodiment of the present invention was fabricated.

The structure of the light-emitting device 2 is listed in the following table.

TABLE 6

| | | Film | |
|---|---|---|---|
| | | thickness | Light-emitting |
| | | (nm) | device 2 |
| Second electrode | | 200 | Al |
| Electron-injection layer | | 1 | Liq |
| Electron-transport | 2 | 20 | mPn-mDMePyPTzn:Liq |
| layer | | | (1:1) |
| | 1 | 10 | mFBPTzn |
| Light-emitting layer | | 25 | Bnf(II)PhA:3,10PCA2Nbf(IV)-02 |
| | | | (1:0.015) |
| Hole-transport layer | 2 | 10 | YGTPDBfB |
| | 1 | 30 | dmmtBuopBBAF |

| Hole-injection layer | 10 | dmmtBuopBBAF:OCHD-003 |
|---|---|---|
| | | (1:0.1) |
| First electrode | 55 | ITSO |

The above light-emitting device 2 was sealed using a glass substrate in a glove box including a nitrogen atmosphere so as not to be exposed to the air (a sealing material was applied to surround an element and UV treatment and heat treatment at 80° C. for an hour were performed at the time of sealing). Then, the initial characteristics of the light-emitting elements were measured.

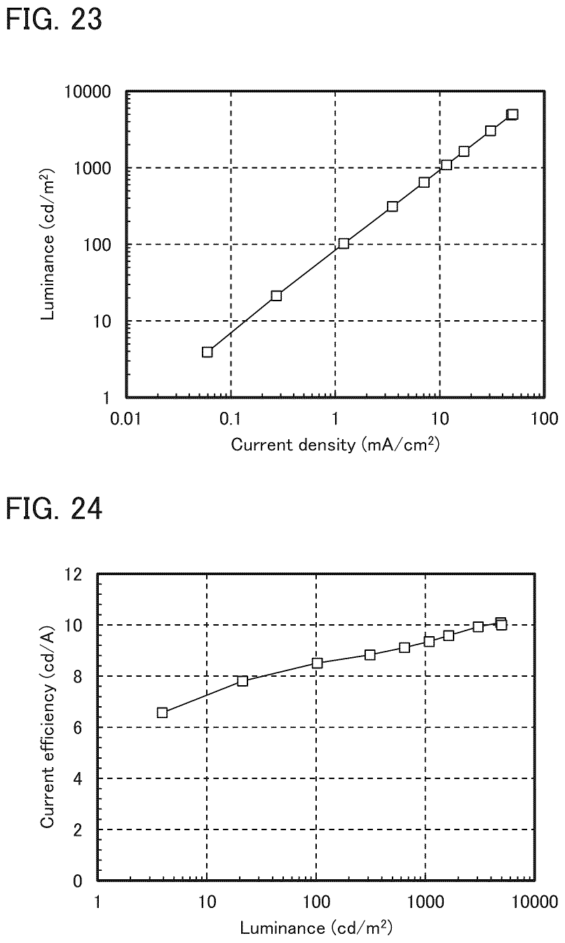

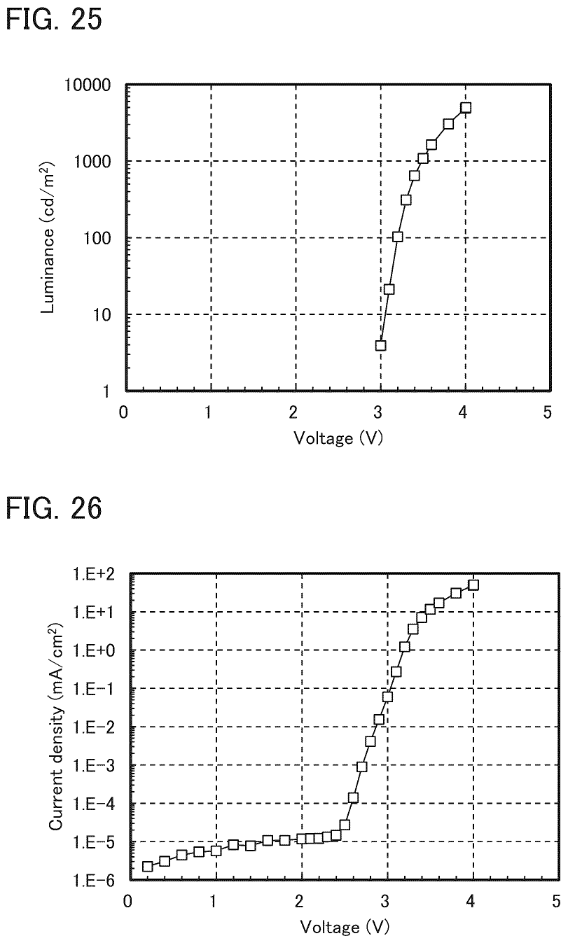

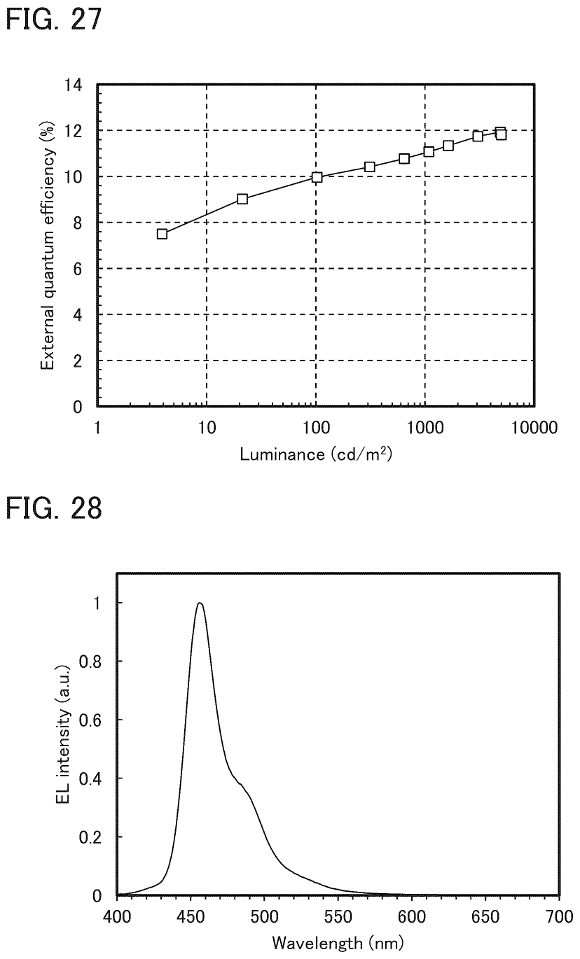

FIG. 23 shows the luminance-current density characteristics of the light-emitting device 2. FIG. 24 shows the current efficiency-luminance characteristics thereof. FIG. 25 shows the luminance-voltage characteristics thereof. FIG. 26 shows the current density-voltage characteristics thereof. FIG. 27 shows the external quantum efficiency-luminance characteristics thereof. FIG. 28 shows the electroluminescence spectrum thereof. Table 7 shows main characteristics of the light-emitting elements at approximately 1000 cd/m². Note that the luminance, CIE chromaticity, and emission spectrum were measured at normal temperature with a spectroradiometer (SR-UL1R produced by TOPCON TECHNOHOUSE CORPORATION). The external quantum efficiency was calculated from the measured luminance and emission spectrum, on the assumption that the light-emitting device had Lambertian light-distribution characteristics.

TABLE 7

| | | | Current | | | Current | External |
|---|---|---|---|---|---|---|---|
| | Voltage | Current | density | Chromaticity | Chromaticity | efficiency | quantum |
| | (V) | (mA) | (mA/cm²) | x | y | (cd/A) | efficiency (%) |
| Light-emitting device 2 | 3.50 | 0.465 | 11.6 | 0.139 | 0.0931 | 9.35 | 11.1 |

FIG. 23 to FIG. 28 and Table 7 show that the light-emitting device 2 using dmmtBuopBBAF, which is the organic compound of one embodiment of the present invention, for a hole-injection layer and a hole-transport layer has high emission efficiency (current efficiency and external quantum efficiency).

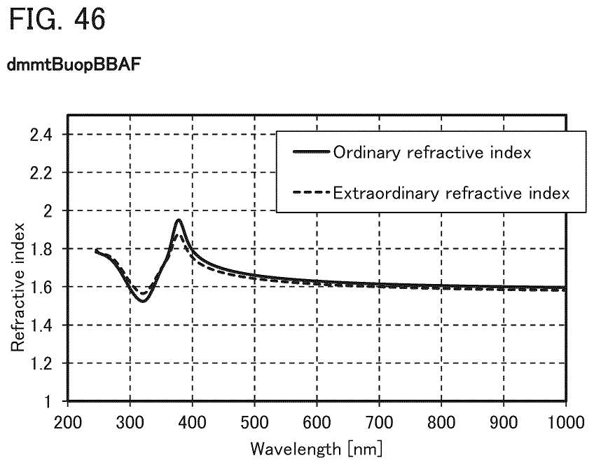

FIG. 46 shows the measurement results of the refractive indices of dmmtBuopBBAF. The measurement was performed with an M-2000U spectroscopic ellipsometer manufactured by J.A. Woollam Japan Corp. To obtain films used as measurement samples, the material for each layer was deposited to a thickness of approximately 50 nm over a quartz substrate by a vacuum evaporation method. As a result, the film of dmmtBuopBBAF had an ordinary refractive index of 1.69 at a wavelength of 455 nm and an ordinary refractive index of lower than or equal to 1.70 at a wavelength of 450 nm to 460 nm.

Accordingly, the light-emitting device 2 can have high emission efficiency when the light extraction efficiency is improved by including dmmtBuopBBAF capable of forming a film with a low refractive index.

### Example 5

In this example, a light-emitting device 3, which is a light-emitting device of one embodiment of the present invention, and comparative light-emitting devices 3-1 to 3-3 for comparison are described in detail. Structural formulae of typical organic compounds used in this example are shown below.

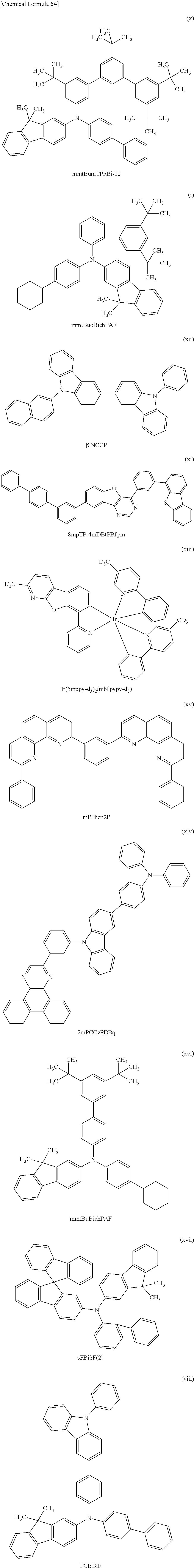

### (Method for Fabricating Light-Emitting Device 3)

First, indium tin oxide containing silicon oxide (ITSO) was deposited over a glass substrate to a thickness of 70 nm by a sputtering method to form the first electrode 101. Note that the electrode area was 2 mm×2 mm.

Next, in pretreatment for fabricating the light-emitting element over the substrate, the surface of the substrate was washed with water, and baking was performed at 200° C. for one hour.

After that, the substrate was transferred into a vacuum evaporation apparatus where the pressure had been reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Next, the substrate provided with the first electrode 101 was fixed to a substrate holder provided in the vacuum evaporation apparatus such that the surface on which the first electrode 101 was formed faced downward. Then, N-(biphenyl-4-yl)-N-(3,3″,5′,5″-tetra-tert-butyl-[1,1′:3′,1″-terphenyl]-5-yl)-9,9-dimethyl-9H-fluoren-2-amine (abbreviation: mmtBumTPFBi-02) represented by Structural Formula (i) above and a fluorine-containing electron acceptor material with a molecular weight of 672 (OCHD-003) were deposited on the first electrode 101 (anode) to a thickness of 10 nm by co-evaporation using a resistance-heating method such that the weight ratio of mmtBumTPFBi-02 to OCHD-003 was 1:0.1, whereby the hole-injection layer 111 was formed.

Subsequently, over the hole-injection layer 111, mmtBumTPFBi-02 was deposited to a thickness of 100 nm by evaporation to form a first hole-transport layer, and then, N-(3′,5′-ditertiarybutylbiphenyl-2-yl)-N-(4-cyclohexylphenyl)-9,9-dimethyl-9H-fluoren-2-amine (abbreviation: mmtBuoBichPAF), which is the organic compound of one embodiment of the present invention represented by Structural Formula (i) above, was deposited to a thickness of 40 nm to forma second hole-transport layer, whereby the hole-transport layer 112 was formed. Note that the second hole-transport layer also functions as an electron-blocking layer.

Then, over the hole-transport layer 112, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm) represented by Structural Formula (xi) above, 9-(2-naphthyl)-9′-phenyl-3,3′-bi-9H-carbazole (abbreviation: βNCCP) represented by Structural Formula (xii) above, and [2-d₃-methyl-8-(2-pyridinyl-κN)benzofuro[2,3-b]pyridine-κC]bis[2-(5-d-methyl-2-pyridinyl-κN²)phenyl-κC]iridium(III) (abbreviation: [Ir(5mppy-d₃)₂(mbfpypy-d₃)]) represented by Structural Formula (xiii) above were deposited by co-evaporation to a thickness of 40 nm such that the weight ratio of 8mpTP-4mDBtPBfpm, βNCCP, and [Ir(5mppy-d₃)₂(mbfpypy-d₃)] was 0.5:0.5:0.1, whereby the light-emitting layer 113 was formed.

After that, 2-{3-[3-(N-phenyl-9H-carbazol-3-yl)-9H-carbazol-9-yl]phenyl}dibenzo[f,h]quinoxaline (abbreviation: 2mPCCzPDBq) represented by Structural Formula (xiv) above was formed over the light-emitting layer 113 to a thickness of 20 nm to form a first electron-transport layer, and then 2,2′-(1,3-phenylene)bis(9-phenyl-1,10-phenanthroline) (abbreviation: mPPhen2P) represented by Structure Formula (xv) above was formed to a thickness of 20 nm to form a second electron-transport layer, whereby the electron-transport layer 114 was formed.

Next, over the electron-transport layer 114, lithium fluoride (LiF) was deposited to a thickness of 1 nm to form the electron-injection layer 115.

Lastly, aluminum was deposited by evaporation to a thickness of 100 nm to form the second electrode 102, whereby the light-emitting device of one embodiment of the present invention was fabricated.

### (Method for Fabricating Comparative Light-Emitting Device 3-1)

The comparative light-emitting device 3-1 was fabricated in a manner similar to that for the light-emitting device 3 except that mmtBuoBichPAF used for the second hole-transport layer was replaced with N-[(3′,5′-ditertiarybutyl)biphenyl-4-yl]-N-(4-cyclohexylphenyl)-9,9-dimethyl-9H-fluoren-2-amine (abbreviation: mmtBuBichPAF) represented by Structural Formula (xvi) above.

### (Method for Fabricating Comparative Light-Emitting Device 3-2)

The comparative light-emitting device 3-2 was fabricated in a manner similar to that of the light-emitting device 3 except that mmtBuoBichPAF used for the second hole-transport layer was replaced with N-(biphenyl-2-yl)-N-(9,9-dimethylfluoren-2-yl)-9,9′-spirobi(9H-fluoren)-2-amine (abbreviation: oFBiSF(2)) represented by Structural Formula (xvii) above.

### (Method for Fabricating Comparative Light-Emitting Device 3-3)

The comparative light-emitting device 3-3 was fabricated in a manner similar to that for the light-emitting device 3 except that mmtBuoBichPAF used for the second hole-transport layer was replaced with N-(biphenyl-4-yl)-N-[4-(9-phenyl-9H-carbazol-3-yl)phenyl]-9,9-dimethyl-9H-fluoren-2-amine (abbreviation: PCBBiF) represented by Structural Formula (viii) above.

The structures of the light-emitting device 3 and the comparative light-emitting devices 3-1 to 3-3 are listed in the following table.

TABLE 8

| | | Film | | | | |
|---|---|---|---|---|---|---|
| | | thickness | Light-emitting | Comparative light- | Comparative light- | Comparative light- |
| | | (nm) | device 3 | emitting device 3-1 | emitting device 3-2 | emitting device 3-3 |
| Second electrode | | 100 | Al | | | |
| Electron-injection layer | | 1 | LiF | | | |
| Electron-transport | 2 | 20 | mPPhen2P | | | |
| layer | 1 | 20 | 2mPCCzPDBq | | | |
| Light-emitting layer | | 40 | 8mpTP-4mDBtPBfpm:βCCP:Ir(5mppy-d₃)₂(mbfpypy-d₃) | | | |
| | | | (0.5:0.5:0.1) | | | |
| Hole-transport layer | 2 | 40 | mmtBuoBichPAF | mmtBuBichPAF | oFBiSF(2) | PCBBiF |

| | | |
|---|---|---|
| 1 | 100 | mmtBumTPFBi-02 |

| Hole-injection layer | 10 | mmtBumTPFBi-02:OCHD-003 |
|---|---|---|
| | | (1:0.1) |
| First electrode | 70 | ITSO |

Each of the light-emitting devices was sealed using a glass substrate in a glove box including a nitrogen atmosphere so as not to be exposed to the air (a sealing material was applied to surround an element and UV treatment and heat treatment at 80° C. for an hour were performed at the time of sealing). Then, the initial characteristics of the light-emitting elements were measured.

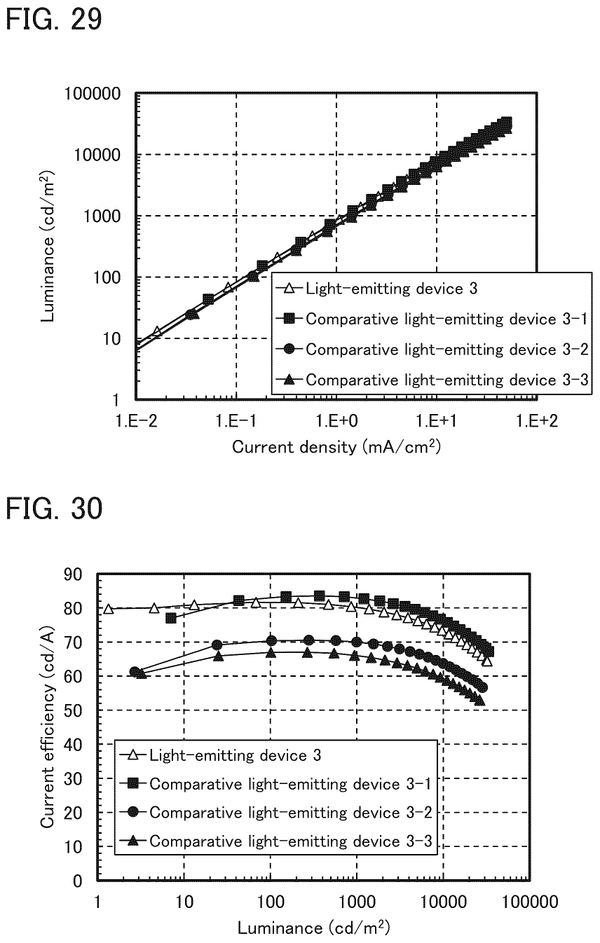

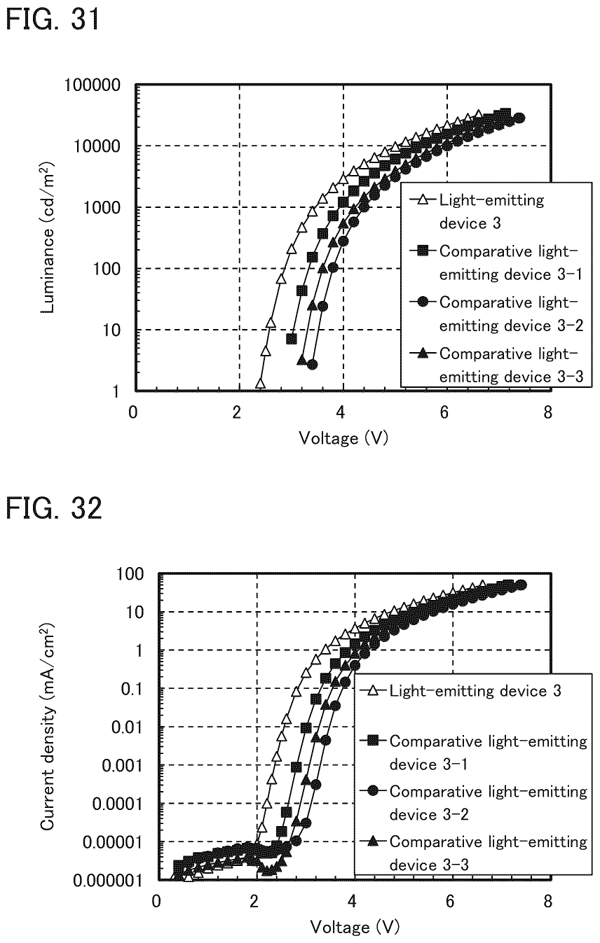

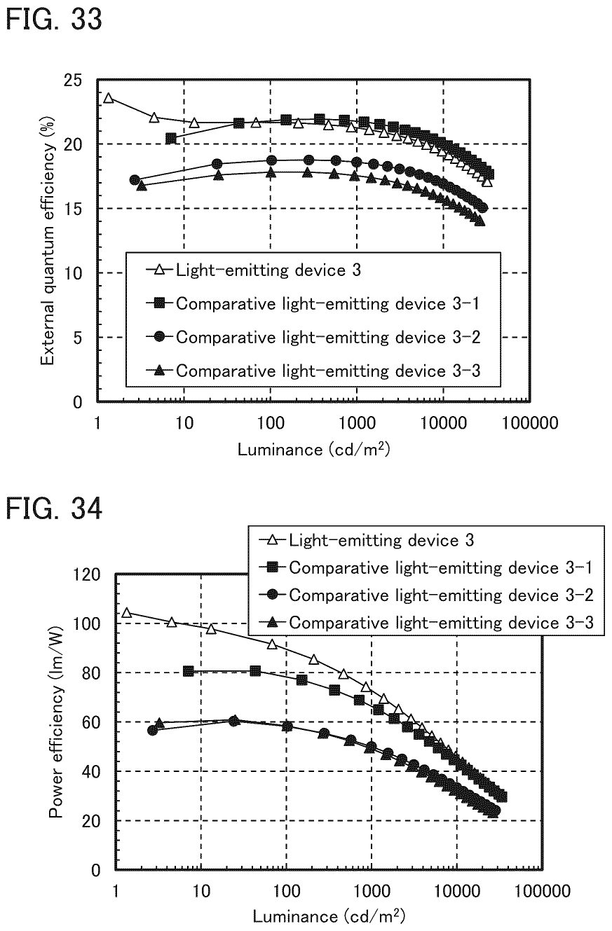

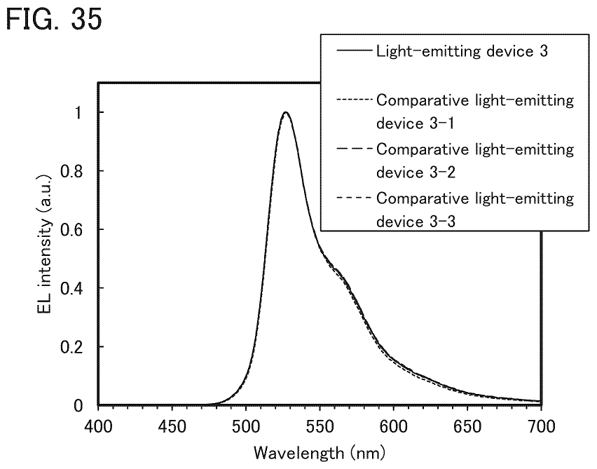

FIG. 29 shows luminance-current density characteristics of the light-emitting device 3 and the comparative light-emitting devices 3-1 to 3-3. FIG. 30 shows current efficiency-luminance characteristics thereof. FIG. 31 shows luminance-voltage characteristics thereof. FIG. 32 shows current density-voltage characteristics thereof. FIG. 33 shows external quantum efficiency-luminance characteristics thereof. FIG. 34 shows power efficiency-luminance characteristics thereof. FIG. 35 shows electroluminescence spectra thereof. Table 9 shows main characteristics of the light-emitting elements at approximately 1000 cd/m². Note that the luminance, CIE chromaticity, and emission spectrum were measured at normal temperature with a spectroradiometer (SR-UL1R produced by TOPCON TECHNOHOUSE CORPORATION). The external quantum efficiency and power efficiency were calculated from the measured luminance and emission spectrum, on the assumption that the light-emitting device had Lambertian light-distribution characteristics.

TABLE 9

| | | | Current | | | Current | |
|---|---|---|---|---|---|---|---|
| | Voltage | Current | density | Chromaticity | Chromaticity | efficiency | External quantum |
| | (V) | (mA) | (mA/cm²) | x | y | (cd/A) | efficiency (%) |
| Light-emitting device 3 | 3.40 | 0.0427 | 1.07 | 0.322 | 0.643 | 80.4 | 21.3 |
| Comparative light- | 4.00 | 0.0582 | 1.46 | 0.318 | 0.647 | 82.7 | 21.7 |
| emitting device 3-1 | | | | | | | |
| Comparative light- | 4.40 | 0.0569 | 1.42 | 0.323 | 0.642 | 70.0 | 18.6 |
| emitting device 3-2 | | | | | | | |
| Comparative light- | 4.20 | 0.0569 | 1.42 | 0.323 | 0.642 | 66.0 | 17.6 |
| emitting device 3-3 | | | | | | | |

FIGS. 29 to 35 and Table 9 show that the light-emitting device 3 using mmtBuoBichPAF, which is the organic compound of one embodiment of the present invention, for the second hole-transport layer and the comparative light-emitting device 3-1 using mmtBuBichPAF for the second hole-transport layer have high emission efficiency (current efficiency and external quantum efficiency).

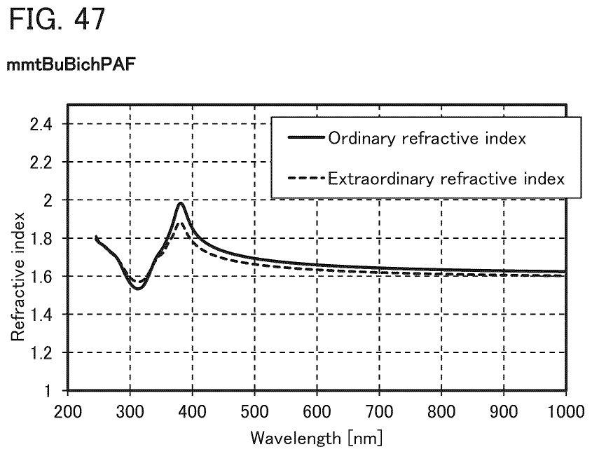

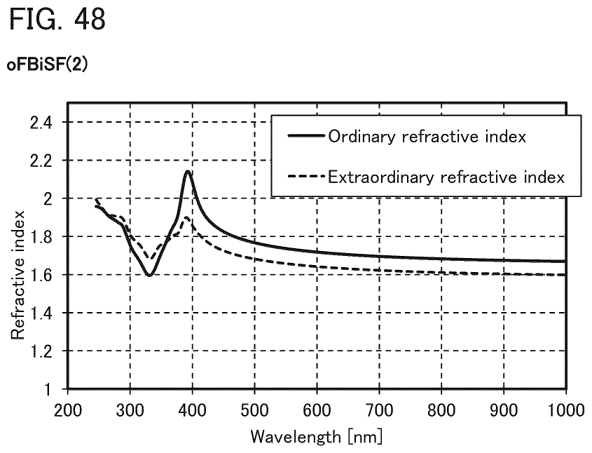

FIGS. 44, 47, 48, and 45 show the measurement results of the refractive indices of mmtBuoBichPAF, mmtBuBichPAF, oFBiSF(2), and PCBBiF. The measurement was performed with a spectroscopic ellipsometer (M-2000U, produced by J.A. Woollam Japan Corp.). To obtain films used as measurement samples, the material for each layer was deposited to a thickness of approximately 50 nm over a quartz substrate by a vacuum evaporation method. As a result, the film of mmtBuoBichPAF had an ordinary refractive index of 1.64 at a wavelength of 530 nm and an ordinary refractive index lower than or equal to 1.65 in the entire wavelength region of 510 nm to 545 nm; the film of mmtBuBichPAF had an ordinary refractive index of 1.68 at a wavelength of 530 nm and an ordinary refractive index lower than or equal to 1.70 at a wavelength of 510 nm to 545 nm; the film of oFBiSF(2) had an ordinary refractive index of 1.75 at a wavelength of 530 nm and an ordinary refractive index of higher than 1.75 in the entire wavelength region of 510 nm to 545 nm; and the film of PCBBiF had an ordinary refractive index of 1.86 at a wavelength of 530 nm. Accordingly, the light-emitting device 3 and the comparative light-emitting device 3-1 can have high emission efficiency when the light extraction efficiency is improved by including a layer with a low refractive index in the organic compound layer.

The GSP_slope of the film of mmtBuoBichPAF was 37.5 (mV/nm), the GSP_slope of the film of mmtBuBichPAF was 31.6 (mV/nm), the GSP_slope of the film of oFBiSF(2) was 20.3 (mV/nm), and the GSP_slope of the film of PCBBiF was 17.3 (mV/nm). Note that the GSP_slope can be obtained by the method described in Embodiment 1. The light-emitting device 3 includes mmtBuoBichPAF capable of forming a film with a large GSP_slope and thus has a low driving voltage, thereby exhibiting extremely high power efficiency.

As described above, the film of mmtBuoBichPAF has a low refractive index and a large GSP_slope. Thus, mmtBuoBichPAF is an organic compound which can provide an extremely favorable light-emitting device with high emission efficiency and a low driving voltage.

It was found that the GSP_slope of the film of mmtBuoBichPAF is greatly different from that of the film of mmtBuBichPAF although mmtBuoBichPAF and mmtBuBichPAF have the same molecular structure except a biphenyl group to which two alkyl groups are bonded at the end. The biphenyl group is an orthobiphenyl group in mmtBuoBichPAF and a parabiphenyl group in mmtBuBichPAF. The film of mmtBuoBichPAF has a large GSP_slope by including an orthobiphenyl group in which two alkyl groups are bonded to its end; as a result, mmtBuoBichPAF is found to be an organic compound which can provide a light-emitting device with high emission efficiency, low driving voltage, and extremely high power efficiency.

### Example 6

In this example, a light-emitting device 4, which is a light-emitting device of one embodiment of the present invention, and comparative light-emitting devices 4-1 to 4-3 for comparison are described in detail. Structural formulae of typical organic compounds used in this example are shown below.

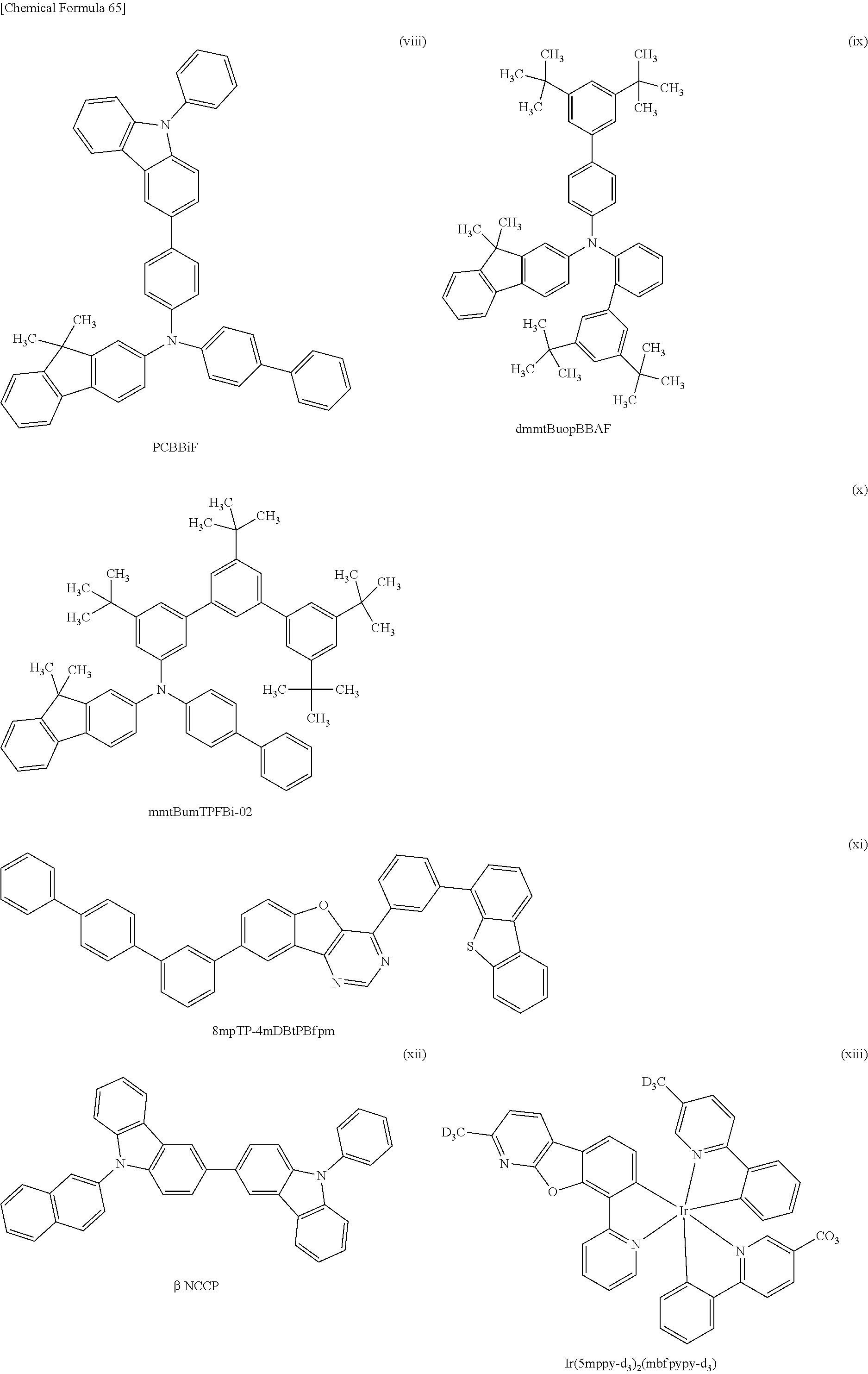

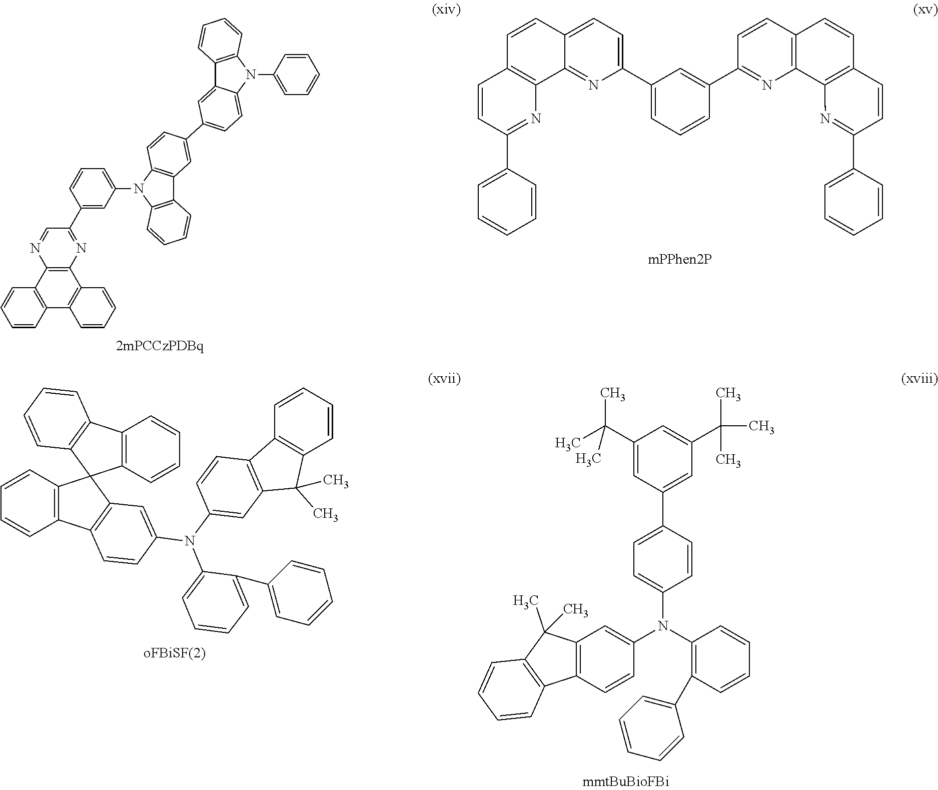 [SMILES: c1ccc(-n2c3ccccc3c3cc(-c4ccc5c(c4)c4ccccc4n5-c4cccc(-c5cnc6c7ccccc7c7ccccc7c6n5)c4)ccc32)cc1 ; c1ccc(-c2ccc3ccc4ccc(-c5cccc(-c6ccc7ccc8ccc(-c9ccccc9)nc8c7n6)c5)nc4c3n2)cc1 ; CC1(C)c2ccccc2-c2ccc(N(c3ccc4c(c3)C3(c5ccccc5-c5ccccc53)c3ccccc3-4)c3ccccc3-c3ccccc3)cc21 ; CC(C)(C)c1cc(-c2ccc(N(c3ccc4c(c3)C(C)(C)c3ccccc3-4)c3ccccc3-c3ccccc3)cc2)cc(C(C)(C)C)c1]

### (Method for Fabricating Light-Emitting Device 4)

First, indium tin oxide containing silicon oxide (ITSO) was deposited over a glass substrate to a thickness of 70 nm by a sputtering method to form the first electrode 101. Note that the electrode area was 2 mm×2 mm.

Next, in pretreatment for fabricating the light-emitting element over the substrate, the surface of the substrate was washed with water, and baking was performed at 200° C. for one hour.

After that, the substrate was transferred into a vacuum evaporation apparatus where the pressure had been reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Next, the substrate provided with the first electrode 101 was fixed to a substrate holder provided in the vacuum evaporation apparatus such that the surface on which the first electrode 101 was formed faced downward. Then, N-(biphenyl-4-yl)-N-(3,3″,5′,5″-tetra-tert-butyl-[1,1′:3′,1″-terphenyl]-5-yl)-9,9-dimethyl-9H-fluoren-2-amine (abbreviation: mmtBumTPFBi-02) represented by Structural Formula (i) above and a fluorine-containing electron acceptor material with a molecular weight of 672 (OCHD-003) were deposited on the first electrode 101 (anode) to a thickness of 10 nm by co-evaporation using a resistance-heating method such that the weight ratio of mmtBumTPFBi-02 to OCHD-003 was 1:0.1, whereby the hole-injection layer 111 was formed.

Subsequently, over the hole-injection layer 111, mmtBumTPFBi-02 was deposited to a thickness of 100 nm by evaporation to form a first hole-transport layer, and then, N-(3′,5′-ditertiarybutylbiphenyl-4-yl)-N-(3′,5′-ditertiarybutylbiphenyl-2-yl)-9,9-dimethyl-9H-fluoren-2-amine (abbreviation: dmmtBuopBBAF), which is the organic compound of one embodiment of the present invention represented by Structural Formula (ix) above, was deposited to a thickness of 40 nm to form a second hole-transport layer, whereby the hole-transport layer 112 was formed. Note that the second hole-transport layer also functions as an electron-blocking layer.

Then, over the hole-transport layer 112, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm) represented by Structural Formula (xi) above, 9-(2-naphthyl)-9′-phenyl-3,3′-bi-9H-carbazole (abbreviation: βNCCP) represented by Structural Formula (xii) above, and [2-d₃-methyl-8-(2-pyridinyl-κN)benzofuro[2,3-b]pyridine-κC]bis[2-(5-d₃-methyl-2-pyridinyl-κN₂)phenyl-κC]iridium(III) (abbreviation: [Ir(5mppy-d₃)₂(mbfpypy-d₃)]) represented by Structural Formula (xiii) above were deposited by co-evaporation to a thickness of 40 nm such that the weight ratio of 8mpTP-4mDBtPBfpm, βNCCP, and Ir(5mppy-d₃)₂(mbfpypy-d₃) was 0.5:0.5:0.1, whereby the light-emitting layer 113 was formed.

After that, 2-{3-[3-(N-phenyl-9H-carbazol-3-yl)-9H-carbazol-9-yl]phenyl}dibenzo[f,h]quinoxaline (abbreviation: 2mPCCzPDBq) represented by Structural Formula (xiv) above was formed over the light-emitting layer 113 to a thickness of 20 nm to form a first electron-transport layer, and then 2,2′-(1,3-phenylene)bis(9-phenyl-1,10-phenanthroline) (abbreviation: mPPhen2P) represented by Structure Formula (xv) above was formed to a thickness of 20 nm to form a second electron-transport layer, whereby the electron-transport layer 114 was formed.

Next, over the electron-transport layer 114, lithium fluoride (LiF) was deposited to a thickness of 1 nm to form the electron-injection layer 115.

Lastly, aluminum was deposited by evaporation to a thickness of 100 nm to form the second electrode 102, whereby the light-emitting device of one embodiment of the present invention was fabricated.

### (Method for Fabricating Comparative Light-Emitting Device 4-1)

The comparative light-emitting device 4-1 was fabricated in a manner similar to that for the light-emitting device 4 except that dmmtBuopBBAF used for the second hole-transport layer was replaced with N-(3′,5′-ditertiarybutylbiphenyl-4-yl)-N-(biphenyl-2-yl)-9,9-dimethyl-9H-fluoren-2-amine (abbreviation: mmtBuBioFBi) represented by Structural Formula (xviii) above.

### (Method for Fabricating Comparative Light-Emitting Device 4-2)

The comparative light-emitting device 4-2 was fabricated in a manner similar to that of the light-emitting device 4 except that dmmtBuopBBAF used for the second hole-transport layer was replaced with N-(biphenyl-2-yl)-N-(9,9-dimethylfluoren-2-yl)-9,9′-spirobi(9H-fluoren)-2-amine (abbreviation: oFBiSF(2)) represented by Structural Formula (xvii) above.

### (Method for Fabricating Comparative Light-Emitting Device 4-3)

The comparative light-emitting device 4-3 was fabricated in a manner similar to that for the light-emitting device 4 except that dmmtBuopBBAF used for the second hole-transport layer was replaced with N-(biphenyl-4-yl)-N-[4-(9-phenyl-9H-carbazol-3-yl)phenyl]-9,9-dimethyl-9H-fluoren-2-amine (abbreviation: PCBBiF) represented by Structural Formula (viii) above.

The structures of the light-emitting device 4 and the comparative light-emitting devices 4-1 to 4-3 are listed in the following table.

TABLE 10

| | | Film | | Comparative | Comparative | Comparative |
|---|---|---|---|---|---|---|
| | | thickness | Light-emitting | light-emitting | light-emitting | light-emitting |
| | | (nm) | device 4 | device 4-1 | device 4-2 | device 4-3 |
| Second electrode | | 100 | Al | | | |
| Electron-injection layer | | 1 | LiF | | | |
| Electron-transport | 2 | 20 | mPPhen2P | | | |
| layer | 1 | 20 | 2mPCCzPDBq | | | |
| Light-emitting layer | | 40 | 8mpTP-4mDBtPBfpm:βNCCP:Ir(5mppy-d₃)₂(mbfpypy-d₃) | | | |
| | | | (0.5:0.5:0.1) | | | |
| Hole-transport layer | 2 | 40 | dmmtBuopBBAF | mmtBuBioFBi | oFBiSF(2) | PCBBiF |

| | | | |
|---|---|---|---|
| | 1 | 100 | mmtBumTPFBi-02 |
| Hole-injection layer | | 10 | mmtBumTPFBi-02:OCHD-003 |
| | | | (1:0.1) |
| First electrode | | 70 | ITSO |

Each of the light-emitting devices was sealed using a glass substrate in a glove box including a nitrogen atmosphere so as not to be exposed to the air (a sealing material was applied to surround an element and UV treatment and heat treatment at 80° C. for an hour were performed at the time of sealing). Then, the initial characteristics of the light-emitting elements were measured.

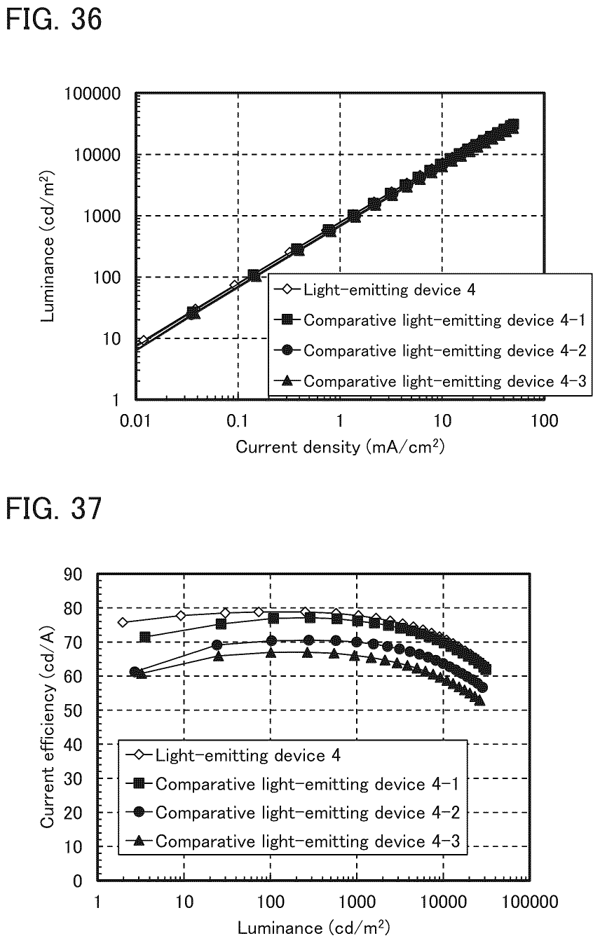

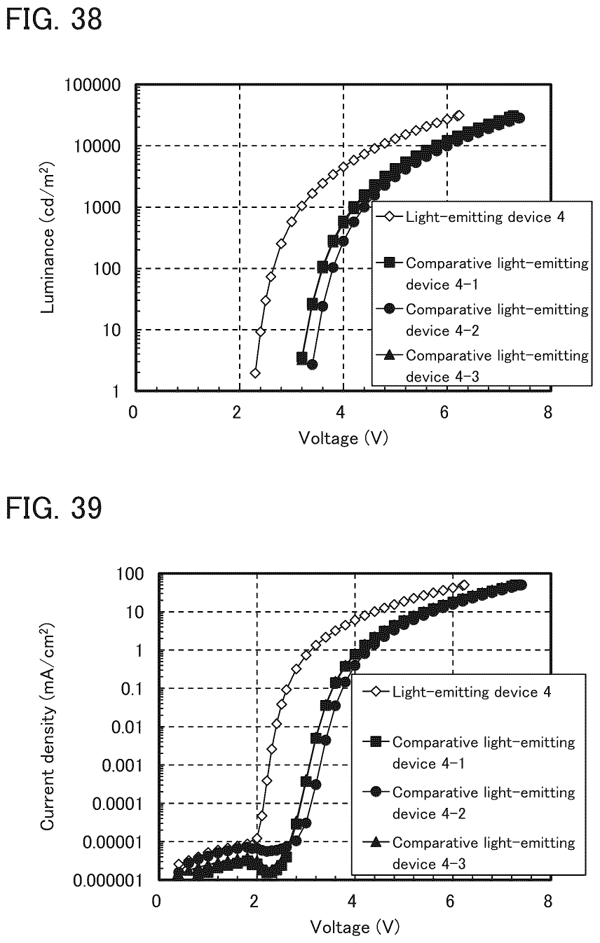

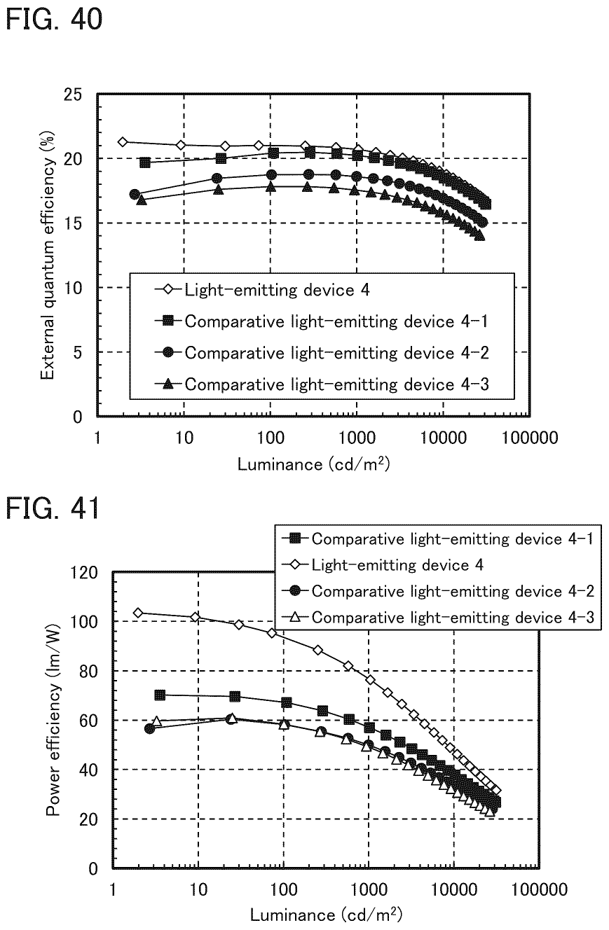

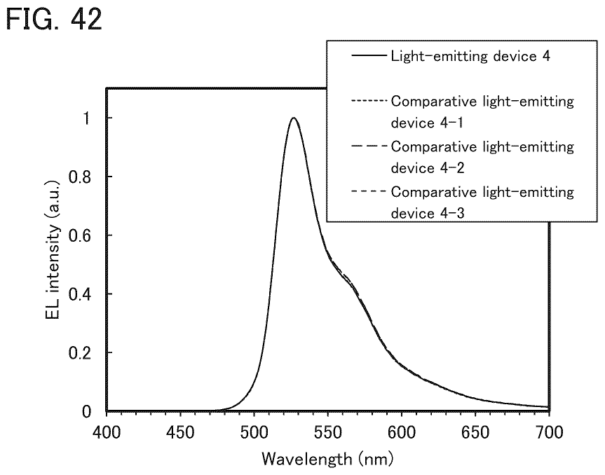

FIG. 36 shows luminance-current density characteristics of the light-emitting device 4 and the comparative light-emitting devices 4-1 to 4-3. FIG. 37 shows current efficiency-luminance characteristics thereof. FIG. 38 shows luminance-voltage characteristics thereof. FIG. 39 shows current density-voltage characteristics thereof. FIG. 40 shows external quantum efficiency-luminance characteristics thereof. FIG. 41 shows power efficiency-luminance characteristics thereof. FIG. 42 shows electroluminescence spectra thereof. Table 11 shows main characteristics of the light-emitting elements at approximately 1000 cd/m². Note that the luminance, CIE chromaticity, and emission spectrum were measured at normal temperature with a spectroradiometer (SR-ULIR produced by TOPCON TECHNOHOUSE CORPORATION). The external quantum efficiency and power efficiency were calculated from the measured luminance and emission spectrum, on the assumption that the light-emitting device had Lambertian light-distribution characteristics.

TABLE 11

| | | | | | | Current | External |
|---|---|---|---|---|---|---|---|
| | Voltage | Current | Current density | Chromaticity | Chromaticity | efficiency | quantum |
| | (V) | (mA) | (mA/cm²) | x | y | (cd/A) | efficiency (%) |
| Light-emitting device | 3.20 | 0.0536 | 1.34 | 0.321 | 0.643 | 77.8 | 20.7 |
| 4 | | | | | | | |
| Comparative light- | 4.20 | 0.0536 | 1.34 | 0.321 | 0.644 | 76.2 | 20.2 |
| emitting device 4-1 | | | | | | | |
| Comparative light- | 4.40 | 0.0569 | 1.42 | 0.323 | 0.642 | 70.0 | 18.6 |
| emitting device 4-2 | | | | | | | |
| Comparative light- | 4.20 | 0.0569 | 1.42 | 0.323 | 0.642 | 66.0 | 17.6 |
| emitting device 4-3 | | | | | | | |

FIG. 36 to FIG. 42 and Table 11 show that the light-emitting device 4 using dmmtBuopBBAF, which is the organic compound of one embodiment of the present invention, for the second hole-transport layer and the comparative light-emitting device 4-1 using mmtBuBioFBi for the second hole-transport layer have high emission efficiency (current efficiency and external quantum efficiency).

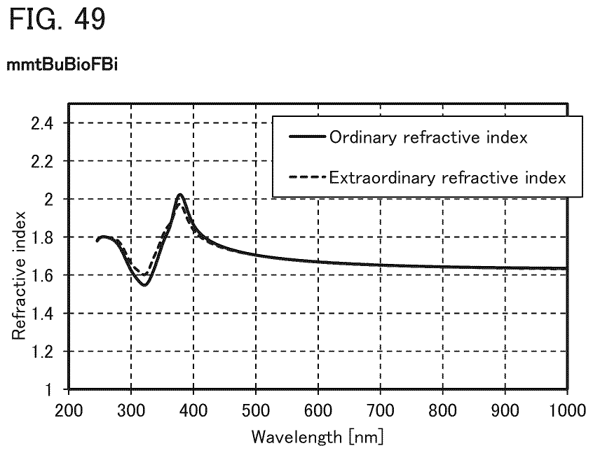

FIGS. 46, 49, 48, and 45 show the measurement results of the refractive indices of dmmtBuopBBAF, mmtBuBioFBi, oFBiSF(2), and PCBBiF. The measurement was performed with a spectroscopic ellipsometer (M-2000U, produced by J.A. Woollam Japan Corp.). To obtain films used as measurement samples, the material for each layer was deposited to a thickness of approximately 50 nm over a quartz substrate by a vacuum evaporation method. As a result, the film of dmmtBuopBBAF had an ordinary refractive index of 1.65 at a wavelength of 530 nm and an ordinary refractive index was lower than or equal to 1.70 at a wavelength of 510 nm to 545 nm; the film of mmtBuBioFBi had an ordinary refractive index of 1.69 at a wavelength of 530 nm and an ordinary refractive index of lower than or equal to 1.70 in the entire wavelength region of 510 nm to 545 nm; the film of oFBiSF (2) had an ordinary refractive index of 1.75 at a wavelength of 530 nm and an ordinary refractive index of higher than 1.75 in the entire wavelength region of 510 nm to 545 nm; and the film of PCBBiF had an ordinary refractive index of 1.86 at a wavelength of 530 nm. Accordingly, the light-emitting device 4 and the comparative light-emitting device 4-1 can have high emission efficiency when the light extraction efficiency is improved by including a layer with a low refractive index in the organic compound layer.

The GSP_slope of the film of dmmtBuopBBAF was 46.2 (mV/nm), the GSP_slope of the film of mmtBuBioFBi was 25.5 (mV/nm), the GSP_slope of the film of oFBiSF (2) was 20.3 (mV/nm), and the GSP_slope of the film of PCBBiF was 17.3 (mV/nm). Note that a GSP_slope can be obtained by the method described in Embodiment 1. The light-emitting device 4 includes dmmtBuopBBAF capable of forming a film with a large GSP_slope and thus has a low driving voltage, thereby exhibiting extremely high power efficiency.

As described above, the film of dmmtBuopBBAF has a low refractive index and a large GSP_slope. Thus, dmmtBuopBBAF is an organic compound which can provide an extremely favorable light-emitting device with high emission efficiency and a low driving voltage.

It was found that the GSP_slope of the film of dmmtBuopBBAF is greatly different from that of the film of mmtBuBioFBi although dmmtBuopBBAF and mmtBuBioFBi have the same molecular structure except the presence of two alkyl groups at end of an orthobiphenyl group included in the molecular structures. The film of dmmtBuopBBAF has a large GSP_slope by including an orthobiphenyl group in which two alkyl groups are bonded to its end; as a result, dmmtBuopBBAF is found to be an organic compound which can provide a light-emitting device with high emission efficiency, low driving voltage, and extremely high power efficiency.

This application is based on Japanese Patent Application Serial No. 2024-105618 filed with Japan Patent Office on Jun. 28, 2024, the entire contents of which are hereby incorporated by reference.

What is claimed is:

1. An organic compound represented by General Formula (G1): 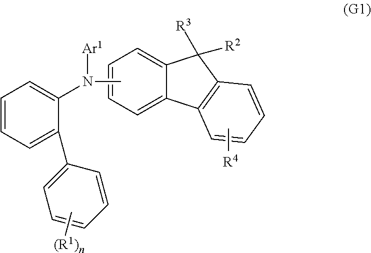
wherein Ar¹ represents a phenyl group or a biphenyl group comprising at least one alkyl group having 1 to 6 carbon atoms,
wherein R¹ represents an alkyl group having 3 to 7 carbon atoms and n represents 2 or 3,
wherein a plurality of R¹s are the same or different from each other,
wherein R² and R³ each independently represent any one of an alkyl group having 1 to 6 carbon atoms, an unsubstituted phenyl group, and a phenyl group comprising an alkyl group having 1 to 6 carbon atoms, or R² and R³ are bonded to each other to form a ring, and
wherein R⁴ represents hydrogen or an alkyl group having 1 to 6 carbon atoms.

2. An organic compound represented by General Formula (G2): 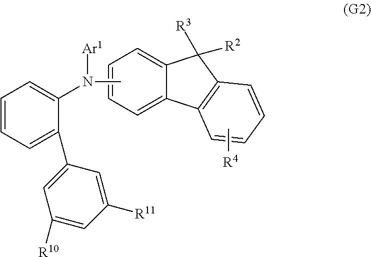
wherein Ar¹ represents a phenyl group or a biphenyl group comprising at least one alkyl group having 1 to 6 carbon atoms,
wherein R¹⁰ and R¹¹ each independently represent an alkyl group having 3 to 7 carbon atoms,
wherein R² and R³ each independently represent any one of an alkyl group having 1 to 6 carbon atoms, an unsubstituted phenyl group, and a phenyl group comprising an alkyl group having 1 to 6 carbon atoms, or R² and R³ are bonded to each other to form a ring, and
wherein R⁴ represents hydrogen or an alkyl group having 1 to 6 carbon atoms.

3. An organic compound represented by General Formula (G3): 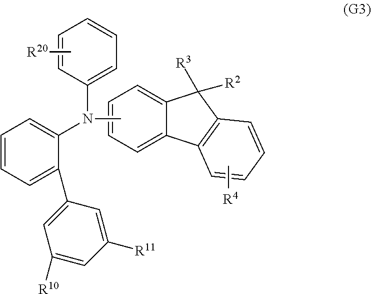
wherein R¹⁰ and R¹¹ each independently represent an alkyl group having 3 to 7 carbon atoms,
wherein R²⁰ represents an alkyl group having 1 to 6 carbon atoms or a phenyl group comprising an alkyl group having 1 to 6 carbon atoms,
wherein R² and R³ each independently represent any one of an alkyl group having 1 to 6 carbon atoms, an unsubstituted phenyl group, and a phenyl group comprising an alkyl group having 1 to 6 carbon atoms, or R² and R³ are bonded to each other to form a ring, and
wherein R⁴ represents hydrogen or an alkyl group having 1 to 6 carbon atoms.

4. The organic compound according to claim 3,
wherein the organic compound is represented by General Formula (G4):

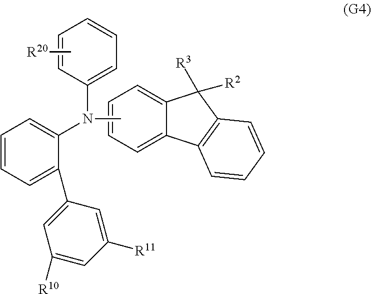

5. A film comprising the organic compound according to claim 1.

6. The organic compound according to claim 1,
wherein a film of the organic compound has a GSP_slope greater than or equal to 30 mV/nm.

7. The organic compound according to claim 1,
wherein a film of the organic compound is evaporated at a rate greater than or equal to 3 nm/min and less than or equal to 600 nm/min,
wherein the film has a thickness greater than or equal to 3 nm and less than or equal to 500 nm, and
wherein a GSP_slope of the film is greater than or equal to 30 mV/nm.

8. The organic compound according to claim 1,
wherein an ordinary refractive index of a film of the organic compound at a wavelength of 450 nm to 460 nm is lower than or equal to 1.75.

9. The organic compound according to claim 1,
wherein an ordinary refractive index of a film of the organic compound at a wavelength of 510 nm to 545 nm is lower than or equal to 1.70.

10. An electronic device comprising the film according to claim 5.

11. A light-emitting device comprising:
a first electrode;
a second electrode; and
an organic compound layer,
wherein the organic compound layer is between the first electrode and the second electrode, and
wherein the organic compound layer comprises the organic compound according to claim 1.

12. A light-emitting device comprising:
a first electrode;
a second electrode; and
an organic compound layer,
wherein the organic compound layer is between the first electrode and the second electrode,
wherein the organic compound layer comprises a light-emitting layer and a hole-transport layer,
wherein the hole-transport layer is between the light-emitting layer and the first electrode, and
wherein the hole-transport layer comprises the organic compound according to claim 1.

13. The light-emitting device according to claim 12,
wherein the hole-transport layer comprises a first hole-transport layer and a second hole-transport layer,
wherein the second hole-transport layer is between the first hole-transport layer and the light-emitting layer, and
wherein the second hole-transport layer comprises the organic compound.

14. The light-emitting device according to claim 13,
wherein the second hole-transport layer and the light-emitting layer are in contact with each other.

15. The light-emitting device according to claim 13,
wherein a GSP_slope of the second hole-transport layer is greater than a GSP_slope of the first hole-transport layer.

16. A film comprising the organic compound according to claim 2.

17. The organic compound according to claim 2,
wherein a film of the organic compound has a GSP_slope greater than or equal to 30 mV/nm.

18. A film comprising the organic compound according to claim 3.

19. The organic compound according to claim 3,
wherein a film of the organic compound has a GSP_slope greater than or equal to 30 mV/nm.
